(19) United States
(12) Patent Application Publication
(10) Pub. No.: US 2004/0132634 A1
(43) Pub. Date: Jul. 8, 2004

(54) Compositions and methods for regulating the kinase domain of receptor tyrosine kinases

(76) Inventors: Frank Sicheri, Toronto (CA); Leanne Wybenga-Groot, Etobicoke (CA); Tony Pawson, Toronto (CA)

Correspondence Address: Ropes & Gray, One International Place, Boston, MA, 02110-2624, US

(21) Appl. No.: 10/470,840

(22) PCT Filed: Jan. 31, 2002
(86) PCT No.: PCT/CA02/00114

(30) Foreign Application Priority Data
Jan. 31, 2001 (US) 60265510

(51) Int. Cl. A61K 31/00; G06F 19/00; G01N 33/48; G01N 33/50; C12N 9/12

(52) U.S. Cl. 514/1; 435/194; 702/19

(57) ABSTRACT

The present invention relates to binding pockets of receptor tyrosine kinases (RTKs). The binding pockets may regulate the kinase domain of the receptor tyrosine kinases. In particular, the invention relates to a crystal comprising a binding pocket of a receptor tyrosine kinase that regulates the kinase domain of the receptor tyrosine kinase EphB2. The crystal may be useful for modeling and/or synthesizing mimetics of a binding pocket or ligands that associate with the binding pocket. Such mimetics or ligands may be capable of acting as modulators of receptor tyrosine kinase receptor activity, and they may be useful for treating, inhibiting, or preventing diseases modulated by such receptors. Methods are also provided for regulating the kinase domain of an RTK by changing a binding pocket of the RTK that regulates the kinase domain from an autoinhibited state to an active state or from an active state to an autoinhibited state.

A portion of the disclosure of this patent document contains material that is subject to copyright protection. The copyright owner has no objection to the facsimile reproduction by anyone of the patent document or patent disclosure, as it appears in the Patent and Trademark Office patent file or records, but otherwise reserves all copyright rights whatsoever.

### FIELD OF THE INVENTION

The present invention relates to binding pockets of receptor tyrosine kinases (RTKs). The binding pockets may regulate the kinase domain of the receptor tyrosine kinases. In particular, the invention relates to a crystal comprising a binding pocket of a receptor tyrosine kinase that regulates the kinase domain of the receptor tyrosine kinase. The crystal may be useful for modeling and/or synthesizing mimetics of a binding pocket or ligands that associate with the binding pocket. Such mimetics or ligands may be capable of acting as modulators of receptor tyrosine kinase receptor activity, and they may be useful for treating, inhibiting, or preventing diseases modulated by such receptors.

Methods are also provided for regulating the kinase domain of an RTK by changing a binding pocket of the RTK that regulates the kinase domain from an autoinhibited state to an active state or from an active state to an autoinhibited state.

### BACKGROUND

Cell surface receptors with protein-tyrosine kinase activity mediate the biological effects of many extracellular signaling proteins, and thereby regulate aspects of normal cellular behavior such as growth and differentiation, movement, metabolism and survival (van der Geer and Hunter, 1994). The profound consequences of phosphotyrosine signaling on cellular function are emphasized by the effects of mutations that deregulate receptor tyrosine kinase activity, which are frequently associated with malignant transformation or developmental abnormalities. Under normal circumstances, the activation of receptor tyrosine kinases (RTK) requires binding of the appropriate extracellular ligand, which induces either receptor oligomerization or a spatial re-organization of pre-associated receptor chains (Heldin, 1995; Remy et al., 1999; Schlessinger, 2000). As a result, the receptor undergoes autophosphorylation through an intermolecular reaction, both on tyrosine residues which regulate kinase activity, and on residues within non-catalytic regions of the receptor which form binding sites for cytoplasmic targets with SH2 or PTB domains (Pawson and Scott, 1997; Kuriyan and Cowburn, 1997).

The catalytic activity of tyrosine kinases is frequently stimulated by autophosphorylation within a region of the kinase domain termed the activation segment (Weinnmaster et al., 1984), and indeed this has been viewed as the principal mechanism through which RTKs are activated (Hubbard and Till, 2000; Hubbard, 1997). Structural analysis of the isolated kinase domains of several receptors has revealed how the activation segment represses kinase activity, and the means by which phosphorylation releases this autoinhibition. In the case of the inactive insulin receptor, Tyr 1162 in the activation segment protrudes into the active site, and the activation segment blocks access to the ATP-binding site (Hubbard et al., 1994). Autophosphorylation of Tyr 1162 and two adjacent tyrosine residues repositions the activation segment, thereby freeing the active site to engage exogenous substrates and reorganizing the residues required for catalysis into a functional conformation (Hubbard, 1997). In contrast, the activation segment of the fibroblast growth factor (FGF) receptor is relatively mobile and the tyrosines which become phosphorylated upon receptor activation do not occupy the active site. However, the C-terminal end of the FGFR1 activation segment appears to block access to substrate (Mohammadi et al., 1996).

Despite the evident importance of the kinase domain activation segment, it remains possible that other mechanisms are important in regulating RTK activity, which might have been missed through an exclusive focus on the kinase domain itself. In particular, recent biochemical and mutational analysis has suggested that Eph receptors may be regulated through a more complex mechanism, involving the juxtamembrane region (Binns et al., 2000; Zisch et al., 1998; Zisch et al., 2000).

There is only a single Eph receptor tyrosine kinase encoded by the C. elegans genome (VAB-1) (George et al., 1998; Wang et al., 1999a), but the subfamily has undergone a remarkable expansion during metazoan evolution to include at least 14 mammalian members, which therefore represent the largest class of vertebrate RTKs (Holder and Klein, 1999). These Eph receptors fall into two groups, A and B, based on their ability to bind ligands (ephrins), which are themselves cell surface proteins anchored to the plasma membrane either through a GPI linkage (A-type ephrins) or a transmembrane region (B-type) (Eph Nomenclature Committee, 1997; Gale et al., 1996). Signaling between Eph receptors and ephrins generally involves direct cell-cell interactions (Holland et al., 1996; Bruckner et al., 1997), and frequently results in the repulsion of these cells one from another (Drescher et al., 1995; Wang and Anderson, 1997; Mellitzer et al., 1999). Eph receptors are implicated in morphogenetic cell movements (Wang et al., 1999a; Chin-Sang et al., 1999), in defining cell boundaries in structures such as the rhombomeres of the embryonic hindbrain (Xu et al., 1999), in controlling axon guidance and the establishment of topographic maps in the central nervous system (Nakamoto et al., 1996; Brown et al., 2000), and in determining the trajectories of migrating neural crest cells (Krull et al., 1997). Signaling between ephrin and Eph receptor-expressing cells is also essential for angiogenesis, and in conferring distinct arterial and venous identities to developing blood vessels (Wang et al., 1999b; Adams et al., 1999; Gerety et al., 1999).

The extracellular region of Eph receptors contains an N-terminal ephrin-binding domain (Labrador et al., 1997), that folds into a jellyroll β-sandwich (Himanen et al., 1998), followed by a cysteine-rich region and two fibronectin type III repeats (Pasquale, 1991; Henkemeyer et al., 1994). A single membrane-spanning sequence is followed by a relatively lengthy juxtamembrane region, an uninterrupted kinase domain, an o-helical sterile alpha motif (SAM) domain implicated in receptor oligomerization (Stapleton et al., 1999; Thanos et al., 1999), and a C-terminal motif capable of binding PDZ domain proteins (Hock et al., 1998; Torres et al., 1998). Activation of receptors such as EphB2 or EphA4 is accompanied by autophosphorylation on multiple residues, most notably on two tyrosines within a highly conserved juxtamembrane motif (YIDPFTYEDP in EphB2) and on a tyrosine within the activation segment of the kinase domain (Holland et al., 1997; Choi and Park, 1999; Ellis et al., 1996; Kalo and Pasquale, 1999; Zisch et al., 1998; Binns et al., 2000). By analogy with other RTKs, it might be expected that autophosphorylation of the activation segment tyrosine would stimulate kinase activity, while the juxtamembrane phosphotyrosine sites would recruit cytoplasmic targets. Indeed, the juxtamembrane phosphotyrosine motifs do bind SH2 domain signaling proteins, including p120-RasGAP, Nck, phosphatidylinositol 3′-kinase, SHEP-1 and Src family kinases among others, which can potentially direct cellular responses to ephrin stimulation (Dodelet et al., 1999; Ellis et al., 1996; Holland et al., 1997; Holland et al., 1998; Zisch et al., 1998).

Consistent with the possibility that phosphorylation of the conserved juxtamembrane tyrosines is important for signaling, substitution of these residues in EphB2 with phenylalanine abrogates EphB2-mediated growth cone collapse upon stimulation of NG108 neuronal cells with ephrin B1. However, this loss of biological activity is apparently not due solely to a failure to engage SH2-containing targets, since substitution of the juxtamembrane tyrosines in EphB2 and EphA4 with phenylalanine leads to a severe loss of ephrin-induced kinase activity (Binns et al., 2000).

### SUMMARY OF THE INVENTION

Applicants have solved the x-ray crystal structure of an Eph receptor tyrosine kinase domain and juxtamembrane region in an autoinhibited state. The results show that in its unphosphorylated state, the juxtamembrane region adopts a helical structure that distorts the conformation of the small lobe of the kinase domain, thereby disrupting the active site. These results indicate a novel mechanism for the regulation of RTKs.

Solving the crystal structure has enabled the determination of key structural features of the kinase domain and juxtamembrane region, particularly the shape of binding pockets, or parts thereof, that permit the juxtamembrane region and kinase domain to associate resulting in an autoinhibited state. The crystal structure has also enabled the determination of key structural features in molecules or ligands that interact or associate (e.g. nucleotides, cofactors, inhibitors, and substrates) with the binding pockets.

Knowledge of the autoinhibited conformation of binding pockets of RTKs that regulate the kinase domain is of significant utility in drug discovery. The association of natural ligands and substrates with the binding pockets of RTKs is the basis of many biological mechanisms. In addition, many drugs exert their effects through association with the binding pockets of RTKs. The associations may occur with all or any parts of a binding pocket. An understanding of the association of a drug with the active and autoinhibited conformations of binding pockets of RTKs, will lead to the design and optimization of drugs having more favorable associations with their target RTKs and thus provide improved biological effects. Therefore, information about the shape and structure of binding pockets of RTKs in their autoinhibited and activated states, is invaluable in designing potential modulators of the receptors for use in treating diseases and conditions associated with or modulated by the receptors.

The present invention relates to a binding pocket of a receptor tyrosine kinase (RTK). In an aspect of the invention, the binding pocket regulates the kinase domain of the receptor tyrosine kinase or is involved in maintaining an autoinhibited state or active state of an RTK.

The invention also relates to a crystal comprising a binding pocket of an RTK that regulates the kinase domain of the RTK. The binding pocket may be in an autoinhibited state, or active state. Thus, a binding pocket may be involved in maintaining an autoinhibited state or active state of an RTK.

In an embodiment, the invention, provides a crystal comprising a juxtamembrane region and/or kinase domain of an RTK, or part thereof. The invention contemplates a crystal formed by a juxtamembrane region and a kinase domain of an RTK in an autoinhibited state or active state.

The invention also contemplates a crystal comprising a binding pocket of a receptor tyrosine kinase that regulates the kinase domain of the receptor tyrosine kinase in association with a ligand.

The present invention also contemplates molecules or molecular complexes that comprise all or parts of either one or more binding pockets of the invention, or homologs of these binding pockets that have similar structure and shape.

The present invention also provides a crystal comprising a binding pocket of an RTK of the invention and at least one ligand. A ligand may be complexed or associated with a binding pocket. Ligands include a nucleotide or analogue or part thereof, a substrate or analogue thereof, a cofactor, and/or heavy metal atom. A ligand may be a modulator of the activity of an RTK.

In an aspect the invention contemplates a crystal comprising a binding pocket of an RTK of the invention complexed with a nucleotide or analogue thereof from which it is possible to derive structural data for the nucleotide or analogue thereof.

The shape and structure of a binding pocket may be defined by selected atomic contacts in the pocket. In an embodiment, the binding pocket is defined by one or more atomic interactions or enzyme atomic contacts as set forth in Table 2. Each of the atomic interactions is defined in Table 2 by an atomic contact (more preferably, a specific atom where indicated) on the juxtamembrane region and by an atomic contact (more preferably a specific atom where indicated) on the kinase domain, juxtamembrane region, or ligand.

An isolated polypeptide comprising a binding pocket with the shape and structure of a binding pocket described herein is also within the scope of the invention.

The invention also provides a method for preparing a crystal of the invention, preferably a crystal of a binding pocket of an Eph receptor, or a complex of such a binding pocket and a ligand.

Crystal structures of the invention enable a model to be produced for a binding pocket of the invention, or complexes or parts thereof. The models will provide structural information about the autoinhibited or active state of a binding pocket of a RTK or a ligand and its interactions with a binding pocket. Models may also be produced for ligands. A model and/or the crystal structure of the present invention may be stored on a computer-readable medium.

The present invention includes a model of a binding pocket of the present invention that substantially represents the structural coordinates specified in Table 3. The invention also includes a model that comprises modifications of the model substantially represented by the structural coordinates specified in Table 3. A modification may represent a binding pocket that is involved in maintaining an autoinhibited state or active state of an RTK or regulates the kinase domain of an RTK. A model is a representation or image that predicts the actual structure of the binding pocket. As such, a model is a tool that can be used to probe the relationship between a binding pocket's structure and function at the atomic level and to design molecules that can modulate the binding site and accordingly RTK activity.

Thus, the invention provides a model of: (a) a binding pocket of an RTK that is involved in maintaining an autoinhibited state or active state of an RTK or regulates the kinase domain of an RTK; and (b) a modification of the model of (a).

A method is also provided for producing a model of the invention representing a binding pocket of an RTK that is involved in maintaining an autoinhibited state or active state of an RTK or regulates the kinase domain of an RTK, comprising representing amino acids of the binding pocket at substantially the structural coordinates specified in Table 3.

A crystal and/or model of the invention may be used in a method of determining the secondary and/or tertiary structures of a polypeptide or binding pocket with incompletely characterised structure. Thus, a method is provided for determining at least a portion of the secondary and/or tertiary structure of molecules or molecular complexes which contain at least some structurally similar features to a binding pocket of the invention. This is achieved by using at least some of the structural coordinates set out in Table 3.

A crystal of the invention may be useful for designing, modeling, identifying, evaluating, and/or synthesizing mimetics of a binding pocket or ligands that associate with a binding pocket. Such mimetics or ligands may be capable of acting as modulators of receptor tyrosine kinase activity, and they may be useful for treating, inhibiting, or preventing diseases modulated by such receptors.

Thus, the present invention contemplates a method of identifying a modulator of an RTK comprising the step of applying the structural coordinates of a binding pocket, or atomic interactions, or atomic contacts of a binding pocket, to computationally evaluate a test ligand for its ability to associate with the binding pocket, or part thereof. Use of the structural coordinates of a binding pocket, or atomic interactions, or atomic contacts of a binding pocket to design or identify a modulator is also provided.

In an embodiment, the invention contemplates a method of identifying a modulator of an RTK comprising determining if a test agent inhibits or potentiates an autoinhibited state or active state of a kinase domain of the RTK.

The invention further contemplates classes of modulators of RTKs based on the shape and structure of a ligand defined in relation to the molecule's spatial association with a binding pocket of the invention. Generally, a method is provided for designing potential inhibitors of RTKs comprising the step of applying the structural coordinates of a ligand defined in relation to its spatial association with a binding pocket, or a part thereof, to generate a compound that is capable of associating with the binding pocket.

It will be appreciated that a modulator of an RTK may be identified by generating an actual secondary or three-dimensional model of a binding pocket, synthesizing a compound, and examining the components to find whether the required interaction occurs.

A potential modulator of an RTK identified by a method of the present invention may be confirmed as a modulator by synthesizing the compound, and testing its effect on the RTK in an assay for that receptor's enzymatic activity. Such assays are known in the art (e.g. phosphorylation assays).

A modulator of the invention may be converted using customary methods into pharmaceutical compositions. A modulator may be formulated into a pharmaceutical composition containing a modulator either alone or together with other active substances.

Therefore, the methods of the invention for identifying modulators may comprise one or more of the following additional steps:

(a) testing whether the modulator is a modulator of the activity of a RTK, preferably testing the activity of the modulator in cellular assays and animal model assays;

(b) modifying the modulator;

(c) optionally rerunning steps (a) or (b); and (d) preparing a pharmaceutical composition comprising the modulator.

Steps (a), (b) (c) and (d) may be carried out in any order, at different points in time, and they need not be sequential.

Still another aspect of the present invention provides a method of conducting a drug discovery business comprising:

(a) providing one or more systems employing the atomic interactions, atomic contacts, or structural coordinates of a binding pocket of an RTK, for identifying agents by their ability to inhibit or potentiate the atomic interactions or atomic contacts of a binding pocket; and

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

A further aspect of the present invention provides a method of conducting a drug discovery business comprising:

(a) providing one or more systems for identifying agents by their ability to inhibit or potentiate an autoinhibited state or active state of a kinase domain of an RTK; and

(b) conducting therapeutic profiling of agents identified in step (a), or further analogs thereof, for efficacy and toxicity in animals; and

(c) formulating a pharmaceutical preparation including one or more agents identified in step (b) as having an acceptable therapeutic profile.

In certain embodiments, the subject methods can also include a step of establishing a distribution system for distributing the pharmaceutical preparation for sale, and may optionally include establishing a sales group for marketing the pharmaceutical preparation.

Yet another aspect of the invention provides a method of conducting a target discovery business comprising:

(a) providing one or more systems employing the atomic interactions, atomic contacts, or structural coordinates of a binding pocket of an RTK, for identifying agents by their ability to inhibit or potentiate the atomic interactions or atomic contacts, or providing one or more systems for identifying agents by their ability to inhibit or potentiate an autoinhibited state or active state of a kinase domain of an RTK;

(b) (optionally) conducting therapeutic profiling of agents identified in step (a) for efficacy and toxicity in animals; and

(c) licensing, to a third party, the rights for further drug development and/or sales for agents identified in step (a), or analogs thereof.

Methods are also provided for regulating the kinase domain of an RTK by changing a binding domain or pocket of a RTK that regulates the kinase domain from an autoinhibited state to an active state or from an active state to an autoinhibited state. A binding domain or pocket of a RTK may be changed from an autoinhibited state by altering amino acid residues forming the binding pocket (e.g. introducing mutations) or using a modulator.

In an aspect the invention provides a method for inhibiting kinase activity of an RTK comprising maintaining the RTK or a binding pocket thereof involved in regulating the kinase domain in an autoinhibited state, or potentiating an autoinhibited state for the RTK or binding pocket thereof involved in regulating the kinase domain. An autoinhibited state may be maintained or potentiated by inhibiting phosphorylation of phosphoregulatory sites of the juxtamembrane segment and/or kinase domain (e.g. activation segment). Inhibition may be accomplished using modulators, or altering the structure of a binding pocket of the RTK comprising the phosphoregulatory sites, to prevent phosphorylation of the sites.

The invention contemplates a method for altering the stability of an autoinhibited state of an RTK comprising phosphorylating phosphoregulatory sites of a juxtamembrane region of the RTK.

In an aspect the invention relates to a method for changing an RTK from an autoinhibited state to an active state comprising phosphorylating phosphoregulatory sites of a juxtamembrane region of the RTK.

In another aspect the invention provides a method for activating kinase activity of an RTK comprising phosphorylating phosphoregulatory sites of a juxtamembrane region and kinase domain (e.g. activation segment) of the RTK involved in maintaining the RTK in an autoinhibited state.

The invention also contemplates a method of treating or preventing a condition or disease associated with an RTK in a cellular organism, comprising:

(a) administering a modulator of the invention in an acceptable pharmaceutical preparation; and

(b) activating or inhibiting the RTK to treat or prevent the disease.

In an aspect the invention provides a method for treating or preventing a condition or disease involving increased RTK activity comprising maintaining the RTK or a binding pocket thereof involved in regulating the kinase domain of the RTK in an autoinhibited state. An autoinhibited state may be maintained as described herein. In an embodiment the condition or disease is cancer.

The invention provides for the use of a modulator identified by the methods of the invention in the preparation of a medicament to treat or prevent a disease in a cellular organism. Use of modulators of the invention to manufacture a medicament is also provided.

These and other aspects of the present invention will become evident upon reference to the following detailed description and Tables, and attached drawings.

### DESCRIPTION OF THE DRAWINGS AND TABLES

The present invention will now be described only by way of example, in which reference will be made to the following Figures:

FIG. 1. Structure-based sequence alignment of the juxtamembrane segments and kinase domains of murine and human EphB2, murine EphA4 and cAPK, and human IRK, FGFR1, Hck, Kit, PDGFRβ, and Flt3. The secondary structure elements of murine EphB2 are indicated, with the juxtamembrane segment, the N-terminal kinase, the g-loop, and the C-terminal lobe coloured red, green, orange, and blue, respectively. Residues Phe 620 and Tyr 750 and those marked with a star are involved in the juxtamembrane/kinase domain interface. The two juxtamembrane tyrosines (604 and 610) that were mutated to phenylalanine are highlighted in light blue. Additional tyrosines identified by Kalo and Pasquale (1999) as in vivo phosphorylation sites are highlighted in purple. The solid triangle indicates the site of a 16 amino acid insertion in chicken EphB2 resulting from alternate RNA processing (Connor and Pasquale, 1995). For Kit and PDGFRβ, tyrosines highlighted in yellow denote autophosphorylation sites, while sites of activating point mutations and deletions are shaded gray (Tsujimura et al., 1996; Irusta and DiMaio, 1998; Kitayama et al., 1995; Hirota et al., 1998). The locations and regions of duplicated sequence for activating Flt3 mutations are indicated by solid black triangles and underlining (Hayakawa et al, 2000).

FIG. 2. Overview of the autoinhibited EphB2 structure. (a) Ribbon diagram of the EphB2 crystal structure in complex with AMP-PNP. The juxtamembrane region, N-terminal kinase lobe, C-terminal kinase lobe, and g-loop are coloured red, green, blue and orange, respectively. Phosphoregulatory residues Tyr/Phe 604 and Tyr/Phe 610 are coloured light blue, Tyr667 is coloured purple, and the adenine moiety of AMP-PNP is coloured red. (b) Ribbon representation of EphB2 colored as in (a), rotated 90° about the vertical axis. (c) and (d) The juxtamembrane regions in (a) and (b), respectively, have been magnified to detail the interactions between the juxtamembrane region and helix αC of the N-terminal kinase lobe. Carbon, oxygen, nitrogen and sulfur atoms are shown in yellow, red, blue, and green, respectively. Residues involved in the juxtamembrane/kinase domain interface but not shown include Ala616, Ala621, Leu676, Leu693, and Val696. All ribbon diagrams were prepared with RIBBONS (Carson, 1991b).

FIG. 3. Comparison of autoinhibited EphB2 RTK with the active insulin receptor kinase. (a) Superposition of EphB2 with active insulin receptor kinase (Protein Data Bank ID code 1ir3). The backbone of the juxtamembrane region of EphB2 is shown in red, with the side chains of Tyr/Phe 604 and Tyr/Phe 610 coloured light blue. The EphB2 kinase domain, g-loop and bound adenine moiety are colored blue, orange and red, respectively. The backbone of active IRK is coloured dark green with its activation segment, g loop, and bound nucleotide shown in purple, pink, and light green respectively. The two receptors were aligned using all elements of the C-terminal lobes except the kinase insert region, the activation segment, helix αJ, and the C-terminal tail (rms fit=1.91 Å). (b) Stereo view of the boxed region in (a), with EphB2 phosphorylation sites shown in purple, other EphB2 side chain atoms coloured as in FIGS. 2c and 2d and IRK side chains shown in green and pink. (c) Stereo view of the boxed region in A), highlighting the kinase catalytic region. This panel is colored as in (b). (d) Stereo view of boxed region in A) highlighting switch region 1. Inactive IRK (Protein Data Band ID code 1irk), shown in yellow, is also superimposed. All side chains are colored according to their respective backbones. IRK residue labeled Thr776 corresponds to Ser776 in EphB2.

FIG. 4. Electrostatic surface representation of EphB2. Blue and red regions indicate positive and negative potential, respectively (10 to −10 k_{B}T). Phosphoregulatory residues Tyr/Phe 604 and Tyr/Phe 610 are coloured light blue. The molecular surface of EphB2 is oriented as in FIG. 2a and was generated using GRASP (Nicholls et al., 1991)

FIG. 5. Comparison of the kinase activities of EphA4 and EphB2 wild-type and mutant proteins. (a) GST-EphA4 proteins were expressed in E. coli, and cell lysates were subjected to immunoblot analysis with anti-pTyr antibody (top panel) and anti-GST antibody (lower panel). (b) Equal quantities of GST-EphA4 proteins bound to glutathione sepharose were assessed for their ability to autophosphorylate and phosphorylate enolase by an in vitro kinase assay (top panel). Immunblot analysis of GST-EphA4 proteins with anti-GST antibody (lower panel). (c) Histogram of the specific activities of EphA4 wild-type and mutant proteins as measured by the spectrophotometric coupling assay at 1 mM S-1 peptide and 0.5 μM EphA4 proteins. The velocities represent the mean of triplicate reactions and have been normalized to the specific activity of wild-type EphA4 (top panel). Coomassie stained SDS-PAGE analysis of EphA4 proteins (lower panel). (d) EphB2 and its mutants were expressed in COS-1 cells and immunoprecipitated. The immunoprecipitates were resolved by SDS-PAGE, immunoblotted with anti-pTyr (top panel) or anti-EphB2 (middle panel) antibodies, and assessed for their ability to autophosphorylate and phosphorylate enolase by an in vitro kinase assay (bottom panel).

FIG. 6. Schematic diagram highlighting differences between the autoinhibited (left) and active (right) states of the Eph receptor family of tyrosine kinases. The active configuration is based on the crystal structure of active IRK (Protein Data Bank ID code 1ir3). Dashed lines indicate regions of activation segment disorder. The numbering scheme corresponds to murine EphB2.

The present invention will now be described only by way of example, in which reference will be made to the following Tables:

Table 1 shows the data collection, structure determination and refinement statistics

Table 2 shows intermolecular contacts in a binding pocket of the invention.

Table 3 shows the structural coordinates of the juxtamembrane region and kinase domain of an EphB2 receptor.

In Table 3, from the left, the second column identifies the atom number; the third identifies the atom type; the fourth identifies the amino acid type; the sixth identifies the residue number; the seventh identifies the x coordinates; the eighth identifies the y coordinates; the ninth identifies the z coordinates; the tenth identifies the occupancy; and the eleventh identifies the temperature factor.

### DETAILED DESCRIPTION OF THE INVENTION

Unless otherwise indicated, all terms used herein have the same meaning as they would to one skilled in the art of the present invention. Practitioners are particularly directed to Current Protocols in Molecular Biology (Ansubel) for definitions and terms of the art.

In accordance with the present invention there may be employed conventional biochemistry, enzymology, molecular biology, crystallography, bioinformatics, microbiology, and recombinant DNA techniques within the skill of the art. Such techniques are explained fully in the literature. See for example, Sambrook, Fritsch, & Maniatis, Molecular Cloning: A Laboratory Manual, Second Edition (1989) Cold Spring Harbor Laboratory Press, Cold Spring Harbor, N.Y); DNA Cloning: A Practical Approach, Volumes I and II (D. N. Glover ed. 1985); Oligonucleotide Synthesis (M. J. Gait ed. 1984); Nucleic Acid Hybridization B. D. Hames & S. J. Higgins eds. (1985); Transcription and Translation B. D. Hames & S. J. Higgins eds (1984); Animal Cell Culture R. I. Freshney, ed. (1986); Immobilized Cells and enzymes IRL Press, (1986); and B. Perbal, A Practical Guide to Molecular Cloning (1984).

For ease of reference the murine numbering scheme for EphB2 is employed herein to describe specific amino acid residues in aspects of the invention. However, a person skilled in the art could readily determine the corresponding amino acid residues in other RTKs, more particularly in Eph receptors.

Receptor Tyrosine Kinases (RTKs)

The invention generally relates to RTKs. RTKs mediate pathways involving multiple extracellular and intracellular signals, integration and amplification of these signals by second messengers, and the activation of cellular processes including cell proliferation, cell division, cell growth, the cell cycle, cell differentiation, cell migration, axonogenesis, nerve cell interactions, and regeneration. Signaling pathways mediated by receptor tyrosine kinases may be initiated by growth factors binding to specific RTKs on cell surfaces. The binding of a growth factor to its receptor activates RTK signaling pathways. The RTKs have an extracellular N-terminal domain that binds the growth factor and a cytoplasmic C-terminal domain containing a protein tyrosine kinase that is capable of autophosphorylation, and the phosphorylation of other protein substrates. Autophosphorylation takes place within a region of the kinase domain of the RTK termed the “activation segment” (Weinnmaster et al., 1984). The binding of a growth factor to its receptor activates the tyrosine kinase which phosphorylates a variety of signaling molecules thereby initiating signaling pathways that can lead to DNA replication, RNA and protein synthesis, and cell division.

Receptor tyrosine kinases within the scope of the present invention include but are not limited to epidermal growth factor receptor (EGFR), PDGF receptor, insulin receptor tyrosme kinase (IRK), Met receptor tyrosine kinase, fibroblast growth factor (FGF) receptor, insulin receptor, insulin growth factor (IGF-1) receptor, TrkA receptor, IL-3 receptor, B cell receptor, TIE-1, Tek/Tie2, Flt-1, Flk, VEGFR3, EFGR/Erbb, Erb2/neu, Erb3, Ret, Kit, Alk, Ax1, FGFR1, FGFR2, FGFR3, Hck, cAPK, keratinocyte growth factor (KGF) receptor, and Eph receptors.

The invention preferably contemplates Eph receptors, more preferably EphB2 receptors.

The term “Eph receptor” refers to a subfamily of closely related transmembrane receptor tyrosine kinases related to Eph, a receptor named for its expression in an erythropoietin-producing human hepatocellular carcinomas cell line. The receptors contain cell adhesion-like domains on their extracellular surface. The N-terminal extracellular region of all Eph family members contains a domain necessary for ligand binding and specificity, followed by a cysteine-rich domain and two fibronectin type II repeats. The cytoplasmic region has a centrally located tyrosine kinase domain. C-terminal to the catalytic region is a sterile alpha motif (SAM) domain, which forms dimers of oligomers in solution and may contribute to regulation of receptor clustering. Localization of clustering of Eph proteins may also be influenced by PDZ domain effectors which potentially interact with specific C-terminal receptor motifs.

N-terminal to the kinase domain is the juxtamembrane domain. Two invariant tyrosine residues (tyrosines 596 and 602 of EphA4; tyrosines 604 and 610 of EphB2) in the juxtamembrane domain are embedded in a characteristic and highly conserved ˜10 amino acid sequence motif. These tyrosine residues are major sites for autophosphorylation and they have been found to associate with a number of SH2 domain-containing cytoplasmic proteins such as Ras GTPase-activating protein (RasGAP), the p85 subunit of phosphatidylinositol 3′ kinase, Src family kinases, the adapter protein Nck, and SHEP-1 which binds the R-Ras and Rap1A GTPases. Signaling mediated by such SH2 domain-containing proteins may contribute to the physiological effects of Eph receptor stimulation on cell adhesion and cytoskeletal structures.

There are currently 14 related vertebrate members of the Eph receptor family including receptors in Caenorhabditis elegans and Drosophila. Eph receptors are activated by ephrins. Ephrins are attached to the plasma membrane either via a glycosylphosphatidylinositol linkage (A class) or a transmembrane sequence (B class). Eph receptors are also divided into A and B classes corresponding to their ligand binding specificities and phylogenetic relationships. Class A receptors generally bind A class ephrins, whereas B class ephrins stimulate B class receptors. However, EphA4 is an exception in that it binds and responds to B as well as A class ephrins.

The group that includes receptors interacting preferentially with ephrin A proteins is called EphA and includes EphA1 (also known as Eph and Esk), EphA2 (also known as Eck, Myk2, Sek2), EphA3 (also known as Cek4, Mek4, Hek, Tyro4, Hek4), EphA4 (also known as Sek, Sek1, Cek8, Hek8, Tyro1), EphA5 (also known as Ehk1, Bsk, Cek7, Hek7, and Rek7), EphA6 (Ehk2, and Hek12) EphA7 (also known as Mdk1, Hek11, Ehk3, Ebk, Cek11), and EphA8 (also known as Eek, Hek3). The group that includes receptors interacting preferentially with ephrin B proteins is called Eph B and includes EphB1 (also known as Elk Cek6, Net, Hek6), EphB2 (also known as Cek5, Nuk, Erk, Qek5, Tyro5, Sek3, hek5, Drt), EphB3 (also known as Cek10, Hek2, Mdks, Tyro6, and Sek4), EphB4 (also known as Htk, Myk1, Tyrol 1, Mdk2), EphB5 (also known as Cek9, Hek9), and EphB6 (also known as Mep).

“Ephrin” refers to a class of ligands which are anchored to the cell membrane through a transmembrane domain, and bind to the extracellular domain of an Eph receptor, facilitating dimerization and autophosphorylation of the receptor and autophosphorylation of the ligand. The ephrin-A ligands (GPI-anchored ligands) are ephrin-A (also known as B61, LERK1, EFL-1), ephrin-A2 (also known as LERK6, Elf1, mCek7-L, cElf1), ephrin-A3 (also known as LERK3, Ehk1-L, and EFL-2), ephrin-A4 (also known as LERK4, EFL-4, mLERK4), ephrin-A5 (AL1, LERK7, EFL-5, mAL1, [rLERK7], RAGS). The ephrin-B ligands (transmembrane ligands) are ephrin-B1 (also known as LEKR2, ELK-L, EFL-3, Cek5-L, Stra1, [LERK2]), ephrin-B2 (also known as LERK5, HTK-L, NLERK1, Elf2, Htk-L), and ephrin-B3 (also known as LERK8, ELK-L3, NLERK2, EFL-6, Elf3, [rELK-L3]).

RTKs may be derivable from a variety of sources, including viruses, bacteria, fungi, plants and animals. In a preferred embodiment an RTK is derivable from a mammal, for example, a human.

An RTK in the present invention may be a wild type enzyme, or part thereof, or a mutant, variant or homolog of such an enzyme.

The term “wild type” refers to a polypeptide having a primary amino acid sequence which is identical with the native enzyme (for example, the human enzyme).

The term “mutant” refers to a polypeptide having a primary amino acid sequence which differs from the wild type sequence by one or more amino acid additions, substitutions or deletions. Preferably, the mutant has at least 90% sequence identity with the wild type sequence. Preferably, the mutant has 20 mutations or less over the whole wild-type sequence. More preferably the mutant has 10 mutations or less, most preferably 5 mutations or less over the whole wild-type sequence.

The term “variant” refers to a naturally occurring polypeptide which differs from a wild-type sequence. A variant may be found within the same species (i.e. if there is more than one isoform of the enzyme) or may be found within a different species. Preferably the variant has at least 90% sequence identity with the wild type sequence. Preferably, the variant has 20 mutations or less over the whole wild-type sequence. More preferably, the variant has 10 mutations or less, most preferably 5 mutations or less over the whole wild-type sequence.

The term “part” indicates that the polypeptide comprises a fraction of the wild-type amino acid sequence. It may comprise one or more large contiguous sections of sequence or a plurality of small sections. The “part” may comprise a binding pocket as described herein. The polypeptide may also comprise other elements of sequence, for example, it may be a fusion protein with another protein (such as one which aids isolation or crystallisation of the polypeptide). Preferably the polypeptide comprises at least 50%, more preferably at least 65%, most preferably at least 80% of the wild-type sequence.

The term “homolog” means a polypeptide having a degree of homology with the wild-type amino acid sequence. The term “homology” refers to a degree of complementarity. There may be partial homology or complete homology. In an embodiment of the invention a RTK is substantially homologous to a wild type enzyme. A sequence that is “substantially homologous” refers to a partially complementary sequence that at least partially inhibits an identical sequence from hybridizing to a target nucleic acid. Inhibition of hybridization of a completely complementary sequence to the target sequence may be examined using a hybridization assay (e.g. Southern or northern blot, solution hybridization, etc.) under conditions of reduced stringency. A sequence that is substantially homologous or a hybridization probe will compete for and inhibit the binding of a completely homologous sequence to the target sequence under conditions of reduced stringency. However, conditions of reduced stringency can be such that non-specific binding is permitted, as reduced stringency conditions require that the binding of two sequences to one another be a specific (i.e., a selective) interaction. The absence of non-specific binding may be tested using a second target sequence which lacks even a partial degree of complementarity (e.g., less than about 30% homology or identity). The substantially homologous sequence or probe will not hybridize to the second non-complementary target sequence in the absence of non-specific binding.

A sequence of an RTK may have at least 60%, 65%, 70%, 75%, 80%, 85%, 90%, 95%, or 99% identity. The phrase “percent identity” or “% identity” refers to the percentage of sequence similarity found in a comparison of two or more amino acid sequences. Percent identity can be determined electronically using conventional programs, e.g., by using the MEGALIGN program (LASERGENE software package, DNASTAR). The MEGALIGN program can create alignments between two or more amino acid sequences according to different methods, e.g., the Clustal Method. (Higgins, D. G. and P. M. Sharp (1988) Gene 73:237-244.) Gaps of low or of no homology between the two amino acid sequences are not included in determining percentage similarity.

In the present context, a homologous sequence is taken to include an amino acid sequence which may have at least 75, 85 or 90% identity, preferably at least 95 or 98% identity to the wild-type sequence. The homologs will comprise the same sites (for example, binding pocket) as the subject amino acid sequence.

A sequence may have deletions, insertions or substitutions of amino acid residues which produce a silent change and result in a functionally equivalent enzyme. Deliberate amino acid substitutions may be made on the basis of similarity in polarity, charge, solubility, hydrophobicity, hydrophilicity, and/or the amphipathic nature of the residues as long as the secondary binding activity of the substance is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

The polypeptide may also have a homologous substitution (substitution and replacement are both used herein to mean the interchange of an existing amino acid residue, with an alternative residue) i.e. like-for-like substitution such as basic for basic, acidic for acidic, polar for polar etc. Non-homologous substitution may also occur i.e. from one class of residue to another or alternatively involving the inclusion of unnatural amino acids such as ornithine (hereinafter referred to as Z), diaminobutyric acid ornithine (hereinafter referred to as B), norleucine ornithine (hereinafter referred to as 0), pyriylalanine, thienylalanine, naphthylalanine and phenylglycine.

Binding Pocket “Binding pocket” refers to a region or site of a RTK or molecular complex thereof that as a result of its shape, associates with another region of the RTK or with a ligand or a part thereof. A binding pocket may regulate the kinase domain of the RTK. A binding pocket may be involved in maintaining an autoinhibited state or active state of an RTK For example, a binding pocket may comprise part of a juxtamembrane region of an RTK that associates with a kinase domain of the RTK (e.g. strand segment Ex1), a site formed by interacting amino acid residues in the juxtamembrane region (e.g. switch region 2), a site formed by interacting amino acid residues in the juxtamembrane region and kinase domain (switch region 1), or a region responsible for binding a ligand.

The invention contemplates a binding pocket of an RTK in an autoinhibited state or an active state.

A “ligand” refers to a compound or entity that associates with a binding pocket including nucleotides or analogues or parts thereof, substrates or analogues or parts thereof, or modulators of RTKs, including inhibitors. A ligand may be designed rationally by using a model according to the present invention.

In an aspect of the invention a binding pocket comprises one or more of the residues involved in coordination of a nucleotide or analog thereof, in particular the amino acid residues involved in coordinating the sugar and phosphate groups of the nucleotide.

In an aspect of the invention the binding pocket comprises phosphoregulatory sites of a juxtamembrane region or kinase domain. Phosphoregulatory sites are sites that are autophosphorylated following ligand binding of an RTK and that potentiate binding of cytoplasmic signalling targets such as SH2 or SH3 domain signalling proteins. In a specific aspect the binding pocket comprises invariant tyrosine residues (e.g. tyrosines 596 and 602 of EphA4; tyrosines 604 and 610 of EphB2) within a conserved amino acid sequence (e.g. YIDPFTYEPD in EphB2) in the juxtamembrane region A binding pocket may comprise one or more of the amino acid residues for an Eph receptor crystal identified as numbers 1 through 49 shown in Table 2. In an aspect the binding pocket comprises the atomic contacts of atomic interactions 1 to 24 (juxtamembrane-kinase interactions) or interactions 25 to 49 (juxtamembrane-juxtamembrane interactions) identified in Table 2. In a preferred embodiment the binding pocket comprises atomic interactions or atomic contacts 27, 28, 29, and 38; 39 and 40; or 9, 13, 14, 16, 18, 19, 32, 39, 40, and 42 in Table 2. In an aspect of the invention the binding pocket comprises all of the amino acid residues identified in Table 2.

A binding pocket may be involved in coordination of a ligand or substrate. For example a binding pocket may be involved in coordination of a nucleotide, or part or analog thereof. Therefore, a binding pocket may comprise two or more of the amino acid residues Phe 709, Met 710 Glu 708, Thr 707, Leu 761, Gly 713, (Lys 661), Ala 659, Ile 691, and (Ser 771) of an RTK structure as described herein, that are capable of associating with or coordinating a nucleotide as described herein.

The term “binding pocket” (BP) also includes a homolog of the binding pocket or a portion thereof. As used herein, the term “homolog” in reference to a binding pocket refers to a binding pocket or a portion thereof which may have deletions, insertions or substitutions of amino acid residues as long as the binding specificity is retained. In this regard, deliberate amino acid substitutions may be made on the basis of similarity in polarity, charge, solubility, hydrophobicity, hydrophilicity, and/or the amphipathic nature of the residues as long as the binding specificity of the binding pocket is retained.

As used herein, the term “portion thereof” means the structural coordinates corresponding to a sufficient number of amino acid residues of a binding pocket (or homologs thereof) that are capable of providing an autoinhibited or active state or for associating with a ligand For example, the structural coordinates provided in a crystal structure may contain a subset of the amino acid residues in a binding pocket which may be useful in the modelling and design of compounds that bind to the binding pocket.

Autoinhbited/Active State

An RTK or a binding pocket thereof may be in an autoinhibited state or active state. An “autoinhibited state” refers to the state of a RTK or a binding pocket that results in disruption of the activation segment of the kinase domain and effective coordination of bound nucleotide. The autoinhibited state results in perturbed catalytic function of an RTK. An autoinhibited state typically occurs in the absence of phosphorylation of the RTK.

An “active state” refers to the state of a RTK or a binding pocket that does not result in disruption of the activation segment of the kinase domain and effective coordination of bound nucleotide. In the active state the RTK is catalytically active and the juxtamembrane segment is free to bind to signalling proteins such as SH2 domain containing proteins, including p120-RasGAP, Nck, phosphatidylinositol 3′-kinase, SHEP-1, Src family kinases, and the adapter protein Nck. An active state typically occurs in the presence of phosphorylation of the RTK.

Crystal

The invention provides crystal structures. As used herein, the term “crystap” or “crystalline” means a structure (such as a three dimensional (3D) solid aggregate) in which the plane faces intersect at definite angles and in which there is a regular structure (such as internal structure) of the constituent chemical species. Thus, the term “crystal” can include any one of: a solid physical crystal form such as an experimentally prepared crystal, a crystal structure derivable from the crystal (including secondary and/or tertiary and/or quaternary structural elements), a 2D and/or 3D model based on the crystal structure, a representation thereof such as a schematic representation thereof or a diagrammatic representation thereof, or a data set thereof for a computer.

In one aspect the crystal is usable in X-ray crystallography techniques. Here, the crystals used can withstand exposure to X-ray beams used to produce a diffraction pattern data necessary to solve the X-ray crystallographic structure. A crystal may be characterized as being capable of diffracting x-rays in a pattern defined by one of the crystal forms depicted in Blundel et al 1976, Protein Crystallography, Academic Press.

A crystal of the invention is generally produced in a laboratory; that is, it is an isolated crystal produced by an individual.

The invention contemplates a crystal comprising a binding pocket of the invention, in particular a binding pocket that regulates the kinase domain of the receptor tyrosine kinase. The binding pocket may be of an autoinhibited state RTK or an active RTK.

In an aspect of the invention a crystal is provided that comprises the juxtamembrane region and kinase domain of an RTK. In an embodiment the RTK is an Eph Receptor, preferably an EphB receptor. In a preferred embodiment the crystal comprises the juxtamembrane region and the catalytic domain (amino acid residues 595 to 906) of EphB2. The juxtamembrane region and the catalytic domain may be in an autoinhibited state.

A crystal of the invention may be characterized by one or more of the following characteristics:

(a) an N-terminal lobe for binding and coordinating ATP for transfer of an α-phosphate to a substrate, comprising a twisted 5-strand β-sheet (denoted β1 to β5) and a single helix αC; and optionally further characterized by (i) a flexible loop that interacts with the adenine base, ribose sugar and the non-hydrolyzable phosphate groups of ATP which loop is formed by β-strands 1 and 2 and a connecting glycine rich segment (g-loop) and

(ii) an invariant salt bridge between a lysine side chain in 3 strand 3 and a glutamic acid side chain in helix αC that coordinates the position of the P-phosphate of ATP; and

(b) a C-terminal lobe comprising two β-strands (β7 and β8) and a series of α-helices (αD to αI which is further characterized by (i) strands β7 and β8 in the cleft region between the N- and C-terminal lobes where they contribute side chains that participate in catalysis and the binding of magnesium for the coordination of ATP phosphate groups, (ii) an activation segment flanked by the sequence Asp-Phe-Gly of sub-domain VII and Pro-Ile-Arg of sub-domain VIII, and (iii) a helix αI adjacent to helix αH.

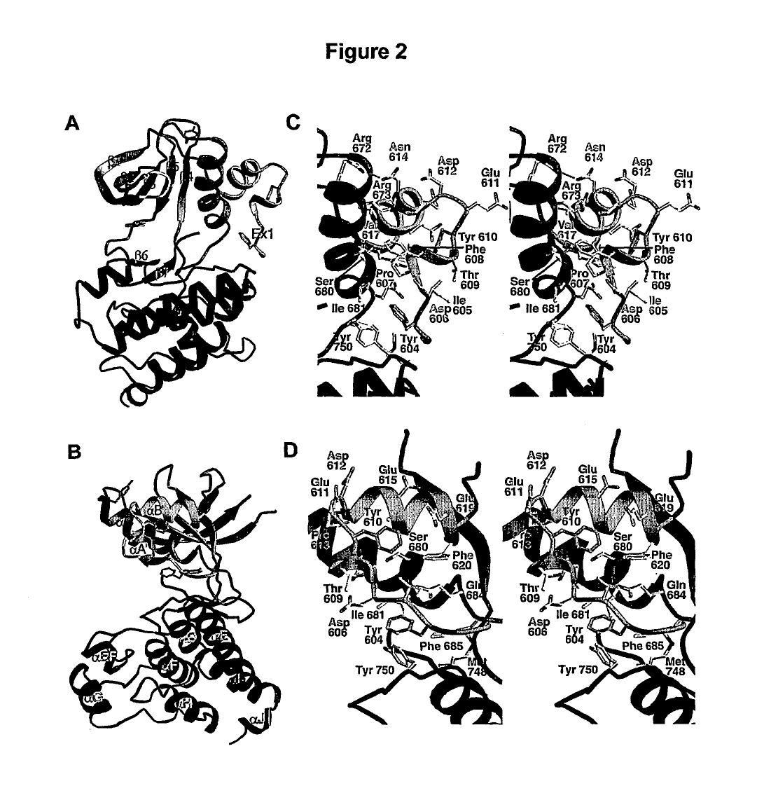

A crystal of the invention comprising a juxtamembrane region of an RTK, in particular an Eph receptor, more particularly an EphB receptor, most particularly an EphB2 receptor, may be characterized as comprising a single-turn helix αA⁺ (i.e. a 3/10 helix), and a four-turn helix αB′ from the amino terminus of an extended strand segment Ex1. The crystal may also comprise this juxtamemembrane region in association with interacting amino acid residues on the N- and C-terminal lobes of the RTK. (See FIGS. 2-4, and Table 2.)

A crystal of the invention may comprise a juxtamembrane strand segment Ex1 comprising amino acid residues Lys 602 to Ile 605 which strand extends along the cleft region between the N-and C-terminal lobes of an RTK. The strand is stabilized by hydrogen bonding interactions involving the amide group of Phe 604 with the carbonyl group of Met 748 and the Gln 684 side chain with the backbone amide and carbonyl groups of Ile 605.

In a further aspect of the invention a crystal is provided comprising a hydrophobic interface site (referred to herein as switch region 1) comprising side chains of Met 748 and Tyr 750 of the C-terminal kinase lobe; Phe 685 and Ile 681 from helix αC, and Pro 607 from the juxtamembrane helix αA^{t}, and the phosphoregulatory site or residue Phe 604 which orients into the site.

A crystal of the invention may comprise helix aA′ which is more particularly characterized by one or more of the following characteristics:

(a) it is composed of a single rigid turn initiated by Asp 606 and Pro 607 and terminated by Thr 609;

(b) it is stabilized by the conformational regidity of Pro 607 and the capping interactions involving Asp 606 and Thr 609 with the free backbone amino group and carbonly groups of Phe 608 and Asp 606.

A crystal of the invention may comprise helix αB¹ which is more particularly characterized by one or more of the following characteristics:

(a) it is initiated by an Asp Pro sequence (residues 612 and 613); and

(b) Asp 612 makes capping interactions with the backbone amino and side chain of Asn 614.

A crystal of the invention may comprise helices αA′ and αB′ of a juxtamembrane region of an RTK and the portion of the N-terminal lobe of the kinase domain centering on helix αC of the RTK which forms an interface with helices αA′ and αB′ and is further characterized as follows:

(a) hydrophobic side chains projecting from αA′ and αB′ include Pro 607, Phe 608, Pro 613, Val 617, Phe620 and Ala 621 which residues associate intimately with the side chains of Arg 673, Leu 676, and Ile 681 from helix αC and the side chains of Leu 693 and Val 696 from 1-strand 4;

(b) a hydrogen bond interaction (2.9 Å) between Asn 614 and Arg 672; and

(c) the small side chains at positions 616 (Ala), 677 (Ser) and 680 (Ser) facilitate the close packing of helices αA′, αB′ and αC.

A crystal of the invention may comprise a hydrophobic interface site (also referred to herein as “switch region 2”) formed by association of helix αC, strand Ex1 and helices αA′ and αB′ of the juxtamembrane region of an RTK. The interface is characterized as follows:

(a) projection of the side chain of the phosphoregulatory residue Tyr/Phe 610 onto the surface of the site;

(b) composed of the side chains of Ile 605 from strand Ex1 and the side chains of Ala 616 and Phe 620 from helix αB′; and

(c) an electrostatic environment dominated by Asp 606, Glu 611, Asp 612, Glu 615, and Glu 619.

A crystal of the invention may comprise the following amino acids residues:

(a) Arg 672, Phe Arg 672, Phe 675, and Leu 676 from helix αC, Tyr 667 from the 13/αC linker and Leu 663, Val 696, Thr 698, Val 703, and Ile 705; or

(b) Met 748, Tyr 750, Phe 685, Ile 681, Pro 607, and Phe 604; or

(c) Phe 709, Met 710, Glu 708, Thr 707, Leu 761, Gly 713, Ala 659, Ile 691, Lys 661, and Ser 771; or

(d) Asp 606, Pro 607, Thr 609, Phe 608 and Asp 606; or

(e) Asp 612, Pro 613, Asp 612, and Asn 614; or

(f) Pro 607, Phe 608, Pro 613, Val 617, Phe 620, Als 621, Arg 673, Leu 676, Ile 681, Leu 693, Val 696, Asn 614, Arg 672, Ala 616, Ser 677, and Ser 680; or

(g) Tyr/Phe 610, Ile 605, Ala 616, Phe 620, Asp 606, Glu 611, Asp 615, and Glu 619.

Preferably the atoms of the amino acid residues in (a) to (g) have the structural coordinates as set out in Table 3.

In an embodiment, a crystal of a Eph receptor of the invention belongs to space group P2₁ or P1. The term “space group” refers to the lattice and symmetry of the crystal. In a space group designation the capital letter indicates the lattice type and the other symbols represent symmetry operations that can be carried out on the contents of the asymmetric unit without changing its appearance.

A crystal of the invention may comprise a unit cell having the following unit dimensions: a=47.05 (±0.05) Å, b=57.62 (±0.05) Å, c=67.74 (±0.05) Å, or a=47.86 (±0.05) Å, b=98.09 (±0.05) Å, c=68.18 (±0.05) Å. The term “unit cell” refers to the smallest and simplest volume element (i.e. parallelpiped-shaped block) of a crystal that is completely representative of the unit of pattern of the crystal. The unit cell axial lengths are represented by a, b, and c. Those of skill in the art understand that a set of atomic coordinates determined by X-ray crystallography is not without standard error.

In a preferred embodiment, a crystal of the invention has the structural coordinates as shown in Table 3. As used herein, the term “structural coordinates” refers to a set of values that define the position of one or more amino acid residues with reference to a system of axes. The term refers to a data set that defines the three dimensional structure of a molecule or molecules (e.g. Cartesian coordinates, temperature factors, and occupancies). Structural coordinates can be slightly modified and still render nearly identical three dimensional structures. A measure of a unique set of structural coordinates is the root-mean-square deviation of the resulting structure. Structural coordinates that render three dimensional structures (in particular a three dimensional structure of a ligand binding pocket) that deviate from one another by a root-mean-square deviation of less than 5 Å, 4 Å, 3 Å, 2 Å, 1.5 Å. 1.0 Å, or 0.5 Å may be viewed by a person of ordinary skill in the art as very similar.

Variations in structural coordinates may be generated because of mathematical manipulations of the structural coordinates of a glycosyltransferase described herein. For example, the structural coordinates of Table 3 may be manipulated by crystallographic permutations of the structural coordinates, fractionalization of the structural coordinates, integer additions or substractions to sets of the structural coordinates, inversion of the structural coordinates or any combination of the above.

Variations in the crystal structure due to mutations, additions, substitutions, and/or deletions of the amino acids, or other changes in any of the components that make up the crystal may also account for modifications in structural coordinates. If such modifications are within an acceptable standard error as compared to the original structural coordinates, the resulting structure may be the same. Therefore, a ligand that bound to a binding pocket of an RTK, in particular an Eph receptor, would also be expected to bind to another binding pocket whose structural coordinates defined a shape that fell within the acceptable error. Such modified structures of a binding pocket thereof are also within the scope of the invention.

Various computational analyses may be used to determine whether a molecule or the binding pocket thereof is sufficiently similar to all or parts of an RTK or a binding pocket thereof. Such analyses may be carried out using conventional software applications and methods as described herein.

A crystal of the invention may also be specifically characterised by the parameters, diffraction statistics and/or refinement statistics set out in Tables 1.

With reference to a crystal of the present invention, residues in a binding pocket may be defined by their spatial proximity to a ligand in the crystal structure. For example, a binding pocket may be defined by its proximity to a nucleotide, substrate molecule, or modulator.

A crystal of the invention may comprise a binding pocket that is involved in coordination of a nucleotide, or part or analog thereof. Therefore, a crystal may comprise a binding pocket comprising two or more of the amino acid residues Phe 709, Met 710 Glu 708, Thr 707, Leu 761, Gly 713, (Lys 661), Ala 659, Ile 691, and (Ser 771) of an RTK structure as described herein, that are capable of associating with or coordinating a nucleotide as described herein.

A crystal or secondary or three-dimensional structure of a binding pocket of an RTK, in particular an EphB2 receptor, may be specifically defined by one or more of the atomic contacts of the atomic interactions identified in Table 2. The atomic interactions in Table 2 are defined therein by an atomic contact (more preferably, a specific atom of an amino acid residue where indicated) on the juxtamembrane region, and an atomic contact (more preferably, a specific atom of an amino acid residue where indicated) on the kinase domain, juxtamembrane region, or ligand. In certain embodiments, a crystal of the invention comprises the atomic contacts of atomic interactions 1 to 24 (juxtamembrane-kinase interactions) or atomic interactions 25 to 49 (juxtamembrane-juxtamembrane interactions) identified in Table 2. In certain particular embodiments a crystal is provided comprising the atomic contacts of atomic interactions 27, 28, 29, and 38; 39 and 40; or 9, 13, 14, 16, 18, 19, 32, 39, 40, and 42.

Preferably, a crystal is defined by the atoms of the atomic contacts in the binding pocket having the structural coordinates for the atoms listed in Table 3.

A crystal of the invention includes a binding pocket in association with one or more moieties, including heavy-metal atoms i.e. a derivative crystal, or one or more ligands or molecules i.e. a co-crystal.

The term “associate”, “association” or “associating” refers to a condition of proximity between a moiety (i.e. chemical entity or compound or portions or fragments thereof), and a binding pocket. The association may be non-covalent i.e. where the juxtaposition is energetically favored by for example, hydrogen-bonding, van der Waals, or electrostatic or hydrophobic interactions, or it may be covalent.

The term “heavy-metal atoms” refers to an atom that can be used to solve an x-ray crystallography phase problem, including but not limited to a transition element a lanthamide metal, or an actinide metal. Lanthamide metals include elements with atomic numbers between 57 and 71, inclusive. Actinide metals include elements with atomic numbers between 89 and 103, inclusive.

Multiwavelength anomalous diffraction (MAD) phasing may be used to solve protein structures using selenomethionyl (SeMet) proteins. Therefore, a complex of the invention may comprise a crystalline binding pocket with selenium on the methionine residues of the protein.

A crystal may comprise a complex between a binding pocket and one or more ligands or molecules. In other words the binding pocket may be associated with one or more ligands or molecules in the crystal. The ligand may be any compound that is capable of stably and specifically associating with the binding pocket. A ligand may, for example, be a modulator of an Eph receptor, or a nucleotide or substrate or analogue thereof.

In an embodiment of the invention, a binding pocket is in association with a cofactor in the crystal. A “cofactor” refers to a molecule required for RTK enzyme activity and/or stability. For example, the cofactor may be a metal ion, including magnesium and other similar atoms or metals.

In an embodiment, a crystal of the invention comprises a complex between a binding pocket, and a nucleotide or analogue thereof and/or a substrate or analogue thereof. A “nucleotide” includes ATP, ADP, AMP, or analogues thereof, for example, β,γ-imidoadenosine-5′-triphosphate (AMP-PNP, STI-571, and quercetin. A substrate may be for example, a signalling protein, or another portion of the same RTK (e.g juxtamembrane-kinase domain complex). An analog of a nucleotide or substrate is one which mimics the nucleotide or substrate molecule, binding in the binding pocket, but which is incapable (or has a significantly reduced capacity) to take part in a kinase reaction.

Therefore, the present invention also provides:

(a) a crystal comprising a binding pocket of an RTK and a nucleotide or analogue thereof;

(b) a crystal comprising a binding pocket of an RTK and a substrate or analogue thereof;

(c) a crystal comprising a binding pocket of an RTK and a nucleotide or analogue thereof, and a substrate or analogue thereof.

A complex may comprise one or more of the intermolecular interactions identified in Table 2. A structure of a complex of the invention may be defined by selected intermolecular contacts, preferably the structural coordinates of the intermolecular contacts as defined in Table 3.

A crystal of the invention may enable the determination of structural data for a ligand. In order to be able to derive structural data for a ligand, it is necessary for the molecule to have sufficiently strong electron density to enable a model of the molecule to be built using standard techniques. For example, there should be sufficient electron density to allow a model to be built using XTALVIEW (McRee 1992 J. Mol. Graphics. 10 44-46).

Illustrations of particular crystals of the invention are shown in FIGS. 2, 3, and 4.

Method of Making a Crystal

The present invention also provides a method of making a crystal according to the invention. The crystal may be formed from an aqueous solution comprising a purified polypeptide comprising an RTK, in particular an Eph receptor including a variant, part, homolog, or fragment thereof (e.g. a binding pocket). A method may utilize a purified polypeptide comprising a binding pocket to form a crystal. A method may utilize a purified polypeptide comprising a juxtamembrane region and kinase domain of an RTK, in particular an Eph receptor, preferably an EphB receptor, or more preferably an EphB2 receptor.

The term “purified” in reference to a polypeptide, does not require absolute purity such as a homogenous preparation rather it represents an indication that the polypeptide is relatively purer than in the natural environment. Generally, a purified polypeptide is substantially free of other proteins, lipids, carbohydrates, or other materials with which it is naturally associated, preferably at a functionally significant level for example at least 85% pure, more preferably at least 95% pure, most preferably at least 99% pure. A skilled artisan can purify a polypeptide comprising using standard techniques for protein purification. A substantially pure polypeptide will yield a single major band on a non-reducing polyacrylamide gel. Purity of the polypeptide can also be determined by amino-terminal amino acid sequence analysis.

A polypeptide used in the method may be chemically synthesized in whole or in part using techniques that are well-known in the art. Alternatively, methods are well known to the skilled artisan to construct expression vectors containing a native or mutated RTK coding sequence and appropriate transcriptional/translational control signals. These methods include in vitro recombinant DNA techniques, synthetic techniques, and in vivo recombination/genetic recombination. See for example the techniques described in Sambrook et al. (Molecular Cloning: A Laboratory Manual, 2nd Edition, Cold Spring Harbor Laboratory press (1989)), and other laboratory textbooks. (See also Sarker et al, Glycoconjugate J. 7:380, 1990; Sarker et al, Proc. Natl. Acad, Sci. USA 88:234-238, 1991, Sarker et al, Glycoconjugate J. 11: 204-209, 1994; Hull et al, Biochem Biophys Res Commun 176:608, 1991 and Pownall et al, Genomics 12:699-704, 1992).

Crystals may be grown from an aqueous solution containing the purified polypeptide by a variety of conventional processes. These processes include batch, liquid, bridge, dialysis, vapor diffusion, and hanging drop methods. (See for example, McPherson, 1982 John Wiley, New York; McPherson, 1990, Eur. J. Biochem. 189: 1-23; Webber. 1991, Adv. Protein Chem. 41:1-36). Generally, native crystals of the invention are grown by adding precipitants to the concentrated solution of the polypeptide. The precipitants are added at a concentration just below that necessary to precipitate the protein. Water is removed by controlled evaporation to produce precipitating conditions, which are maintained until crystal growth ceases.

Derivative crystals of the invention can be obtained by soaking native crystals in a solution containing salts of heavy metal atoms. A complex of the invention can be obtained by soaking a native crystal in a solution containing a compound that binds the polypeptide, or they can be obtained by co-crystallizing the polypeptide in the presence of one or more compounds. In order to obtain co-crystals with a compound which binds deep within the tertiary structure of the polypeptide it is necessary to use the second method.

In a preferred embodiment, the polypeptide is co-crystallised with a compound which stabilises the polypeptide (e.g. AMP-PNP).

Once the crystal is grown it can be placed in a glass capillary tube and mounted onto a holding device connected to an X-ray generator and an X-ray detection device. Collection of X-ray diffraction patterns are well documented by those skilled in the art (See for example, Ducruix and Geige, 1992, IRL Press, Oxford, England). A beam of X-rays enter the crystal and diffract from the crystal. An X-ray detection device can be utilized to record the diffraction patterns emanating from the crystal. Suitable devices include the Marr 345 imaging plate detector system with an RU200 rotating anode generator.

Multiwavelength anomalous diffraction (MAD) phasing using selenomethionyl (SeMet) proteins may be used to determine a crystal of the invention. Thus, the invention contemplates a method for determining a crystal structure of the invention using a selenomethionyl derivative of an RTK, including a variant, part, homolog or fragement thereof.

Methods for obtaining the three dimensional structure of the crystalline form of a molecule or complex are described herein and known to those skilled in the art (see Ducruix and Geige 1992, IRL Press, Oxford, England). Generally, the x-ray crystal structure is given by the diffraction patterns. Each diffraction pattern reflection is characterized as a vector and the data collected at this stage determines the amplitude of each vector. The phases of the vectors may be determined by the isomorphous replacement method where heavy atoms soaked into the crystal are used as reference points in the X-ray analysis (see for example, Otwinowski, 1991, Daresbury, United Kingdom, 80-86). The phases of the vectors may also be determined by molecular replacement (see for example, Naraza, 1994, Proteins 11:281-296). The amplitudes and phases of vectors from the crystalline form determined in accordance with these methods can be used to analyze other related crystalline polypeptides.

The unit cell dimensions and symmetry, and vector amplitude and phase information can be used in a Fourier transform function to calculate the electron density in the unit cell i.e. to generate an experimental electron density map. This may be accomplished using the PHASES package (Furey, 1990). Amino acid sequence structures are fit to the experimental electron density map (i.e. model building) using computer programs (e.g. Jones, TA. et al, Acta Crystallogr A47, 100-119, 1991). This structure can also be used to calculate a theoretical electron density map. The theoretical and experimental electron density maps can be compared and the agreement between the maps can be described by a parameter referred to as R-factor. A high degree of overlap in the maps is represented by a low value R-factor. The R-factor can be minimized by using computer programs that refine the structure to achieve agreement between the theoretical and observed electron density map. For example, the XPLOR program, developed by Brunger (1992, Nature 355:472-475) can be used for model refinement A three dimensional structure of the molecule or complex may be described by atoms that fit the theoretical electron density characterized by a minimum R value. Files can be created for the structure that defines each atom by coordinates in three dimensions.

Model

A crystal structure of the present invention may be used to make a model of a binding pocket of an RTK, preferably an Eph receptor, more preferably an EphB receptor. A model may, for example, be a structural model or a computer model. A model may represent the secondary, tertiary and/or quaternary structure of the binding pocket. The model itself may be in two or three dimensions. It is possible for a computer model to be in three dimensions despite the constraints imposed by a conventional computer screen, if it is possible to scroll along at least a pair of axes, causing “rotation” of the image.

As used herein, the term “modelling” includes the quantitative and qualitative analysis of molecular structure and/or function based on atomic structural information and interaction models. The term “modelling” includes conventional numeric-based molecular dynamic and energy minimization models, interactive computer graphic models, modified molecular mechanics models, distance geometry and other structure-based constraint models.

Preferably, modelling is performed using a computer and may be further optimized using known methods. This is called modelling optimisation.

An integral step to an approach of the invention for designing modulators (e.g. inhibitors) of a subject receptor involves construction of computer graphics models of the binding pocket of a receptor which can be used to design pharmacophores by rational drug design. For instance, for an inhibitor to interact optimally with the subject binding pocket, it will generally be desirable that it have a shape which is at least partly complimentary to that of a particular binding pocket of the receptor, as for example those binding pockets of the receptor which are involved in recognition of a ligand, regulating the kinase domain, or regulating signal transduction. Additionally, other factors, including electrostatic interactions, hydrogen bonding, hydrophobic interactions, desolvation effects, and cooperative motions of ligand and receptor, all influence the binding effect and should be taken into account in attempts to design bioactive modulators (e.g. inhibitors).

As described herein, a computer-generated molecular model of the subject receptors can be created. In preferred embodiments, at least the Ca-carbon positions of the RTK sequence of interest are mapped to a particular coordinate pattern, such as the coordinates for a binding pocket of an EphB2 shown in Table 3, by homology modeling, and the structure of the protein and velocities of each atom are calculated at a simulation temperature (Tₒ) at which the docking simulation is to be determined. Typically, such a protocol involves primarily the prediction of side-chain conformations in the modeled protein, while assuming a main-chain trace taken from a tertiary structure such as provided in Table 3 and the Figures. Computer programs for performing energy minimization routines are commonly used to generate molecular models. For example, both the CHARMM (Brooks et al. (1983) J Comput Chem 4:187-217) and AMBER (Weiner et al (1981) J. Comput. Chem. 106: 765) algorithms handle all of the molecular system setup, force field calculation, and analysis (see also, Eisenfield et al. (1991) Am J Physiol 261:C376-386; Lybrand (1991) J Pharm Belg 46:49-54; Froimowitz (1990) Biotechniques 8:640-644; Burbam et al. (1990) Proteins 7:99-111; Pedersen (1985) Environ Health Perspect 61:185-190; and Kini et al. (1991) J Biomol Struct Dyn 9:475-488). At the heart of these programs is a set of subroutines that, given the position of every atom in the model, calculate the total potential energy of the system and the force on each atom. These programs may utilize a starting set of atomic coordinates, such as the coordinates provided in Table 3, the parameters for the various terms of the potential energy function, and a description of the molecular topology (the covalent structure). Common features of such molecular modeling methods include: provisions for handling hydrogen bonds and other constraint forces; the use of periodic boundary conditions; and provisions for occasionally adjusting positions, velocities, or other parameters in order to maintain or change temperature, pressure, volume, forces of constraint, or other externally controlled conditions.

Most conventional energy minimization methods use the input data described above and the fact that the potential energy function is an explicit, differentiable function of Cartesian coordinates, to calculate the potential energy and its gradient (which gives the force on each atom) for any set of atomic positions. This information can be used to generate a new set of coordinates in an effort to reduce the total potential energy and, by repeating this process over and over, to optimize the molecular structure under a given set of external conditions. These energy minimization methods are routinely applied to molecules similar to the subject RTK proteins as well as nucleic acids, polymers and zeolites.

In general, energy minimization methods can be carried out for a given temperature, Tᵢ, which may be different than the docking simulation temperature, Tₒ. Upon energy minimization of the molecule at Tᵢ, coordinates and velocities of all the atoms in the system are computed. Additionally, the normal modes of the system are calculated. It will be appreciated by those skilled in the art that each normal mode is a collective, periodic motion, with all parts of the system moving in phase with each other, and that the motion of the molecule is the superposition of all normal modes. For a given temperature, the mean square amplitude of motion in a particular mode is inversely proportional to the effective force constant for that mode, so that the motion of the molecule will often be dominated by the low frequency vibrations.

After the molecular model has been energy minimized at Tᵢ, the system is “heated” or “cooled” to the simulation temperature, Tₒ, by carrying out an equilibration run where the velocities of the atoms are scaled in a step-wise manner until the desired temperature, Tₒ, is reached. The system is further equilibrated for a specified period of time until certain properties of the system, such as average kinetic energy, remain constant. The coordinates and velocities of each atom are then obtained from the equilibrated system.

Further energy minimization routines can also be carried out. For example, a second class of methods involves calculating approximate solutions to the constrained EOM for the protein. These methods use an iterative approach to solve for the Lagrange multipliers and, typically, only need a few iterations if the corrections required are small. The most popular method of this type, SHAKE (Ryckaert et al. (1977) J Comput Phys 23:327; and Van Gunsteren et al. (1977) Mol Phys 34:1311) is easy to implement and scales as O(N) as the number of constraints increases. Therefore, the method is applicable to macromolecules such as the RTK proteins of the present invention. An alternative method, RATTLE (Anderson (1983) J Comput Phys 52:24) is based on the velocity version of the Verlet algorithm. Like SHAKE, RATTLE is an iterative algorithm and can be used to energy minimize the model of the subject protein.

Overlays and super positioning with a three dimensional model of a binding pocket of the invention may be used for modelling optimisation. Additionally alignment and/or modelling can be used as a guide for the placement of mutations on a binding pocket to characterize the nature of the site in the context of a cell.

The three dimensional structure of a new crystal may be modelled using molecular replacement The term “molecular replacement” refers to a method that involves generating a preliminary model of a molecule or complex whose structural coordinates are unknown, by orienting and positioning a molecule whose structural coordinates are known within the unit cell of the unknown crystal, so as best to account for the observed diffraction pattern of the unknown crystal. Phases can then be calculated from this model and combined with the observed amplitudes to give an approximate Fourier synthesis of the structure whose coordinates are unknown. This, in turn, can be subject to any of the several forms of refinement to provide a final, accurate structure of the unknown crystal. Lattman, E., “Use of the Rotation and Translation Functions”, in Methods in Enzymology, 115, pp. 55-77 (1985); M. G. Rossmann, ed., “The Molecular Replacement Method”, Int. Sci. Rev. Ser., No. 13, Gordon & Breach, New York, (1972).

Commonly used computer software packages for molecular replacement are X-PLOR (Brunger 1992, Nature 355: 472-475), AMoRE (Navaza, 1994, Acta Crystallogr. A50:157-163), the CCP4 package (Collaborative Computational Project, Number 4, “The CCP4 Suite: Programs for Protein Crystallography”, Acta Cryst., Vol. D50, pp. 760-763, 1994), the MERLOT package (P. M. D. Fitzgerald, J. Appl. Cryst., Vol. 21, pp. 273-278, 1988) and XTALVIEW (McCree et al (1992) J. Mol. Graphics 10: 44-46. It is preferable that the resulting structure not exhibit a root-mean-square deviation of more than 3 Å.

Molecular replacement computer programs generally involve the following steps: (1) determining the number of molecules in the unit cell and defining the angles between them (self rotation function); (2) rotating the known structure against diffraction data to define the orientation of the molecules in the unit cell (rotation function); (3) translating the known structure in three dimensions to correctly position the molecules in the unit cell (translation function); (4) determining the phases of the X-ray diffraction data and calculating an R-factor calculated from the reference data set and from the new data wherein an R-factor between 30-50% indicates that the orientations of the atoms in the unit cell have been reasonably determined by the method; and (5) optionally, decreasing the R-factor to about 20% by refining the new electron density map using iterative refinement techniques known to those skilled in the art (refinement).

The quality of the model may be analysed using a program such as PROCHECK or 3D-Profiler [Laskowski et al 1993 J. Appl. Cryst. 26:283-291; Luthy R et al, Nature 356: 83-85, 1992; and Bowie, J. U. et al, Science 253: 164-170, 1991]. Once any irregularities have been resolved, the entire structure may be further refined.

Other molecular modelling techniques may also be employed in accordance with this invention. See, e.g., Cohen, N. C. et al, “Molecular Modelling Software and Methods for Medicinal Chemistry”, J. Med. Chem., 33, pp. 883-894 (1990). See also, Navia, M. A. and M. A. Murcko, “The Use of Structural Information in Drug Design”, Current Opinions in Structural Biology, 2, pp. 202-210 (1992).

Using the structural coordinates of crystal provided by the invention, molecular modelling may be used to determine the structural coordinates of a crystalline mutant or homolog of an RTK binding pocket By the same token a crystal of the invention can be used to provide a model of a ligand. Modelling techniques can then be used to approximate the three dimensional structure of ligand derivatives and other components which may be able to mimic the atomic contacts between a ligand and binding pocket.

Computer Format of Crystals/Models

Information derivable from a crystal of the present invention (for example the structural coordinates) and/or the model of the present invention may be provided in a computer-readable format Therefore, the invention provides a computer readable medium or a machine readable storage medium which comprises the structural coordinates of a binding pocket of an RTK including all or any parts thereof, or ligands including portions thereof. Such storage medium or storage medium encoded with these data are capable of displaying on a computer screen or similar viewing device, a three-dimensional graphical representation of a molecule or molecular complex which comprises such binding pockets or similarly shaped homologous binding pockets. Thus, the invention also provides computerized representations of the secondary or three-dimensional structures of a binding pocket of the invention, including any electronic, magnetic, or electromagnetic storage forms of the data needed to define the structures such that the data will be computer readable for purposes of display and/or manipulation.

In an aspect the invention provides a computer for producing a three-dimensional representation of a molecule or molecular complex, wherein said molecule or molecular complex comprises a binding pocket defined by structural coordinates of a binding pocket or structural coordinates of atoms of a ligand, or a three-dimensional representation of a homolog of said molecule or molecular complex, wherein said homolog comprises a binding pocket or ligand that has a root mean square deviation from the backbone atoms not more than 1.5 angstroms wherein said computer comprises:

(a) a machine-readable data storage medium comprising a data storage material encoded with machine readable data wherein said data comprises the structural coordinates of a binding pocket of an RTK or a ligand according to Table 3;

(b) a working memory for storing instructions for processing said machine-readable data;

(c) a central-processing unit coupled to said working memory and to said machine-readable data storage medium for processing said machine readable data into said three-dimensional representation; and

(d) a display coupled to said central-processing unit for displaying said three-dimensional representation.

The invention also provides a computer for determining at least a portion of the structural coordinates corresponding to an X-ray diffraction pattern of a molecule or molecular complex wherein said computer comprises:

(a) a machine-readable data storage medium comprising a data storage material encoded with machine readable data wherein said data comprises the structural coordinates according to Table 3;

(b) a machine-readable data storage medium comprising a data storage material encoded with machine readable data wherein said data comprises an X-ray diffraction pattern of said molecule or molecular complex;

(c) a working memory for storing instructions for processing said machine-readable data of (a) and (b);

(d) a central-processing unit coupled to said working memory and to said machine-readable data storage medium of (a) and (b) for performing a Fourier transform of the machine readable data of (a) and for processing said machine readable data of (b) into structural coordinates; and

(e) a display coupled to said central-processing unit for displaying said structural coordinates of said molecule or molecular complex.

Structural Studies

The present invention also provides a method for determining the secondary and/or tertiary structures of a polypeptide or part thereof by using a crystal, or a model according to the present invention. The polypeptide or part thereof may be any polypeptide or part thereof for which the secondary and or tertiary structure is uncharacterised or incompletely characterised. In a preferred embodiment the polypeptide shares (or is predicted to share) some structural or functional homology to a crystal of the present invention. For example, the polypeptide may show a degree of structural homology over some or all parts of the primary amino acid sequence.

The polypeptide may be an RTK, preferably an Eph receptor with a different specificity for a nucleotide, or substrate. The polypeptide may be an RTK preferably an Eph receptor which requires a different metal cofactor. Alternatively (or in addition) the polypeptide may be an RTK, preferably an Eph receptor from a different species.

The polypeptide may be a mutant of a wild-type RTK, in particular an Eph receptor. A mutant may arise naturally, or may be made artificially (for example using molecular biology techniques). The mutant may also not be “made” at all in the conventional sense, but merely tested theoretically using the model of the present invention. A mutant may or may not be functional.

Thus, using a model of the present invention, the effect of a particular mutation on the overall two and/or three dimensional structure of an RTK, in particular an Eph receptor, the autoinhibited state or active state, and/or the interaction between a binding pocket of the enzyme and a ligand can be investigated.

Alternatively, the polypeptide may perform an analogous function or be suspected to show a similar catalytic mechanism to an RTK, in particular an Eph receptor.

The polypeptide may also be the same as the polypeptide of the crystal, but in association with a different ligand (for example, modulator or inhibitor) or cofactor. In this way it is possible to investigate the effect of altering the ligand or compound with which the polypeptide is associated on the structure of the binding pocket.

Secondary or tertiary structure may be determined by applying the structural coordinates of the crystal or model of the present invention to other data such as an amino acid sequence, X-ray crystallographic diffraction data, or nuclear magnetic resonance (NMR) data. Homology modeling, molecular replacement, and nuclear magnetic resonance methods using these other data sets are described below.

Homology modeling (also known as comparative modeling or knowledge-based modeling) methods develop a three dimensional model from a polypeptide sequence based on the structures of known proteins (i.e. an RTK, in particular an Eph receptor, of the crystal). The method utilizes a computer model of a crystal of the present invention (the “known structure”), a computer representation of the amino acid sequence of the polypeptide with an unknown structure, and standard computer representations of the structures of amino acids. The method in particular comprises the steps of; (a) identifying structurally conserved and variable regions in the known structure; (b) aligning the amino acid sequences of the known structure and unknown structure (c) generating co-ordinates of main chain atoms and side chain atoms in structurally conserved and variable regions of the unknown structure based on the coordinates of the known structure thereby obtaining a homology model; and (d) refining the homology model to obtain a three dimensional structure for the unknown structure. This method is well known to those skilled in the art (Greer, 1985, Science 228, 1055; Bundell et al 1988, Eur. J. Biochem. 172, 513; Knighton et al., 1992, Science 258:130-135, http://biochem.vtedu/courses/modeling/homology.htn). Computer programs that can be used in homology modelling are Quanta and the Homology module in the Insight II modelling package distributed by Molecular Simulations Inc, or MODELLER (Rockefeller University, www.iucr.ac.uk/sinris-top/logical/prg-modeller.html).

In step (a) of the homology modelling method, a known structure is examined to identify the structurally conserved regions (SCRs) from which an average structure, or framework, can be constructed for these regions of the protein. Variable regions (VRs), in which known structures may differ in conformation, also must be identified. SCRs generally correspond to the elements of secondary structure, such as alpha-helices and beta-sheets, and to ligand- and substrate-binding sites (e.g. nucleotide binding sites). The VRs usually lie on the surface of the proteins and form the loops where the main chain turns.

Many methods are available for sequence alignment of known structures and unknown structures. Sequence alignments generally are based on the dynamic programming algorithm of Needleman and Wunsch [J. Mol. Biol. 48: 442-453, 1970]. Current methods include FASTA, Smith-Waterman, and BLASTP, with the BLASTP method differing from the other two in not allowing gaps. Scoring of alignments typically involves construction of a 20×20 matrix in which identical amino acids and those of similar character (i.e., conservative substitutions) may be scored higher than those of different character. Substitution schemes which may be used to score alignments include the scoring matrices PAM (Dayhoff et al., Meth. Enzymol. 91: 524-545, 1983), and BLOSUM (Henikoff and Henikoff, Proc. Nat. Acad. Sci. USA 89: 10915-10919, 1992), and the matrices based on alignments derived from three-dimensional structures including that of Johnson and Overington (JO matrices) (J. Mol. Biol. 233: 716-738,1993).

Alignment based solely on sequence may be used; however, other structural features also may be taken into account. In Quanta, multiple sequence alignment algorithms are available that may be used when aligning a sequence of the unknown with the known structures. Four scoring systems (i.e. sequence homology, secondary structure homology, residue accessibility homology, CA-CA distance homology) are available, each of which may be evaluated during an alignment so that relative statistical weights may be assigned.

When generating coordinates for the unknown structure, main chain atoms and side chain atoms, both in SCRs and VRs need to be modelled A variety of approaches known to those skilled in the art may be used to assign co-ordinates to the unknown. In particular, the co-ordinates of the main chain atoms of SCRs will be transferred to the unknown structure. VRs correspond most often to the loops on the surface of the polypeptide and if a loop in the known structure is a good model for the unknown, then the main chain co-ordinates of the known structure may be copied. Side chain coordinates of SCRs and VRs are copied if the residue type in the unknown is identical to or very similar to that in the known structure. For other side chain coordinates, a side chain rotamer library may be used to define the side chain coordinates. When a good model for a loop cannot be found fragment databases may be searched for loops in other proteins that may provide a suitable model for the unknown. If desired, the loop may then be subjected to conformational searching to identify low energy conformers if desired.

Once a homology model has been generated it is analyzed to determine its correctness. A computer program available to assist in this analysis is the Protein Health module in Quanta which provides a variety of tests. Other programs that provide structure analysis along with output include PROCHECK and 3D-Profiler [Luthy R et al, Nature 356: 83-85, 1992; and Bowie, J. U. et al, Science 253: 164-170, 1991]. Once any irregularities have been resolved, the entire structure may be further refined. Refinement may consist of energy minimization with restraints, especially for the SCRs. Restraints may be gradually removed for subsequent minimizations. Molecular dynamics may also be applied in conjunction with energy minimization.

Molecular replacement involves applying a known structure to solve the X-ray crystallographic data set of a polypeptide of unknown structure. The method can be used to define the phases describing the X-ray diffraction data of a polypeptide of unknown structure when only the amplitudes are known. Thus in an embodiment of the invention, a method is provided for determining three dimensional structures of polypeptides with unknown structure by applying the structural coordinates of a crystal of the present invention to provide an X-ray crystallographic data set for a polypeptide of unknown structure, and (b) determining a low energy conformation of the resulting structure.

The structural coordinates of a crystal of the present invention may be applied to nuclear magnetic resonance (NMR) data to determine the three dimensional structures of polypeptides with uncharacterised or incompletely characterised sturcture. (See for example, Wuthrich, 1986, John Wiley and Sons, New York: 176-199; Pflugrath et al., 1986, J. Molecular Biology 189: 383-386; Kline et al., 1986 J. Molecular Biology 189:377-382). While the secondary structure of a polypeptide may often be determined by NMR data, the spatial connections between individual pieces of secondary structure are not as readily determined. The structural coordinates of a polypeptide defined by X-ray crystallography can guide the NMR spectroscopist to an understanding of the spatial interactions between secondary structural elements in a polypeptide of related structure. Information on spatial interactions between secondary structural elements can greatly simplify Nuclear Overhauser Effect (NOE) data from two-dimensional NMR experiments. In addition, applying the structural coordinates after the determination of secondary structure by NMR techniques simplifies the assignment of NOE's relating to particular amino acids in the polypeptide sequence and does not greatly bias the NMR analysis of polypeptide structure.

In an embodiment, the invention relates to a method of determining three dimensional structures of polypeptides with unknown structures, by applying the structural coordinates of a crystal of the present invention to nuclear magnetic resonance (NMR) data of the unknown structure. This method comprises the steps of: (a) determining the secondary structure of an unknown structure using NMR data; and (b) simplifying the assignment of through-space interactions of amino acids. The term “through-space interactions” defines the orientation of the secondary structural elements in the three dimensional structure and the distances between amino acids from different portions of the amino acid sequence. The term “assigmnent” defines a method of analyzing NMR data and identifying which amino acids give rise to signals in the NMR spectrum.

Screening Methods

Another aspect of the present invention is the design and identification of agents that inhibit or potentiate an autoinhibition state or active state of an RTK. The rationale design and identification of agents can be accomplished by utilizing the structural coordinates that define a binding pocket of an RTK.

The structures described herein, and the structures of other polypeptides determined by homology modeling, molecular replacement, and NMR techniques described herein can also be applied to modulator design and identification methods.

The invention contemplates molecular models, in particular three-dimensional molecular models of RTK proteins, and their use as templates for the design of agents able to mimic or inhibit ligand activation or autophosphorylation or phoshorylation of the proteins (e.g. modulators). A modulator may inhibit or potentiate an autoinhibited state or alternatively an active state.

In certain embodiments, the present invention provides a method of screening for a ligand that associates with a binding pocket and/or modulates the function of an Eph receptor by using a crystal or a model according to the present invention. The method may involve investigating whether a test compound is capable of associating with or binding a binding pocket, and/or inhibiting or enhancing interactions of atomic contacts in a binding pocket.

In accordance with an aspect of the present invention, a method is provided for screening for a ligand capable of binding to a binding pocket, wherein the method comprises using a crystal or model according to the invention.

In another aspect, the invention relates to a method of screening for a ligand capable of binding to a binding pocket, wherein the binding pocket is defined by the structural coordinates given herein, the method comprising contacting the binding pocket with a test compound and determining if the test compound binds to the binding pocket. The binding pocket may be a binding pocket of an autoinhibited state or an active state. In the case of an autoinhibited state binding pocket the screening method may potentially identify an inhibitor that may disrupt catalytic activity of an RTK, for example, by maintaining the RTK in an autoinhibited state. A disruption of catalytic activity may be useful in the treatment of conditions involving increased RTK activity e.g. cancer.

In one embodiment, the present invention provides a method of screening for a test compound capable of interacting with one or more key amino acid residues of a binding pocket of an RTK. For example, a test compound that interacts with one or more of Tyr/Phe604, Tyr/Phe 610, Tyr 667, Tyr 744, and Tyr 750 of EphB2 receptor may prevent phosphorylation of one or more of the tyrosines and thereby promote the autoinhibited state of the receptor.

Another aspect of the invention provides a process comprising the steps of:

(a) performing a method of screening for a ligand described above;

(b) identifying one or more ligands capable of binding to a binding pocket; and

(c) preparing a quantity of said one or more ligands.

A further aspect of the invention provides a process comprising the steps of;

(a) performing a method of screening for a ligand as described above;

(b) identifying one or more ligands capable of binding to a binding pocket; and

(c) preparing a pharmaceutical composition comprising said one or more ligands.

Once a test compound capable of interacting with one or more key amino acid residues in a binding pocket of an RTK has been identified, further steps may be carried out either to select and/or modify compounds and/or to modify existing compounds, to modulate the interaction with the key amino acid residues in the binding pocket.

Yet another aspect of the invention provides a process comprising the steps of;

(a) performing the method of screening for a ligand as described above;

(b) identifying one or more ligands capable of binding to a binding pocket;

(c) modifying said one or more ligands capable of binding to a binding pocket;

(d) performing said method of screening for a ligand as described above; and

(e) optionally preparing a pharmaceutical composition comprising said one or more ligands.

In another aspect of the invention, a method of screening for a test compound is provided comprising screening for test compounds that affect (inhibit or potentiate) a juxtamembrane-juxtamembrane interaction (e.g. interactions 25 to 49 in Table 2) or juxtamembrane-kinase interactions (e.g. interactions 1 to 24 in Table 2) described herein.

As used herein, the term “test compound” means any compound which is potentially capable of associating with a binding pocket, inhibiting or enhancing interactions of atomic contacts in a binding pocket, and/or inhibiting or potentiating an autoinhibited state or active state of an RTK. If, after testing, it is determined that the test compound does bind to the binding pocket, inhibits or enhances interactions of atomic contacts in a binding pocket, and/or inhibits or potentiates an autoinhibited or active state of an RTK, it is known as a “ligand”.

The test compound may be designed or obtained from a library of compounds which may comprise peptides, as well as other compounds, such as small organic molecules and particularly new lead compounds. By way of example, the test compound may be a natural substance, a biological macromolecule, or an extract made from biological materials such as bacteria, fungi, or animal particularly mammalian) cells or tissues, an organic or an inorganic molecule, a synthetic test compound, a semi-synthetic test compound, a carbohydrate, a monosaccharide, an oligosaccharide or polysaccharide, a glycolipid, a glycopeptide, a saponin, a heterocyclic compound, a structural or functional mimetic, a peptide, a peptidomimetic, a derivatised test compound, a peptide cleaved from a whole protein, or a peptide synthesised synthetically (such as, by way of example, either using a peptide synthesizer or by recombinant techniques or combinations thereof), a recombinant test compound, a natural or a non-natural test compound, a fusion protein or equivalent thereof and mutants, derivatives or combinations thereof.

The increasing availability of biomacromolecule structures of potential pharmacophoric molecules that have been solved crystallographically has prompted the development of a variety of direct computational methods for molecular design, in which the steric and electronic properties of substrate binding sites are use to guide the design of potential ligands (Cohen et al. (1990) J. Med. Cam. 33: 883-894; Kuntz et al. (1982) J. Mol. Biol 161: 269-288; DesJarlais (1988) J. Med. Cam. 31: 722-729; Bartlett et al. (1989) (Spec. Publ., Roy. Soc. Chem.) 78: 182-196; Goodford et al. (1985) J. Med. Cam. 28: 849-857; DesJarlais et al. J. Med. Cam. 29: 2149-2153). Directed methods generally fall into two categories: (1) design by analogy in which 3-D structures of known molecules (such as from a crystallographic database) are docked to the receptor structure and scored for goodness-of-fit; and (2) de novo design, in which the ligand model is constructed piece-wise in the receptor. The latter approach, in particular, can facilitate the development of novel molecules, uniquely designed to bind to the subject receptor.

The test compound may be screened as part of a library or a data base of molecules. Modulators of inactivated/activated states of an RTK or binding pocket thereof may be identified by docking a computer representation of compounds from one or more data base of molecules. Data bases which may be used include ACD (Molecular Designs Limited), NCI (National Cancer Institute), CCDC (Cambridge Crystallographic Data Center), CAST (Chemical Abstract Service), Derwent (Derwent Information Limited), Maybridge (Maybridge Chemical Company Ltd), Aldrich (Aldrich Chemical Company), DOCK University of California in San Francisco), and the Directory of Natural Products (Chapman & Hall). Computer programs such as CONCORD (Tripos Associates) or DB-Converter (Molecular Simulations Limited) can be used to convert a data set represented in two dimensions to one represented in three dimensions.

Test compounds may tested for their capacity to fit spatially into a binding pocket. As used herein, the term “fits spatially” means that the three-dimensional structure of the test compound is accommodated geometrically in a cavity of a binding pocket. The test compound can then be considered to be a ligand.

A favourable geometric fit occurs when the surface area of the test compound is in close proximity with the surface area of the cavity of a binding pocket without forming unfavorable interactions. A favourable complementary interaction occurs where the test compound interacts by hydrophobic, aromatic, ionic, dipolar, or hydrogen donating and accepting forces. Unfavourable interactions may be steric hindrance between atoms in the test compound and atoms in the binding pocket.

If a model of the present invention is a computer model, the test compounds may be positioned in a binding pocket through computational docking. If, on the other hand, the model of the present invention is a structural model, the test compounds may be positioned in the binding pocket by, for example, manual docking.

As used herein the term “docking” refers to a process of placing a compound in close proximity with a binding pocket, or a process of finding low energy conformations of a test compound/binding pocket complex.

In an illustrative embodiment, the design of potential RTK, in particular EphB2 ligands begins from the general perspective of shape complimentarity for an active site and substrate specificity subsites of the receptor, and a search algorithm is employed which is capable of scanning a database of small molecules of known three-dimensional structure for candidates which fit geometrically into the target protein site. It is not expected that the molecules found in the shape search will necessarily be leads themselves, since no evaluation of chemical interaction need necessarily be made during the initial search. Rather, it is anticipated that such candidates might act as the framework for further design, providing molecular skeletons to which appropriate atomic replacements can be made. Of course, the chemical complimentarily of these molecules can be evaluated, but it is expected that atom types will be changed to maximize the electrostatic, hydrogen bonding, and hydrophobic interactions with the receptor. Most algorithms of this type provide a method for finding a wide assortment of chemical structures that are complementary to the shape of a binding site of the subject receptor. Each of a set of small molecules from a particular data-base, such as the Cambridge Crystallographic Data Bank (CCDB) (Allen et al. (1973) J. Chem. Doc. 13: 119), is individually docked to the binding pocket or site of an RTK, in particular an EphB2 receptor, in a number of geometrically permissible orientations with use of a docking algorithm. In a preferred embodiment, a set of computer algorithms called DOCK, can be used to characterize the shape of invaginations and grooves that form active sites and recognition surfaces of a subject receptor (Kuntz et al. (1982) J. Mol. Biol 161: 269-288). The program can also search a database of small molecules for templates whose shapes are complementary to particular binding pockets or sites of a receptor (DesJarlais et al. (1988) J Med Chem 31: 722-729). These templates normally require modification to achieve good chemical and electrostatic interactions (DesJarlais et al. (1989) ACS Symp Ser 413: 60-69). However, the program has been shown to position accurately known cofactors for ligands based on shape constraints alone.

The orientations are evaluated for goodness-of-fit and the best are kept for further examination using molecular mechanics programs, such as AMBER or CHARMM. Such algorithms have previously proven successful in finding a variety of molecules that are complementary in shape to a given binding site of a receptor, and have been shown to have several attractive features. First, such algorithms can retrieve a remarkable diversity of molecular architectures. Second, the best structures have, in previous applications to other proteins, demonstrated impressive shape complementarity over an extended surface area. Third, the overall approach appears to be quite robust with respect to small uncertainties in positioning of the candidate atoms.

Goodford (1985, J Med Chem 28:849-857) and Boobbyer et al. (1989, J Med Chem 32:1083-1094) have produced a computer program (GRID) which seeks to determine regions of high affinity for different chemical groups (termed probes) on the molecular surface of the binding site. GRID hence provides a tool for suggesting modifications to known ligands that might enhance binding. It may be anticipated that some of the sites discerned by GRID as regions of high affinity correspond to “pharmacophoric patterns” determined inferentially from a series of known ligands. As used herein, a pharmacophoric pattern is a geometric arrangement of features of the anticipated ligand that is believed to be important for binding. Attempts have been made to use pharmacophoric patterns as a search screen for novel ligands (Jakes et al. (1987) J Mol Graph 5:41-48; Brint et al. (1987) J Mol Graph 5:49-56; Jakes et al. (1986) J Mol Graph 4:12-20); however, the constraint of steric and “chemical” fit in the putative (and possibly unknown) receptor binding pocket or site is ignored. Goodsell and Olson (1990, Proteins: Struct Funct Genet 8:195-202) have used the Metropolis (simulated annealing) algorithm to dock a single known ligand into a target protein. They allow torsional flexibility in the ligand and use GRID interaction energy maps as rapid lookup tables for computing approximate interaction energies. Given the large number of degrees of freedom available to the ligand, the Metropolis algorithm is time-consuming and is unsuited to searching a candidate database of a few thousand small molecules.

Yet a further embodiment of the present invention utilizes a computer algorithm such as CLIX which searches such databases as CCDB for small molecules which can be oriented in a receptor binding pocket or site in a way that is both sterically acceptable and has a high likelihood of achieving favorable chemical interactions between the candidate molecule and the surrounding amino acid residues. The method is based on characterizing a binding pocket in terms of an ensemble of favorable binding positions for different chemical groups and then searching for orientations of the candidate molecules that cause maximum spatial coincidence of individual candidate chemical groups with members of the ensemble. The current availability of computer power dictates that a computer-based search for novel ligands follows a breadth-first strategy. A breadth-first strategy aims to reduce progressively the size of the potential candidate search space by the application of increasingly stringent criteria, as opposed to a depth-first strategy wherein a maximally detailed analysis of one candidate is performed before proceeding to the next. CLIX conforms to this strategy in that its analysis of binding is rudimentary it seeks to satisfy the necessary conditions of steric fit and of having individual groups in “correct” places for bonding, without imposing the sufficient condition that favorable bonding interactions actually occur. A ranked “shortlist” of molecules, in their favored orientations, is produced which can then be examined on a molecule-by-molecule basis, using computer graphics and more sophisticated molecular modeling techniques. CLIX is also capable of suggesting changes to the substituent chemical groups of the candidate molecules that might enhance binding.

The algorithmic details of CLIX is described in Lawerence et al. (1992) Proteins 12:31-41, and the CLIX algorithm can be summarized as follows. The GRID program is used to determine discrete favorable interaction positions (termed target sites) in the binding pocket or site of the protein for a wide variety of representative chemical groups. For each candidate ligand in the CCDB an exhaustive attempt is made to make coincident, in a spatial sense in the binding site of the protein, a pair of the candidate's substituent chemical groups with a pair of corresponding favorable interaction sites proposed by GRID. All possible combinations of pairs of ligand groups with pairs of GRID sites are considered during this procedure. Upon locating such coincidence, the program rotates the candidate ligand about the two pairs of groups and checks for steric hindrance and coincidence of other candidate atomic groups with appropriate target sites. Particular candidate/orientation combinations that are good geometric fits in the binding site and show sufficient coincidence of atomic groups with GRID sites are retained.

Consistent with the breadth-first strategy, this approach involves simplifying assumptions. Rigid protein and small molecule geometry is maintained throughout. As a first approximation rigid geometry is acceptable as the energy minimized coordinates of an RTK, in particular an EphB2 deduced structure, as described herein, describe an energy minimum for the molecule, albeit a local one. If the surface residues of the site of interest are not involved in crystal contacts then the crystal configuration of those residues is used merely as a starting point for energy minimization, and potential solution structures for those residues determined. The deduced structure described herein should reasonably mimic the mean solution configuration.

A further assumption implicit in CLIX is that the potential ligand, when introduced into the binding pocket or site of a receptor, does not induce change in the protein's stereochemistry or partial charge distribution and so alter the basis on which the GRID interaction energy maps were computed. It must also be stressed that the interaction sites predicted by GRID are used in a positional and type sense only, i.e., when a candidate atomic group is placed at a site predicted as favorable by GRID, no check is made to ensure that the bond geometry, the state of protonation, or the partial charge distribution favors a strong interaction between the protein and that group. Such detailed analysis should form part of more advanced modeling of candidates identified in the CLIX shortlist Yet another embodiment of a computer-assisted molecular design method for identifying ligands of a binding pocket of an RTK comprises the de novo synthesis of potential ligands by algorithmic connection of small molecular fragments that will exhibit the desired structural and electrostatic complementarity with an active site or binding pocket of the receptor. The methodology employs a large template set of small molecules with are iteratively pieced together in a model of an RTK active site or binding pocket. Each stage of ligand growth is evaluated according to a molecular mechanics-based energy function, which considers van der Waals and coulombic interactions, internal strain energy of the lengthening ligand, and desolvation of both ligand and receptor. The search space can be managed by use of a data tree which is kept under control by pruning according to the binding criteria In an illustrative embodiment, the search space is limited to consider only amino acids and amino acid analogs as the molecular building blocks. Such a methodology generally employs a large template set of amino acid conformations, though need not be restricted to just the 20 natural amino acids, as it can easily be extended to include other related fragments of interest to the medicinal chemist, e.g. amino acid analogs. The putative ligands that result from this construction method are peptides and peptide-like compounds rather than the small organic molecules that are typically the goal of drug design research. The appeal of the peptide building approach is not that peptides are preferable to organics as potential pharmaceutical agents, but rather that: (1) they can be generated relatively rapidly de novo; (2) their energetics can be studied by well-parameterized force field methods; (3) they are much easier to synthesize than are most organics; and (4) they can be used in a variety of ways, for peptidomimetic ligand design, protein-protein binding studies, and even as shape templates in the more commonly used 3D organic database search approach described above.

Such a de novo peptide design method has been incorporated in a software package called GROW (Moon et al. (1991) Proteins 11:314-328). In a typical design session, standard interactive graphical modeling methods are employed to define the structural environment in which GROW is to operate. For instance, environment could be an active site binding pocket of an RTK, in particular an EphB2, or it could be a set of features on the protein's surface to which the user wishes to bind a peptide-like molecule. The GROW program then operates to generate a set of potential ligand molecules. Interactive modeling methods then come into play again, for examination of the resulting molecules, and for selection of one or more of them for further refinement.

To illustrate, GROW operates on an atomic coordinate file generated by the user in the interactive modeling session, such as the coordinates provided in Table 3, or the coordinates of a binding pocket or active site as described in Table 2 and 3 plus a small fragment (e.g., an acetyl group) positioned in the active site to provide a starting point for peptide growth. These are referred to as “site” atoms and “seed” atoms, respectively. A second file provided by the user contains a number of control parameters to guide the peptide growth (Moon et al. (1991) Proteins 11:314-328).

The operation of the GROW algorithm is conceptually fairly simple. GROW proceeds in an iterative fashion, to systematically attach to the seed fragment each amino acid template in a large preconstructed library of amino acid conformations. When a template has been attached, it is scored for goodness-of-fit to the receptor site or binding pocket, and then the next template in the library is attached to the seed. After all the templates have been tested, only the highest scoring ones are retained for the next level of growth. This procedure is repeated for the second growth level; each library template is attached in turn to each of the bonded seed/amino acid molecules that were retained from the first step, and is then scored. Again, only the best of the bonded seed/dipeptide molecules that result are retained for the third level of growth. The growth of peptides can proceed in the N-to-C direction only, the reverse direction only, or in alternating directions, depending on the initial control specifications supplied by the user. Successive growth levels therefore generate peptides that are lengthened by one residue. The procedure terminates when the user-defined peptide length has been reached, at which point the user can select from the constructed peptides those to be studied further. The resulting data provided by the GROW procedure includes not only residue sequences and scores, but also atomic coordinates of the peptides, related directly to the coordinate system of the receptor site atoms.

In yet another embodiment, potential pharmacophoric compounds can be determined using a method based on an energy minimization-quenched molecular dynamics algorithm for determining energetically favorable positions of functional groups in the binding pockets of the subject receptor. The method can aid in the design of molecules that incorporate such functional groups by modification of known ligands or de novo construction.

For example, the multiple copy simultaneous search method (MCSS) described by Miranker et al. (1991) Proteins 11: 29-34 may be employed. To determine and characterize a local minima of a functional group in the forcefield of the protein, multiple copies of selected functional groups are first distributed in a binding pocket of interest on the RTK protein. Energy minimization of these copies by molecular mechanics or quenched dynamics yields the distinct local minima. The neighborhood of these minima can then be explored by a grid search or by constrained minimization. In one embodiment, the MCSS method uses the classical time dependent Hartee (TDH) approximation to simultaneously minimize or quench many identical groups in the forcefield of the protein.

Implementation of the MCSS algorithm requires a choice of functional groups and a molecular mechanics model for each of them. Groups must be simple enough to be easily characterized and manipulated (3-6 atoms, few or no dihedral degrees of freedom), yet complex enough to approximate the steric and electrostatic interactions that the functional group would have in binding to the pocket or site of interest in the RTK protein. A preferred set is, for example, one in which most organic molecules can be described as a collection of such groups (Patai's Guide to the Chemistry of Functional Groups, ed. S. Patai (New York: John Wiley, and Sons, (1989)). This includes fragments such as acetonitrile, methanol, acetate, methyl ammonium, dimethyl ether, methane, and acetaldehyde.

Determination of the local energy minima in the binding pocket or site requires that many starting positions be sampled. This can be achieved by distributing, for example, 1,000-5,000 groups at random inside a sphere centered on the binding site; only the space not occupied by the protein needs to be considered. If the interaction energy of a particular group at a certain location with the protein is more positive than a given cut-off (e.g. 5.0 kcal/mole) the group is discarded from that site. Given the set of starting positions, all the fragments are minimized simultaneously by use of the TDH approximation (Elber et al. (1990) J Am Chem Soc 112: 9161-9175). In this method, the forces on each fragment consist of its internal forces and those due to the protein. The essential element of this method is that the interactions between the fragments are omitted and the forces on the protein are normalized to those due to a single fragment. In this way simultaneous minimization or dynamics of any number of functional groups in the field of a single protein can be performed.

Minimization is performed successively on subsets of, for example 100, of the randomly placed groups. After a certain number of step intervals, such as 1,000 intervals, the results can be examined to eliminate groups converging to the same minimum. This process is repeated until minimization is complete (e.g. RMS gradient of 0.01 kcal/mole/C). Thus the resulting energy minimized set of molecules comprises what amounts to a set of disconnected fragments in three dimensions representing potential pharmacophores.

The next step then is to connect the pharmacophoric pieces with spacers assembled from small chemical entities (atoms, chains, or ring moieties). In a preferred embodiment, each of the disconnected can be linked in space to generate a single molecule using such computer programs as, for example, NEWLEAD (Tschinke et al. (1993) J Med Chem 36: 3863, 3870). The procedure adopted by NEWLEAD executes the following sequence of commands (1) connect two isolated moieties, (2) retain the intermediate solutions for further processing, (3) repeat the above steps for each of the intermediate solutions until no disconnected units are found, and (4) output the final solutions, each of which is a single molecule. Such a program can use for example, three types of spacers: library spacers, single-atom spacers, and fuse-ring spacers. The library spacers are optimized structures of small molecules such as ethylene, benzene and methylamide. The output produced by programs such as NEWLEAD consist of a set of molecules containing the original fragments now connected by spacers. The atoms belonging to the input fragments maintain their original orientations in space. The molecules are chemically plausible because of the simple makeup of the spacers and functional groups, and energetically acceptable because of the rejection of solutions with van-der Waals radii violations.

A screening method of the present invention may comprise the following steps:

(i) generating a computer model of a binding pocket using a crystal according to the invention;

(ii) docking a computer representation of a test compound with the computer model;

(iii) analysing the fit of the compound in the binding pocket.

In an aspect of the invention, a method is provided comprising the following steps:

(a) docking a computer representation of a structure of a test compound into a computer representation of a binding pocket of an RTK defined in accordance with the invention using a computer program, or by interactively moving the representation of the test compound into the representation of the binding pocket;

(b) characterizing the geometry and the complementary interactions formed between the atoms of the binding pocket and the compound; optionally

(c) searching libraries for molecular fragments which can fit into the empty space between the compound and the binding pocket and can be linked to the compound; and

(d) linking the fragments found in (c) to the compound and evaluating the new modified compound.

In an embodiment of the invention, a method is provided which comprises the following steps:

(a) docking a computer representation of a test compound from a computer data base with a computer representation of a selected binding pocket on an RTK defined in accordance with the invention to define a complex;

(b) determining a conformation of the complex with a favorable fit and favourable complementary interactions; and

(c) identifying test compounds that best fit the selected binding pocket as potential modulators of the RTK.

In another embodiment of the invention, a method is provided which comprises docking a computer representation of a selected binding pocket of an RTK defined by the atomic interactions, atomic contacts, or structural coordinates in accordance with the invention to define a complex. In particular a method is provided comprising:

(a) docking a computer representation of a test compound from a computer database with a computer representation of a selected binding pocket of an RTK defined by the atomic interactions, atomic contacts, or structural coordinates described herein;

(b) determining a conformation of the complex with a favorable fit and favourable complementary interactions; and

(c) identifying test compounds that best fit the selected binding pocket as potential modulators of the RTK.

A model used in a screening method may comprise a binding pocket either alone or in association with one or more ligands and/or cofactors. For example, the model may comprise the binding pocket in association with a nucleotide (or analogue thereof), a substrate (or analogue thereof), and/or modulator.

If the model comprises an unassociated binding pocket, then the selected site under investigation may be the binding pocket itself. The test compound may, for example, mimic a known ligand (e.g. nucleotide or substrate) for an RTK in order to interact with the binding pocket The selected site may alternatively be another site on the RTK.

If the model comprises an associated binding pocket, for example a binding pocket in association with a ligand, the selected site may be the binding pocket or a site made up of the binding pocket and the complexed ligand, or a site on the ligand itself. The test compound may be investigated for its capacity to modulate the interaction with the associated molecule.

The screening methods described herein may be applied to a plurality of test compounds, to identify those that best fit the selected site. The screening methods may be used to identify a modulator that changes an autoinhibited state of an RTK to an active state, or an active state to an autoinhibited state.

A test compound (or plurality of test compounds) may be selected on the basis of their similarity to a known ligand for an RTK, in particular an Eph receptor. For example, the screening method may comprise the following steps:

(i) generating a computer model of a binding pocket in complex with a ligand;

(ii) searching for a test compound with a similar three dimensional structure and/or similar chemical groups as the ligand; and

(iii) evaluating the fit of the test compound in the binding pocket

Searching may be carried out using a database of computer representations of potential compounds, using methods known in the art.

The present invention also provides a method for designing ligands for RTKs. It is well known in the art to use a screening method as described above to identify a test compound with promising fit, but then to use this test compound as a starting point to design a ligand with improved fit to the model. Such techniques are known as “structure-based ligand design” (See Kuntz et al., 1994, Acc. Chem. Res. 27:117; Guida, 1994, Current Opinion in Struc. Biol. 4: 777; and Colman, 1994, Current Opinion in Struc. Biol. 4: 868, for reviews of structure-based drug design and identification; and Kuntz et al 1982, J. Mol. Biol. 162:269; Kuntz et al., 1994, Acc. Chem. Res. 27: 117; Meng et al., 1992, J. Compt. Chem. 13: 505; Bohm, 1994, J. Comp. Aided Molec. Design 8: 623 for methods of structure-based modulator design).

Examples of computer programs that may be used for structure-based ligand design are CAVEAT (Bartlett et al., 1989, in “Chemical and Biological Problems in Molecular Recognition”, Roberts, S. M. Ley, S. V.; Campbell, N. M. eds; Royal Society of Chemistry: Cambridge, pp 182-196); FLOG (Miller et al., 1994, J. Comp. Aided Molec. Design 8:153); PRO Modulator (Clark et al., 1995 J. Comp. Aided Molec. Design 9:13); MCSS (Miranker and Karplus, 1991, Proteins: Structure, Fuction, and Genetics 8:195); and, GRID (Goodford, 1985, J. Med. Chem. 28:849).

The method may comprise the following steps:

(i) docking a model of a test compound with a model of a binding pocket;

(ii) identifying one or more groups on the test compound which may be modified to improve their fit in the binding pocket;

(iii) replacing one or more identified groups to produce a modified test compound model; and

(iv) docking the modified test compound model with the model of the binding pocket.

Evaluation of fit may comprise the following steps:

(a) mapping chemical features of a test compound such as by hydrogen bond donors or acceptors, hydrophobic/lipophilic sites, positively ionizable sites, or negatively ionizable sites; and

(b) adding geometric constraints to selected mapped features.

The fit of the modified test compound may then be evaluated using the same criteria.

The chemical modification of a group may either enhance or reduce hydrogen bonding interaction, charge interaction, hydrophobic interaction, Van Der Waals interaction or dipole interaction between the test compound and the key amino acid residue(s) of the binding pocket. Preferably the group modifications involve the addition removal or replacement of substituents onto the test compound such that the substituents are positioned to collide or to bind preferentially with one or more amino acid residues that correspond to the key amino acid residues of the binding pocket.

If a modified test compound model has an improved fit, then it may bind to a binding pocket and be considered to be a “ligand”. Rational modification of groups may be made with the aid of libraries of molecular fragments which may be screened for their capacity to fit into the available space and to interact with the appropriate atoms. Databases of computer representations of libraries of chemical groups are available commercially, for this purpose.

The test compound may also be modified “in situ” (i.e. once docked into the potential binding pocket), enabling immediate evaluation of the effect of replacing selected groups. The computer representation of the test compound may be modified by deleting a chemical group or groups, or by adding a chemical group or groups. After each modification to a compound, the atoms of the modified compound and potential binding pocket can be shifted in conformation and the distance between the modulator and the binding pocket atoms may be scored on the basis of geometric fit and favourable complementary interactions between the molecules. This technique is described in detail in Molecular Simulations User Manual, 1995 in LUDI.

Examples of ligand building and/or searching computer programs include programs in the Molecular Simulations Package (Catalyst), ISIS/HOST, ISIS/BASE, and ISIS/DRAW (Molecular Designs Limited), and UNITY (Tripos Associates).

The “starting point” for rational ligand design may be a known ligand for the enzyme. For example, in order to identify potential modulators of an RTK, in particular an Eph receptor, a logical approach would be to start with a known ligand (for example a nucleotide or known kinase inhibitors) to produce a molecule which mimics the binding of the ligand. Such a molecule may, for example, act as a competitive inhibitor for the true ligand, or may bind so strongly that the interaction (and inhibition) is effectively irreversible.

Such a method may comprise the following steps:

(i) generating a computer model of a binding pocket in complex with a ligand;

(ii) replacing one or more groups on the ligand model to produce a modified ligand; and

(iii) evaluating the fit of the modified ligand in the binding pocket.

The replacement groups could be selected and replaced using a compound construction program which replaces computer representations of chemical groups with groups from a computer database, where the representations of the compounds are defined by structural coordinates.

In an embodiment, a screening method is provided for identifying a ligand of an RTK, in particular an Eph receptor, comprising the step of using the structural coordinates of a nucleotide or component thereof, defined in relation to its spatial association with a binding pocket of the invention, to generate a compound that is capable of associating with the binding pocket In an embodiment of the invention, a screening method is provided for identifying a ligand of an RTK, in particular an Eph receptor, comprising the step of using the structural coordinates of adenosine adenine, or ATP listed in Table 3 to generate a compound for associating with a binding pocket of RTK, in particular an Eph receptor as described herein. The following steps are employed in a particular method of the invention: (a) generating a computer representation of adenosine adenine, or ATP, defined by its structural coordinates listed in Table 3; (b) searching for molecules in a data base that are structurally or chemically similar to the defined adenosine adenine, or ATP, using a searching computer program, or replacing portions of the adenosine adenine, or ATP with similar chemical structures from a database using a compound building computer program.

A screening method is provided for identifying a ligand of an RTK, in particular an Eph receptor, comprising the step of using the structural coordinates of a binding pocket comprising a juxtamembrane region or part thereof listed in Table 3 to generate a compound for associating with a kinase domain of an RTK, in particular an Eph receptor. The following steps are employed in a particular method of the invention: (a) generating a computer representation of a binding pocket comprising a juxtamembrane region or part thereof defined by its structural coordinates listed in Table 3; and (b) searching for molecules in a data base that are structurally or chemically similar to the defined binding pocket using a searching computer program, or replacing portions of the binding pocket with structures from a database using a compound building computer program.

The screening methods of the present invention may be used to identify compounds or entities that associate with a molecule that associates with an RTK, in particular an Eph receptor (for example, a nucleotide).

Compounds and entities (e.g. ligands) of RTKs, in particular Eph receptors, identified using the above-described methods may be prepared using methods described in standard reference sources utilized by those skilled in the art. For example, organic compounds may be prepared by organic synthetic methods described in references such as March, 1994, Advanced Organic Chemistry: Reactions, Mechanisms, and Structure, New York, McGraw Hill.

Test compounds and ligands which are identified using a crystal or model of the present invention can be screened in assays such as those well known in the art. Screening may be for example in vitro, in cell culture, and/or in vivo. Biological screening assays preferably centre on activity-based response models, binding assays (which measure how well a compound binds to a binding pocket of a receptor), and bacterial, yeast, and animal cell lines (which measure the biological effect of a compound in a cell). The assays may be automated for high throughput screening in which large numbers of compounds can be tested to identify compounds with the desired activity. The biological assay may also be an assay for the binding activity of a compound that selectively binds to the binding pocket compared to other receptors.

Ligands/Compounds Identified by Screening Methods

The present invention provides a ligand or compound identified by a screening method of the present invention. A ligand or compound may have been designed rationally by using a model according to the present invention. A ligand or compound identified using the screening methods of the invention may specifically associate with a target compound, or part thereof (e.g. a binding pocket). In the present invention the target compound may be the RTK (e.g. Eph receptor) or part thereof, or a molecule that is capable of associating with the RTK or part thereof (for example a nucleotide). In an embodiment, the ligand is capable of binding to phosphoregulatory sites of a binding pocket, in particular phosphoregulatory sites of a juxtamembrane region or kinase domain. In another embodiment, the ligand is capable of binding to the activation segment of a kinase domain of an Eph receptor.

A ligand or compound identified using a screening method of the invention may act as a “modulator”, i.e. a compound which affects the activity of an RTK in particular an Eph receptor. A modulator may reduce, enhance or alter the biological function of an RTK, in particular an Eph receptor. For example a modulator may modulate the capacity of the RTK to autophosphorylate. An alteration in biological function may be characterised by a change in specificity. For example, a modulator may cause the RTK to accept a different nucleotide, to phosphorylate a different amino acid residue, or to work with a different metal cofactor. A modulator may dispose an RTK to favor the autoinhibited state or active state. In order to exert its function, the modulator commonly binds to a binding pocket.

A “modulator” which is capable of reducing the biological function of the enzyme may also be known as an inhibitor. Preferably an inhibitor reduces or blocks the capacity of the enzyme to autophosphorylate. An inhibitor may promote the autoinhibition state of an RTK. The inhibitor may mimic the binding of a nucleotide or substrate, for example, it may be a nucleotide or substrate analogue. A nucleotide analogue may be designed by considering the interactions between the nucleotide and the RTK (for example, by using information derivable from the crystal of the invention) and specifically altering one or more groups (as described above).

The present invention also provides a method for modulating the activity of an RTK, in particular an Eph receptor, using a modulator according to the present invention. The invention also provides a method for inhibiting autophosphorylation of an RTK, preferably an Eph receptor, by potentiating the autoinhibition state of an RTK, or inhibiting the active state of the RTK. Inhibition of phosphorylation of an RTK may decrease signaling by the RTK and inhibit cellular processes that may be involved in disease. It would be possible to monitor receptor activity following such treatments by a number of methods known in the art.

A modulator may be an agonist, partial agonist, partial inverse agonist or antagonist of an RTK.

As used herein, the term “agonist” means any ligand, which is capable of binding to a binding pocket and which is capable of increasing a proportion of the receptor that is in an active form, resulting in an increased biological response. The term includes partial agonists and inverse agonists.

As used herein, the term “partial agonist” means an agonist that is unable to evoke the maximal response of a biological system, even at a concentration sufficient to saturate the specific receptors.

As used herein, the term “partial inverse agonist” is an inverse agonist that evokes a submaximal response to a biological system, even at a concentration sufficient to saturate the specific receptors. At high concentrations, it will diminish the actions of a full inverse agonist.

As used herein, the term “antagonist” means any agent that reduces the action of another agent, such as an agonist. The antagonist may act at the same site as the agonist (competitive antagonism). The antagonistic action may result from a combination of the substance being antagonised (chemical antagonism) or the production of an opposite effect through a different receptor (functional antagonism or physiological antagonism) or as a consequence of competition for the binding site of an intermediate that links receptor activation to the effect observed (indirect antagonism).

As used herein, the term “competitive antagonism” refers to the competition between an agonist and an antagonist for a binding pocket of a receptor that occurs when the binding of agonist and antagonist becomes mutually exclusive. This may be because the agonist and antagonist compete for the same binding sites or pockets, or combine with adjacent but overlapping sites. A third possibility is that different sites are involved but that they influence the receptor macromolecules in such a way that agonist and antagonist molecules cannot be bound at the same time. If the agonist and antagonist form only short lived combinations with a binding pocket of a receptor so that equilibrium between agonist, antagonist and receptor is reached during the presence of the agonist, the antagonism will be surmountable over a wide range of concentrations. In contrast, some antagonists, when in close enough proximity to their binding site, may form a stable covalent bond with it and the antagonism becomes insurmountable when no spare receptors remain.

As mentioned above, an identified ligand or compound may act as a ligand model (for example, a template) for the development of other compounds. A modulator may be a mimetic of a ligand.

Like the test compound (see above) a modulator may be one or a variety of different sorts of molecule. (See examples herein.) A modulator may be an endogenous physiological compound, or it may be a natural or synthetic compound. The modulators of the present invention may be natural or synthetic. The term “modulator” also refers to a chemically modified ligand or compound.

The technique suitable for preparing a modulator will depend on its chemical nature. For example, peptides can be synthesized by solid phase techniques (Roberge J Y et al (1995) Science 269: 202-204) and automated synthesis may be achieved, for example, using the ABI 43 1 A Peptide Synthesizer (Perlin Elmer) in accordance with the instructions provided by the manufacturer. Once cleaved from the resin, the peptide may be purified by preparative high performance liquid chromatography (e.g., Creighton (1983) Proteins Structures and Molecular Principles, W H Freeman and Co, New York N.Y.). The composition of the synthetic peptides may be confirmed by amino acid analysis or sequencing (e.g., the Edman degradation procedure; Creighton, supra).

If a modulator is a nucleotide, or a polypeptide expressable therefrom, it may be synthesized, in whole or in part, using chemical methods well known in the art (see Caruthers M H et al (1980) Nuc Acids Res Symp Ser 215-23, Horn T et al (1980) Nuc Acids Res Symp Ser 225-232), or it may be prepared using recombinant techniques well known in the art.

Organic compounds may be prepared by organic synthetic methods described in references such as March, 1994, Advanced Organic Chemistry: Reactions, Mechanisms, and Structure, New York, McGraw Hill.

The invention also relates to classes of modulators of RTKs based on the structure and shape of a nucleotide, or component thereof, or a substrate or component thereof; defined in relation to the nucleotide's or substrate's spatial association with a crystal structure of the invention or part thereof.

A class of modulators may comprise a compound containing a structure of adenine, adenosine, ribose, pyrophosphate, or ATP, and having one or more, preferably all, of the structural coordinates of adenine, adenosine, ribose, pyrophosphate, or ATP of Table 4. Functional groups in the adenine, adenosine, ribose, pyrophosphate, or ATP modulators may be substituted with, for example, alkyl, alkoxy, hydroxyl, aryl, cycloalkyl, alkenyl, alkynyl, thiol, thioalkyl, thioaryl, amino, or halo, or they may be modified using techniques known in the art.

Another class of modulators defined by the invention are compounds comprising an adenine triphosphate group having the structural coordinates of adenine triphosphate in the active site binding pocket of an Eph receptor.

The invention contemplates all optical isomers and racemic forms of the modulators of the invention.

Pharmaceutical Composition

The present invention also provides for the use of a modulator according to the invention, in the manufacture of a medicament to treat and/or prevent a disease in a mammalian patient. There is also provided a pharmaceutical composition comprising such a modulator and a method of treating and/or preventing a disease comprising the step of administering such a modulator or pharmaceutical composition to a subject, preferably a mammalian patient.

The pharmaceutical compositions may be for human or animal usage in human and veterinary medicine and will typically comprise a pharmaceutically acceptable carrier, diluent, excipient, adjuvant or combination thereof.

Acceptable carriers or diluents for therapeutic use are well known in the pharmaceutical art, and are described, for example, in Remington's Pharmaceutical Sciences, Mack Publishing Co. (A. R1 Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s), solubilising agent(s).

Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may also be used.

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, vaginal, epidural, sublingual.

Where the pharmaceutical composition is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by inhalation, in the form of a suppository or pessary, topically in the form of a lotion, gel, hydrogel, solution, cream, ointment or dusting powder, by use of a skin patch, orally in the form of tablets containing excipients such as starch or lactose or chalk, or in capsules or ovules either alone or in admixture with excipients, or in the form of elixirs, solutions or suspensions containing flavouring or colouring agents, or they can be injected parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

If the agent of the present invention is administered parenterally, then examples of such administration include one or more of intravenously, intra-arterially, intraperitoneally, intrathecally, intraventricularly, intraurethrally, intrastemally, intracranially, intramuscularly or subcutaneously administering the agent; and/or by using infusion techniques.

For buccal or sublingual administration the compositions may be administered in the form of tablets or lozenges which can be formulated in a conventional manner.

The tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia. Additionally, lubricating agents such as magnesium stearate, stearic acid, glyceryl behenate and talc may be included.

Solid compositions of a similar type may also be employed as fillers in gelatin capsules. Preferred excipients in this regard include lactose, starch, cellulose, milk sugar or high molecular weight polyethylene glycols. For aqueous suspensions and/or elxirs, the agent may be combined with various sweetening or flavouring agents, colouring matter or dyes, with emulsifying and/or suspending agents and with diluents such as water, ethanol, propylene glycol and glycerin, and combinations thereof.

As indicated, a therapeutic agent (e.g. modulator) of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134 μm) or 1,1,1,2,3,3,3-heptafluoropropane (HFA 227EA™), carbon dioxide or other suitable gas. In the case of a pressurised aerosol, the dosage unit may be determined by providing a valve to deliver a metered amount The pressurised container, pump, spray or nebuliser may contain a solution or suspension of the active compound, e.g. using a mixture of ethanol and the propellant as the solvent, which may additionally contain a lubricant, e.g. sorbitan trioleate. Capsules and cartridges (made, for example, from gelatin) for use in an inhaler or insufflator may be formulated to contain a powder mix of the agent and a suitable powder base such as lactose or starch.

Therapeutic administration of polypeptide modulators may also be accomplished using gene therapy. A nucleic acid including a promoter operatively linked to a heterologous polypeptide may be used to produce high-level expression of the polypeptide in cells transfected with the nucleic acid. DNA or isolated nucleic acids may be introduced into cells of a subject by conventional nucleic acid delivery systems. Suitable delivery systems include liposomes, naked DNA, and receptor-mediated delivery systems, and viral vectors such as retroviruses, herpes viruses, and adenoviruses.

Applications

The invention provides a method for inhibiting kinase activity of an RTK comprising maintaining the RTK or a binding pocket thereof involved in regulating the kinase domain in an autoinhibited state, or potentiating an autoinhibited state for the RTK or binding pocket thereof involved in regulating the kinase domain. An autoinhibited state may be maintained or potentiated by inhibiting phosphorylation of phosphoregulatory sites of the juxtamembrane segment and/or kinase domain (e.g. activation segment). Inhibition may be accomplished using modulators, or altering the structure of a binding pocket of the RTK comprising the phosphoregulatory sites, to prevent phosphorylation of the sites.

The invention contemplates a method for altering the stability of an autoinhibited state of an RTK comprising phosphorylating phosphoregulatory sites of a juxtamembrane region of the RTK.

In an aspect the invention relates to a method for changing an RTK from an autoinhibited state to an active state comprising phosphorylating phosphoregulatory sites of a juxtamembrane region of the RTK.

In another aspect the invention provides a method for activating kinase activity of an RTK comprising phosphorylating phosphoregulatory sites of a juxtamembrane region and kinase domain (e.g. activation segment) of the RTK.

The invention further provides a method of treating a mammal, the method comprising administering to a mammal a modulator or pharmaceutical composition of the present invention.

In particular, the invention contemplates a method of treating or preventing a condition or disease associated with an RTK in a cellular organism, comprising:

(c) administering a modulator of the invention in an acceptable pharmaceutical preparation; and

(d) activating or inhibiting the RTK to treat or prevent the disease.

In an aspect the invention provides a method for treating or preventing a condition or disease involving increased RTK activity comprising maintaining the RTK or a binding pocket thereof involved in regulating the kinase domain of the RTK in an autoinhibited state. An autoinhibited state may be maintained as described herein. In an embodiment the condition or disease is cancer.

The invention provides for the use of a modulator identified by the methods of the invention in the preparation of a medicament to treat or prevent a disease in a cellular organism. Use of modulators of the invention to manufacture a medicament is also provided.

Typically, a physician will determine the actual dosage of a modulator or pharmaceutical composition of the invention that will be most suitable for an individual subject and it will vary with the age, weight and response of the particular patient and severity of the condition. There can, of course, be individual instances where higher or lower dosage ranges are merited.

The specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. By way of example, the pharmaceutical composition of the present invention may be administered in accordance with a regimen of 1 to 10 times per day, such as once or twice per day.

For oral and parenteral administration to human patients, the daily dosage level of the agent may be in single or divided doses.

The modulators and compositions of the invention may be useful in the prevention and treatment of conditions involving aberrant RTKs.

Conditions which may be prevented or treated in accordance with the invention include but are not limited to lymphoproliferative conditions, malignant and pre-malignant conditions, arthritis, inflammation, and autoimmune disorders. Malignant and pre-malignant conditions may include solid tumors, B cell lymphomas, chronic lymphocytic leukemia, chronic myelogenous leukemia, prostate hypertrophy, Hirschsprung disease, glioblastoma, breast and ovarian cancer, adenocarcinoma of the salivary gland, premyelocytic leukemia, prostate cancer, multiple endocrine neoplasia type IIA and IIB, medullary thyroid carcinoma, papillary carcinoma, papillary renal carcinoma, hepatocellular carcinoma, gastrointestinal stromal tumors, sporadic mastocytosis, acute myeloid leukemia, large cell lymphoma or Alk lymphoma, chronic myeloid leukemia, hematological/solid tumors, papillary thyroid carcinoma, stem cell leukemia/lymphoma syndrome, acute myelogenous leukemia, osteosarcoma, multiple myeloma, preneoplastic liver foci, and resistance to chemotherapy. Diseases associated with increased cell survival, or the inhibition of apoptosis, include cancers (e.g. follicular lymphomas, carcinomas with p53 mutations, hormone-dependent tumors such as breast cancer, prostate cancer, Kaposi's sarcoma and ovarian cancer); autoimmune disorders (such as lupus erythematosus and immune-related glomerulonephritis rheumatoid arthritis) and viral infections (such as herpes viruses, pox viruses, and adenoviruses); inflammation, graft vs. host disease, acute graft rejection and chronic graft rejection.

Eph receptors and ephrins mediate contact-dependent repulsive guidance of migrating cells and axons in culture and in vivo. Many Eph family members are prominently expressed in the developing nervous system, and epbrin stimulation of growing primary axons in vitro results in axonal retraction or repulsion, characterized by a collapse of actin-rich growth cone structures at the leading edge of the cell. Mice bearing homozygous null mutations in EphA8 or in both EphB2 and EphB3 exhibit abnormal migration of axon tracts in the brain. Ephrin-induced retraction of exploratory actin filopodia has also been described in vivo in migrating Eph receptor-expressing neural crest cells.

The Eph receptors and ephrins have also been implicated in cell sorting and boundary formation. Eph-receptor signaling is able to modulate both cell-cell and cell-substrate attachment. Bidirectional Eph receptor-ephrin signaling is important for the formation of boundaries between rhombomeres of the hind brain. These cellular responses to Eph receptor stimulation indicate that they may regulate signaling events which control cytoskeletal architecture and cell adhesion functions.

Therefore, modulators of Eph receptors may be used to modulate axonogenesis, nerve cell interactions and regeneration, to treat conditions such as neurodegenerative diseases and conditions involving trauma and injury to the nervous system, for example Alzheimer's disease, Parkinson's disease, Huntington's disease, demylinating diseases, such as multiple sclerosis, amyotrophic lateral sclerosis, bacterial and viral infections of the nervous system, deficiency diseases, such as Wernicke's disease and nutritional polyneuropathy, progressive supranuclear palsy, Shy Drager's syndrome, multistem degeneration and olivo ponto cerebellar atrophy, peripheral nerve damage, trauma and ischemia resulting from stroke.

Therapeutic efficacy and toxicity of compositions and modulators of the invention may be determined by standard pharmaceutical procedures in cell cultures or with experimental animals, such as by calculating the ED₅₀ (the dose therapeutically effective in 50% of the population) or LD₅₀ (the dose lethal to 50% of the population) statistics. The therapeutic index is the dose ratio of therapeutic to toxic effects and it can be expressed as the ED₅₀/LD₅₀ ratio. Pharmaceutical compositions that exhibit large therapeutic indices are preferred.

The invention will now be illustrated by the following non-limiting example:

### EXAMPLE

The following methods were used in the investigation described in the example:

Methods

Protein Expression and Purification

Mutagenesis of the juxtamembrane tyrosines (Y604/61° F.) of murine EphB2 was performed using a PCR-based approach. The amplified cDNA sequence, corresponding to the receptor's juxtamembrane region and kinase domain (residues 595-906), was cloned into pGEX-4T-1 (Pharmacia). The glutathione-S transferase (GST)-EphB2 construct was transformed into Escherichia coli B834 cells and the cells grown in minimal media supplemented with selenomethionine, with overnight induction at 15° C., and 0.15 mM IPTG (isopropyl-β-D-thiogalactopyranoside, BioShop). Cells were lysed by homogenization and sonication in 25 mM HEPES (pH 7.5), 50 mM NaCl, 20% glycerol, 2 mM DTT, 2 mM phenyl-methyl sulphonyl fluoride. Purification of the selenomethionyl derivative of EphB2 was performed as previously described (Binns et al., 2000), with the exception that the buffer used for gel filtration (buffer C) was 10 mM HEPES (pH 7.5), 50 mM NaCl, 1 mM DTT.

Crystallization, Data Collection, and Structure Determination

Hanging drops containing 1 μl of 12.5 mg/ml protein in buffer C were mixed with equal volumes of reservoir buffer containing 0.1 M HEPES (pH 7.0), 0.2 M magnesium chloride, 10% (w/v) PEG 4000, 10% (v/v) isopropanol, and 15% (v/v) ethylene glycol. Rod-like crystals were obtained overnight at 28° C. after streak seeding with smaller crystals obtained initially. The crystals belong to primitive space group P2₁, (a=47.86 Å, b=98.09 Å, c=68.18 Å, α=γ=90°, β=104.97°), with two molecules of EphB2 in the asymmetric unit. Crystals were flash frozen by immersion in liquid nitrogen. A MAD experiment was performed on a frozen crystal at APS beamline BM 14-D (λ1=0.9790 Å, λ2=0.9788 Å, λ3=0.9770 Å) using a Quantum 4 ADSC CCD detector. Data processing and reduction was carried out with the HKL program suite (Otwinowski and Minor, 1997). The programs SHARP (La Fortelle and Bricogne, 1997) and SnB (Miller et al., 1994) were used in combination to locate and subsequently refine positions for 22 of the possible 30 Se sites. Following density modification with Solomon (Abrahams and Leslie, 1996), a partial model was generated using 0 (Jones et al., 1991) and refined using CNS (Brunger et al., 1998) (R-factors>40%). Consequently, crystals of EphB2 in complex with 2 μM AMP-PNP were grown as described above (space group —P1, a=47.05 Å, b=57.62 Å, c=67.74 Å, α=112.95°, β=103.170, γ=91.58°), with two molecules per asymmetric unit Diffraction data was collected to 1.9 Å at APS beamline BM 14-C (λ=1.00 Å) using a Quantum 4 ADSC CCD detector and processed with the HKL program suite. Molecular replacement solutions were determined with AMoRe (Navaza, 1994; CCP4, 1994), using one monomer of the P2₁-derived model as a search molecule. The two AMoRe solutions, which correspond to the two EphB2 molecules in the asymmetric unit, refined readily in CNS. With minimal modification to the starting model, the model has been refined to a working R value of 24.1% and a free R value of 27.7%. As defined in PROCHECK (Laskowski et al., 1993), 90.8% of protein residues are in the most favored regions of the Ramachandran plot, with none in the disallowed regions. Pertinent statistics for data collection and refinement are shown in Table 1.

Mutagenesis

The cDNA sequence of the juxtamembrane region and kinase domain of murine EphA4 (amino acids 591-896), corresponding to residues 599-906 of murine EphB2, was cloned into pGEX-4T-2 (Pharmacia). The murine EphB2 numbering scheme was employed, and the corresponding EphA4 residue numbers are listed in parentheses. Using a PCR-based approach, Tyr 604 (Tyr596) and Tyr 610 (Tyr602) were mutated to phenylalanine. The following site-directed mutants were then generated using this doubly mutated construct: (1) ΔJXₐₗₗ; deletion of 599-621 (591-613), (2) ΔJX1; deletion of 599-606 (591-598), (3) ΔJX1+2; deletion of 599-610 (591-602), (4) Pro607Gly (Pro599Gly), (5) Phe608Asp (Phe600Asp), (6) Phe620Asp (Phe612Asp), (7) Ser680Trp (Ser672Trp), (8) ΔJX1+2 plus Phe620Asp, and (9) Tyr604/610 Glu (Tyr596/602Glu). The GST-EphA4 constructs were transformed into E. coli BL21 codon plus cells and grown in LB supplemented with ampicillin, with overnight induction at 15° C., 0.15 mM IPTG. Purification was performed as described for EphB2. The mutations Tyr604Phe, Tyr610Phe, Pro607Gly, Phe620Asp, Ser680Trp, Gln684Trp, deletion of 599-606 (ΔJX1), deletion of 599-610 (ΔJX1+2), and deletion of 600-621 (ΔJX1ₐₗₗ) in murine EphB2 were generated by site-directed mutagenesis using overlapping oligonucleotide primers containing the above indicated point mutations or deletions. All mutations were confirmed by DNA sequencing.

Western Blotting

GST-EphA4 proteins expressed in E. coli (BL21 codon plus), and EphB2 proteins transiently expressed in COS-1 cells, were harvested as previously described (Binns et al, 2000; Holland et al, 1997). Proteins were resolved using 12% denaturing polyacrylamide gel electrophoresis (PAGE), transferred onto a polyvinylidene difluoride membrane (Millipore), blotted with anti-pTyr (Upstate Biotechnology), anti-GST (Santa Cruz Laboratories), or anti-EphB2 antibodies (Holland et al., 1997), and visualized using enhanced chemiluminescence (ECL Plus; Amersham).

In Vitro Kinase Reactions

In vitro kinase reactions using GST fusion EphA4 proteins bound to glutathione sepharose or immunoprecipitated EphB2 proteins transiently expressed in COS-1 cells were performed with 5 μg and 2 μg of acid-denatured enolase, respectively, and 5 μCi of [γ³²P]ATP at room temperature as previously described (Binns et al., 2000).

Spectrophotometric Coupling Assay

Kinetic analysis of the bacterial expressed EphA4 proteins was performed using a coupled in vitro spectrophotometric kinase assay where production of ADP is coupled to the oxidation of NADH through pyruvate kinase and lactic dehydrogenase (Barker et al., 1995; Binns et al, 2000). The 100-μl reaction volume contained 1 U lactic dehydrogenase, 1 U pyruvate kinase, 1 mM phosphoenolpyruvate, 0.2 mM NADH, and 0.5 mM ATP (in 20 mM MgCl₂, 0.1 mM DTT, 60 mM HEPES [pH 7.5], 20 μg/mL bovine serum albumin). Wild type and mutant EphA4 activity was measured by monitoring absorbance at 340 nm (Varian UV-Visible spectrophotometer) for 90 minutes at a fixed enzyme concentration (0.5 μM) and 1 mM S-1 synthetic peptide (GEEIYGEFD; amide at carboxy terminus) concentrations. For accuracy, protein concentrations were determined by UV spectrometry at 280 nm using molar extinction coefficients. (Andersson, 1998; Collaborative Computational Project, 1994).

Results and Discussions

Structure Determination

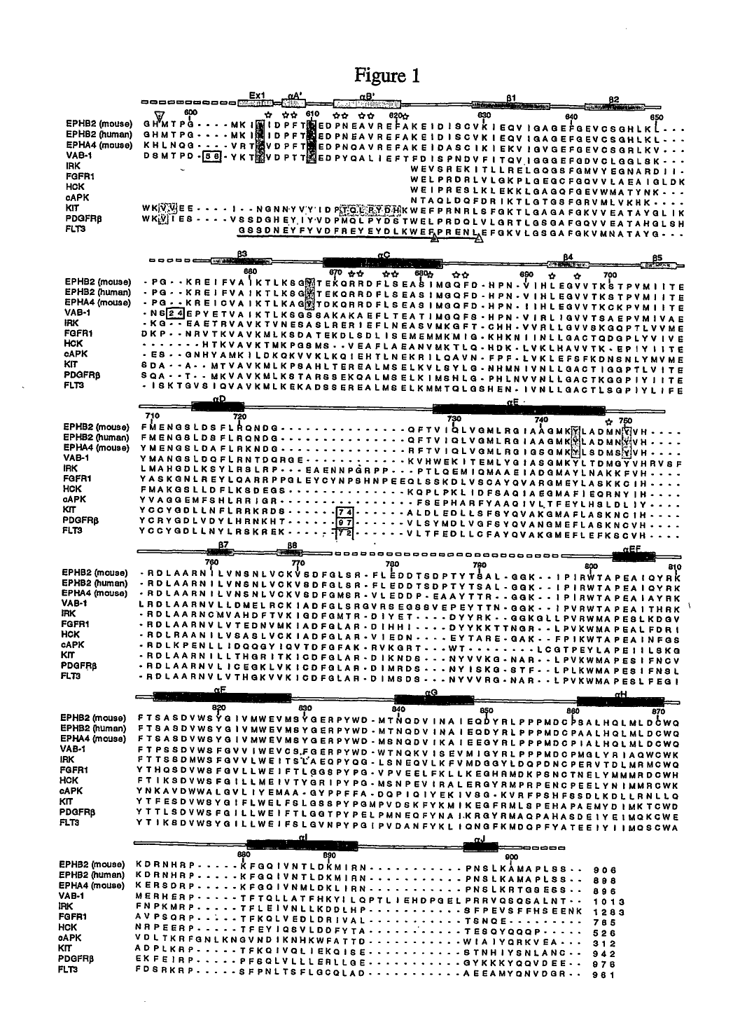

Since the expression of active EphB2 polypeptides in E. coli is toxic, efforts were focused on the catalytically repressed Tyr 604/610 Phe double mutant. For the purposes of discussion, these sites are referred to as Tyr/Phe 604 and Tyr/Phe 610. A cytoplasmic fragment (residues 595 to 906) of the murine EphB2 RTK consisting of the latter half of the juxtamembrane region and the entire catalytic domain was expressed as a GST fusion in E. coli and purified to homogeneity (see Methods). The predicted boundaries of the juxtamembrane region are residues 573-620, while those of the kinase and SAM domains are residues 621-892 and residues 919-994, respectively. Protein crystals of two different space groups were grown and the EphB2 structure was determined using a combination of seleno-methionine multiwavelength anomalous dispersion (SeMet MAD) and molecular replacement (MR) methods (see Methods). The EphB2 crystal structure reported here corresponds to the juxtamembrane-catalytic domain fragment in complex with AMP-PNP (β,γ-imidoadenosine-5′-triphosphate). Overall, the EphB2 structure is well ordered except for the first seven and last six amino acid residues, kinase domain residues 651 to 653 connecting β-strands 2 and 3 of the N-terminal catalytic lobe, and residues 774 to 796 corresponding to the kinase activation segment within the C-terminal lobe. Only the adenine ring of AMP-PNP is ordered in experimental and model based electron density maps, and hence the sugar and phosphate groups have not been modeled. Data collection and refinement statistics are listed in Table 1 and a representative alignment of the EphB2 receptor and other protein kinase family members is provided in FIG. 1.

Overall Description of the Autoinhibited Structure

The structure of the catalytic domain of EphB2 conforms to that generally observed for protein kinases, consisting of two lobes, a smaller N-terminal lobe and larger C-terminal lobe (FIGS. 2a,b). Protein kinases are capable of a range of conformations owing to an inherent inter-lobe flexibility that allows for both open and closed conformations. However, the catalytically competent conformation is generally a closed structure in which the two catalytic lobes clamp together to form an interfacial nucleotide binding site and catalytic cleft. Surprisingly, the autoinhibited EphB2 catalytic domain adopts a closed conformation that resembles an ‘active’ state.

The N-terminal lobe of protein kinases consists minimally of a twisted 5-strand β-sheet (denoted β1 to β5 as first described for the cAMP dependent protein kinase (cAPK) and a single helix αC (Knighton et al., 1991). The N-terminal lobe functions to assist in the binding and coordination of ATP for the productive transfer of the y-phosphate to a substrate oriented by the C-terminal lobe. In this regard, β-strands 1 and 2 and the glycine rich connecting segment (g-loop) form a flexible flap that interacts with the adenine base, ribose sugar and the non-hydrolyzable phosphate groups of ATP. Furthermore, an invariant salt bridge between a lysine side chain (sub-domain 2 in the protein kinase nomenclature of Hanks et al., 1988) in β-strand 3 and a glutamic acid side chain (sub-domain 3) in helix αC coordinates the β-phosphate of ATP. In the EphB2 crystal structure, all N-terminal lobe elements implicated in nucleotide binding are well ordered and adopt a prototypical protein kinase arrangement. However, distortions in helix αC and the g-loop arising from interactions with the juxtamembrane segment are evident.

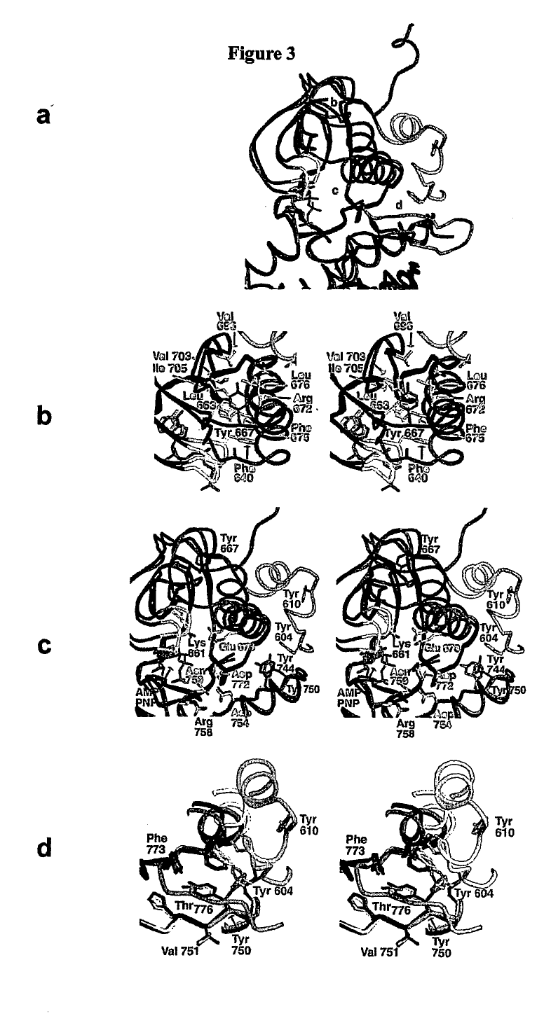

The C-terminal lobe of protein kinases consists minimally of two β-strands (β7 and β8) and a series of α-helices (αD to αI). Strands β7 and β8 locate to the cleft region between the N- and C-terminal lobes where they contribute side chains that participate in catalysis and the binding of magnesium for the coordination of ATP phosphate groups. In the EphB2 crystal structure, all lower lobe residues implicated in catalysis and ATP coordination appear optimally oriented (FIG. 3c). The activation segment, which is also located in the large catalytic lobe, is disordered as in several other protein kinase structures in which the activation segment is not phosphorylated (reviewed by Johnson et al., 1996). The remaining C-terminal lobe elements, including α-helices αD to αI, are well ordered and adopt the prototypical protein kinase configuration. Terminating the catalytic domain structure is a short helix αJ.

The EphB2 juxtamembrane region preceding the catalytic domain is highly ordered and adopts an identical conformation in the four unique environments sampled in the two different crystal forms studied. From the amino-terminus, the conformation consists of an extended strand segment Ex1, a single turn 3/10 helix αA′, and a four-turn helix αB′. These elements associate intimately with helix αC of the N-terminal catalytic lobe and also make limited interactions with the C-terminal lobe. As a consequence of the association of the juxtamembrane segment with the N-terminal kinase lobe, significant curvature is imposed on helix αC. This distortion couples directly to local distortions in other N-terminal lobe elements, most critically the g-loop and the invariant lysine-glutamate salt bridge. Together the N-terminal lobe distortions appear to impinge on catalytic function by adversely affecting the coordination of the sugar and phosphate groups of the bound nucleotide.

With limited contacts to the lower lobe of the catalytic domain, the juxtamembrane segment also sterically impedes the activation segment from adopting the productive conformation that typifies the active state of protein-serine/threonine and tyrosine kinases. Together, the effects on nucleotide coordination and the activation segment form the basis for autoinhibition of EphB2 by the juxtamembrane segment.

Depending on the splice variant of EphB2, there are 29-45 juxtamembrane residues between the start of strand Ex1 (Lys 602) and the plasma membrane (Connor and Pasquale, 1995). This relatively lengthy sequence makes it impossible to predict whether the autoinhibited structure observed here would be oriented in a specific fashion with respect to the inner surface of the membrane.

Detailed Analysis of Juxtamembrane Structure

The juxtamembrane strand segment Ex1, corresponding to amino acid residues Lys 602 to lie 605, extends along the cleft region between the N- and C-terminal lobes (FIGS. 2c,d). The phosphoregulatory residue Tyr/Phe 604 orients into a solvent-exposed hydrophobic pocket composed of the side chains of Met 748 and Tyr 750 of the C-terminal kinase lobe, Ile 681 and Phe 685 from helix αC and Pro 607 from the juxtamembrane helix αA′. This site has been termed ‘switch region 1’ since Tyr/Phe 604 appears well placed to influence the association of the juxtamembrane region with the catalytic domain. Further stabilizing the interaction of strand Ex1 with the lower catalytic lobe are hydrogen bonds between the amide group of Tyr/Phe 604 and the carbonyl group of Met 748 and between the side chain of Gln 684 and the backbone amide and carbonyl groups of lie 605.

Helix αA′ is composed of a single rigid turn initiated by an Asp606Pro607 sequence and terminated by Thr 609. This helix appears stabilized by the conformational rigidity of Pro 607 and the capping interactions involving the side chains of Asp 606 and Thr 609 with the free backbone amino group and carbonyl groups of Phe 608 and Asp 606. A short linker and then a three-turn helix αB′, initiated by Asp 612Pro613 and extending to Phe 620, follow helix αA′. Helix αB′ is also initiated by an Asp Pro sequence (residues 612 and 613) and Asp 612 makes similar capping interactions with the backbone amino and side chain of Asn 614. Helices αA′ and αB′ form an interface with the N-terminal lobe of the kinase that centers on helix αC. Hydrophobic side chains projecting from αA′ and αB′ include Pro 607, Phe 608, Pro 613, Val 617, Phe620 and Ala 621. These residues associate intimately with Arg 673, Leu 676, and Ile 681 from helix αC and Leu 693 and Val 696 from α-strand 4. In addition, a hydrogen bond interaction (2.9 Å) is observed between Asn 614 and Arg 672 (FIG. 2c), and the small side chains at positions 616 (Ala), 677 (Ser) and 680 (Ser) facilitate the close packing of helices αA′, αB′ and αC.

Opposite to, but contiguous with, the site of association with helix αC, strand Ex1 and helices αA′ and αB′ form an interface composed primarily of hydrophobic interactions. The side chain of the phosphoregulatory residue Tyr/Phe 610 projects onto the surface of this site and appears well positioned to exert an influence on the local juxtamembrane structure. This interface, termed ‘switch region 2’, is composed of the side chains of Ile 605 from strand Ex1 and the side chains of Ala 616 and Phe 620 from helix αB′.

Effect of the Juxtamembrane Engagement on the N-Terminal Lobe Structure

Comparison of the EphB2 crystal structure with that of the ‘active’ triply phosphorylated insulin receptor tyrosine kinase (active IRK (Hubbard, 1997)) indicates the mechanism by which the juxtamembrane region of EphB2 inhibits the catalytic domain. Superposition of C-terminal kinase lobe elements places the majority of N-terminal lobe elements into close correspondence (FIGS. 3a-d). A distinguishing feature of the EphB2 structure is a 14° kink midway along helix αC centered at Glu 678. This kink, which coincides with the site of association with the juxtamembrane elements Ex1, αA′ and αB′, displaces the forward facing N-terminus of helix αC 6.8 Å upward and outward from the equivalent position observed in IRK (FIGS. 3a,c). Stabilizing this kink internally are side chain/main chain interactions involving Ser 677 and Ser 680.

The kink in helix αC places its forward projecting terminus in close proximity to β-strands 3, 4, and 5, forming a tighter interface than that observed in active IRK (FIG. 3b). Residues participating in this interface include Arg 672, Phe 675, and Leu 676 from helix αC, Tyr 667 from the β3/αC linker and Leu 663, Val 696, Thr 698, Val 703, and Ile 705 from the β-strands. Interestingly, tyrosine 667, which is centrally positioned within this interface and is highly conserved amongst the Eph receptor family members, has been identified as an in vivo site of phosphorylation (Kalo and Pasquale, 1999), suggesting a possible phosphoregulatory role.

The close association of helix αC with β-strands 3, 4 and 5 is achieved with a local alteration to the twist of the forward projecting termini of β-strands 1, 2 and 3 that leaves the bulk of the N-terminal sheet structure unperturbed. The g-loop side chain Phe 640 plays a role in coupling the β-strand movements to that of helix αC through a direct interaction with Phe 675. The altered twist of the β1, β2 and β3-strand termini displace main chain atoms at the end of the g-loop (Glu 639 and Phe 640) by approximately 3.3 Å. In addition, together with the kink in helix αC, the altered twist of the β-strands displaces the invariant glutamate and lysine side chains by 2.4 and 2.1 Å, respectively, relative to their positions in active IRK (FIG. 3c). As a consequence, the ability of the catalytic domain to coordinate the sugar and phosphate groups of bound nucleotide is compromised (FIGS. 3a-c). Since the domain closure and the bulk of the N-terminal β-sheet structure is not perturbed, the adenine binding pocket is well formed and indeed the adenine base of bound AMP-PNP is ordered and orients in a manner similar to that in the crystal structure of active IRK.

Steric Influence of the Juxtamembrane Region on the Activation Segment

While the majority of interactions between the juxtamembrane segment and the catalytic domain are directed towards the N-terminal lobe, strand Ex1 forms a limited set of interactions with the C-terminal lobe that may serve a regulatory role. Superposition of EphB2 with active IRK illustrates how the side chain of the phosphoregulatory residue Tyr/Phe 604 impedes the activation segment from adopting a productive conformation (FIG. 3d). In autoinhibited EphB2, the side chain of Tyr 750 adopts an alternate conformation from that of the corresponding residue Phe 1128 in active IRK. This avoids a steric clash with the side chain of Tyr/Phe604. The alternate conformation of Tyr 750, in turn, impedes the activation segment from adopting a path observed in active IRK due to a steric clash with Ser 776 (Thr 1154 in IRK). Interestingly, the side chain conformation of Tyr 750 in EphB2, Tyr 382 in Src and Hck, and Phe 1128 in IRK all correlate with their activation segments adopting non-productive conformations. This may be indicative of a more general function in protein kinases for position 750 in regulating the conformation of the activation segment.

The Phosphoregulatory Switch

The ability to oscillate between catalytically active and repressed states in a regulated manner is the key to the function of protein kinases as versatile molecular switches. In EphB2, EphA4, and most likely Eph RTKs in general, the switch to an active state is coordinated by phosphorylation at highly conserved sites within both the juxtamembrane region and the catalytic domain. The mechanism by which phosphorylation at sites within the activation segment stimulate protein kinases is relatively well understood (reviewed by Johnson et al., 1996) and by inference, phosphorylation of EphB2 at Tyr 788 likely promotes the ordering of the activation segment to a catalytically competent conformation.

In contrast, phosphorylation at Tyr/Phe 604 and 610 may serve to destabilize the juxtamembrane structure and cause it to dissociate from the catalytic domain. This would allow for a return of the N-terminal lobe to an undistorted active conformation.

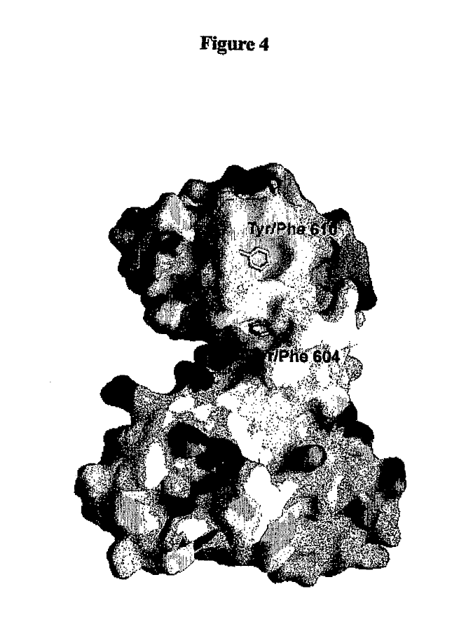

The EphB2 crystal structure helps to explain how phosphorylation at each of the two phosphoregulatory sites could destabilize the juxtamembrane structure and cause its release from the catalytic domain. The environment around each of the two switch regions is hydrophobic, but solvent exposed, and thus could accommodate either tyrosine or phenylalanine at positions 604 and 610 with little or no reorganization of the juxtamembrane structure. However, substitution with phosphotyrosine appears less tolerable due to steric and electrostatic clashes involving the bulky anionic phosphate group. In ‘switch region 1’, the phosphorylation of Tyr/Phe 604 would place a phosphate group within van der Waals contact of Asp 606, Pro 607 and Ile 681. Furthermore, the side chain of Asp 606 dominates the electrostatic environment around Tyr/Phe 604 such that the introduction of a phosphate group would generate repulsive electrostatic forces (FIG. 4). The electrostatic environment around ‘switch region 2’ is also dominated by negatively charged amino acids, namely Asp 606, Glu 611, Asp 612, Glu 615, and Glu 619. Thus, phosphorylation of Tyr 610 would also generate repulsive electrostatic forces, which are likely essential for the expulsion of this residue from its binding pocket since a phosphate group could be accommodated sterically.

Three other highly conserved tyrosine residues have been identified as in vivo phosphorylation sites in EphB2 and EphB5, namely tyrosines 667, 744 and 750 (FIG. 3c). Although their roles in regulating Eph receptor kinase activity have not been probed by mutagenesis, all three sites appear well positioned to influence the stability of the autoinhibited structure and hence Eph receptor activity (FIG. 3). For example, phosphorylation of Tyr 667 could promote a catalytically competent state by destabilizing the tight association of helix αC with β-strands 3, 4 and 5 observed in the autoinhibited state. In addition, phosphorylation of Tyr 744 and Tyr 750, which line the cleft region through which the juxtamembrane strand Ex1 navigates, could amplify the effect of phosphorylation at Tyr 604.

Function of the EphA4 Juxtamembrane Segment Probed by Mutagenesis

Previously, a cytoplasmic fragment of the EphA4 receptor tyrosine kinase, consisting of the juxtamembrane segment, the catalytic domain and the SAM domain, has been shown to require autophosphorylation for maximal activation (Binns et al., 2000). The importance of autophosphorylation was revealed by a lag period at the start of in vitro kinase reactions employing the dephosphorylated form of the EphA4 enzyme. This lag period was greatly reduced by pre-incubation of the EphA4 fragment with ATP or by deletion of the entire juxtamembrane segment. In contrast, mutation to phenylalanine of either Tyr 604 or Tyr 610 reduced the specific activity of the enzyme, while mutation of both sites in tandem drastically impaired catalytic function (<10% relative to WT). These results are consistent with the mechanism of autoinhibition suggested by the EphB2 crystal structure.

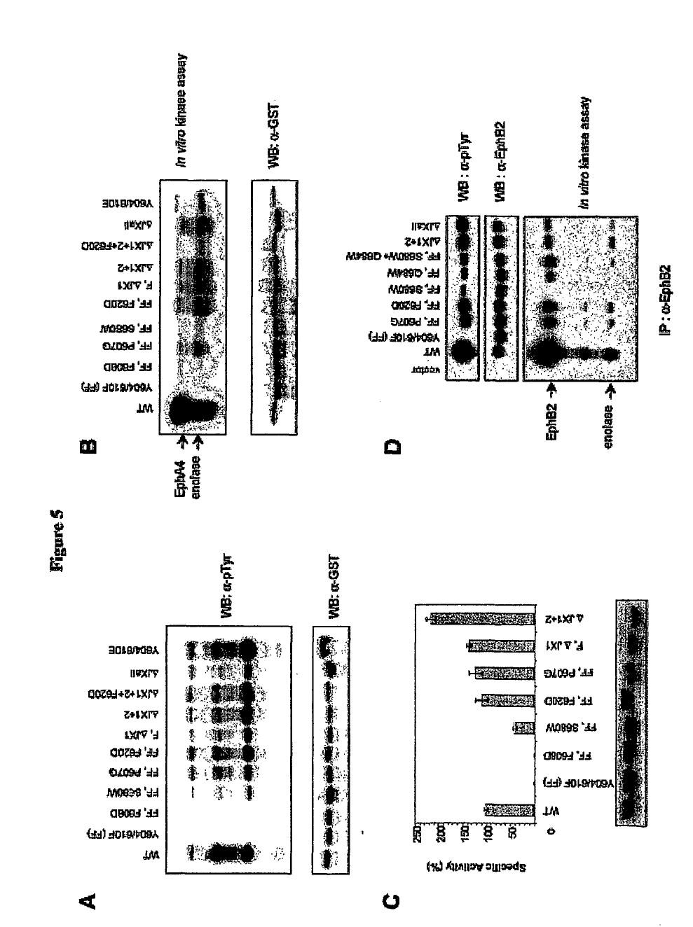

In order to test the crystallographic findings and to probe the regulation of Eph receptor catalytic activity in more detail, additional site-directed mutations were generated in the full-length murine EphB2 receptor expressed in COS-1 cells and in a murine EphA4 receptor fragment expressed in bacteria, corresponding in content to the EphB2 construct used for the structure determination. For the sake of discussion, the murine EphB2 numbering scheme has been employed for all mutants and the corresponding EphA4 residue numbers are listed in parentheses. Each mutation was generated in the catalytically repressed Tyr 604/610 Phe double mutant background and was tested for its ability to restore catalytic function. The mutations include a small N-terminal deletion of residues 595 to 606 (ΔJX1) encompassing strand Ex1 and the first phosphoregulatory site, an intermediate N-terminal deletion of residues 599 to 610 (ΔJX1+2) that encompasses strand Ex1, the first phosphoregulatory site, helix αA′ and the second phosphoregulatory site, and a full juxtamembrane segment deletion of residues 599 to 621 (ΔJXₐₗₗ). In addition, six separate point mutations were generated in both the juxtamembrane region and the kinase domain (Pro607Gly, Phe608Asp, Phe620Asp, Tyr604/610Glu, Ser680Trp, Gln684Trp) that were predicted to destabilize the interaction of the kinase domain with the juxtamembrane segment. Lastly, the ΔJX1+2 mutation was combined with the Phe620Asp mutation (ΔJX1+2 plus Phe620Asp) and the Ser680Trp mutation was combined with the Gln684Trp mutation (Ser680Trp/Gln684Trp). The Tyr604/610Phe double mutant and the wild type proteins were analyzed concomitantly as reference points for the fully repressed (0%) and active (100%) states, respectively. The activities of the EphA4 proteins expressed in bacteria were tested for their ability to induce protein tyrosine phosphorylation in vivo (FIG. 5a), and to autophosphorylate and to phosphorylate enolase in vitro (FIG. 5b). EphA4 proteins were also tested for their ability to phosphorylate a peptide substrate using a continuous spectophotometric assay (FIG. 5c). Lastly, full-length EphB2 proteins expressed in COS-1 cells were tested for their ability to autophosphorylate in vivo and to autophosphorylate and phosphorylate enolase in vitro (FIG. 5d).

The two partial N-terminal juxtamembrane deletions when introduced into the EphA4 construct significantly increased kinase activity in all four assays, restoring catalytic function as measured by the spectrophotometric assay to 136% and 216% of wild-type activity in the case of ΔJX1 and the ΔJX1+2 deletions, respectively. A similar effect was observed for the ΔJX1+2 deletion introduced into full-length EphB2.

Mutation of Phe 608 in EphA4, which locates to helix αA′, gave very weak restoration of catalytic function. This result is consistent with the variability of position 608 amongst the Eph receptor family members (42% identity). In contrast, mutation in both EphA4 and EphB2 constructs of the highly conserved Pro 607 (95% identity), which initiates helix αA′, to Gly greatly enhanced catalytic function in all four assays, quantitated at 122% of wild-type activity by the spectrophotometric assay. This result is consistent with a role for Pro607, suggested by the crystal structure, in stabilizing helix αA′ by imposing conformational rigidity, or in promoting the association of juxtamembrane and N-terminal kinase lobe elements through hydrophobic interactions. Similarly, mutation of the highly conserved Phe 620 (95% identity) at the terminus of helix aB′ to Asp also restored catalytic function in the four assays tested. Phe 620 is notable because it contributes to the hydrophobic pocket into which the phosphoregulatory residue Tyr/Phe 610 binds; its substitution with Asp is predicted to disrupt the hydrophobic interaction with Tyr/Phe 610, and to clash electrostatically with the surrounding negatively charged groups in a manner mimicking phosphorylation of Tyr/Phe 610.

The introduction of point mutations into the kinase domain at the interface with the juxtamembrane region also restored catalytic function. Mutation of Ser680 (82% identity) to Trp in both EphA4 and EphB2 constructs gave modest restoration with the phosphorylation of peptide substrate being restored to 41% of wild-type activity. Mutation of the absolutely conserved Gln684 (100% identity) to Trp in EphB2 resulted in a greater increase in kinase activity, as did the double mutation Ser 680Trp/Gln684Trp. Both mutations map to helix αC and are predicted to sterically perturb the association of the juxtamembrane region with the N-terminal catalytic lobe.

Robust restoration of activity was also observed for the EphA4 and EphB2 mutants ΔJXₐₗₗ, Tyr604/610Glu, and ΔJX1+2 plus Phe620Asp, although the relative restoration as measured by the various assays differed to a small degree. The restoration of activity by the ΔJX1 mutant confirms that the juxtamembrane segment is not absolutely required for kinase function, the restoration by the Tyr604/610 Glu mutation suggests that the addition of negative charges at positions 604 and 610 is an important component of juxatmembrane destabilization and the relief of autoinhibition. Lastly, the finding that none of the EphB2 mutants are as active as the wild-type enzyme may indicate that these mutants have perturbed some aspect of the oligomerization event that is needed for maximal activation of the full-length receptor.

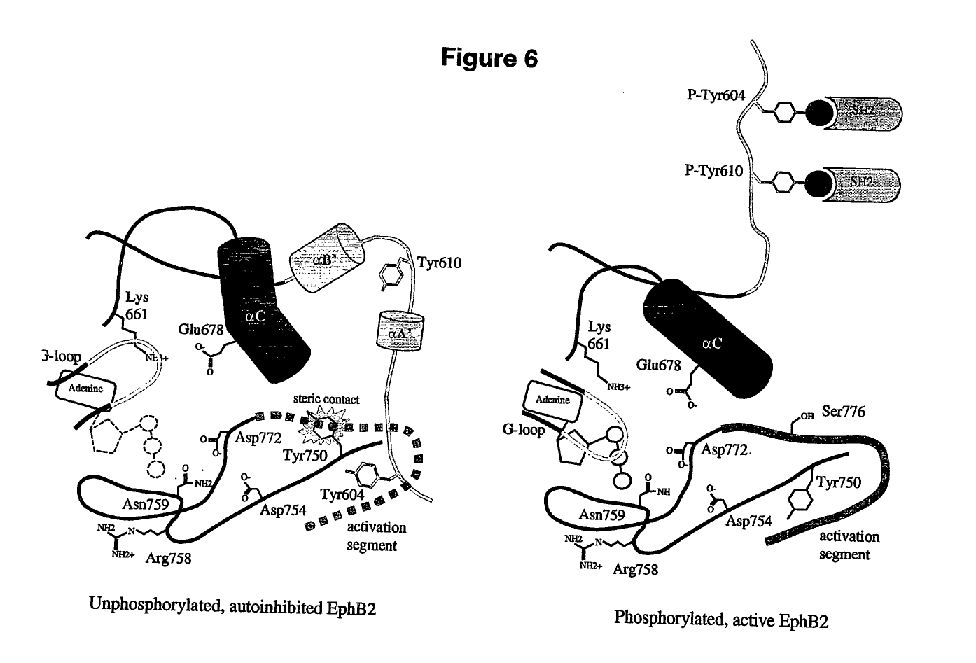

Overall, the mutagenesis results support a model for the regulation of receptor catalytic function by the juxtamembrane segment, shown in FIG. 6. Strand Ex1 and helix αA′ of the juxtamembrane segment contribute to the inhibitory effect on the catalytic domain, and the release of these elements from their association with the catalytic domain is a requirement for catalytic activation. Physiologically, this would be accomplished by phosphorylation at the Tyr 604 and 610 regulatory sites and potentially at additional sites. The strong conservation of residues involved in the inhibitory interaction suggests that this regulatory mechanism is conserved for all Eph receptor family members.

Comparison of Autoinhibitory Mechanisms of EphB2 and TGFβR1 Receptor Kinase

Analysis of the TGFβR1 serine/threonine kinase has revealed a role for the juxtamembrane Gly/Ser/Thr-rich motif (“GS segment”) in regulating catalytic activity. As with Eph receptor tyrosine kinases, TGFβR1 kinases require phosphorylation at sites within the juxtamembrane segment for subsequent phosphorylation of target Smad proteins (Macias-Silva et al, 1996). The regulatory mechanism revealed by the X-ray crystal structure of a cytoplasmic fragment of TGFβR1 in complex with FKBP12 (Huse et al, 1999) shows some parallels to EphB2. In both structures, the intramolecular engagement of the juxtamembrane segment induces conformational distortions in the catalytic domain that impinge on kinase function. In addition, the induced distortions impact on the relative positioning and/or conformation of helix αC. Beyond these similarities, however, the inhibitory mechanisms, including the mode of juxtamembrane association with the catalytic domain and the resulting basis for inhibition, diverge. Perhaps the most significant difference relates to the potential involvement of FKBP12 in stabilizing the inhibited structure of TGFβR1, whereas EphB2 achieves an autoinhibited state independently. Nonetheless, the data for EphB2 indicate that receptor tyrosine kinases and receptor serine/threonine kinases have in some cases converged on a related regulatory mechanism in which the juxtamembrane region inhibits the kinase domain in the inactive state, and is potentially liberated to interact with downstream targets upon autophosphorylation.

Discussion

Why does EphB2 employ a rather complex mechanism of autoregulation, involving the non-catalytic juxtamembrane region? One possible benefit may be to block any potential signaling activity intrinsic to the juxtamembrane sequence. In particular, phosphorylation of tyrosines 604 and 610 in EphB2 creates docking sites for SH2 domain proteins. Sequestering these tyrosines decreases their chance of becoming adventitiously phosphorylated and thereby inappropriately transmitting a signal through the recruitment of downstream targets. The coordination of kinase activation with the release of binding sites for targets is reminiscent of Src family cytoplasmic tyrosine kinase, in which the SH2 and SH3 domains engage internal ligands in a fashion that both inhibits the activity of the kinase domain and hinders interactions of the SH2 and SH3 domains with other binding partners (Sicheri et al., 1997; Xu et al., 1997).

The involvement of the juxtamembrane sequence in autoregulation of EphB2 activity may also set a phosphorylation threshold that must be exceeded to induce receptor activation. Full stimulation of Eph receptors apparently requires autophosphorylation at multiple sites within both the activation segment and juxtamembrane region. The use of at least two distinct phosphoregulatory steps may preclude inappropriate Eph receptor activation resulting from basal levels of kinase activity. Since Eph receptors have powerful biological activities during embryogenesis and postnatally, their aberrant activation would be expected to have severe phenotypic consequences, which could be avoided by requiring multi-site phosphorylation of the receptor.

Are the Eph receptors unique among RTKs in employing cytoplasmic elements outside the catalytic domain to regulate kinase activity? A variety of data obtained for the platelet-derived growth factor β receptor (PDGFR), the closely related colony stimulating factor-1 receptor (c-Fms), stem cell factor receptor (Kit), and the Flt3 receptor raise the possibility that this may in fact be a more widespread phenomenon. Biochemical analysis and mutagenesis of the PDGFR-β has suggested that autophosphorylation of juxtamembrane tyrosines 579 and 581 is required for stimulation of receptor kinase activity by PDGF, potentially by allowing subsequent phosphorylation of tyrosine 857 in the activation segment (Baxter et al., 1998). Conversion of these juxtamembrane tyrosines to phenylalanine inhibits receptor activation, while their phosphorylation creates a binding site for the Src SH2 domain, resulting in Src recruitment to the receptor. Thus, autophosphorylation within the juxtamembrane region of the PDGFR-β may couple receptor activation to the exposure of SH2 domain-binding sites, as appears to be the case for Eph receptors. Consistent with the notion that the juxtamembrane region of the PDGFR-β exerts an inhibitory influence on kinase activity, substitution of a valine residue, just N-terminal to the regulatory tyrosines, results in constitutive receptor activation in vitro and in vivo (Irusta and DiMaio, 1998). In addition to the PDGFR-β, the juxtamembrane regions of c-Fms (Myles et al., 1994), Kit, and Flt3 receptors have been implicated in regulation of tyrosine kinase activity. Oncogenic variants of Kit identified in human and murine mast cell leukemias carry either amino acid substitutions or deletions in the juxtamembrane region, which result in constitutive activation of the kinase domain (Tsujimura et al., 1996)(see FIG. 1). Remarkably a majority of human gastrointestinal stromal tumors (GIST) have activating Kit mutations that introduce substitutions or deletions into a short segment of the juxtamembrane region, and are strongly implicated in the etiology of these tumors (Hirota et al, 1998; Nakahara et al; 1998; Anderson, 1998). Furthermore, approximately 20% of acute myeloid leukemias have internal tandem duplications of Flt3 that create in-frame insertions of variable length in the juxtamembrane region, leading to ligand-independent kinase activity and oncogenic acitvation (Nakao et al, 1996; Yokota et al, 1997; Hayakawa et al, 2000). Thus, Kit and Flt3 juxtamembrane regions may repress kinase activity, and juxtamembrane mutations that relieve this inhibition can result in human cancers.

A similar situation may pertain for the insulin receptor, which upon activation becomes autophosphorylated within the juxtamembrane region and consequently binds targets such as IRS-1 and ShcA, which possess PTB domains. Kinetic analysis of wild type and mutant insulin receptors has suggested that the insulin receptor juxtamembrane region acts as an intrasteric inhibitor to block the kinase domain active site, in a fashion that is relieved by autophosphorylation of juxtamembrane tyrosines (Cann et al., 2000).

Many RTKs have C-terminal tails that upon activation become phosphorylated at SH2/PTB domain-binding sites. Structural analysis of the Tie2/Tek receptor cytoplasmic region has indicated that in the inactive state the tail interacts with the kinase domain in a way that partially occludes the C-terminal tyrosines and the peptide binding site (Shewchuk et al., 2000). This raises the possibility that autophosphorylation of the Tie2 tail causes a conformational change that exposes both C-terminal phosphotyrosine sites as well as the substrate binding site of the kinase domain.

Thus the juxtamembrane and C-terminal segments of RTKs may play a pivotal role in regulating the kinase domain, and in coordinating enzymatic activation with the exposure of motifs that bind cytoplasmic targets.

In addition to revealing an unexpected level of complexity in the regulation of RTKs, these observations have interesting implications for the design of RTK inhibitors. The structure of the Ab1 kinase bound to the inhibitor STI-571 suggests that this compound binds selectively to the inactive form of the kinase (Schindler et al., 2000). The unusual structure of autoinhibited EphB2 suggests the possibility of isolating inhibitors that bind specifically to the inactive conformation of the kinase. Indeed, if this mode of intrasteric regulation is a more common feature of RTKs, this might be a general strategy for the identification of selective RTK inhibitors.

The structure of EphB2 reveals an entirely novel mechanism for RTK autoregulation.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in chemistry, biology or related fields are intended to be within the scope of the following claims.

TABLE 1
Data collection, structure determination and refinement statistics

| | SeMet MAD Analysis | | | Native |
|---|---|---|---|---|
| | λ₁ = 0.9790 Å | λ₂ = 0.9788 Å | λ₃ = 0.9770 Å | (AMP-PNP) |
| Spacegroup | P2₁ | P2₁ | P2₁ | P1 |
| Resolution (Å) | 2.3 | 2.3 | 2.3 | 1.9 |
| Reflections total/unique | 185034/52252 | 188170/52750 | 186452/52855 | 80035/43865 |
| Completeness (%)* | 97.5 (91.2) | 97.6 (91.7) | 97.6 (90.8) | 90.3 (55.9) |
| Rsym (%)*^{†} | 7.4 (26.2) | 7.5 (27.9) | 8.5 (38.7) | 3.3 (14.6) |
| <I/σ>* | 15.6 (4.0) | 16.1 (3.9) | 14.9 (3.1) | 20.9 (4.1) |
| Phasing Power^{‡} | 0/2.59 | 2.71/4.11 | 2.08/3.40 | — |
| (ISO/ANO) | | | | |

Refinement (AMP-PNP complex)

| | | | |
|---|---|---|---|
| Resolution Range (Å) | 30-1.9 | Average B value (Å²) | 24.9 |
| Reflections | | Rmsd for B values (Å²) | 1.42 |
| all data | 42903 | Rmsd for bonds (Å) | 0.007 |
| \|F\|>2σ | 39931 | Rmsd for angles (°) | 1.09 |
| R_{factor}/R_{free}(%)** | | Number of non-hydrogen protein atoms | 4311 |
| all data | 24.1/27.7 | Number of non-hydrogen nucleotide atoms | 20 |
| \|F\|>2σ | 23.2/26.9 | Number of water molecules | 263 |

*Numbers given in parentheses refer to data for the highest resolution shell. † Rsym = 100 × Σ|I-<I>|/Σ<I>, where I is the observed intensity and <I> is the average intensity from multiple observations of symmetry-related reflections. ‡ Phasing power for isomorphous and anomalous acentric reflections, where P_{power} = <[|Fₕ(calc)/phase-integrated lack of closure]>.
**Free R-value was calculated with 8.7% of the data.

TABLE 2
Intermolecular contacts of the Juxtamembrane Region
and Kinase Domain of an Eph Receptor

| No. | | Kinase | | |
|---|---|---|---|---|
| of | | Domain/ | | |
| Atom- | | Juxtamembrane | Distance | |
| ic | Juxtamembrane | Region | Between | Atomic |
| Inter- | Region | Atomic | Atomic | Interaction |
| action | Atomic Contact | Contact | Contacts | Property |
| 1 | Phe/Tyr 604 CB | Met 748 CE | 4.58 | hydrophobic |
| 2 | Phe/Tyr 604 N | Met 748 O | 2.83 | H-bond |
| 3 | Phe/Tyr 604 CD2 | Tyr 750 CD1 | 3.78 | Hydrophobic |
| 4 | Phe/Tyr 604 CE2 | Tyr 750 CE1 | 4.12 | Hydrophobic |
| 5 | Phe/Tyr 604 CD1 | Phe 685 CE2 | 4.06 | Hydrophobic |
| 6 | Ile 605 N | Gln 684 OE1 | 2.83 | H-bond |
| 7 | Ile 605 O | Gln 684 NE2 | 3.00 | H-bond |
| 8 | Phe/Tyr 604 CE1 | Gln 684 CD | 4.11 | van der Waal |
| 9 | Pro 607 CD | Ile 681 CG1 | 3.85 | Hydrophobic |
| 10 | Pro 607 CB | Ser 680 OG | 3.16 | van der Waal |
| 11 | Phe 608 CZ | Asp 674 OD1 | 3.27 | van der Waal |
| 12 | Phe 608 CZ | Ser 677 CB | 4.35 | van der Waal |
| 13 | Phe 608 CE2 | Arg 673 CG | 3.79 | Hydrophobic |
| 14 | Pro 613 CB | Arg 673 CD | 3.60 | Hydrophobic |
| 15 | Asn 614 OD1 | Arg 672 NH1 | 2.87 | H-bond |
| 16 | Val 617 CG2 | Leu 676 CD1 | 4.69 | Hydrophobic |
| 17 | Val 617 CG2 | Ser 680 CB | 4.15 | Hydrophobic |
| 18 | Val 617 CG2 | Leu 676 CB | 4.10 | Hydrophobic |
| 19 | Ala 621 CB | Leu 693 CD2 | 3.98 | Hydrophobic |
| 20 | Phe 620 CD1 | Gln 684 CG | 3.60 | Hydrophobic |
| 21 | Phe 620 CE1 | Gln 684 CD | 3.77 | Hydrophobic |
| 22 | Phe 620 CB | Gln 683 O | 4.15 | van der Waal |
| 23 | Phe 620 O | Gln 683 O | 3.41 | H-bond |
| 24 | Ala 616 CA | Ser 680 CB | 3.8 | Hydrophobic |
| 25 | Tyr/Phe 604 CE2 | Asp 606 CB | 4.26 | Hydrophobic |
| 26 | Pro 607 CD | Asp 606 OD1 | 3.28 | van der Waal |
| 27 | Asp 606 O | Thr 609 OG1 | 2.72 | Hydrogen bond |
| 28 | Asp 606 O | Thr 609 N | 2.90 | Hydrogen bond |
| 29 | Asp 606 CB | Thr 609 CG2 | 3.56 | Hydrophobic |
| 30 | Phe 604 CZ | Pro 607 CD | 3.90 | Hydrophobic |
| 31 | Pro 607 O | Phe 610 N | 3.08 | Hydrogen bond |
| 32 | Phe 608 CD2 | Pro 613 CG | 4.43 | Hydrophobic |
| 33 | Phe 610 CE1 | Ile 605 CG2 | 3.48 | Hydrophobic |
| 34 | Phe 610 CZ | Phe 620 CE1 | 3.91 | Hydrophobic |
| 35 | Phe 610 CE1 | Ala 616 CB | 3.91 | Hydrophobic |
| 36 | Phe 610 CD2 | Glu 615 CG | 3.74 | Hydrophobic |
| 37 | Asp 612 O | Glu 615 N | 2.73 | Hydrogen bond |
| 38 | Phe 608 N | Asp 606 OD1 | 2.83 | Hydrogen |
| | | | | Bond |
| 39 | Asp 612 OD1 | Asn 614 ND2 | 3.09 | Hydrogen bond |
| 40 | Asp 612 OD1 | Asn 614 N | 3.11 | Hydrogen bond |
| 41 | Asn 614 O | Arg 618 N | 3.16 | Hydrogen bond |
| 42 | Pro 613 O | Val 617 N | 3.59 | Weak hydrogen |
| | | | | bond, van der |
| | | | | Waal |
| 43 | Glu 615 O | Glu 619 N | 3.06 | Hydrogen |
| | | | | Bond |
| 44 | Glu 615 OE1 | Glu 619 OE1 | 2.64 | Hydrogen |
| | | | | Bond |
| 45 | Phe 620 N | Ala 616 O | 2.96 | Hydorgen |
| | | | | Bond |
| 46 | Glu 619 OE2 | Phe 620 CZ | 4.03 | Van der waals |
| 47 | Ala 621 N | Val 617 O | 2.91 | Hydrogen |
| | | | | Bond |
| 48 | Ala 621 O | Val 617 O | 3.25 | Hydrogen |
| | | | | Bond |
| 49 | Ala 621 CB | Val 617 CG1 | 4.16 | Hydrophobic |

TABLE 3
REMARK coordinates from minimization and B-factor refinement
□
REMARK refinement resolution: 30 - 1.9 A
REMARK starting r= 0.2330 free_r= 0.2672
REMARK final r= 0.2316 free_r= 0.2691
REMARK rmsd bonds= 0.007125 rmsd angles= 1.07641
REMARK B rmsd for bonded mainchain atoms= 1.398 target= 1.5
REMARK B rmsd for bonded sidechain atoms= 1.963 target= 2
REMARK B rmsd for angle mainchain atoms= 2.128 target= 2
REMARK B rmsd for angle sidechain atoms= 2.727 target= 2.5
REMARK target= mlf final wa= 1.79025
REMARK final rweight= 0.1000 (with wa= 1.79025)
REMARK md-method= torsion annealing schedule= slowcool
REMARK starting temperature= 3000 total md steps= 30 * 6
REMARK cycles= 2 coordinate steps= 20 B-factor steps= 10
REMARK sg= P1 a= 47.052 b= 57.616 c= 67.742 alpha= 112.949 beta= 103.173 gamma=
91.577
REMARK topology file 1 : CNS_TOPPAR:protein.top
REMARK topology file 2 : CNS_TOPPAR:dna-rna.top
REMARK topology file 3 : CNS_TOPPAR:water.top
REMARK topology file 4 : CNS_TOPPAR:ion.top
REMARK topology file 5 : adenine.top
REMARK parameter file 1 : CNS_TOPPAR:proteinrep.param
REMARK parameter file 2 : CNS_TOPPAR:dna-rna_rep.param
REMARK parameter file 3 : CNS_TOPPAR:water_rep.param
REMARK parameter file 4 : CNS_TOPPAR:ion.param
REMARK parameter file 5 : adenine.par
REMARK molecular structure file: gen_ab.mtf
REMARK input coordinates: ref7b.pdb
REMARK reflection file= ../cycle1/amp.cv
REMARK ncs= none
REMARK B-correction resolution: 6.0 - 1.9

| | |
|---|---|
| REMARK B-factor correction applied to coordinate arrayB: | 0.210 |

REMARK bulk solvent: density level= 0.37437 e/A\hat{ }3, B-factor= 62.0599 A\hat{ }2
REMARK reflections with |Fobs| /sigma_F < 2 rejected
REMARK reflections with |Fobs| > 10000 * rms(Fobs) rejected

| | |
|---|---|
| REMARK theoretical total number of refl. in resol. range: | 49847 (100.0%) |
| REMARK number of unobserved reflections (no entry or \|F\|=0): | 6944 ( 13.9%) |
| REMARK number of reflections rejected: | 2972 ( 6.0%) |
| REMARK total number of reflections used: | 39931 ( 80.1%) |
| REMARK number of reflections in working set: | 35881 ( 72.0%) |
| REMARK number of reflections in test set: | 4050 ( 8.1%) |

CRYST1 47.052 57.616 67.742 112.95 103.17 91.58 P 1
REMARK FILENAME=′ref7c.pdb′
REMARK DATE:18-Jan-01 11:50:14 created by user: groot
REMARK VERSION:1.0

| | | | | | | | | | | | |
|---|---|---|---|---|---|---|---|---|---|---|---|
| ATOM | 1 | CB | LYS | A | 602 | −9.305 | −0.312 | −16.924 | 1.00 | 36.55 | A |
| ATOM | 2 | CG | LYS | A | 602 | −9.592 | −1.380 | −17.964 | 1.00 | 40.76 | A |
| ATOM | 3 | CD | LYS | A | 602 | −9.801 | −2.735 | −17.332 | 1.00 | 43.15 | A |
| ATOM | 4 | CE | LYS | A | 602 | −10.202 | −3.766 | −18.379 | 1.00 | 46.04 | A |
| ATOM | 5 | NZ | LYS | A | 602 | −10.292 | −5.135 | −17.793 | 1.00 | 47.27 | A |
| ATOM | 6 | C | LYS | A | 602 | −9.501 | 2.125 | −16.413 | 1.00 | 30.61 | A |
| ATOM | 7 | O | LYS | A | 602 | −8.689 | 3.021 | −16.178 | 1.00 | 31.27 | A |
| ATOM | 8 | N | LYS | A | 602 | −7.962 | 1.290 | −18.245 | 1.00 | 34.40 | A |
| ATOM | 9 | CA | LYS | A | 602 | −9.247 | 1.097 | −17.512 | 1.00 | 33.41 | A |
| ATOM | 10 | N | ILE | A | 603 | −10.653 | 1.995 | −15.761 | 1.00 | 26.03 | A |
| ATOM | 11 | CA | ILE | A | 603 | −11.041 | 2.890 | −14.680 | 1.00 | 21.35 | A |
| ATOM | 12 | CB | ILE | A | 603 | −12.110 | 3.916 | −15.127 | 1.00 | 23.23 | A |
| ATOM | 13 | CG2 | ILE | A | 603 | −13.424 | 3.183 | −15.474 | 1.00 | 23.72 | A |
| ATOM | 14 | CG1 | ILE | A | 603 | −12.383 | 4.899 | −13.988 | 1.00 | 22.54 | A |
| ATOM | 15 | CD1 | ILE | A | 603 | −13.398 | 5.974 | −14.316 | 1.00 | 27.41 | A |
| ATOM | 16 | C | ILE | A | 603 | −11.648 | 2.050 | −13.553 | 1.00 | 17.90 | A |
| ATOM | 17 | O | ILE | A | 603 | −12.280 | 1.022 | −13.815 | 1.00 | 16.74 | A |
| ATOM | 18 | N | PHE | A | 604 | −11.460 | 2.501 | −12.313 | 1.00 | 12.95 | A |
| ATOM | 19 | CA | PHE | A | 604 | −11.981 | 1.815 | −11.122 | 1.00 | 14.58 | A |
| ATOM | 20 | CB | PHE | A | 604 | −11.309 | 2.347 | −9.848 | 1.00 | 13.58 | A |
| ATOM | 21 | CG | PHE | A | 604 | −11.978 | 1.890 | −8.569 | 1.00 | 10.12 | A |
| ATOM | 22 | CD1 | PHE | A | 604 | −11.890 | 0.565 | −8.165 | 1.00 | 12.14 | A |
| ATOM | 23 | CD2 | PHE | A | 604 | −12.683 | 2.785 | −7.770 | 1.00 | 12.13 | A |
| ATOM | 24 | CE1 | PHE | A | 604 | −12.493 | 0.132 | −6.972 | 1.00 | 12.82 | A |
| ATOM | 25 | CE2 | PHE | A | 604 | −13.293 | 2.368 | −6.574 | 1.00 | 13.41 | A |
| ATOM | 26 | CZ | PEE | A | 604 | −13.194 | 1.036 | −6.176 | 1.00 | 11.79 | A |
| ATOM | 27 | C | PHE | A | 604 | −13.488 | 2.027 | −10.968 | 1.00 | 14.34 | A |
| ATOM | 28 | O | PEE | A | 604 | −13.972 | 3.155 | −11.068 | 1.00 | 14.17 | A |
| ATOM | 29 | N | ILE | A | 605 | −14.205 | 0.946 | −10.671 | 1.00 | 14.05 | A |
| ATOM | 30 | CA | ILE | A | 605 | −15.658 | 0.985 | −10.471 | 1.00 | 15.51 | A |
| ATOM | 31 | CB | ILE | A | 605 | −16.376 | −0.024 | −11.404 | 1.00 | 14.91 | A |
| ATOM | 32 | CG2 | ILE | A | 605 | −17.892 | 0.062 | −11.203 | 1.00 | 17.40 | A |
| ATOM | 33 | CG1 | ILE | A | 605 | −16.034 | 0.269 | −12.868 | 1.00 | 16.70 | A |
| ATOM | 34 | CD1 | ILE | A | 605 | −16.412 | 1.664 | −13.326 | 1.00 | 20.67 | A |
| ATOM | 35 | C | ILE | A | 605 | −15.976 | 0.616 | −9.010 | 1.00 | 15.81 | A |
| ATOM | 36 | O | ILE | A | 605 | −15.679 | −0.491 | −8.569 | 1.00 | 17.18 | A |
| ATOM | 37 | N | ASP | A | 606 | −16.547 | 1.548 | −8.253 | 1.00 | 16.32 | A |
| ATOM | 38 | CA | ASP | A | 606 | −16.902 | 1.291 | −6.855 | 1.00 | 17.50 | A |
| ATOM | 39 | CB | ASP | A | 606 | −17.542 | 2.550 | −6.253 | 1.00 | 18.47 | A |
| ATOM | 40 | CG | ASP | A | 606 | −17.884 | 2.403 | −4.775 | 1.00 | 19.43 | A |
| ATOM | 41 | OD1 | ASP | A | 606 | −17.942 | 1.262 | −4.272 | 1.00 | 20.86 | A |
| ATOM | 42 | OD2 | ASP | A | 606 | −18.114 | 3.440 | −4.115 | 1.00 | 20.82 | A |
| ATOM | 43 | C | ASP | A | 606 | −17.899 | 0.128 | −6.844 | 1.00 | 18.22 | A |
| ATOM | 44 | O | ASP | A | 606 | −19.001 | 0.249 | −7.371 | 1.00 | 17.14 | A |
| ATOM | 45 | N | PRO | A | 607 | −17.517 | −1.014 | −6.247 | 1.00 | 17.74 | A |
| ATOM | 46 | CD | PRO | A | 607 | −16.268 | −1.278 | −5.509 | 1.00 | 17.77 | A |
| ATOM | 47 | CA | PRO | A | 607 | −18.427 | −2.164 | −6.209 | 1.00 | 18.01 | A |
| ATOM | 48 | CB | PRO | A | 607 | −17.621 | −3.247 | −5.470 | 1.00 | 17.22 | A |
| ATOM | 49 | CG | PRO | A | 607 | −16.645 | −2.465 | −4.633 | 1.00 | 18.76 | A |
| ATOM | 50 | C | PRO | A | 607 | −19.753 | −1.836 | −5.536 | 1.00 | 16.89 | A |
| ATOM | 51 | O | PRO | A | 607 | −20.780 | −2.404 | −5.878 | 1.00 | 17.69 | A |
| ATOM | 52 | N | PEE | A | 608 | −19.744 | −0.897 | −4.602 | 1.00 | 17.34 | A |
| ATOM | 53 | CA | PEE | A | 608 | −20.989 | −0.557 | −3.946 | 1.00 | 19.12 | A |
| ATOM | 54 | CB | PHE | A | 608 | −20.738 | 0.133 | −2.613 | 1.00 | 18.91 | A |
| ATOM | 55 | CG | PEE | A | 608 | −20.319 | −0.799 | −1.511 | 1.00 | 18.72 | A |
| ATOM | 56 | CD1 | PHE | A | 608 | −20.047 | −0.291 | −0.251 | 1.00 | 18.84 | A |
| ATOM | 57 | CD2 | PEE | A | 608 | −20.171 | −2.166 | −1.729 | 1.00 | 18.90 | A |
| ATOM | 58 | CE1 | PHE | A | 608 | −19.632 | −1.114 | 0.776 | 1.00 | 20.59 | A |
| ATOM | 59 | CE2 | PHE | A | 608 | −19.750 | −3.011 | −0.693 | 1.00 | 22.14 | A |
| ATOM | 60 | CZ | PHE | A | 608 | −19.482 | −2.478 | 0.559 | 1.00 | 19.82 | A |
| ATOM | 61 | C | PEE | A | 608 | −21.928 | 0.292 | −4.795 | 1.00 | 18.67 | A |
| ATOM | 62 | O | PHE | A | 608 | −22.993 | 0.678 | −4.319 | 1.00 | 18.02 | A |
| ATOM | 63 | N | TER | A | 609 | −21.546 | 0.609 | −6.031 | 1.00 | 18.97 | A |
| ATOM | 64 | CA | THR | A | 609 | −22.463 | 1.373 | −6.868 | 1.00 | 19.12 | A |
| ATOM | 65 | CB | THR | A | 609 | −21.748 | 2.284 | −7.911 | 1.00 | 17.33 | A |
| ATOM | 66 | OG1 | THR | A | 609 | −20.955 | 1.487 | −8.799 | 1.00 | 17.70 | A |
| ATOM | 67 | CG2 | THR | A | 609 | −20.886 | 3.309 | −7.216 | 1.00 | 18.57 | A |
| ATOM | 68 | C | THR | A | 609 | −23.313 | 0.342 | −7.606 | 1.00 | 19.52 | A |
| ATOM | 69 | O | THR | A | 609 | −24.302 | 0.683 | −8.247 | 1.00 | 19.06 | A |
| ATOM | 70 | N | PHE | A | 610 | −22.925 | −0.928 | −7.524 | 1.00 | 18.09 | A |
| ATOM | 71 | CA | PHE | A | 610 | −23.709 | −1.967 | −8.181 | 1.00 | 19.10 | A |
| ATOM | 72 | CB | PHE | A | 610 | −22.955 | −3.299 | −8.223 | 1.00 | 20.69 | A |
| ATOM | 73 | CG | PHE | A | 610 | −21.861 | −3.357 | −9.240 | 1.00 | 21.23 | A |
| ATOM | 74 | CD1 | PHE | A | 610 | −20.707 | −2.610 | −9.082 | 1.00 | 22.59 | A |
| ATOM | 75 | CD2 | PHE | A | 610 | −21.973 | −4.184 | −10.350 | 1.00 | 23.50 | A |
| ATOM | 76 | CE1 | PHE | A | 610 | −19.678 | −2.681 | −10.007 | 1.00 | 21.63 | A |
| ATOM | 77 | CE2 | PHE | A | 610 | −20.942 | −4.261 | −11.285 | 1.00 | 23.79 | A |
| ATOM | 78 | CZ | PHE | A | 610 | −19.791 | −3.504 | −11.107 | 1.00 | 20.78 | A |
| ATOM | 79 | C | PHE | A | 610 | −25.000 | −2.167 | −7.386 | 1.00 | 20.52 | A |
| ATOM | 80 | O | PHE | A | 610 | −24.986 | −2.148 | −6.150 | 1.00 | 20.43 | A |
| ATOM | 81 | N | GLU | A | 611 | −26.111 | −2.343 | −8.095 | 1.00 | 20.58 | A |
| ATOM | 82 | CA | GLU | A | 611 | −27.404 | −2.571 | −7.459 | 1.00 | 21.53 | A |
| ATOM | 83 | CB | GLU | A | 611 | −28.485 | −2.853 | −8.523 | 1.00 | 22.75 | A |
| ATOM | 84 | CG | GLU | A | 611 | −28.714 | −1.718 | −9.518 | 0.00 | 23.28 | A |
| ATOM | 85 | CD | GLU | A | 611 | −29.783 | −2.041 | −10.554 | 0.00 | 23.79 | A |
| ATOM | 86 | OE1 | GLU | A | 611 | −30.061 | −1.175 | −11.409 | 0.00 | 24.06 | A |
| ATOM | 87 | OE2 | GLU | A | 611 | −30.345 | −3.158 | −10.516 | 0.00 | 24.06 | A |
| ATOM | 88 | C | GLU | A | 611 | −27.257 | −3.790 | −6.546 | 1.00 | 21.14 | A |
| ATOM | 89 | O | GLU | A | 611 | −27.861 | −3.857 | −5.479 | 1.00 | 20.93 | A |
| ATOM | 90 | N | ASP | A | 612 | −26.445 | −4.746 | −6.992 | 1.00 | 21.15 | A |
| ATOM | 91 | CA | ASP | A | 612 | −26.160 | −5.966 | −6.239 | 1.00 | 21.15 | A |
| ATOM | 92 | CB | ASP | A | 612 | −26.738 | −7.203 | −6.946 | 1.00 | 22.41 | A |
| ATOM | 93 | CG | ASP | A | 612 | −26.407 | −8.504 | −6.220 | 1.00 | 26.34 | A |
| ATOM | 94 | OD1 | ASP | A | 612 | −25.869 | −8.451 | −5.091 | 1.00 | 26.08 | A |
| ATOM | 95 | OD2 | ASP | A | 612 | −26.693 | −9.588 | −6.776 | 1.00 | 28.73 | A |
| ATOM | 96 | C | ASP | A | 612 | −24.641 | −6.106 | −6.114 | 1.00 | 20.74 | A |
| ATOM | 97 | O | ASP | A | 612 | −23.967 | −6.530 | −7.051 | 1.00 | 18.43 | A |
| ATOM | 98 | N | PRO | A | 613 | −24.085 | −5.745 | −4.948 | 1.00 | 22.26 | A |
| ATOM | 99 | CD | PRO | A | 613 | −24.796 | −5.172 | −3.790 | 1.00 | 21.09 | A |
| ATOM | 100 | CA | PRO | A | 613 | −22.642 | −5.825 | −4.692 | 1.00 | 23.09 | A |
| ATOM | 101 | CB | PRO | A | 613 | −22.551 | −5.579 | −3.188 | 1.00 | 23.78 | A |
| ATOM | 102 | CG | PRO | A | 613 | −23.662 | −4.598 | −2.957 | 1.00 | 24.95 | A |
| ATOM | 103 | C | PRO | A | 613 | −22.001 | −7.149 | −5.112 | 1.00 | 24.21 | A |
| ATOM | 104 | O | PRO | A | 613 | −20.830 | −7.182 | −5.486 | 1.00 | 24.14 | A |
| ATOM | 105 | N | ASN | A | 614 | −22.764 | −8.238 | −5.060 | 1.00 | 24.32 | A |
| ATOM | 106 | CA | ASN | A | 614 | −22.232 | −9.544 | −5.445 | 1.00 | 24.86 | A |
| ATOM | 107 | CB | ASN | A | 614 | −23.242 | −10.652 | −5.162 | 1.00 | 28.16 | A |
| ATOM | 108 | CG | ASN | A | 614 | −23.520 | −10.813 | −3.699 | 1.00 | 30.99 | A |
| ATOM | 109 | OD1 | ASN | A | 614 | −22.600 | −10.994 | −2.903 | 1.00 | 33.48 | A |
| ATOM | 110 | ND2 | ASN | A | 614 | −24.795 | −10.750 | −3.325 | 1.00 | 34.69 | A |
| ATOM | 111 | C | ASN | A | 614 | −21.866 | −9.598 | −6.912 | 1.00 | 25.02 | A |
| ATOM | 112 | O | ASN | A | 614 | −21.035 | −10.412 | −7.329 | 1.00 | 23.77 | A |
| ATOM | 113 | N | GLU | A | 615 | −22.498 | −8.742 | −7.706 | 1.00 | 22.98 | A |
| ATOM | 114 | CA | GLU | A | 615 | −22.213 | −8.728 | −9.129 | 1.00 | 23.04 | A |
| ATOM | 115 | CB | GLU | A | 615 | −23.144 | −7.762 | −9.863 | 1.00 | 24.89 | A |
| ATOM | 116 | CG | GLU | A | 615 | −22.838 | −7.681 | −11.345 | 1.00 | 29.63 | A |
| ATOM | 117 | CD | GLU | A | 615 | −22.902 | −9.036 | −12.032 | 1.00 | 33.50 | A |
| ATOM | 118 | OE1 | GLU | A | 615 | −22.270 | −9.188 | −13.103 | 1.00 | 35.83 | A |
| ATOM | 119 | OE2 | GLU | A | 615 | −23.589 | −9.949 | −11.511 | 1.00 | 35.71 | A |
| ATOM | 120 | C | GLU | A | 615 | −20.766 | −8.312 | −9.348 | 1.00 | 19.65 | A |
| ATOM | 121 | O | GLU | A | 615 | −20.079 | −8.854 | −10.211 | 1.00 | 19.95 | A |
| ATOM | 122 | N | ALA | A | 616 | −20.306 | −7.340 | −8.569 | 1.00 | 19.06 | A |
| ATOM | 123 | CA | ALA | A | 616 | −18.932 | −6.884 | −8.702 | 1.00 | 16.63 | A |
| ATOM | 124 | CB | ALA | A | 616 | −18.676 | −5.728 | −7.763 | 1.00 | 16.24 | A |
| ATOM | 125 | C | ALA | A | 616 | −17.982 | −8.037 | −8.401 | 1.00 | 15.74 | A |
| ATOM | 126 | O | ALA | A | 616 | −16.929 | −8.180 | −9.031 | 1.00 | 15.81 | A |
| ATOM | 127 | N | VAL | A | 617 | −18.353 | −8.880 | −7.447 | 1.00 | 15.22 | A |
| ATOM | 128 | CA | VAL | A | 617 | −17.493 | −10.001 | −7.106 | 1.00 | 14.30 | A |
| ATOM | 129 | CB | VAL | A | 617 | −18.003 | −10.750 | −5.865 | 1.00 | 14.79 | A |
| ATOM | 130 | CG1 | VAL | A | 617 | −17.028 | −11.869 | −5.501 | 1.00 | 16.38 | A |
| ATOM | 131 | CG2 | VAL | A | 617 | −18.123 | −9.781 | −4.703 | 1.00 | 11.27 | A |
| ATOM | 132 | C | VAL | A | 617 | −17.337 | −10.979 | −8.256 | 1.00 | 14.61 | A |
| ATOM | 133 | O | VAL | A | 617 | −16.215 | −11.372 | −8.608 | 1.00 | 14.99 | A |
| ATOM | 134 | N | ARG | A | 618 | −18.445 | −11.370 | −8.868 | 1.00 | 16.24 | A |
| ATOM | 135 | CA | ARG | A | 618 | −18.353 | −12.322 | −9.964 | 1.00 | 18.27 | A |
| ATOM | 136 | CB | ARG | A | 618 | −19.752 | −12.808 | −10.375 | 1.00 | 20.76 | A |
| ATOM | 137 | CG | ARG | A | 618 | −20.691 | −11.740 | −10.838 | 0.00 | 21.07 | A |
| ATOM | 138 | CD | ARG | A | 618 | −22.044 | −12.351 | −11.112 | 0.00 | 22.19 | A |
| ATOM | 139 | NE | ARG | A | 618 | −22.650 | −12.891 | −9.899 | 0.00 | 22.97 | A |
| ATOM | 140 | CZ | ARG | A | 618 | −23.853 | −13.451 | −9.857 | 0.00 | 23.42 | A |
| ATOM | 141 | NH1 | ARG | A | 618 | −24.575 | −13.545 | −10.965 | 0.00 | 23.69 | A |
| ATOM | 142 | NH2 | ARG | A | 618 | −24.342 | −13.903 | −8.711 | 0.00 | 23.69 | A |
| ATOM | 143 | C | ARG | A | 618 | −17.626 | −11.746 | −11.168 | 1.00 | 18.93 | A |
| ATOM | 144 | O | ARG | A | 618 | −16.988 | −12.479 | −11.928 | 1.00 | 21.33 | A |
| ATOM | 145 | N | GLU | A | 619 | −17.707 | −10.430 | −11.334 | 1.00 | 18.93 | A |
| ATOM | 146 | CA | GLU | A | 619 | −17.059 | −9.777 | −12.463 | 1.00 | 20.86 | A |
| ATOM | 147 | CB | GLU | A | 619 | −17.728 | −8.439 | −12.745 | 1.00 | 23.33 | A |
| ATOM | 148 | CG | GLU | A | 619 | −19.148 | −8.576 | −13.213 | 1.00 | 30.69 | A |
| ATOM | 149 | CD | GLU | A | 619 | −19.640 | −7.325 | −13.876 | 1.00 | 34.05 | A |
| ATOM | 150 | OE1 | GLU | A | 619 | −20.842 | −7.271 | −14.214 | 1.00 | 37.39 | A |
| ATOM | 151 | OE2 | GLU | A | 619 | −18.821 | −6.396 | −14.065 | 1.00 | 36.23 | A |
| ATOM | 152 | C | GLU | A | 619 | −15.564 | −9.548 | −12.338 | 1.00 | 20.77 | A |
| ATOM | 153 | O | GLU | A | 619 | −14.829 | −9.748 | −13.300 | 1.00 | 21.23 | A |
| ATOM | 154 | N | PHE | A | 620 | −15.113 | −9.128 | −11.161 | 1.00 | 19.38 | A |
| ATOM | 155 | CA | PHE | A | 620 | −13.697 | −8.841 | −10.977 | 1.00 | 20.53 | A |
| ATOM | 156 | CB | PHE | A | 620 | −13.544 | −7.472 | −10.326 | 1.00 | 19.14 | A |
| ATOM | 157 | CG | PHE | A | 620 | −14.366 | −6.393 | −10.987 | 1.00 | 18.99 | A |
| ATOM | 158 | CD1 | PHE | A | 620 | −15.303 | −5.672 | −10.258 | 1.00 | 18.77 | A |
| ATOM | 159 | CD2 | PHE | A | 620 | −14.193 | −6.091 | −12.339 | 1.00 | 22.01 | A |
| ATOM | 160 | CE1 | PHE | A | 620 | −16.061 | −4.661 | −10.859 | 1.00 | 20.15 | A |
| ATOM | 161 | CE2 | PHE | A | 620 | −14.947 | −5.082 | −12.951 | 1.00 | 20.05 | A |
| ATOM | 162 | CZ | PHE | A | 620 | −15.879 | −4.369 | −12.205 | 1.00 | 18.18 | A |
| ATOM | 163 | C | PHE | A | 620 | −12.892 | −9.871 | −10.190 | 1.00 | 22.13 | A |
| ATOM | 164 | O | PHE | A | 620 | −11.677 | −9.719 | −10.037 | 1.00 | 21.27 | A |
| ATOM | 165 | N | ALA | A | 621 | −13.562 | −10.915 | −9.704 | 1.00 | 22.03 | A |
| ATOM | 166 | CA | ALA | A | 621 | −12.906 | −11.959 | −8.922 | 1.00 | 23.00 | A |
| ATOM | 167 | CB | ALA | A | 621 | −13.365 | −11.873 | −7.469 | 1.00 | 22.11 | A |
| ATOM | 168 | C | ALA | A | 621 | −13.159 | −13.368 | −9.456 | 1.00 | 24.84 | A |
| ATOM | 169 | O | ALA | A | 621 | −14.300 | −13.748 | −9.731 | 1.00 | 24.07 | A |
| ATOM | 170 | N | LYS | A | 622 | −12.088 | −14.146 | −9.587 | 1.00 | 24.59 | A |
| ATOM | 171 | CA | LYS | A | 622 | −12.195 | −15.517 | −10.074 | 1.00 | 26.44 | A |
| ATOM | 172 | CB | LYS | A | 622 | −10.842 | −16.000 | −10.600 | 1.00 | 29.90 | A |
| ATOM | 173 | CG | LYS | A | 622 | −10.862 | −17.445 | −11.086 | 1.00 | 34.03 | A |
| ATOM | 174 | CD | LYS | A | 622 | −9.455 | −18.030 | −11.189 | 1.00 | 36.96 | A |
| ATOM | 175 | CE | LYS | A | 622 | −8.623 | −17.304 | −12.231 | 1.00 | 39.89 | A |
| ATOM | 176 | NZ | LYS | A | 622 | −7.211 | −17.795 | −12.281 | 1.00 | 41.83 | A |
| ATOM | 177 | C | LYS | A | 622 | −12.647 | −16.453 | −8.956 | 1.00 | 26.32 | A |
| ATOM | 178 | O | LYS | A | 622 | −12.038 | −16.482 | −7.885 | 1.00 | 25.37 | A |
| ATOM | 179 | N | GLU | A | 623 | −13.713 | −17.211 | −9.202 | 1.00 | 25.42 | A |
| ATOM | 180 | CA | GLU | A | 623 | −14.222 | −18.161 | −8.214 | 1.00 | 25.52 | A |
| ATOM | 181 | CB | GLU | A | 623 | −15.657 | −18.582 | −8.566 | 1.00 | 26.37 | A |
| ATOM | 182 | CG | GLU | A | 623 | −16.289 | −19.613 | −7.627 | 1.00 | 26.38 | A |
| ATOM | 183 | CD | GLU | A | 623 | −16.521 | −19.094 | −6.212 | 1.00 | 28.72 | A |
| ATOM | 184 | OE1 | GLU | A | 623 | −16.905 | −17.909 | −6.053 | 1.00 | 28.21 | A |
| ATOM | 185 | OE2 | GLU | A | 623 | −16.328 | −19.876 | −5.253 | 1.00 | 29.13 | A |
| ATOM | 186 | C | GLU | A | 623 | −13.302 | −19.371 | −8.231 | 1.00 | 24.77 | A |
| ATOM | 187 | O | GLU | A | 623 | −13.131 | −20.015 | −9.261 | 1.00 | 25.82 | A |
| ATOM | 188 | N | ILE | A | 624 | −12.686 | −19.663 | −7.094 | 1.00 | 26.18 | A |
| ATOM | 189 | CA | ILE | A | 624 | −11.777 | −20.798 | −6.993 | 1.00 | 25.31 | A |
| ATOM | 190 | CB | ILE | A | 624 | −10.466 | −20.400 | −6.263 | 1.00 | 25.34 | A |
| ATOM | 191 | CG2 | ILE | A | 624 | −9.588 | −21.641 | −6.048 | 1.00 | 23.96 | A |
| ATOM | 192 | CG1 | ILE | A | 624 | −9.730 | −19.327 | −7.070 | 1.00 | 24.39 | A |
| ATOM | 193 | CD1 | ILE | A | 624 | −8.450 | −18.815 | −6.426 | 1.00 | 25.55 | A |
| ATOM | 194 | C | ILE | A | 624 | −12.427 | −21.950 | −6.236 | 1.00 | 25.18 | A |
| ATOM | 195 | O | ILE | A | 624 | −13.012 | −21.763 | −5.170 | 1.00 | 25.10 | A |
| ATOM | 196 | N | ASP | A | 625 | −12.324 | −23.144 | −6.801 | 1.00 | 26.96 | A |
| ATOM | 197 | CA | ASP | A | 625 | −12.890 | −24.323 | −6.167 | 1.00 | 27.95 | A |
| ATOM | 198 | CB | ASP | A | 625 | −12.781 | −25.528 | −7.089 | 1.00 | 30.55 | A |
| ATOM | 199 | CG | ASP | A | 625 | −13.634 | −26.679 | −6.625 | 1.00 | 34.97 | A |
| ATOM | 200 | OD1 | ASP | A | 625 | −14.850 | −26.648 | −6.907 | 1.00 | 37.34 | A |
| ATOM | 201 | OD2 | ASP | A | 625 | −13.095 | −27.597 | −5.963 | 1.00 | 34.92 | A |
| ATOM | 202 | C | ASP | A | 625 | −12.088 | −24.580 | −4.902 | 1.00 | 27.84 | A |
| ATOM | 203 | O | ASP | A | 625 | −10.857 | −24.583 | −4.937 | 1.00 | 26.54 | A |
| ATOM | 204 | N | ILE | A | 626 | −12.781 | −24.807 | −3.791 | 1.00 | 27.43 | A |
| ATOM | 205 | CA | ILE | A | 626 | −12.111 | −25.042 | −2.518 | 1.00 | 28.27 | A |
| ATOM | 206 | CB | ILE | A | 626 | −13.149 | −25.293 | −1.398 | 1.00 | 29.15 | A |
| ATOM | 207 | CG2 | ILE | A | 626 | −13.897 | −26.591 | −1.660 | 1.00 | 28.78 | A |
| ATOM | 208 | CG1 | ILE | A | 626 | −12.455 | −25.330 | −0.040 | 1.00 | 30.32 | A |
| ATOM | 209 | CD1 | ILE | A | 626 | −13.412 | −25.148 | 1.122 | 1.00 | 35.27 | A |
| ATOM | 210 | C | ILE | A | 626 | −11.099 | −26.195 | −2.563 | 1.00 | 27.88 | A |
| ATOM | 211 | O | ILE | A | 626 | −10.116 | −26.198 | −1.825 | 1.00 | 27.54 | A |
| ATOM | 212 | N | SER | A | 627 | −11.319 | −27.164 | −3.442 | 1.00 | 29.17 | A |
| ATOM | 213 | CA | SER | A | 627 | −10.393 | −28.289 | −3.538 | 1.00 | 29.60 | A |
| ATOM | 214 | CB | SER | A | 627 | −10.942 | −29.350 | −4.483 | 1.00 | 29.57 | A |
| ATOM | 215 | OG | SER | A | 627 | −10.885 | −28.887 | −5.818 | 1.00 | 31.25 | A |
| ATOM | 216 | C | SER | A | 627 | −9.009 | −27.858 | −4.028 | 1.00 | 29.72 | A |
| ATOM | 217 | O | SER | A | 627 | −8.072 | −28.657 | −4.025 | 1.00 | 29.55 | A |
| ATOM | 218 | N | CYS | A | 628 | −8.888 | −26.606 | −4.465 | 1.00 | 29.03 | A |
| ATOM | 219 | CA | CYS | A | 628 | −7.616 | −26.075 | −4.951 | 1.00 | 29.50 | A |
| ATOM | 220 | CB | CYS | A | 628 | −7.840 | −25.103 | −6.128 | 1.00 | 28.94 | A |
| ATOM | 221 | SG | CYS | A | 628 | −8.593 | −25.809 | −7.641 | 1.00 | 30.55 | A |
| ATOM | 222 | C | CYS | A | 628 | −6.871 | −25.335 | −3.836 | 1.00 | 29.48 | A |
| ATOM | 223 | O | CYS | A | 628 | −5.665 | −25.115 | −3.928 | 1.00 | 28.60 | A |
| ATOM | 224 | N | VAL | A | 629 | −7.592 | −24.955 | −2.785 | 1.00 | 30.83 | A |
| ATOM | 225 | CA | VAL | A | 629 | −7.004 | −24.218 | −1.671 | 1.00 | 31.02 | A |
| ATOM | 226 | CB | VAL | A | 629 | −7.999 | −23.159 | −1.128 | 1.00 | 32.23 | A |
| ATOM | 227 | CG1 | VAL | A | 629 | −7.317 | −22.303 | −0.052 | 1.00 | 32.29 | A |
| ATOM | 228 | CG2 | VAL | A | 629 | −8.509 | −22.280 | −2.264 | 1.00 | 31.46 | A |
| ATOM | 229 | C | VAL | A | 629 | −6.578 | −25.107 | −0.498 | 1.00 | 32.09 | A |
| ATOM | 230 | O | VAL | A | 629 | −7.324 | −25.985 | −0.063 | 1.00 | 32.06 | A |
| ATOM | 231 | N | LYS | A | 630 | −5.377 | −24.869 | 0.017 | 1.00 | 31.20 | A |
| ATOM | 232 | CA | LYS | A | 630 | −4.889 | −25.641 | 1.148 | 1.00 | 32.57 | A |
| ATOM | 233 | CB | LYS | A | 630 | −3.847 | −26.667 | 0.686 | 1.00 | 32.81 | A |
| ATOM | 234 | CG | LYS | A | 630 | −4.348 | −27.600 | −0.410 | 0.00 | 33.76 | A |
| ATOM | 235 | CD | LYS | A | 630 | −3.253 | −28.548 | −0.871 | 0.00 | 34.37 | A |
| ATOM | 236 | CE | LYS | A | 630 | −3.749 | −29.493 | −1.955 | 0.00 | 34.80 | A |
| ATOM | 237 | NZ | LYS | A | 630 | −4.215 | −28.765 | −3.167 | 0.00 | 35.13 | A |
| ATOM | 238 | C | LYS | A | 630 | −4.286 | −24.708 | 2.190 | 1.00 | 31.65 | A |
| ATOM | 239 | O | LYS | A | 630 | −3.204 | −24.162 | 2.000 | 1.00 | 31.96 | A |
| ATOM | 240 | N | ILE | A | 631 | −5.009 | −24.513 | 3.285 | 1.00 | 32.38 | A |
| ATOM | 241 | CA | ILE | A | 631 | −4.542 | −23.655 | 4.359 | 1.00 | 33.62 | A |
| ATOM | 242 | CB | ILE | A | 631 | −5.700 | −23.282 | 5.314 | 1.00 | 33.44 | A |
| ATOM | 243 | CG2 | ILE | A | 631 | −5.155 | −22.539 | 6.532 | 1.00 | 33.62 | A |
| ATOM | 244 | CG1 | ILE | A | 631 | −6.740 | −22.438 | 4.565 | 1.00 | 33.30 | A |
| ATOM | 245 | CD1 | ILE | A | 631 | −7.916 | −21.992 | 5.416 | 1.00 | 31.54 | A |
| ATOM | 246 | C | ILE | A | 631 | −3.464 | −24.396 | 5.142 | 1.00 | 34.94 | A |
| ATOM | 247 | O | ILE | A | 631 | −3.709 | −25.490 | 5.646 | 1.00 | 34.77 | A |
| ATOM | 248 | N | GLU | A | 632 | −2.278 | −23.797 | 5.237 | 1.00 | 35.73 | A |
| ATOM | 249 | CA | GLU | A | 632 | −1.154 | −24.396 | 5.958 | 1.00 | 37.69 | A |
| ATOM | 250 | CB | GLU | A | 632 | 0.167 | −24.114 | 5.236 | 1.00 | 38.83 | A |
| ATOM | 251 | CG | GLU | A | 632 | 0.312 | −24.785 | 3.892 | 0.00 | 39.80 | A |
| ATOM | 252 | CD | GLU | A | 632 | 0.374 | −26.290 | 4.006 | 0.00 | 40.38 | A |
| ATOM | 253 | OE1 | GLU | A | 632 | 1.254 | −26.793 | 4.735 | 0.00 | 40.70 | A |
| ATOM | 254 | OE2 | GLU | A | 632 | −0.455 | −26.970 | 3.367 | 0.00 | 40.70 | A |
| ATOM | 255 | C | GLU | A | 632 | −1.047 | −23.892 | 7.394 | 1.00 | 38.57 | A |
| ATOM | 256 | O | GLU | A | 632 | −1.118 | −24.681 | 8.342 | 1.00 | 38.93 | A |
| ATOM | 257 | N | GLN | A | 633 | −0.868 | −22.583 | 7.556 | 1.00 | 38.28 | A |
| ATOM | 258 | CA | GLN | A | 633 | −0.744 | −21.995 | 8.889 | 1.00 | 39.08 | A |
| ATOM | 259 | CB | GLN | A | 633 | 0.739 | −21.825 | 9.250 | 1.00 | 40.39 | A |
| ATOM | 260 | CG | GLN | A | 633 | 1.001 | −21.4821 | 0.712 | 0.00 | 41.11 | A |
| ATOM | 261 | CD | GLN | A | 633 | 2.481 | −21.3671 | 1.028 | 0.00 | 41.66 | A |
| ATOM | 262 | OE1 | GLN | A | 633 | 3.235 | −22.3311 | 0.891 | 0.00 | 41.94 | A |
| ATOM | 263 | NE2 | GLN | A | 633 | 2.904 | −20.1831 | 1.455 | 0.00 | 41.94 | A |
| ATOM | 264 | C | GLN | A | 633 | −1.455 | −20.650 | 8.994 | 1.00 | 39.45 | A |
| ATOM | 265 | O | GLN | A | 633 | −1.725 | −20.000 | 7.982 | 1.00 | 36.93 | A |
| ATOM | 266 | N | VAL | A | 634 | −1.762 | −20.238 | 10.221 | 1.00 | 39.17 | A |
| ATOM | 267 | CA | VAL | A | 634 | −2.425 | −18.960 | 10.445 | 1.00 | 41.26 | A |
| ATOM | 268 | CB | VAL | A | 634 | −3.371 | −19.013 | 11.653 | 1.00 | 41.53 | A |
| ATOM | 269 | CG1 | VAL | A | 634 | −4.109 | −17.694 | 11.778 | 1.00 | 42.05 | A |
| ATOM | 270 | CG2 | VAL | A | 634 | −4.344 | −20.164 | 11.501 | 1.00 | 40.57 | A |
| ATOM | 271 | C | VAL | A | 634 | −1.368 | −17.900 | 10.719 | 1.00 | 42.58 | A |
| ATOM | 272 | O | VAL | A | 634 | −0.569 | −18.044 | 11.649 | 1.00 | 43.99 | A |
| ATOM | 273 | N | ILE | A | 635 | −1.365 | −16.842 | 9.910 | 1.00 | 43.60 | A |
| ATOM | 274 | CA | ILE | A | 635 | −0.398 | −15.749 | 10.045 | 1.00 | 44.51 | A |
| ATOM | 275 | CB | ILE | A | 635 | −0.214 | −14.995 | 8.699 | 1.00 | 45.18 | A |
| ATOM | 276 | CG2 | ILE | A | 635 | 0.860 | −13.925 | 8.841 | 0.00 | 45.30 | A |
| ATOM | 277 | CG1 | ILE | A | 635 | 0.154 | −15.979 | 7.586 | 0.00 | 45.40 | A |
| ATOM | 278 | CD1 | ILE | A | 635 | 1.450 | −16.730 | 7.817 | 0.00 | 45.70 | A |
| ATOM | 279 | C | ILE | A | 635 | −0.846 | −14.744 | 11.103 | 1.00 | 45.18 | A |
| ATOM | 280 | O | ILE | A | 635 | −0.281 | −14.678 | 12.197 | 1.00 | 45.07 | A |
| ATOM | 281 | N | GLY | A | 636 | −1.871 | −13.966 | 10.768 | 1.00 | 45.30 | A |
| ATOM | 282 | CA | GLY | A | 636 | −2.385 | −12.968 | 11.689 | 1.00 | 45.00 | A |
| ATOM | 283 | C | GLY | A | 636 | −3.900 | −12.992 | 11.798 | 1.00 | 44.80 | A |
| ATOM | 284 | O | GLY | A | 636 | −4.550 | −13.951 | 11.375 | 1.00 | 44.35 | A |
| ATOM | 285 | N | ALA | A | 637 | −4.462 | −11.934 | 12.373 | 1.00 | 44.33 | A |
| ATOM | 286 | CA | ALA | A | 637 | −5.906 | −11.818 | 12.545 | 1.00 | 43.81 | A |
| ATOM | 287 | CB | ALA | A | 637 | −6.238 | −11.493 | 13.996 | 1.00 | 43.88 | A |
| ATOM | 288 | C | ALA | A | 637 | −6.450 | −10.729 | 11.634 | 1.00 | 43.58 | A |
| ATOM | 289 | O | ALA | A | 637 | −5.828 | −9.677 | 11.465 | 1.00 | 42.65 | A |
| ATOM | 290 | N | GLY | A | 638 | −7.612 | −10.988 | 11.044 | 1.00 | 43.24 | A |
| ATOM | 291 | CA | GLY | A | 638 | −8.218 | −10.012 | 10.157 | 1.00 | 41.84 | A |
| ATOM | 292 | C | GLY | A | 638 | −9.481 | −9.419 | 10.741 | 1.00 | 41.01 | A |
| ATOM | 293 | O | GLY | A | 638 | −9.978 | −9.880 | 11.773 | 1.00 | 41.22 | A |
| ATOM | 294 | N | GLU | A | 639 | −10.006 | −8.397 | 10.075 | 1.00 | 40.04 | A |
| ATOM | 295 | CA | GLU | A | 639 | −11.222 | −7.733 | 10.525 | 1.00 | 37.62 | A |
| ATOM | 296 | CB | GLU | A | 639 | −11.469 | −6.470 | 9.695 | 1.00 | 39.78 | A |
| ATOM | 297 | CG | GLU | A | 639 | −12.702 | −5.688 | 10.127 | 1.00 | 44.07 | A |
| ATOM | 298 | CD | GLU | A | 639 | −13.102 | −4.611 | 9.134 | 1.00 | 46.08 | A |
| ATOM | 299 | OE1 | GLU | A | 639 | −14.145 | −3.961 | 9.358 | 1.00 | 48.25 | A |
| ATOM | 300 | OE2 | GLU | A | 639 | −12.381 | −4.416 | 8.128 | 1.00 | 47.68 | A |
| ATOM | 301 | C | GLU | A | 639 | −12.448 | −8.645 | 10.431 | 1.00 | 35.13 | A |
| ATOM | 302 | O | GLU | A | 639 | −13.392 | −8.509 | 11.219 | 1.00 | 34.00 | A |
| ATOM | 303 | N | PHE | A | 640 | −12.430 | −9.574 | 9.477 | 1.00 | 31.83 | A |
| ATOM | 304 | CA | PHE | A | 640 | −13.560 | −10.482 | 9.278 | 1.00 | 30.48 | A |
| ATOM | 305 | CB | PHE | A | 640 | −14.083 | −10.366 | 7.832 | 1.00 | 29.76 | A |
| ATOM | 306 | CG | PHE | A | 640 | −14.482 | −8.966 | 7.433 | 1.00 | 28.46 | A |
| ATOM | 307 | CD1 | PHE | A | 640 | −13.531 | −8.058 | 6.974 | 1.00 | 28.70 | A |
| ATOM | 308 | CD2 | PHE | A | 640 | −15.802 | −8.545 | 7.548 | 1.00 | 28.68 | A |
| ATOM | 309 | CE1 | PHE | A | 640 | −13.889 | −6.745 | 6.636 | 1.00 | 27.42 | A |
| ATOM | 310 | CE2 | PHE | A | 640 | −16.172 | −7.237 | 7.215 | 1.00 | 26.96 | A |
| ATOM | 311 | CZ | PHE | A | 640 | −15.211 | −6.337 | 6.759 | 1.00 | 27.64 | A |
| ATOM | 312 | C | PHE | A | 640 | −13.242 | −11.952 | 9.591 | 1.00 | 29.44 | A |
| ATOM | 313 | O | PHE | A | 640 | −14.118 | −12.817 | 9.499 | 1.00 | 27.82 | A |
| ATOM | 314 | N | GLY | A | 641 | −11.998 | −12.230 | 9.966 | 1.00 | 28.09 | A |
| ATOM | 315 | CA | GLY | A | 641 | −11.611 | −13.597 | 10.266 | 1.00 | 28.09 | A |
| ATOM | 316 | C | GLY | A | 641 | −10.105 | −13.766 | 10.389 | 1.00 | 28.66 | A |
| ATOM | 317 | O | GLY | A | 641 | −9.402 | −12.833 | 10.777 | 1.00 | 27.98 | A |
| ATOM | 318 | N | GLU | A | 642 | −9.609 | −14.955 | 10.052 | 1.00 | 27.87 | A |
| ATOM | 319 | CA | GLU | A | 642 | −8.185 | −15.249 | 10.140 | 1.00 | 28.48 | A |
| ATOM | 320 | CB | GLU | A | 642 | −7.969 | −16.715 | 10.559 | 1.00 | 31.49 | A |
| ATOM | 321 | CG | GLU | A | 642 | −8.655 | −17.116 | 11.879 | 1.00 | 35.67 | A |
| ATOM | 322 | CD | GLU | A | 642 | −8.289 | −18.526 | 12.345 | 1.00 | 38.58 | A |
| ATOM | 323 | OE1 | GLU | A | 642 | −8.407 | −19.482 | 11.544 | 1.00 | 39.58 | A |
| ATOM | 324 | OE2 | GLU | A | 642 | −7.884 | −18.676 | 13.521 | 1.00 | 39.80 | A |
| ATOM | 325 | C | GLU | A | 642 | −7.419 | −14.989 | 8.844 | 1.00 | 27.74 | A |
| ATOM | 326 | O | GLU | A | 642 | −7.980 | −15.043 | 7.744 | 1.00 | 26.91 | A |
| ATOM | 327 | N | VAL | A | 643 | −6.130 | −14.693 | 8.989 | 1.00 | 27.11 | A |
| ATOM | 328 | CA | VAL | A | 643 | −5.252 | −14.461 | 7.853 | 1.00 | 26.42 | A |
| ATOM | 329 | CB | VAL | A | 643 | −4.478 | −13.134 | 7.996 | 1.00 | 25.95 | A |
| ATOM | 330 | CG1 | VAL | A | 643 | −3.732 | −12.838 | 6.728 | 1.00 | 25.98 | A |
| ATOM | 331 | CG2 | VAL | A | 643 | −5.427 | −12.014 | 8.334 | 1.00 | 25.96 | A |
| ATOM | 332 | C | VAL | A | 643 | −4.268 | −15.620 | 7.891 | 1.00 | 26.37 | A |
| ATOM | 333 | O | VAL | A | 643 | −3.519 | −15.763 | 8.858 | 1.00 | 24.39 | A |
| ATOM | 334 | N | CYS | A | 644 | −4.267 | −16.433 | 6.836 | 1.00 | 25.63 | A |
| ATOM | 335 | CA | CYS | A | 644 | −3.409 | −17.616 | 6.763 | 1.00 | 25.19 | A |
| ATOM | 336 | CB | CYS | A | 644 | −4.275 | −18.883 | 6.718 | 1.00 | 25.46 | A |
| ATOM | 337 | SG | CYS | A | 644 | −5.746 | −18.849 | 7.750 | 1.00 | 28.36 | A |
| ATOM | 338 | C | CYS | A | 644 | −2.483 | −17.655 | 5.551 | 1.00 | 25.37 | A |
| ATOM | 339 | O | CYS | A | 644 | −2.556 | −16.811 | 4.657 | 1.00 | 25.07 | A |
| ATOM | 340 | N | SER | A | 645 | −1.615 | −18.658 | 5.532 | 1.00 | 23.66 | A |
| ATOM | 341 | CA | SER | A | 645 | −0.709 | −18.873 | 4.419 | 1.00 | 24.21 | A |
| ATOM | 342 | CB | SER | A | 645 | 0.750 | −18.801 | 4.862 | 1.00 | 24.10 | A |
| ATOM | 343 | OG | SER | A | 645 | 1.107 | −19.968 | 5.574 | 1.00 | 27.96 | A |
| ATOM | 344 | C | SER | A | 645 | −1.025 | −20.280 | 3.949 | 1.00 | 23.74 | A |
| ATOM | 345 | O | SER | A | 645 | −1.558 | −21.088 | 4.715 | 1.00 | 22.82 | A |
| ATOM | 346 | N | GLY | A | 646 | −0.703 | −20.575 | 2.695 | 1.00 | 24.19 | A |
| ATOM | 347 | CA | GLY | A | 646 | −0.985 | −21.896 | 2.171 | 1.00 | 22.79 | A |
| ATOM | 348 | C | GLY | A | 646 | −0.559 | −22.070 | 0.730 | 1.00 | 22.27 | A |
| ATOM | 349 | O | GLY | A | 646 | 0.255 | −21.304 | 0.208 | 1.00 | 21.87 | A |
| ATOM | 350 | N | HIS | A | 647 | −1.097 | −23.100 | 0.091 | 1.00 | 22.58 | A |
| ATOM | 351 | CA | HIS | A | 647 | −0.776 | −23.367 | −1.296 | 1.00 | 24.03 | A |
| ATOM | 352 | CB | HIS | A | 647 | −0.024 | −24.698 | −1.445 | 1.00 | 25.12 | A |
| ATOM | 353 | CG | HIS | A | 647 | 1.316 | −24.725 | −0.770 | 1.00 | 27.93 | A |
| ATOM | 354 | CD2 | HIS | A | 647 | 2.572 | −24.751 | −1.278 | 1.00 | 28.54 | A |
| ATOM | 355 | ND1 | HIS | A | 647 | 1.460 | −24.753 | 0.602 | 1.00 | 29.81 | A |
| ATOM | 356 | CE1 | HIS | A | 647 | 2.745 | −24.800 | 0.909 | 1.00 | 28.73 | A |
| ATOM | 357 | NE2 | HIS | A | 647 | 3.441 | −24.799 | −0.214 | 1.00 | 30.01 | A |
| ATOM | 358 | C | HIS | A | 647 | −2.054 | −23.392 | −2.125 | 1.00 | 22.29 | A |
| ATOM | 359 | O | HIS | A | 647 | −3.134 | −23.758 | −1.643 | 1.00 | 22.95 | A |
| ATOM | 360 | N | LEU | A | 648 | −1.913 | −22.977 | −3.376 | 1.00 | 22.24 | A |
| ATOM | 361 | CA | LEU | A | 648 | −3.010 | −22.935 | −4.323 | 1.00 | 22.62 | A |
| ATOM | 362 | CB | LEU | A | 648 | −3.302 | −21.488 | −4.743 | 1.00 | 22.09 | A |
| ATOM | 363 | CG | LEU | A | 648 | −4.285 | −21.306 | −5.898 | 1.00 | 21.11 | A |
| ATOM | 364 | CD1 | LEU | A | 648 | −5.639 | −21.893 | −5.505 | 1.00 | 22.47 | A |
| ATOM | 365 | CD2 | LEU | A | 648 | −4.418 | −19.834 | −6.250 | 1.00 | 21.63 | A |
| ATOM | 366 | C | LEU | A | 648 | −2.565 | −23.737 | −5.532 | 1.00 | 24.96 | A |
| ATOM | 367 | O | LEU | A | 648 | −1.525 | −23.447 | −6.131 | 1.00 | 25.51 | A |
| ATOM | 368 | N | LYS | A | 649 | −3.343 | −24.755 | −5.879 | 1.00 | 27.46 | A |
| ATOM | 369 | CA | LYS | A | 649 | −3.029 | −25.596 | −7.024 | 1.00 | 31.13 | A |
| ATOM | 370 | CB | LYS | A | 649 | −2.997 | −27.078 | −6.610 | 1.00 | 31.15 | A |
| ATOM | 371 | CG | LYS | A | 649 | −2.185 | −27.994 | −7.529 | 0.00 | 31.95 | A |
| ATOM | 372 | CD | LYS | A | 649 | −2.818 | −28.177 | −8.903 | 0.00 | 32.48 | A |
| ATOM | 373 | CE | LYS | A | 649 | −1.940 | −29.047 | −9.801 | 0.00 | 32.88 | A |
| ATOM | 374 | NZ | LYS | A | 649 | −1.650 | −30.379 | −9.199 | 0.00 | 33.21 | A |
| ATOM | 375 | C | LYS | A | 649 | −4.115 | −25.373 | −8.066 | 1.00 | 33.65 | A |
| ATOM | 376 | O | LYS | A | 649 | −5.272 | −25.744 | −7.858 | 1.00 | 35.71 | A |
| ATOM | 377 | N | LEU | A | 650 | −3.740 | −24.753 | −9.178 | 1.00 | 35.03 | A |
| ATOM | 378 | CA | LEU | A | 650 | −4.681 | −24.498 | −10.265 | 1.00 | 36.97 | A |
| ATOM | 379 | CB | LEU | A | 650 | −4.654 | −23.021 | −10.659 | 1.00 | 36.46 | A |
| ATOM | 380 | CG | LEU | A | 650 | −5.111 | −22.027 | −9.592 | 1.00 | 37.31 | A |
| ATOM | 381 | CD1 | LEU | A | 650 | −4.809 | −20.616 | −10.058 | 1.00 | 37.80 | A |
| ATOM | 382 | CD2 | LEU | A | 650 | −6.596 | −22.211 | −9.307 | 1.00 | 35.30 | A |
| ATOM | 383 | C | LEU | A | 650 | −4.308 | −25.361 | −11.465 | 1.00 | 36.94 | A |
| ATOM | 384 | O | LEU | A | 650 | −3.274 | −26.027 | −11.458 | 1.00 | 35.61 | A |
| ATOM | 385 | N | ARG | A | 654 | 1.042 | −25.384 | −11.878 | 1.00 | 38.79 | A |
| ATOM | 386 | CA | ARG | A | 654 | 1.946 | −25.673 | −10.770 | 1.00 | 38.06 | A |
| ATOM | 387 | CB | ARG | A | 654 | 3.360 | −25.184 | −11.092 | 1.00 | 40.05 | A |
| ATOM | 388 | CG | ARG | A | 654 | 4.377 | −25.516 | −10.010 | 0.00 | 40.81 | A |
| ATOM | 389 | CD | ARG | A | 654 | 5.712 | −24.843 | −10.271 | 0.00 | 41.95 | A |
| ATOM | 390 | NE | ARG | A | 654 | 6.674 | −25.111 | −9.205 | 0.00 | 42.83 | A |
| ATOM | 391 | CZ | ARG | A | 654 | 7.878 | −24.553 | −9.127 | 0.00 | 43.31 | A |
| ATOM | 392 | NH1 | ARG | A | 654 | 8.273 | −23.692 | −10.055 | 0.00 | 43.60 | A |
| ATOM | 393 | NH2 | ARG | A | 654 | 8.687 | −24.854 | −8.121 | 0.00 | 43.60 | A |
| ATOM | 394 | C | ARG | A | 654 | 1.477 | −25.019 | −9.473 | 1.00 | 36.96 | A |
| ATOM | 395 | O | ARG | A | 654 | 0.953 | −23.905 | −9.479 | 1.00 | 37.48 | A |
| ATOM | 396 | N | GLU | A | 655 | 1.677 | −25.718 | −8.360 | 1.00 | 34.47 | A |
| ATOM | 397 | CA | GLU | A | 655 | 1.279 | −25.210 | −7.055 | 1.00 | 32.62 | A |
| ATOM | 398 | CB | GLU | A | 655 | 1.489 | −26.287 | −5.995 | 1.00 | 34.16 | A |
| ATOM | 399 | CG | GLU | A | 655 | 0.948 | −25.913 | −4.628 | 1.00 | 37.34 | A |
| ATOM | 400 | CD | GLU | A | 655 | 1.042 | −27.056 | −3.638 | 1.00 | 39.34 | A |
| ATOM | 401 | OE1 | GLU | A | 655 | 2.177 | −27.464 | −3.306 | 1.00 | 40.30 | A |
| ATOM | 402 | OE2 | GLU | A | 655 | −0.017 | −27.547 | −3.200 | 1.00 | 40.82 | A |
| ATOM | 403 | C | GLU | A | 655 | 2.075 | −23.963 | −6.672 | 1.00 | 30.39 | A |
| ATOM | 404 | O | GLU | A | 655 | 3.263 | −23.864 | −6.970 | 1.00 | 30.59 | A |
| ATOM | 405 | N | ILE | A | 656 | 1.417 | −23.006 | −6.024 | 1.00 | 26.58 | A |
| ATOM | 406 | CA | ILE | A | 656 | 2.096 | −21.787 | −5.599 | 1.00 | 24.21 | A |
| ATOM | 407 | CB | ILE | A | 656 | 1.779 | −20.575 | −6.522 | 1.00 | 22.73 | A |
| ATOM | 408 | CG2 | ILE | A | 656 | 2.273 | −20.843 | −7.949 | 1.00 | 21.90 | A |
| ATOM | 409 | CG1 | ILE | A | 656 | 0.271 | −20.283 | −6.494 | 1.00 | 22.82 | A |
| ATOM | 410 | CD1 | ILE | A | 656 | −0.124 | −18.937 | −7.096 | 1.00 | 20.25 | A |
| ATOM | 411 | C | ILE | A | 656 | 1.680 | −21.399 | −4.185 | 1.00 | 22.57 | A |
| ATOM | 412 | O | ILE | A | 656 | 0.593 | −21.758 | −3.731 | 1.00 | 21.28 | A |
| ATOM | 413 | N | PHE | A | 657 | 2.552 | −20.669 | −3.493 | 1.00 | 22.56 | A |
| ATOM | 414 | CA | PHE | A | 657 | 2.251 | −20.190 | −2.147 | 1.00 | 21.07 | A |
| ATOM | 415 | CB | PHE | A | 657 | 3.516 | −19.648 | −1.462 | 1.00 | 27.21 | A |
| ATOM | 416 | CG | PHE | A | 657 | 4.424 | −20.719 | −0.909 | 1.00 | 32.40 | A |
| ATOM | 417 | CD1 | PHE | A | 657 | 5.499 | −21.203 | −1.652 | 1.00 | 36.01 | A |
| ATOM | 418 | CD2 | PHE | A | 657 | 4.188 | −21.258 | 0.355 | 1.00 | 35.51 | A |
| ATOM | 419 | CE1 | PHE | A | 657 | 6.327 | −22.209 | −1.144 | 1.00 | 36.52 | A |
| ATOM | 420 | CE2 | PHE | A | 657 | 5.009 | −22.265 | 0.872 | 1.00 | 37.58 | A |
| ATOM | 421 | CZ | PHE | A | 657 | 6.080 | −22.740 | 0.120 | 1.00 | 37.93 | A |
| ATOM | 422 | C | PHE | A | 657 | 1.240 | −19.054 | −2.292 | 1.00 | 20.47 | A |
| ATOM | 423 | O | PHE | A | 657 | 1.295 | −18.298 | −3.261 | 1.00 | 19.30 | A |
| ATOM | 424 | N | VAL | A | 658 | 0.314 | −18.941 | −1.347 | 1.00 | 17.78 | A |
| ATOM | 425 | CA | VAL | A | 658 | −0.676 | −17.873 | −1.392 | 1.00 | 16.97 | A |
| ATOM | 426 | CB | VAL | A | 658 | −2.004 | −18.311 | −2.112 | 1.00 | 15.03 | A |
| ATOM | 427 | CG1 | VAL | A | 658 | −1.779 | −18.443 | −3.615 | 1.00 | 13.58 | A |
| ATOM | 428 | CG2 | VAL | A | 658 | −2.504 | −19.636 | −1.546 | 1.00 | 17.13 | A |
| ATOM | 429 | C | VAL | A | 658 | −1.034 | −17.421 | 0.012 | 1.00 | 17.80 | A |
| ATOM | 430 | O | VAL | A | 658 | −0.782 | −18.140 | 0.990 | 1.00 | 16.73 | A |
| ATOM | 431 | N | ALA | A | 659 | −1.596 | −16.218 | 0.102 | 1.00 | 14.11 | A |
| ATOM | 432 | CA | ALA | A | 659 | −2.060 | −15.671 | 1.367 | 1.00 | 15.67 | A |
| ATOM | 433 | CB | ALA | A | 659 | −1.810 | −14.162 | 1.433 | 1.00 | 14.67 | A |
| ATOM | 434 | C | ALA | A | 659 | −3.552 | −15.965 | 1.283 | 1.00 | 16.07 | A |
| ATOM | 435 | O | ALA | A | 659 | −4.150 | −15.830 | 0.207 | 1.00 | 16.48 | A |
| ATOM | 436 | N | ILE | A | 660 | −4.145 | −16.377 | 2.401 | 1.00 | 17.28 | A |
| ATOM | 437 | CA | ILE | A | 660 | −5.557 | −16.756 | 2.453 | 1.00 | 20.16 | A |
| ATOM | 438 | CB | ILE | A | 660 | −5.691 | −18.307 | 2.608 | 1.00 | 20.13 | A |
| ATOM | 439 | CG2 | ILE | A | 660 | −7.149 | −18.725 | 2.565 | 1.00 | 22.79 | A |
| ATOM | 440 | CG1 | ILE | A | 660 | −4.938 | −19.019 | 1.483 | 1.00 | 21.87 | A |
| ATOM | 441 | CD1 | ILE | A | 660 | −4.887 | −20.538 | 1.654 | 1.00 | 22.50 | A |
| ATOM | 442 | C | ILE | A | 660 | −6.309 | −16.101 | 3.609 | 1.00 | 22.05 | A |
| ATOM | 443 | O | ILE | A | 660 | −5.937 | −16.254 | 4.774 | 1.00 | 22.45 | A |
| ATOM | 444 | N | LYS | A | 661 | −7.367 | −15.372 | 3.274 | 1.00 | 22.33 | A |
| ATOM | 445 | CA | LYS | A | 661 | −8.201 | −14.707 | 4.267 | 1.00 | 23.44 | A |
| ATOM | 446 | CB | LYS | A | 661 | −8.507 | −13.270 | 3.850 | 1.00 | 26.63 | A |
| ATOM | 447 | CG | LYS | A | 661 | −7.426 | −12.272 | 4.175 | 1.00 | 30.21 | A |
| ATOM | 448 | CD | LYS | A | 661 | −7.924 | −10.863 | 3.894 | 1.00 | 32.40 | A |
| ATOM | 449 | CE | LYS | A | 661 | −6.980 | −9.824 | 4.450 | 1.00 | 34.22 | A |
| ATOM | 450 | NZ | LYS | A | 661 | −7.561 | −8.466 | 4.315 | 1.00 | 34.03 | A |
| ATOM | 451 | C | LYS | A | 661 | −9.509 | −15.479 | 4.376 | 1.00 | 23.79 | A |
| ATOM | 452 | O | LYS | A | 661 | −10.109 | −15.837 | 3.366 | 1.00 | 22.44 | A |
| ATOM | 453 | N | THR | A | 662 | −9.940 | −15.751 | 5.599 | 1.00 | 22.77 | A |
| ATOM | 454 | CA | THR | A | 662 | −11.174 | −16.485 | 5.800 | 1.00 | 23.53 | A |
| ATOM | 455 | CB | THR | A | 662 | −10.938 | −17.758 | 6.637 | 1.00 | 25.15 | A |
| ATOM | 456 | OG1 | THR | A | 662 | −10.286 | −17.399 | 7.860 | 1.00 | 26.34 | A |
| ATOM | 457 | CG2 | THR | A | 662 | −10.065 | −18.748 | 5.872 | 1.00 | 25.34 | A |
| ATOM | 458 | C | THR | A | 662 | −12.183 | −15.608 | 6.518 | 1.00 | 24.19 | A |
| ATOM | 459 | O | THR | A | 662 | −11.814 | −14.694 | 7.256 | 1.00 | 25.54 | A |
| ATOM | 460 | N | LEU | A | 663 | −13.459 | −15.887 | 6.282 | 1.00 | 24.00 | A |
| ATOM | 461 | CA | LEU | A | 663 | −14.542 | −15.146 | 6.904 | 1.00 | 26.09 | A |
| ATOM | 462 | CB | LEU | A | 663 | −15.667 | −14.907 | 5.887 | 1.00 | 23.14 | A |
| ATOM | 463 | CG | LEU | A | 663 | −16.886 | −14.132 | 6.383 | 1.00 | 24.98 | A |
| ATOM | 464 | CD1 | LEU | A | 663 | −16.448 | −12.756 | 6.855 | 1.00 | 22.54 | A |
| ATOM | 465 | CD2 | LEU | A | 663 | −17.931 | −14.012 | 5.263 | 1.00 | 23.69 | A |
| ATOM | 466 | C | LEU | A | 663 | −15.067 | −15.977 | 8.076 | 1.00 | 27.30 | A |
| ATOM | 467 | O | LEU | A | 663 | −15.493 | −17.115 | 7.888 | 1.00 | 28.82 | A |
| ATOM | 468 | N | LYS | A | 664 | −15.026 | −15.401 | 9.275 | 1.00 | 28.84 | A |
| ATOM | 469 | CA | LYS | A | 664 | −15.490 | −16.061 | 10.490 | 1.00 | 30.02 | A |
| ATOM | 470 | CB | LYS | A | 664 | −15.417 | −15.086 | 11.674 | 1.00 | 31.14 | A |
| ATOM | 471 | CG | LYS | A | 664 | −16.325 | −13.865 | 11.549 | 0.00 | 31.57 | A |
| ATOM | 472 | CD | LYS | A | 664 | −16.193 | −12.935 | 12.752 | 0.00 | 32.18 | A |
| ATOM | 473 | CE | LYS | A | 664 | −17.158 | −11.760 | 12.657 | 0.00 | 32.50 | A |
| ATOM | 474 | NZ | LYS | A | 664 | −16.945 | −10.972 | 11.413 | 0.00 | 32.80 | A |
| ATOM | 475 | C | LYS | A | 664 | −16.923 | −16.572 | 10.334 | 1.00 | 30.86 | A |
| ATOM | 476 | O | LYS | A | 664 | −17.802 | −15.841 | 9.880 | 1.00 | 31.20 | A |
| ATOM | 477 | N | SER | A | 665 | −17.152 | −17.829 | 10.711 | 1.00 | 31.16 | A |
| ATOM | 478 | CA | SER | A | 665 | −18.479 | −18.439 | 10.628 | 1.00 | 30.42 | A |
| ATOM | 479 | CB | SER | A | 665 | −18.431 | −19.861 | 11.178 | 1.00 | 32.90 | A |
| ATOM | 480 | OG | SER | A | 665 | −18.013 | −19.840 | 12.532 | 1.00 | 35.02 | A |
| ATOM | 481 | C | SER | A | 665 | −19.487 | −17.629 | 11.439 | 1.00 | 30.07 | A |
| ATOM | 482 | O | SER | A | 665 | −19.128 | −17.026 | 12.455 | 1.00 | 29.38 | A |
| ATOM | 483 | N | GLY | A | 666 | −20.743 | −17.622 | 10.993 | 1.00 | 28.66 | A |
| ATOM | 484 | CA | GLY | A | 666 | −21.781 | −16.876 | 11.689 | 1.00 | 27.97 | A |
| ATOM | 485 | C | GLY | A | 666 | −21.789 | −15.423 | 11.250 | 1.00 | 27.26 | A |
| ATOM | 486 | O | GLY | A | 666 | −22.273 | −14.535 | 11.958 | 1.00 | 25.74 | A |
| ATOM | 487 | N | TYR | A | 667 | −21.239 | −15.186 | 10.065 | 1.00 | 27.08 | A |
| ATOM | 488 | CA | TYR | A | 667 | −21.156 | −13.847 | 9.494 | 1.00 | 25.72 | A |
| ATOM | 489 | CB | TYR | A | 667 | −20.169 | −13.854 | 8.325 | 1.00 | 25.23 | A |
| ATOM | 490 | CG | TYR | A | 667 | −20.563 | −14.793 | 7.207 | 1.00 | 24.76 | A |
| ATOM | 491 | CD1 | TYR | A | 667 | −21.567 | −14.447 | 6.301 | 1.00 | 23.91 | A |
| ATOM | 492 | CR1 | TYR | A | 667 | −21.951 | −15.314 | 5.284 | 1.00 | 25.60 | A |
| ATOM | 493 | CD2 | TYR | A | 667 | −19.947 | −16.040 | 7.067 | 1.00 | 23.97 | A |
| ATOM | 494 | CE2 | TYR | A | 667 | −20.323 | −16.917 | 6.051 | 1.00 | 25.46 | A |
| ATOM | 495 | CZ | TYR | A | 667 | −21.325 | −16.546 | 5.163 | 1.00 | 26.43 | A |
| ATOM | 496 | OH | TYR | A | 667 | −21.691 | −17.392 | 4.143 | 1.00 | 28.78 | A |
| ATOM | 497 | C | TYR | A | 667 | −22.515 | −13.345 | 9.004 | 1.00 | 25.35 | A |
| ATOM | 498 | O | TYR | A | 667 | −23.358 | −14.125 | 8.569 | 1.00 | 24.49 | A |
| ATOM | 499 | N | THR | A | 668 | −22.720 | −12.037 | 9.081 | 1.00 | 24.59 | A |
| ATOM | 500 | CA | THR | A | 668 | −23.963 | −11.444 | 8.612 | 1.00 | 24.16 | A |
| ATOM | 501 | CB | THR | A | 668 | −24.265 | −10.131 | 9.344 | 1.00 | 25.06 | A |
| ATOM | 502 | OG1 | THR | A | 668 | −23.182 | −9.209 | 9.132 | 1.00 | 28.51 | A |
| ATOM | 503 | CG2 | THR | A | 668 | −24.432 | −10.381 | 10.837 | 1.00 | 24.37 | A |
| ATOM | 504 | C | THR | A | 668 | −23.801 | −11.147 | 7.119 | 1.00 | 24.62 | A |
| ATOM | 505 | O | THR | A | 668 | −22.689 | −11.224 | 6.585 | 1.00 | 21.64 | A |
| ATOM | 506 | N | GLU | A | 669 | −24.908 | −10.809 | 6.459 | 1.00 | 22.42 | A |
| ATOM | 507 | CA | GLU | A | 669 | −24.901 | −10.493 | 5.031 | 1.00 | 23.71 | A |
| ATOM | 508 | CB | GLU | A | 669 | −26.315 | −10.102 | 4.578 | 1.00 | 24.53 | A |
| ATOM | 509 | CG | GLU | A | 669 | −26.450 | −9.796 | 3.099 | 0.00 | 25.34 | A |
| ATOM | 510 | CD | GLU | A | 669 | −26.084 | −10.978 | 2.226 | 0.00 | 25.88 | A |
| ATOM | 511 | OE1 | GLU | A | 669 | −26.743 | −12.033 | 2.345 | 0.00 | 26.22 | A |
| ATOM | 512 | 0E2 | GLU | A | 669 | −25.137 | −10.853 | 1.422 | 0.00 | 26.22 | A |
| ATOM | 513 | C | GLU | A | 669 | −23.926 | −9.351 | 4.744 | 1.00 | 21.59 | A |
| ATOM | 514 | O | GLU | A | 669 | −23.152 | −9.408 | 3.797 | 1.00 | 23.09 | A |
| ATOM | 515 | N | LYS | A | 670 | −23.956 | −8.329 | 5.589 | 1.00 | 22.19 | A |
| ATOM | 516 | CA | LYS | A | 670 | −23.092 | −7.171 | 5.439 | 1.00 | 20.44 | A |
| ATOM | 517 | CB | LYS | A | 670 | −23.522 | −6.073 | 6.415 | 1.00 | 19.94 | A |
| ATOM | 518 | CG | LYS | A | 670 | −22.615 | −4.846 | 6.417 | 1.00 | 19.99 | A |
| ATOM | 519 | CD | LYS | A | 670 | −23.150 | −3.762 | 7.315 | 1.00 | 19.07 | A |
| ATOM | 520 | CE | LYS | A | 670 | −22.130 | −2.654 | 7.524 | 1.00 | 21.46 | A |
| ATOM | 521 | NZ | LYS | A | 670 | −21.542 | −2.169 | 6.255 | 1.00 | 21.44 | A |
| ATOM | 522 | C | LYS | A | 670 | −21.622 | −7.527 | 5.668 | 1.00 | 22.01 | A |
| ATOM | 523 | O | LYS | A | 670 | −20.750 | −7.100 | 4.913 | 1.00 | 18.77 | A |
| ATOM | 524 | N | GLN | A | 671 | −21.339 | −8.305 | 6.707 | 1.00 | 20.25 | A |
| ATOM | 525 | CA | GLN | A | 671 | −19.957 | −8.678 | 6.969 | 1.00 | 21.57 | A |
| ATOM | 526 | CB | GLN | A | 671 | −19.857 | −9.540 | 8.226 | 1.00 | 19.59 | A |
| ATOM | 527 | CG | GLN | A | 671 | −20.174 | −8.772 | 9.489 | 1.00 | 23.04 | A |
| ATOM | 528 | CD | GLN | A | 671 | −20.090 | −9.646 | 10.715 | 1.00 | 23.12 | A |
| ATOM | 529 | OE1 | GLN | A | 671 | −20.591 | −10.765 | 10.716 | 1.00 | 25.03 | A |
| ATOM | 530 | NE2 | GLN | A | 671 | −19.454 | −9.141 | 11.766 | 1.00 | 26.08 | A |
| ATOM | 531 | C | GLN | A | 671 | −19.360 | −9.412 | 5.774 | 1.00 | 19.09 | A |
| ATOM | 532 | O | GLN | A | 671 | −18.203 | −9.188 | 5.419 | 1.00 | 19.45 | A |
| ATOM | 533 | N | ARG | A | 672 | −20.159 | −10.263 | 5.143 | 1.00 | 17.49 | A |
| ATOM | 534 | CA | ARG | A | 672 | −19.711 | −11.014 | 3.978 | 1.00 | 18.83 | A |
| ATOM | 535 | CB | ARG | A | 672 | −20.775 | −12.044 | 3.584 | 1.00 | 18.14 | A |
| ATOM | 536 | CG | ARG | A | 672 | −20.482 | −12.799 | 2.295 | 1.00 | 19.76 | A |
| ATOM | 537 | CD | ARG | A | 672 | −21.620 | −13.754 | 1.961 | 1.00 | 21.40 | A |
| ATOM | 538 | NE | ARG | A | 672 | −21.459 | −14.337 | 0.633 | 1.00 | 23.81 | A |
| ATOM | 539 | CZ | ARG | A | 672 | −21.574 | −13.656 | −0.506 | 1.00 | 23.75 | A |
| ATOM | 540 | NH1 | ARG | A | 672 | −21.863 | −12.361 | −0.486 | 1.00 | 24.13 | A |
| ATOM | 541 | NH2 | ARG | A | 672 | −21.377 | −14.267 | −1.665 | 1.00 | 23.85 | A |
| ATOM | 542 | C | ARG | A | 672 | −19.461 | −10.053 | 2.813 | 1.00 | 19.29 | A |
| ATOM | 543 | O | AEG | A | 672 | −18.476 | −10.172 | 2.080 | 1.00 | 17.76 | A |
| ATOM | 544 | N | ARG | A | 673 | −20.376 | −9.102 | 2.655 | 1.00 | 19.04 | A |
| ATOM | 545 | CA | ARG | A | 673 | −20.280 | −8.113 | 1.592 | 1.00 | 18.53 | A |
| ATOM | 546 | CB | ARG | A | 673 | −21.499 | −7.192 | 1.599 | 1.00 | 16.97 | A |
| ATOM | 547 | CG | ARG | A | 673 | −21.472 | −6.171 | 0.481 | 1.00 | 16.95 | A |
| ATOM | 548 | CD | ARG | A | 673 | −22.763 | −5.428 | 0.403 | 1.00 | 18.42 | A |
| ATOM | 549 | NE | ARG | A | 673 | −22.963 | −4.625 | 1.595 | 1.00 | 23.56 | A |
| ATOM | 550 | CZ | ARG | A | 673 | −24.042 | −4.692 | 2.366 | 1.00 | 20.84 | A |
| ATOM | 551 | NE1 | ARG | A | 673 | −25.022 | −5.533 | 2.066 | 1.00 | 22.79 | A |
| ATOM | 552 | NH2 | ARG | A | 673 | −24.140 | −3.912 | 3.434 | 1.00 | 21.92 | A |
| ATOM | 553 | C | ARG | A | 673 | −19.028 | −7.269 | 1.740 | 1.00 | 17.35 | A |
| ATOM | 554 | O | ARG | A | 673 | −18.245 | −7.137 | 0.802 | 1.00 | 18.98 | A |
| ATOM | 555 | N | ASP | A | 674 | −18.852 | −6.678 | 2.915 | 1.00 | 15.56 | A |
| ATOM | 556 | CA | ASP | A | 674 | −17.690 | −5.846 | 3.151 | 1.00 | 17.33 | A |
| ATOM | 557 | CB | ASP | A | 674 | −17.810 | −5.182 | 4.527 | 1.00 | 18.55 | A |
| ATOM | 558 | CG | ASP | A | 674 | −19.012 | −4.227 | 4.609 | 1.00 | 22.12 | A |
| ATOM | 559 | OD1 | ASP | A | 674 | −19.522 | −3.825 | 3.542 | 1.00 | 19.41 | A |
| ATOM | 560 | OD2 | ASP | A | 674 | −19.441 | −3.870 | 5.722 | 1.00 | 23.46 | A |
| ATOM | 561 | C | ASP | A | 674 | −16.376 | −6.635 | 2.991 | 1.00 | 16.55 | A |
| ATOM | 562 | O | ASP | A | 674 | −15.400 | −6.145 | 2.412 | 1.00 | 14.92 | A |
| ATOM | 563 | N | PHE | A | 675 | −16.362 | −7.864 | 3.476 | 1.00 | 14.43 | A |
| ATOM | 564 | CA | PHE | A | 675 | −15.190 | −8.711 | 3.345 | 1.00 | 15.13 | A |
| ATOM | 565 | CB | PHE | A | 675 | −15.483 | −10.070 | 3.969 | 1.00 | 14.23 | A |
| ATOM | 566 | CG | PHE | A | 675 | −14.376 | −11.054 | 3.821 | 1.00 | 14.93 | A |
| ATOM | 567 | CD1 | PHE | A | 675 | −13.181 | −10.873 | 4.501 | 1.00 | 17.64 | A |
| ATOM | 568 | CD2 | PHE | A | 675 | −14.543 | −12.188 | 3.040 | 1.00 | 16.15 | A |
| ATOM | 569 | CE1 | PHE | A | 675 | −12.164 | −11.815 | 4.414 | 1.00 | 17.02 | A |
| ATOM | 570 | CE2 | PHE | A | 675 | −13.536 | −13.135 | 2.944 | 1.00 | 19.09 | A |
| ATOM | 571 | CZ | PHE | A | 675 | −12.342 | −12.949 | 3.636 | 1.00 | 18.72 | A |
| ATOM | 572 | C | PHE | A | 675 | −14.812 | −8.912 | 1.873 | 1.00 | 14.71 | A |
| ATOM | 573 | O | PHE | A | 675 | −13.672 | −8.655 | 1.464 | 1.00 | 13.96 | A |
| ATOM | 574 | N | LEU | A | 676 | −15.780 | −9.363 | 1.080 | 1.00 | 13.97 | A |
| ATOM | 575 | CA | LEU | A | 676 | −15.550 | −9.638 | −0.331 | 1.00 | 12.30 | A |
| ATOM | 576 | CB | LEU | A | 676 | −16.721 | −10.465 | −0.913 | 1.00 | 13.83 | A |
| ATOM | 577 | CG | LEU | A | 676 | −16.885 | −11.904 | −0.364 | 1.00 | 13.35 | A |
| ATOM | 578 | CD1 | LEU | A | 676 | −18.132 | −12.553 | −0.923 | 1.00 | 14.68 | A |
| ATOM | 579 | CD2 | LEU | A | 676 | −15.665 | −12.732 | −0.726 | 1.00 | 14.56 | A |
| ATOM | 580 | C | LEU | A | 676 | −15.318 | −8.387 | −1.172 | 1.00 | 12.89 | A |
| ATOM | 581 | O | LEU | A | 676 | −14.816 | −8.488 | −2.292 | 1.00 | 14.97 | A |
| ATOM | 582 | N | SER | A | 677 | −15.663 | −7.212 | −0.651 | 1.00 | 11.83 | A |
| ATOM | 583 | CA | SER | A | 677 | −15.448 | −5.999 | −1.438 | 1.00 | 14.06 | A |
| ATOM | 584 | CB | SER | A | 677 | −16.016 | −4.758 | −0.733 | 1.00 | 12.26 | A |
| ATOM | 585 | OG | SER | A | 677 | −15.253 | −4.398 | 0.404 | 1.00 | 15.09 | A |
| ATOM | 586 | C | SER | A | 677 | −13.952 | −5.848 | −1.665 | 1.00 | 14.36 | A |
| ATOM | 587 | O | SER | A | 677 | −13.524 | −5.330 | −2.685 | 1.00 | 14.36 | A |
| ATOM | 588 | N | GLU | A | 678 | −13.144 | −6.304 | −0.715 | 1.00 | 16.37 | A |
| ATOM | 589 | CA | GLU | A | 678 | −11.705 | −6.200 | −0.916 | 1.00 | 15.57 | A |
| ATOM | 590 | CB | GLU | A | 678 | −10.946 | −6.832 | 0.246 | 1.00 | 19.69 | A |
| ATOM | 591 | CG | GLU | A | 678 | −9.443 | −6.939 | −0.008 | 1.00 | 24.61 | A |
| ATOM | 592 | CD | GLU | A | 678 | −8.694 | −7.521 | 1.166 | 1.00 | 27.65 | A |
| ATOM | 593 | OE1 | GLU | A | 678 | −9.219 | −8.448 | 1.807 | 1.00 | 29.80 | A |
| ATOM | 594 | OE2 | GLU | A | 678 | −7.571 | −7.052 | 1.440 | 1.00 | 32.17 | A |
| ATOM | 595 | CG | GLU | A | 678 | −11.328 | −6.885 | −2.240 | 1.00 | 14.92 | A |
| ATOM | 596 | O | GLU | A | 678 | −10.498 | −6.386 | −2.996 | 1.00 | 16.05 | A |
| ATOM | 597 | N | ALA | A | 679 | −11.958 | −8.015 | −2.532 | 1.00 | 12.66 | A |
| ATOM | 598 | CA | ALA | A | 679 | −11.659 | −8.737 | −3.761 | 1.00 | 13.34 | A |
| ATOM | 599 | CB | ALA | A | 679 | −12.111 | −10.186 | −3.650 | 1.00 | 9.98 | A |
| ATOM | 600 | C | ALA | A | 679 | −12.296 | −8.084 | −4.981 | 1.00 | 13.80 | A |
| ATOM | 601 | O | ALA | A | 679 | −11.699 | −8.078 | −6.059 | 1.00 | 12.31 | A |
| ATOM | 602 | N | SER | A | 680 | −13.503 | −7.544 | −4.829 | 1.00 | 14.31 | A |
| ATOM | 603 | CA | SER | A | 680 | −14.156 | −6.915 | −5.975 | 1.00 | 14.70 | A |
| ATOM | 604 | CB | SER | A | 680 | −15.637 | −6.606 | −5.666 | 1.00 | 13.71 | A |
| ATOM | 605 | OG | SER | A | 680 | −15.802 | −5.683 | −4.610 | 1.00 | 19.60 | A |
| ATOM | 606 | C | SER | A | 680 | −13.369 | −5.665 | −6.365 | 1.00 | 15.81 | A |
| ATOM | 607 | O | SER | A | 680 | −13.449 | −5.192 | −7.496 | 1.00 | 17.05 | A |
| ATOM | 608 | N | ILE | A | 681 | −12.566 | −5.160 | −5.431 | 1.00 | 15.14 | A |
| ATOM | 609 | CA | ILE | A | 681 | −11.739 | −3.998 | −5.695 | 1.00 | 14.43 | A |
| ATOM | 610 | CB | ILE | A | 681 | −11.583 | −3.167 | −4.412 | 1.00 | 15.35 | A |
| ATOM | 611 | CG2 | ILE | A | 681 | −10.483 | −2.107 | −4.589 | 1.00 | 13.45 | A |
| ATOM | 612 | CG1 | ILE | A | 681 | −12.955 | −2.582 | −4.050 | 1.00 | 12.39 | A |
| ATOM | 613 | CD1 | ILE | A | 681 | −12.965 | −1.725 | −2.820 | 1.00 | 13.10 | A |
| ATOM | 614 | C | ILE | A | 681 | −10.382 | −4.441 | −6.241 | 1.00 | 15.72 | A |
| ATOM | 615 | O | ILE | A | 681 | −10.014 | −4.091 | −7.371 | 1.00 | 15.31 | A |
| ATOM | 616 | N | MET | A | 682 | −9.658 | −5.247 | −5.465 | 1.00 | 14.71 | A |
| ATOM | 617 | CA | MET | A | 682 | −8.349 | −5.746 | −5.871 | 1.00 | 14.74 | A |
| ATOM | 618 | CE | MET | A | 682 | −7.862 | −6.775 | −4.835 | 1.00 | 15.19 | A |
| ATOM | 619 | CG | MET | A | 682 | −6.417 | −7.225 | −5.012 | 1.00 | 17.99 | A |
| ATOM | 620 | SD | MET | A | 682 | −5.958 | −8.505 | −3.763 | 1.00 | 18.16 | A |
| ATOM | 621 | CE | MET | A | 682 | −6.407 | −7.626 | −2.305 | 1.00 | 8.10 | A |
| ATOM | 622 | C | MET | A | 682 | −8.384 | −6.381 | −7.267 | 1.00 | 13.42 | A |
| ATOM | 623 | O | MET | A | 682 | −7.472 | −6.179 | −8.076 | 1.00 | 12.39 | A |
| ATOM | 624 | N | GLY | A | 683 | −9.463 | −7.108 | −7.563 | 1.00 | 11.77 | A |
| ATOM | 625 | CA | GLY | A | 683 | −9.598 | −7.780 | −8.856 | 1.00 | 10.35 | A |
| ATOM | 626 | C | GLY | A | 683 | −9.632 | −6.903 | −10.105 | 1.00 | 11.65 | A |
| ATOM | 627 | O | GLY | A | 683 | −9.492 | −7.388 | −11.229 | 1.00 | 10.47 | A |
| ATOM | 628 | N | GLN | A | 684 | −9.816 | −5.607 | −9.911 | 1.00 | 11.57 | A |
| ATOM | 629 | CA | GLN | A | 684 | −9.862 | −4.670 | −11.032 | 1.00 | 13.82 | A |
| ATOM | 630 | CE | GLN | A | 684 | −10.759 | −3.487 | −10.680 | 1.00 | 14.52 | A |
| ATOM | 631 | CG | GLN | A | 684 | −12.201 | −3.851 | −10.377 | 1.00 | 13.27 | A |
| ATOM | 632 | CD | GLN | A | 684 | −13.003 | −2.641 | −9.965 | 1.00 | 13.03 | A |
| ATOM | 633 | OE1 | GLN | A | 684 | −12.961 | −1.601 | −10.637 | 1.00 | 14.53 | A |
| ATOM | 634 | NE2 | GLN | A | 684 | −13.730 | −2.754 | −8.857 | 1.00 | 9.41 | A |
| ATOM | 635 | C | GLN | A | 684 | −8.475 | −4.129 | −11.345 | 1.00 | 14.70 | A |
| ATOM | 636 | O | GLN | A | 684 | −8.274 | −3.438 | −12.347 | 1.00 | 13.46 | A |
| ATOM | 637 | N | PHE | A | 685 | −7.521 | −4.425 | −10.469 | 1.00 | 14.39 | A |
| ATOM | 638 | CA | PHE | A | 685 | −6.156 | −3.925 | −10.639 | 1.00 | 14.62 | A |
| ATOM | 639 | CE | PHE | A | 685 | −5.644 | −3.368 | −9.313 | 1.00 | 13.37 | A |
| ATOM | 640 | CG | PHE | A | 685 | −6.545 | −2.344 | −8.696 | 1.00 | 13.64 | A |
| ATOM | 641 | CD1 | PHE | A | 685 | −6.742 | −2.327 | −7.318 | 1.00 | 10.36 | A |
| ATOM | 642 | CD2 | PHE | A | 685 | −7.187 | −1.385 | −9.483 | 1.00 | 12.29 | A |
| ATOM | 643 | CE1 | PHE | A | 685 | −7.573 | −1.361 | −6.724 | 1.00 | 12.32 | A |
| ATOM | 644 | CE2 | PHE | A | 685 | −8.016 | −0.419 | −8.899 | 1.00 | 11.59 | A |
| ATOM | 645 | CE | PHE | A | 685 | −8.210 | −0.406 | −7.520 | 1.00 | 13.91 | A |
| ATOM | 646 | C | PHE | A | 685 | −5.187 | −4.968 | −11.146 | 1.00 | 14.76 | A |
| ATOM | 647 | O | PHE | A | 685 | −5.306 | −6.144 | −10.836 | 1.00 | 15.69 | A |
| ATOM | 648 | N | ASP | A | 686 | −4.231 | −4.531 | −11.952 | 1.00 | 15.75 | A |
| ATOM | 649 | CA | ASP | A | 686 | −3.230 | −5.441 | −12.477 | 1.00 | 16.52 | A |
| ATOM | 650 | CB | ASP | A | 686 | −3.648 | −6.006 | −13.833 | 1.00 | 17.91 | A |
| ATOM | 651 | CG | ASP | A | 686 | −2.696 | −7.075 | −14.319 | 1.00 | 20.72 | A |
| ATOM | 652 | OD1 | ASP | A | 686 | −2.813 | −7.517 | −15.481 | 1.00 | 22.99 | A |
| ATOM | 653 | OD2 | ASP | A | 686 | −1.820 | −7.480 | −13.526 | 1.00 | 21.44 | A |
| ATOM | 654 | C | ASP | A | 686 | −1.929 | −4.668 | −12.626 | 1.00 | 16.22 | A |
| ATOM | 655 | O | ASP | A | 686 | −1.645 | −4.100 | −13.681 | 1.00 | 13.75 | A |
| ATOM | 656 | N | HIS | A | 687 | −1.143 | −4.636 | −11.557 | 1.00 | 15.08 | A |
| ATOM | 657 | CA | HIS | A | 687 | 0.114 | −3.912 | −11.585 | 1.00 | 14.22 | A |
| ATOM | 658 | CB | HIS | A | 687 | −0.119 | −2.460 | −11.160 | 1.00 | 13.14 | A |
| ATOM | 659 | CG | HIS | A | 687 | 1.084 | −1.585 | −11.315 | 1.00 | 16.93 | A |
| ATOM | 660 | CD2 | HIS | A | 687 | 1.406 | −0.672 | −12.264 | 1.00 | 15.49 | A |
| ATOM | 661 | HD1 | HIS | A | 687 | 2.141 | −1.610 | −10.431 | 1.00 | 15.21 | A |
| ATOM | 662 | CE1 | HIS | A | 687 | 3.062 | −0.748 | −10.828 | 1.00 | 17.79 | A |
| ATOM | 663 | NE2 | HIS | A | 687 | 2.639 | −0.166 | −11.937 | 1.00 | 16.56 | A |
| ATOM | 664 | C | HIS | A | 687 | 1.120 | −4.598 | −10.671 | 1.00 | 14.95 | A |
| ATOM | 665 | O | HIS | A | 687 | 0.766 | −5.125 | −9.617 | 1.00 | 13.04 | A |
| ATOM | 666 | N | PRO | A | 688 | 2.394 | −4.612 | −11.076 | 1.00 | 15.37 | A |
| ATOM | 667 | CD | PRO | A | 688 | 2.933 | −4.106 | −12.355 | 1.00 | 13.76 | A |
| ATOM | 668 | CA | PRO | A | 688 | 3.441 | −5.250 | −10.275 | 1.00 | 14.14 | A |
| ATOM | 669 | CB | PRO | A | 688 | 4.716 | −4.909 | −11.048 | 1.00 | 16.35 | A |
| ATOM | 670 | CG | PRO | A | 688 | 4.244 | −4.857 | −12.483 | 1.00 | 16.48 | A |
| ATOM | 671 | C | PRO | A | 688 | 3.496 | −4.785 | −8.816 | 1.00 | 14.10 | A |
| ATOM | 672 | O | PRO | A | 688 | 3.868 | −5.558 | −7.936 | 1.00 | 15.51 | A |
| ATOM | 673 | N | ASN | A | 689 | 3.107 | −3.543 | −8.545 | 1.00 | 12.52 | A |
| ATOM | 674 | CA | ASN | A | 689 | 3.171 | −3.048 | −7.173 | 1.00 | 12.26 | A |
| ATOM | 675 | CB | ASN | A | 689 | 3.949 | −1.741 | −7.144 | 1.00 | 11.33 | A |
| ATOM | 676 | CG | ASN | A | 689 | 5.370 | −1.919 | −7.629 | 1.00 | 13.08 | A |
| ATOM | 677 | OD1 | ASN | A | 689 | 6.237 | −2.433 | −6.907 | 1.00 | 14.93 | A |
| ATOM | 678 | ND2 | ASN | A | 689 | 5.618 | −1.524 | −8.864 | 1.00 | 10.50 | A |
| ATOM | 679 | C | ASN | A | 689 | 1.840 | −2.910 | −6.458 | 1.00 | 9.30 | A |
| ATOM | 680 | O | ASN | A | 689 | 1.685 | −2.116 | −5.543 | 1.00 | 9.38 | A |
| ATOM | 681 | N | VAL | A | 690 | 0.872 | −3.696 | −6.901 | 1.00 | 10.15 | A |
| ATOM | 682 | CA | VAL | A | 690 | −0.438 | −3.738 | −6.275 | 1.00 | 10.17 | A |
| ATOM | 683 | CB | VAL | A | 690 | −1.523 | −3.159 | −7.189 | 1.00 | 11.34 | A |
| ATOM | 684 | CG1 | VAL | A | 690 | −2.907 | −3.458 | −6.593 | 1.00 | 5.73 | A |
| ATOM | 685 | CG2 | VAL | A | 690 | −1.320 | −1.643 | −7.296 | 1.00 | 8.17 | A |
| ATOM | 686 | C | VAL | A | 690 | −0.655 | −5.232 | −6.053 | 1.00 | 10.12 | A |
| ATOM | 687 | O | VAL | A | 690 | −0.445 | −6.038 | −6.959 | 1.00 | 10.79 | A |
| ATOM | 688 | N | ILE | A | 691 | −1.030 | −5.601 | −4.835 | 1.00 | 12.68 | A |
| ATOM | 689 | CA | ILE | A | 691 | −1.225 | −7.005 | −4.482 | 1.00 | 12.48 | A |
| ATOM | 690 | CB | ILE | A | 691 | −1.833 | −7.136 | −3.061 | 1.00 | 15.22 | A |
| ATOM | 691 | CG2 | ILE | A | 691 | −2.079 | −8.597 | −2.729 | 1.00 | 17.18 | A |
| ATOM | 692 | CG1 | ILE | A | 691 | −0.876 | −6.555 | −2.027 | 1.00 | 18.01 | A |
| ATOM | 693 | CD1 | ILE | A | 691 | 0.426 | −7.349 | −1.935 | 1.00 | 24.97 | A |
| ATOM | 694 | C | ILE | A | 691 | −2.122 | −7.724 | −5.478 | 1.00 | 14.49 | A |
| ATOM | 695 | O | ILE | A | 691 | −3.213 | −7.256 | −5.798 | 1.00 | 14.87 | A |
| ATOM | 696 | N | HIS | A | 692 | −1.662 | −8.877 | −5.948 | 1.00 | 14.15 | A |
| ATOM | 697 | CA | HIS | A | 692 | −2.409 | −9.658 | −6.922 | 1.00 | 14.37 | A |
| ATOM | 698 | CB | HIS | A | 692 | −1.444 | −10.529 | −7.729 | 1.00 | 17.27 | A |
| ATOM | 699 | CG | HIS | A | 692 | −2.113 | −11.404 | −8.745 | 1.00 | 19.90 | A |
| ATOM | 700 | CD2 | HIS | A | 692 | −2.301 | −12.743 | −8.775 | 1.00 | 19.49 | A |
| ATOM | 701 | ND1 | HIS | A | 692 | −2.671 | −10.913 | −9.909 | 1.00 | 21.64 | A |
| ATOM | 702 | CE1 | HIS | A | 692 | −3.172 | −11.914 | −10.610 | 1.00 | 20.32 | A |
| ATOM | 703 | NE2 | HIS | A | 692 | −2.961 | −13.035 | −9.944 | 1.00 | 21.85 | A |
| ATOM | 704 | C | HIS | A | 692 | −3.472 | −10.542 | −6.286 | 1.00 | 14.88 | K |
| ATOM | 705 | O | HIS | A | 692 | −3.212 | −11.229 | −5.294 | 1.00 | 14.72 | A |
| ATOM | 706 | N | LEU | A | 693 | −4.673 | −10.513 | −6.854 | 1.00 | 11.54 | A |
| ATOM | 707 | CA | LEU | A | 693 | −5.759 | −11.355 | −6.369 | 1.00 | 11.27 | A |
| ATOM | 708 | CB | LEU | A | 693 | −7.113 | −10.655 | −6.518 | 1.00 | 11.71 | A |
| ATOM | 709 | CG | LEU | A | 693 | −8.362 | −11.527 | −6.311 | 1.00 | 12.06 | A |
| ATOM | 710 | CD1 | LEU | A | 693 | −8.584 | −11.777 | −4.808 | 1.00 | 13.97 | A |
| ATOM | 711 | CD2 | LEU | A | 693 | −9.568 | −10.827 | −6.903 | 1.00 | 12.36 | A |
| ATOM | 712 | C | LEU | A | 693 | −5.766 | −12.629 | −7.202 | 1.00 | 12.80 | A |
| ATOM | 713 | O | LEU | A | 693 | −5.744 | −12.569 | −8.432 | 1.00 | 12.66 | A |
| ATOM | 714 | N | GLU | A | 694 | −5.749 | −13.784 | −6.541 | 1.00 | 9.91 | A |
| ATOM | 715 | CA | GLU | A | 694 | −5.803 | −15.043 | −7.275 | 1.00 | 12.11 | A |
| ATOM | 716 | CB | GLU | A | 694 | −5.190 | −16.197 | −6.463 | 1.00 | 12.53 | A |
| ATOM | 717 | CG | GLU | A | 694 | −3.663 | −16.209 | −6.417 | 1.00 | 15.99 | A |
| ATOM | 718 | CD | GLU | A | 694 | −3.024 | −16.390 | −7.786 | 1.00 | 19.23 | A |
| ATOM | 719 | OE1 | GLU | A | 694 | −3.596 | −17.118 | −8.633 | 1.00 | 22.30 | A |
| ATOM | 720 | OE2 | GLU | A | 694 | −1.939 | −15.817 | −8.019 | 1.00 | 21.66 | A |
| ATOM | 721 | C | GLU | A | 694 | −7.284 | −15.311 | −7.497 | 1.00 | 12.27 | A |
| ATOM | 722 | O | GLU | A | 694 | −7.706 | −15.664 | −8.589 | 1.00 | 14.65 | A |
| ATOM | 723 | N | GLY | A | 695 | −8.072 | −15.126 | −6.446 | 1.00 | 12.00 | A |
| ATOM | 724 | CA | GLY | A | 695 | −9.501 | −15.363 | −6.554 | 1.00 | 13.93 | A |
| ATOM | 725 | C | GLY | A | 695 | −10.162 | −15.460 | −5.191 | 1.00 | 14.27 | A |
| ATOM | 726 | O | GLY | A | 695 | −9.562 | −15.139 | −4.165 | 1.00 | 15.08 | A |
| ATOM | 727 | N | VAL | A | 696 | −11.407 | −15.914 | −5.185 | 1.00 | 16.25 | A |
| ATOM | 728 | CA | VAL | A | 696 | −12.160 | −16.051 | −3.959 | 1.00 | 18.13 | A |
| ATOM | 729 | CB | VAL | A | 696 | −13.213 | −14.920 | −3.813 | 1.00 | 20.10 | A |
| ATOM | 730 | CG1 | VAL | A | 696 | −12.523 | −13.577 | −3.711 | 1.00 | 18.53 | A |
| ATOM | 731 | CG2 | VAL | A | 696 | −14.164 | −14.947 | −5.011 | 1.00 | 18.11 | A |
| ATOM | 732 | C | VAL | A | 696 | −12.900 | −17.385 | −3.944 | 1.00 | 20.73 | A |
| ATOM | 733 | O | VAL | A | 696 | −13.078 | −18.040 | −4.984 | 1.00 | 20.59 | A |
| ATOM | 734 | N | VAL | A | 697 | −13.324 | −17.776 | −2.752 | 1.00 | 20.83 | A |
| ATOM | 735 | CA | VAL | A | 697 | −14.086 | −18.990 | −2.564 | 1.00 | 22.28 | A |
| ATOM | 736 | CB | VAL | A | 697 | −13.365 | −19.983 | −1.621 | 1.00 | 24.02 | A |
| ATOM | 737 | CG1 | VAL | A | 697 | −14.184 | −21.265 | −1.499 | 1.00 | 22.46 | A |
| ATOM | 738 | CG2 | VAL | A | 697 | −11.960 | −20.284 | −2.156 | 1.00 | 22.98 | A |
| ATOM | 739 | C | VAL | A | 697 | −15.344 | −18.486 | −1.880 | 1.00 | 22.94 | A |
| ATOM | 740 | O | VAL | A | 697 | −15.268 | −17.994 | −0.758 | 1.00 | 22.04 | A |
| ATOM | 741 | N | THR | A | 698 | −16.484 | −18.568 | −2.568 | 1.00 | 25.19 | A |
| ATOM | 742 | CA | THR | A | 698 | −17.751 | −18.113 | −2.006 | 1.00 | 27.59 | A |
| ATOM | 743 | CB | THR | A | 698 | −18.298 | −16.854 | −2.736 | 1.00 | 25.83 | A |
| ATOM | 744 | OG1 | THR | A | 698 | −18.578 | −17.176 | −4.099 | 1.00 | 23.74 | A |
| ATOM | 745 | CG2 | THR | A | 698 | −17.287 | −15.713 | −2.687 | 1.00 | 24.82 | A |
| ATOM | 746 | C | THR | A | 698 | −18.828 | −19.193 | −2.076 | 1.00 | 31.39 | A |
| ATOM | 747 | O | THR | A | 698 | −19.826 | −19.119 | −1.362 | 1.00 | 32.32 | A |
| ATOM | 748 | N | LYS | A | 699 | −18.634 | −20.186 | −2.939 | 1.00 | 34.91 | A |
| ATOM | 749 | CA | LYS | A | 699 | −19.606 | −21.265 | −3.085 | 1.00 | 38.78 | A |
| ATOM | 750 | CB | LYS | A | 699 | −19.632 | −21.763 | −4.533 | 1.00 | 39.53 | A |
| ATOM | 751 | CG | LYS | A | 699 | −20.129 | −20.728 | −5.533 | 1.00 | 41.69 | A |
| ATOM | 752 | CD | LYS | A | 699 | −20.157 | −21.281 | −6.953 | 1.00 | 43.87 | A |
| ATOM | 753 | CE | LYS | A | 699 | −20.685 | −20.252 | −7.943 | 1.00 | 44.93 | A |
| ATOM | 754 | NZ | LYS | A | 699 | −20.775 | −20.824 | −9.328 | 1.00 | 47.77 | A |
| ATOM | 755 | C | LYS | A | 699 | −19.295 | −22.422 | −2.145 | 1.00 | 40.28 | A |
| ATOM | 756 | O | LYS | A | 699 | −19.761 | −23.544 | −2.342 | 1.00 | 42.43 | A |
| ATOM | 757 | N | SER | A | 700 | −18.505 | −22.139 | −1.117 | 1.00 | 41.32 | A |
| ATOM | 758 | CA | SER | A | 700 | −18.129 | −23.146 | −0.139 | 1.00 | 41.46 | A |
| ATOM | 759 | CE | SER | A | 700 | −16.927 | −23.949 | −0.642 | 1.00 | 41.67 | A |
| ATOM | 760 | OG | SER | A | 700 | −17.243 | −24.643 | −1.836 | 1.00 | 42.85 | A |
| ATOM | 761 | C | SER | A | 700 | −17.776 | −22.463 | 1.176 | 1.00 | 41.64 | A |
| ATOM | 762 | O | SER | A | 700 | −17.550 | −21.252 | 1.215 | 1.00 | 41.74 | A |
| ATOM | 763 | N | THR | A | 701 | −17.725 | −23.250 | 2.246 | 1.00 | 40.19 | A |
| ATOM | 764 | CA | THR | A | 701 | −17.400 | −22.745 | 3.575 | 1.00 | 40.36 | A |
| ATOM | 765 | CB | THR | A | 701 | −18.451 | −23.208 | 4.618 | 1.00 | 41.83 | A |
| ATOM | 766 | OG1 | THR | A | 701 | −19.763 | −22.837 | 4.175 | 1.00 | 44.63 | A |
| ATOM | 767 | CG2 | THR | A | 701 | −18.190 | −22.558 | 5.973 | 1.00 | 42.59 | A |
| ATOM | 768 | C | THR | A | 701 | −16.024 | −23.256 | 4.011 | 1.00 | 38.95 | A |
| ATOM | 769 | O | THR | A | 701 | −15.702 | −24.434 | 3.844 | 1.00 | 39.42 | A |
| ATOM | 770 | N | PRO | A | 702 | −15.185 | −22.370 | 4.565 | 1.00 | 37.14 | A |
| ATOM | 771 | CD | PRO | A | 702 | −13.936 | −22.766 | 5.239 | 1.00 | 37.05 | A |
| ATOM | 772 | CA | PRO | A | 702 | −15.465 | −20.949 | 4.792 | 1.00 | 34.67 | A |
| ATOM | 773 | CB | PRO | A | 702 | −14.551 | −20.611 | 5.955 | 1.00 | 35.95 | A |
| ATOM | 774 | CG | PRO | A | 702 | −13.338 | −21.429 | 5.630 | 1.00 | 36.79 | A |
| ATOM | 775 | C | PRO | A | 702 | −15.158 | −20.095 | 3.569 | 1.00 | 31.84 | A |
| ATOM | 776 | O | PRO | A | 702 | −14.339 | −20.469 | 2.734 | 1.00 | 31.01 | A |
| ATOM | 777 | N | VAL | A | 703 | −15.831 | −18.952 | 3.472 | 1.00 | 30.06 | A |
| ATOM | 778 | CA | VAL | A | 703 | −15.620 | −18.017 | 2.372 | 1.00 | 26.08 | A |
| ATOM | 779 | CB | VAL | A | 703 | −16.592 | −16.830 | 2.475 | 1.00 | 27.78 | A |
| ATOM | 780 | CG1 | VAL | A | 703 | −16.399 | −15.886 | 1.292 | 1.00 | 26.07 | A |
| ATOM | 781 | CG2 | VAL | A | 703 | −18.028 | −17.343 | 2.525 | 1.00 | 27.11 | A |
| ATOM | 782 | C | VAL | A | 703 | −14.183 | −17.510 | 2.493 | 1.00 | 23.65 | A |
| ATOM | 783 | O | VAL | A | 703 | −13.727 | −17.199 | 3.591 | 1.00 | 22.73 | A |
| ATOM | 784 | N | MET | A | 704 | −13.480 | −17.422 | 1.367 | 1.00 | 21.87 | A |
| ATOM | 785 | CA | MET | A | 704 | −12.091 | −16.985 | 1.371 | 1.00 | 19.85 | A |
| ATOM | 786 | CE | MET | A | 704 | −11.154 | −18.194 | 1.287 | 1.00 | 20.58 | A |
| ATOM | 787 | CG | MET | A | 704 | −11.394 | −19.298 | 2.324 | 1.00 | 22.83 | A |
| ATOM | 788 | SD | MET | A | 704 | −10.199 | −20.637 | 2.110 | 1.00 | 25.21 | A |
| ATOM | 789 | CE | MET | A | 704 | −11.006 | −21.626 | 0.904 | 1.00 | 26.56 | A |
| ATOM | 790 | C | MET | A | 704 | −11.702 | −16.055 | 0.226 | 1.00 | 18.73 | A |
| ATOM | 791 | O | MET | A | 704 | −12.348 | −16.027 | −0.817 | 1.00 | 18.27 | A |
| ATOM | 792 | N | ILE | A | 705 | −10.611 | −15.327 | 0.446 | 1.00 | 16.10 | A |
| ATOM | 793 | CA | ILE | A | 705 | −10.020 | −14.433 | −0.541 | 1.00 | 14.53 | A |
| ATOM | 794 | CB | ILE | A | 705 | −10.033 | −12.980 | −0.079 | 1.00 | 14.98 | A |
| ATOM | 795 | CG2 | ILE | A | 705 | −9.219 | −12.088 | −1.066 | 1.00 | 13.23 | A |
| ATOM | 796 | CG1 | ILE | A | 705 | −11.473 | −12.508 | 0.019 | 1.00 | 13.64 | A |
| ATOM | 797 | CD1 | ILE | A | 705 | −11.593 | −11.116 | 0.560 | 1.00 | 15.61 | A |
| ATOM | 798 | C | ILE | A | 705 | −8.588 | −14.917 | −0.598 | 1.00 | 14.95 | A |
| ATOM | 799 | O | ILE | A | 705 | −7.921 | −14.999 | 0.437 | 1.00 | 16.93 | A |
| ATOM | 800 | N | ILE | A | 706 | −8.125 | −15.247 | −1.797 | 1.00 | 14.61 | A |
| ATOM | 801 | CA | ILE | A | 706 | −6.776 | −15.761 | −1.995 | 1.00 | 14.22 | A |
| ATOM | 802 | CB | ILE | A | 706 | −6.799 | −17.054 | −2.837 | 1.00 | 13.99 | A |
| ATOM | 803 | CG2 | ILE | A | 706 | −5.448 | −17.747 | −2.748 | 1.00 | 15.86 | A |
| ATOM | 804 | CG1 | ILE | A | 706 | −7.914 | −17.987 | −2.353 | 1.00 | 16.58 | A |
| ATOM | 805 | CD1 | ILE | A | 706 | −7.755 | −18.443 | −0.919 | 1.00 | 23.47 | A |
| ATOM | 806 | C | ILE | A | 706 | −5.952 | −14.726 | −2.741 | 1.00 | 14.50 | A |
| ATOM | 807 | O | ILE | A | 706 | −6.346 | −14.300 | −3.829 | 1.00 | 12.53 | A |
| ATOM | 808 | N | THR | A | 707 | −4.806 | −14.342 | −2.179 | 1.00 | 12.32 | A |
| ATOM | 809 | CA | THR | A | 707 | −3.930 | −13.344 | −2.810 | 1.00 | 14.46 | A |
| ATOM | 810 | CB | THR | A | 707 | −3.926 | −12.026 | −2.005 | 1.00 | 16.58 | A |
| ATOM | 811 | OG1 | TER | A | 707 | −3.435 | −12.288 | −0.685 | 1.00 | 18.64 | A |
| ATOM | 812 | CG2 | THR | A | 707 | −5.334 | −11.434 | −1.895 | 1.00 | 15.55 | A |
| ATOM | 813 | C | THR | A | 707 | −2.486 | −13.847 | −2.921 | 1.00 | 16.08 | A |
| ATOM | 814 | O | THR | A | 707 | −2.153 | −14.876 | −2.337 | 1.00 | 15.13 | A |
| ATOM | 815 | N | GLU | A | 708 | −1.631 | −13.150 | 3.673 | 1.00 | 14.63 | A |
| ATOM | 816 | CA | GLU | A | 708 | −0.240 | −13.603 | −3.798 | 1.00 | 16.45 | A |
| ATOM | 817 | CB | GLU | A | 708 | 0.576 | −12.730 | −4.779 | 1.00 | 16.75 | A |
| ATOM | 818 | CG | GLU | A | 708 | 0.855 | −11.308 | −4.315 | 1.00 | 17.77 | A |
| ATOM | 819 | CD | GLU | A | 708 | 1.522 | −10.440 | −5.399 | 1.00 | 18.66 | A |
| ATOM | 820 | OE1 | GLU | A | 708 | 0.897 | −9.447 | −5.806 | 1.00 | 17.59 | A |
| ATOM | 821 | OE2 | GLU | A | 708 | 2.670 | −10.747 | −5.833 | 1.00 | 18.27 | A |
| ATOM | 822 | C | GLU | A | 708 | 0.412 | −13.574 | −2.428 | 1.00 | 15.05 | A |
| ATOM | 823 | O | GLU | A | 708 | 0.091 | −12.730 | −1.582 | 1.00 | 12.21 | A |
| ATOM | 824 | N | PHE | A | 709 | 1.319 | −14.516 | −2.203 | 1.00 | 15.72 | A |
| ATOM | 825 | CA | PHE | A | 709 | 2.001 | −14.599 | −0.920 | 1.00 | 16.73 | A |
| ATOM | 826 | CE | PHE | A | 709 | 2.486 | −16.035 | −0.678 | 1.00 | 18.37 | A |
| ATOM | 827 | CG | PHE | A | 709 | 3.127 | −16.238 | 0.661 | 1.00 | 20.59 | A |
| ATOM | 828 | CD1 | PHE | A | 709 | 2.423 | −15.975 | 1.829 | 1.00 | 21.91 | A |
| ATOM | 829 | CD2 | PHE | A | 709 | 4.433 | −16.690 | 0.756 | 1.00 | 23.57 | A |
| ATOM | 830 | CE1 | PHE | A | 709 | 3.011 | −16.160 | 3.074 | 1.00 | 24.66 | A |
| ATOM | 831 | CE2 | PHE | A | 709 | 5.031 | −16.881 | 1.997 | 1.00 | 23.90 | A |
| ATOM | 832 | CZ | PHE | A | 709 | 4.315 | −16.614 | 3.160 | 1.00 | 25.45 | A |
| ATOM | 833 | C | PHE | A | 709 | 3.178 | −13.630 | −0.890 | 1.00 | 15.59 | A |
| ATOM | 834 | O | PHE | A | 709 | 3.928 | −13.541 | −1.853 | 1.00 | 16.82 | A |
| ATOM | 835 | N | MET | A | 710 | 3.316 | −12.906 | 0.219 | 1.00 | 14.56 | A |
| ATOM | 836 | CA | MET | A | 710 | 4.393 | −11.933 | 0.410 | 1.00 | 16.05 | A |
| ATOM | 837 | CB | MET | A | 710 | 3.793 | −10.539 | 0.610 | 1.00 | 15.84 | A |
| ATOM | 838 | CG | MET | A | 710 | 2.896 | −10.105 | −0.547 | 1.00 | 16.83 | A |
| ATOM | 839 | SD | MET | A | 710 | 3.759 | −9.890 | −2.108 | 1.00 | 16.56 | A |
| ATOM | 840 | CE | MET | A | 710 | 4.355 | −8.267 | −1.864 | 1.00 | 17.49 | A |
| ATOM | 841 | C | MET | A | 710 | 5.198 | −12.365 | 1.642 | 1.00 | 15.49 | A |
| ATOM | 842 | O | MET | A | 710 | 4.828 | −12.075 | 2.774 | 1.00 | 13.99 | A |
| ATOM | 843 | N | GLU | A | 711 | 6.301 | −13.065 | 1.400 | 1.00 | 17.57 | A |
| ATOM | 844 | CA | GLU | A | 711 | 7.130 | −13.597 | 2.478 | 1.00 | 20.55 | A |
| ATOM | 845 | CB | GLU | A | 711 | 8.369 | −14.275 | 1.906 | 1.00 | 23.66 | A |
| ATOM | 846 | CG | GLU | A | 711 | 8.052 | −15.543 | 1.150 | 1.00 | 32.78 | A |
| ATOM | 847 | CD | GLU | A | 711 | 9.241 | −16.479 | 1.065 | 1.00 | 35.60 | A |
| ATOM | 848 | OE1 | GLU | A | 711 | 9.088 | −17.579 | 0.478 | 1.00 | 37.62 | A |
| ATOM | 849 | OE2 | GLU | A | 711 | 10.319 | −16.112 | 1.589 | 1.00 | 36.45 | A |
| ATOM | 850 | C | GLU | A | 711 | 7.554 | −12.624 | 3.551 | 1.00 | 20.06 | A |
| ATOM | 851 | O | GLU | A | 711 | 7.577 | −12.970 | 4.728 | 1.00 | 20.45 | A |
| ATOM | 852 | N | ASN | A | 712 | 7.879 | −11.403 | 3.165 | 1.00 | 16.84 | A |
| ATOM | 853 | CA | ASN | A | 712 | 8.322 | −10.459 | 4.160 | 1.00 | 15.71 | A |
| ATOM | 854 | CB | ASN | A | 712 | 9.368 | −9.544 | 3.547 | 1.00 | 17.27 | A |
| ATOM | 855 | CG | ASN | A | 712 | 10.627 | −10.303 | 3.222 | 1.00 | 17.78 | A |
| ATOM | 856 | OD1 | ASN | A | 712 | 11.034 | −11.157 | 4.005 | 1.00 | 16.07 | A |
| ATOM | 857 | ND2 | ASN | A | 712 | 11.236 | −10.026 | 2.081 | 1.00 | 18.84 | A |
| ATOM | 858 | C | ASN | A | 712 | 7.237 | −9.685 | 4.868 | 1.00 | 15.77 | A |
| ATOM | 859 | O | ASN | A | 712 | 7.515 | −8.845 | 5.711 | 1.00 | 13.23 | A |
| ATOM | 860 | N | GLY | A | 713 | 5.991 | −9.995 | 4.537 | 1.00 | 16.18 | A |
| ATOM | 861 | CA | GLY | A | 713 | 4.884 | −9.350 | 5.207 | 1.00 | 13.90 | A |
| ATOM | 862 | C | GLY | A | 713 | 4.785 | −7.846 | 5.109 | 1.00 | 14.53 | A |
| ATOM | 863 | O | GLY | A | 713 | 5.179 | −7.237 | 4.108 | 1.00 | 13.01 | A |
| ATOM | 864 | N | SER | A | 714 | 4.258 | −7.252 | 6.173 | 1.00 | 10.95 | A |
| ATOM | 865 | CA | SER | A | 714 | 4.068 | −5.822 | 6.231 | 1.00 | 15.09 | A |
| ATOM | 866 | CB | SER | A | 714 | 3.195 | −5.470 | 7.424 | 1.00 | 16.14 | A |
| ATOM | 867 | OG | SER | A | 714 | 1.949 | −6.125 | 7.292 | 1.00 | 17.72 | A |
| ATOM | 868 | C | SER | A | 714 | 5.383 | −5.075 | 6.288 | 1.00 | 15.86 | A |
| ATOM | 869 | O | SER | A | 714 | 6.290 | −5.427 | 7.041 | 1.00 | 12.51 | A |
| ATOM | 870 | N | LEU | A | 715 | 5.457 | −4.030 | 5.476 | 1.00 | 14.56 | A |
| ATOM | 871 | CA | LEU | A | 715 | 6.639 | −3.206 | 5.352 | 1.00 | 15.61 | A |
| ATOM | 872 | CB | LEU | A | 715 | 6.399 | −2.141 | 4.284 | 1.00 | 12.49 | A |
| ATOM | 873 | CG | LEU | A | 715 | 7.574 | −1.209 | 4.012 | 1.00 | 15.37 | A |
| ATOM | 874 | CD1 | LEU | A | 715 | 8.786 | −2.009 | 3.583 | 1.00 | 12.76 | A |
| ATOM | 875 | CD2 | LEU | A | 715 | 7.161 | −0.200 | 2.907 | 1.00 | 12.52 | A |
| ATOM | 876 | C | LEU | A | 715 | 7.102 | −2.540 | 6.637 | 1.00 | 14.75 | A |
| ATOM | 877 | O | LEU | A | 715 | 8.300 | −2.500 | 6.905 | 1.00 | 17.56 | A |
| ATOM | 878 | N | ASP | A | 716 | 6.180 | −2.018 | 7.444 | 1.00 | 15.82 | A |
| ATOM | 879 | CA | ASP | A | 716 | 6.631 | −1.369 | 8.674 | 1.00 | 17.28 | A |
| ATOM | 880 | CB | ASP | A | 716 | 5.462 | −0.652 | 9.389 | 1.00 | 16.92 | A |
| ATOM | 881 | CG | ASP | A | 716 | 4.361 | −1.603 | 9.849 | 1.00 | 19.44 | A |
| ATOM | 882 | OD1 | ASP | A | 716 | 3.995 | −2.546 | 9.109 | 1.00 | 17.35 | A |
| ATOM | 883 | OD2 | ASP | A | 716 | 3.850 | −1.389 | 10.964 | 1.00 | 22.24 | A |
| ATOM | 884 | C | ASP | A | 716 | 7.317 | −2.397 | 9.587 | 1.00 | 17.91 | A |
| ATOM | 885 | O | ASP | A | 716 | 8.431 | −2.169 | 10.059 | 1.00 | 16.46 | A |
| ATOM | 886 | N | SER | A | 717 | 6.677 | −3.542 | 9.802 | 1.00 | 17.08 | A |
| ATOM | 887 | CA | SER | A | 717 | 7.266 | −4.593 | 10.654 | 1.00 | 17.05 | A |
| ATOM | 888 | CB | SER | A | 717 | 6.283 | −5.747 | 10.807 | 1.00 | 18.18 | A |
| ATOM | 889 | OG | SER | A | 717 | 5.131 | −5.296 | 11.484 | 1.00 | 24.48 | A |
| ATOM | 890 | C | SER | A | 717 | 8.568 | −5.136 | 10.081 | 1.00 | 14.71 | A |
| ATOM | 891 | O | SER | A | 717 | 9.537 | −5.363 | 10.807 | 1.00 | 12.74 | A |
| ATOM | 892 | N | PHE | A | 718 | 8.576 | −5.340 | 8.766 | 1.00 | 13.61 | A |
| ATOM | 893 | CA | PHE | A | 718 | 9.742 | −5.854 | 8.061 | 1.00 | 11.92 | A |
| ATOM | 894 | CB | PHE | A | 718 | 9.456 | −5.913 | 6.566 | 1.00 | 12.48 | A |
| ATOM | 895 | CG | PHE | A | 718 | 10.624 | −6.331 | 5.736 | 1.00 | 12.92 | A |
| ATOM | 896 | CD1 | PHE | A | 718 | 11.172 | −7.605 | 5.873 | 1.00 | 13.91 | A |
| ATOM | 897 | CD2 | PHE | A | 718 | 11.155 | −5.465 | 4.780 | 1.00 | 13.01 | A |
| ATOM | 898 | CE1 | PHE | A | 718 | 12.229 | −8.021 | 5.069 | 1.00 | 14.75 | A |
| ATOM | 899 | CE2 | PHE | A | 718 | 12.209 | −5.864 | 3.969 | 1.00 | 10.87 | A |
| ATOM | 900 | CZ | PHE | A | 718 | 12.752 | −7.153 | 4.110 | 1.00 | 14.74 | A |
| ATOM | 901 | C | PHE | A | 718 | 10.947 | −4.968 | 8.294 | 1.00 | 12.69 | A |
| ATOM | 902 | O | PHE | A | 718 | 12.044 | −5.456 | 8.563 | 1.00 | 13.46 | A |
| ATOM | 903 | N | LEU | A | 719 | 10.736 | −3.662 | 8.176 | 1.00 | 13.46 | A |
| ATOM | 904 | CA | LEU | A | 719 | 11.806 | −2.698 | 8.358 | 1.00 | 16.37 | A |
| ATOM | 905 | CB | LEU | A | 719 | 11.357 | −1.299 | 7.923 | 1.00 | 14.77 | A |
| ATOM | 906 | CG | LEU | A | 719 | 11.232 | −1.091 | 6.407 | 1.00 | 18.77 | A |
| ATOM | 907 | CD1 | LEU | A | 719 | 10.819 | 0.339 | 6.130 | 1.00 | 16.45 | A |
| ATOM | 908 | CD2 | LEU | A | 719 | 12.556 | −1.395 | 5.718 | 1.00 | 20.57 | A |
| ATOM | 909 | C | LEU | A | 719 | 12.280 | −2.655 | 9.797 | 1.00 | 14.90 | A |
| ATOM | 910 | O | LEU | A | 719 | 13.465 | −2.497 | 10.052 | 1.00 | 14.07 | A |
| ATOM | 911 | N | ARG | A | 720 | 11.360 | −2.792 | 10.742 | 1.00 | 16.00 | A |
| ATOM | 912 | CA | ARG | A | 720 | 11.776 | −2.759 | 12.137 | 1.00 | 16.42 | A |
| ATOM | 913 | CB | ARG | A | 720 | 10.563 | −2.702 | 13.062 | 1.00 | 18.72 | A |
| ATOM | 914 | CG | ARG | A | 720 | 10.012 | −1.296 | 13.208 | 1.00 | 19.57 | A |
| ATOM | 915 | CD | ARG | A | 720 | 8.967 | −1.164 | 14.300 | 1.00 | 22.51 | A |
| ATOM | 916 | NE | ARG | A | 720 | 7.624 | −1.499 | 13.843 | 1.00 | 28.80 | A |
| ATOM | 917 | CZ | ARG | A | 720 | 7.146 | −2.734 | 13.738 | 1.00 | 31.29 | A |
| ATOM | 918 | NH1 | ARG | A | 720 | 7.902 | −3.777 | 14.063 | 1.00 | 33.67 | A |
| ATOM | 919 | NH2 | ARG | A | 720 | 5.903 | −2.922 | 13.311 | 1.00 | 31.71 | A |
| ATOM | 920 | C | ARG | A | 720 | 12.662 | −3.951 | 12.463 | 1.00 | 17.27 | A |
| ATOM | 921 | O | ARG | A | 720 | 13.634 | −3.826 | 13.215 | 1.00 | 18.44 | A |
| ATOM | 922 | N | GLN | A | 721 | 12.348 | −5.098 | 11.870 | 1.00 | 16.09 | A |
| ATOM | 923 | CA | GLN | A | 721 | 13.116 | −6.313 | 12.107 | 1.00 | 18.82 | A |
| ATOM | 924 | CB | GLN | A | 721 | 12.287 | −7.532 | 11.709 | 1.00 | 20.62 | A |
| ATOM | 925 | CG | GLN | A | 721 | 10.946 | −7.586 | 12.403 | 1.00 | 25.99 | A |
| ATOM | 926 | CD | GLN | A | 721 | 10.026 | −8.623 | 11.801 | 1.00 | 29.22 | A |
| ATOM | 927 | OE1 | GLN | A | 721 | 10.338 | −9.231 | 10.768 | 1.00 | 31.08 | A |
| ATOM | 928 | NE2 | GLN | A | 721 | 8.875 | −8.827 | 12.435 | 1.00 | 30.60 | A |
| ATOM | 929 | C | GLN | A | 721 | 14.426 | −6.339 | 11.332 | 1.00 | 18.64 | A |
| ATOM | 930 | O | GLN | A | 721 | 15.242 | −7.261 | 11.481 | 1.00 | 17.28 | A |
| ATOM | 931 | N | ASN | A | 722 | 14.619 | −5.326 | 10.499 | 1.00 | 17.52 | A |
| ATOM | 932 | CA | ASN | A | 722 | 15.813 | −5.236 | 9.676 | 1.00 | 17.80 | A |
| ATOM | 933 | CB | ASN | A | 722 | 15.469 | −5.642 | 8.241 | 1.00 | 17.53 | A |
| ATOM | 934 | CG | ASN | A | 722 | 15.262 | −7.143 | 8.100 | 1.00 | 20.53 | A |
| ATOM | 935 | OD1 | ASN | A | 722 | 16.227 | −7.901 | 8.085 | 1.00 | 22.10 | A |
| ATOM | 936 | ND2 | ASN | A | 722 | 13.997 | −7.582 | 8.015 | 1.00 | 17.67 | A |
| ATOM | 937 | C | ASN | A | 722 | 16.347 | −3.820 | 9.719 | 1.00 | 17.61 | A |
| ATOM | 938 | O | ASN | A | 722 | 16.752 | −3.268 | 8.697 | 1.00 | 18.91 | A |
| ATOM | 939 | N | ASP | A | 723 | 16.361 | −3.247 | 10.919 | 1.00 | 17.15 | A |
| ATOM | 940 | CA | ASP | A | 723 | 16.817 | −1.887 | 11.101 | 1.00 | 19.13 | A |
| ATOM | 941 | CB | ASP | A | 723 | 16.702 | −1.498 | 12.582 | 1.00 | 23.22 | A |
| ATOM | 942 | CG | ASP | A | 723 | 17.064 | −0.043 | 12.844 | 1.00 | 26.94 | A |
| ATOM | 943 | OD1 | ASP | A | 723 | 16.687 | 0.837 | 12.049 | 1.00 | 28.14 | A |
| ATOM | 944 | OD2 | ASP | A | 723 | 17.728 | 0.220 | 13.869 | 1.00 | 30.54 | A |
| ATOM | 945 | C | ASP | A | 723 | 18.244 | −1.685 | 10.569 | 1.00 | 19.80 | A |
| ATOM | 946 | O | ASP | A | 723 | 19.168 | −2.433 | 10.906 | 1.00 | 15.82 | A |
| ATOM | 947 | N | GLY | A | 724 | 18.374 | −0.682 | 9.698 | 1.00 | 16.08 | A |
| ATOM | 948 | CA | GLY | A | 724 | 19.644 | −0.327 | 9.089 | 1.00 | 15.30 | A |
| ATOM | 949 | C | CLY | A | 724 | 20.264 | −1.401 | 8.220 | 1.00 | 14.31 | A |
| ATOM | 950 | O | GLY | A | 724 | 21.430 | −1.314 | 7.855 | 1.00 | 13.94 | A |
| ATOM | 951 | N | GLN | A | 725 | 19.481 | −2.402 | 7.843 | 1.00 | 14.27 | A |
| ATOM | 952 | CA | GLN | A | 725 | 20.030 | −3.503 | 7.064 | 1.00 | 14.66 | A |
| ATOM | 953 | CB | GLN | A | 725 | 19.286 | −4.784 | 7.418 | 1.00 | 16.87 | A |
| ATOM | 954 | CG | GLN | A | 725 | 19.316 | −5.104 | 8.912 | 1.00 | 18.98 | A |
| ATOM | 955 | CD | GLN | A | 725 | 20.744 | −5.203 | 9.445 | 1.00 | 20.91 | A |
| ATOM | 956 | OE1 | GLN | A | 725 | 21.198 | −4.338 | 10.202 | 1.00 | 25.17 | A |
| ATOM | 957 | NE2 | GLN | A | 725 | 21.456 | −6.248 | 9.044 | 1.00 | 18.71 | A |
| ATOM | 958 | C | GLN | A | 725 | 20.092 | −3.349 | 5.557 | 1.00 | 14.70 | A |
| ATOM | 959 | O | GLN | A | 725 | 20.722 | −4.162 | 4.895 | 1.00 | 13.92 | A |
| ATOM | 960 | N | PHE | A | 726 | 19.454 | −2.321 | 5.011 | 1.00 | 13.07 | A |
| ATOM | 961 | CA | PHE | A | 726 | 19.459 | −2.151 | 3.561 | 1.00 | 15.29 | A |
| ATOM | 962 | CB | PHE | A | 726 | 18.016 | −1.995 | 3.085 | 1.00 | 14.57 | A |
| ATOM | 963 | CG | PHE | A | 726 | 17.140 | −3.143 | 3.477 | 1.00 | 15.22 | A |
| ATOM | 964 | CD1 | PHE | A | 726 | 16.094 | −2.966 | 4.375 | 1.00 | 14.78 | A |
| ATOM | 965 | CD2 | PHE | A | 726 | 17.399 | −4.419 | 2.984 | 1.00 | 14.88 | A |
| ATOM | 966 | CE1 | PHE | A | 726 | 15.325 | −4.042 | 4.776 | 1.00 | 13.36 | A |
| ATOM | 967 | CE2 | PHE | A | 726 | 16.630 | −5.505 | 3.386 | 1.00 | 17.90 | A |
| ATOM | 968 | CZ | PHE | A | 726 | 15.594 | −5.314 | 4.285 | 1.00 | 14.76 | A |
| ATOM | 969 | C | PHE | A | 726 | 20.300 | −0.997 | 3.050 | 1.00 | 13.71 | A |
| ATOM | 970 | O | PHE | A | 726 | 20.627 | −0.070 | 3.794 | 1.00 | 16.24 | A |
| ATOM | 971 | N | TEE | A | 727 | 20.669 | −1.066 | 1.776 | 1.00 | 17.14 | A |
| ATOM | 972 | CA | THE | A | 727 | 21.443 | 0.020 | 1.184 | 1.00 | 17.33 | A |
| ATOM | 973 | CB | THR | A | 727 | 22.177 | −0.429 | −0.095 | 1.00 | 17.31 | A |
| ATOM | 974 | OG1 | THR | A | 727 | 21.233 | −0.637 | −1.148 | 1.00 | 16.76 | A |
| ATOM | 975 | CG2 | THR | A | 727 | 22.934 | −1.744 | 0.153 | 1.00 | 17.77 | A |
| ATOM | 976 | C | THR | A | 727 | 20.438 | 1.121 | 0.851 | 1.00 | 19.38 | A |
| ATOM | 977 | O | THR | A | 727 | 19.224 | 0.881 | 0.853 | 1.00 | 18.71 | A |
| ATOM | 978 | N | VAL | A | 728 | 20.941 | 2.326 | 0.601 | 1.00 | 17.57 | A |
| ATOM | 979 | CA | VAL | A | 728 | 20.090 | 3.465 | 0.257 | 1.00 | 18.19 | A |
| ATOM | 980 | CB | VAL | A | 728 | 20.924 | 4.776 | 0.134 | 1.00 | 19.55 | A |
| ATOM | 981 | CG1 | VAL | A | 728 | 20.029 | 5.939 | −0.320 | 1.00 | 20.05 | A |
| ATOM | 982 | CG2 | VAL | A | 728 | 21.550 | 5.118 | 1.487 | 1.00 | 18.70 | A |
| ATOM | 983 | C | VAL | A | 728 | 19.367 | 3.184 | −1.066 | 1.00 | 16.00 | A |
| ATOM | 984 | O | VAL | A | 728 | 18.181 | 3.482 | −1.216 | 1.00 | 15.83 | A |
| ATOM | 985 | N | ILE | A | 729 | 20.085 | 2.603 | −2.019 | 1.00 | 16.06 | A |
| ATOM | 986 | CA | ILE | A | 729 | 19.490 | 2.274 | −3.306 | 1.00 | 16.24 | A |
| ATOM | 987 | CB | ILE | A | 729 | 20.565 | 1.765 | −4.297 | 1.00 | 15.78 | A |
| ATOM | 988 | CG2 | ILE | A | 729 | 19.949 | 0.856 | −5.350 | 1.00 | 14.65 | A |
| ATOM | 989 | CG1 | ILE | A | 729 | 21.272 | 2.962 | −4.948 | 1.00 | 18.50 | A |
| ATOM | 990 | CD1 | ILE | A | 729 | 20.387 | 3.784 | −5.889 | 1.00 | 16.84 | A |
| ATOM | 991 | C | ILE | A | 729 | 18.375 | 1.247 | −3.137 | 1.00 | 17.15 | A |
| ATOM | 992 | O | ILE | A | 729 | 17.377 | 1.274 | −3.870 | 1.00 | 13.45 | A |
| ATOM | 993 | N | GLN | A | 730 | 18.521 | 0.342 | −2.172 | 1.00 | 16.67 | A |
| ATOM | 994 | CA | GLN | A | 730 | 17.461 | −0.649 | −1.962 | 1.00 | 16.00 | A |
| ATOM | 995 | CB | GLN | A | 730 | 17.922 | −1.763 | −1.015 | 1.00 | 16.68 | A |
| ATOM | 996 | CG | GLN | A | 730 | 18.885 | −2.733 | −1.646 | 1.00 | 15.89 | A |
| ATOM | 997 | CD | GLN | A | 730 | 19.389 | −3.777 | −0.661 | 1.00 | 16.94 | A |
| ATOM | 998 | OE1 | GLN | A | 730 | 19.843 | −3.437 | 0.441 | 1.00 | 15.33 | A |
| ATOM | 999 | NE2 | GLN | A | 730 | 19.312 | −5.049 | −1.052 | 1.00 | 13.76 | A |
| ATOM | 1000 | C | GLN | A | 730 | 16.202 | 0.008 | −1.397 | 1.00 | 15.38 | A |
| ATOM | 1001 | O | GLN | A | 730 | 15.084 | −0.284 | −1.838 | 1.00 | 15.30 | A |
| ATOM | 1002 | N | LEU | A | 731 | 16.385 | 0.896 | −0.424 | 1.00 | 14.09 | A |
| ATOM | 1003 | CA | LEU | A | 731 | 15.256 | 1.589 | 0.193 | 1.00 | 14.64 | A |
| ATOM | 1004 | CB | LEU | A | 731 | 15.721 | 2.437 | 1.377 | 1.00 | 13.73 | A |
| ATOM | 1005 | CG | LEU | A | 731 | 16.298 | 1.671 | 2.577 | 1.00 | 15.51 | A |
| ATOM | 1006 | CD1 | LEU | A | 731 | 16.848 | 2.669 | 3.569 | 1.00 | 15.48 | A |
| ATOM | 1007 | CD2 | LEU | A | 731 | 15.227 | 0.797 | 3.228 | 1.00 | 15.71 | A |
| ATOM | 1008 | C | LEU | A | 731 | 14.570 | 2.480 | −0.828 | 1.00 | 15.58 | A |
| ATOM | 1009 | O | LEU | A | 731 | 13.341 | 2.582 | −0.851 | 1.00 | 13.60 | A |
| ATOM | 1010 | N | VAL | A | 732 | 15.368 | 3.137 | −1.665 | 1.00 | 14.16 | A |
| ATOM | 1011 | CA | VAL | A | 732 | 14.798 | 4.003 | −2.687 | 1.00 | 13.56 | A |
| ATOM | 1012 | CB | VAL | A | 732 | 15.881 | 4.746 | −3.497 | 1.00 | 12.54 | A |
| ATOM | 1013 | CG1 | VAL | A | 732 | 15.225 | 5.497 | −4.664 | 1.00 | 13.40 | A |
| ATOM | 1014 | CG2 | VAL | A | 732 | 16.596 | 5.748 | −2.598 | 1.00 | 12.18 | A |
| ATOM | 1015 | C | VAL | A | 732 | 13.980 | 3.137 | −3.636 | 1.00 | 12.77 | A |
| ATOM | 1016 | O | VAL | A | 732 | 12.917 | 3.548 | −4.097 | 1.00 | 16.70 | A |
| ATOM | 1017 | N | GLY | A | 733 | 14.479 | 1.932 | −3.896 | 1.00 | 12.87 | A |
| ATOM | 1018 | CA | GLY | A | 733 | 13.796 | 1.003 | −4.776 | 1.00 | 11.87 | A |
| ATOM | 1019 | C | GLY | A | 733 | 12.438 | 0.597 | −4.231 | 1.00 | 12.83 | A |
| ATOM | 1020 | O | GLY | A | 733 | 11.485 | 0.367 | −4.989 | 1.00 | 10.23 | A |
| ATOM | 1021 | N | MET | A | 734 | 12.347 | 0.499 | −2.908 | 1.00 | 11.75 | A |
| ATOM | 1022 | CA | MET | A | 734 | 11.086 | 0.146 | −2.263 | 1.00 | 11.65 | A |
| ATOM | 1023 | CB | MET | A | 734 | 11.298 | −0.137 | −0.773 | 1.00 | 11.18 | A |
| ATOM | 1024 | CG | MET | A | 734 | 12.101 | −1.393 | −0.479 | 1.00 | 13.32 | A |
| ATOM | 1025 | SD | MET | A | 734 | 12.561 | −1.491 | 1.265 | 1.00 | 17.57 | A |
| ATOM | 1026 | CE | MET | A | 734 | 13.565 | −2.978 | 1.245 | 1.00 | 14.96 | A |
| ATOM | 1027 | C | MET | A | 734 | 10.096 | 1.297 | −2.419 | 1.00 | 12.17 | A |
| ATOM | 1028 | O | MET | A | 734 | 8.916 | 1.093 | −2.732 | 1.00 | 12.13 | A |
| ATOM | 1029 | N | LEU | A | 735 | 10.590 | 2.510 | −2.211 | 1.00 | 10.98 | A |
| ATOM | 1030 | CA | LEU | A | 735 | 9.751 | 3.692 | −2.312 | 1.00 | 12.80 | A |
| ATOM | 1031 | CB | LEU | A | 735 | 10.509 | 4.919 | −1.789 | 1.00 | 13.81 | A |
| ATOM | 1032 | CG | LEU | A | 735 | 10.854 | 4.931 | −0.283 | 1.00 | 14.22 | A |
| ATOM | 1033 | CD1 | LEU | A | 735 | 11.767 | 6.115 | 0.009 | 1.00 | 15.97 | A |
| ATOM | 1034 | CD2 | LEU | A | 735 | 9.592 | 5.036 | 0.554 | 1.00 | 14.12 | A |
| ATOM | 1035 | C | LEU | A | 735 | 9.277 | 3.909 | −3.756 | 1.00 | 12.46 | A |
| ATOM | 1036 | O | LEU | A | 735 | 8.162 | 4.387 | −3.991 | 1.00 | 12.21 | A |
| ATOM | 1037 | N | ARG | A | 736 | 10.126 | 3.535 | −4.707 | 1.00 | 11.75 | A |
| ATOM | 1038 | CA | ARG | A | 736 | 9.822 | 3.640 | −6.141 | 1.00 | 13.68 | A |
| ATOM | 1039 | CB | ARG | A | 736 | 11.061 | 3.264 | −6.961 | 1.00 | 15.62 | A |
| ATOM | 1040 | CG | ARG | A | 736 | 10.798 | 2.724 | −8.368 | 1.00 | 22.11 | A |
| ATOM | 1041 | CD | ARG | A | 736 | 10.163 | 3.773 | −9.223 | 1.00 | 21.46 | A |
| ATOM | 1042 | NE | ARG | A | 736 | 10.472 | 3.653 | −10.651 | 1.00 | 25.32 | A |
| ATOM | 1043 | CZ | ARG | A | 736 | 9.737 | 3.010 | −11.560 | 1.00 | 26.66 | A |
| ATOM | 1044 | NE1 | ARG | A | 736 | 8.621 | 2.386 | −11.212 | 1.00 | 27.03 | A |
| ATOM | 1045 | NH2 | ARG | A | 736 | 10.092 | 3.040 | −12.848 | 1.00 | 26.16 | A |
| ATOM | 1046 | C | ARG | A | 736 | 8.674 | 2.697 | −6.484 | 1.00 | 14.97 | A |
| ATOM | 1047 | O | ARG | A | 736 | 7.713 | 3.078 | −7.155 | 1.00 | 14.53 | A |
| ATOM | 1048 | N | GLY | A | 737 | 8.788 | 1.462 | −6.010 | 1.00 | 14.21 | A |
| ATOM | 1049 | CA | GLY | A | 737 | 7.757 | 0.474 | −6.256 | 1.00 | 13.57 | A |
| ATOM | 1050 | C | GLY | A | 737 | 6.422 | 0.906 | −5.684 | 1.00 | 12.80 | A |
| ATOM | 1051 | O | GLY | A | 737 | 5.390 | 0.716 | −6.314 | 1.00 | 11.94 | A |
| ATOM | 1052 | N | ILE | A | 738 | 6.437 | 1.492 | −4.490 | 1.00 | 14.09 | A |
| ATOM | 1053 | CA | ILE | A | 738 | 5.208 | 1.943 | −3.855 | 1.00 | 11.56 | A |
| ATOM | 1054 | CB | ILE | A | 738 | 5.467 | 2.392 | −2.405 | 1.00 | 11.50 | A |
| ATOM | 1055 | CG2 | ILE | A | 738 | 4.195 | 3.031 | −1.825 | 1.00 | 9.80 | A |
| ATOM | 1056 | CG1 | ILE | A | 738 | 5.917 | 1.175 | −1.558 | 1.00 | 8.91 | A |
| ATOM | 1057 | CD1 | ILE | A | 738 | 6.389 | 1.544 | −0.153 | 1.00 | 8.26 | A |
| ATOM | 1058 | C | ILE | A | 738 | 4.584 | 3.110 | −4.640 | 1.00 | 13.67 | A |
| ATOM | 1059 | O | ILE | A | 738 | 3.374 | 3.141 | −4.871 | 1.00 | 11.58 | A |
| ATOM | 1060 | N | ALA | A | 739 | 5.416 | 4.055 | −5.070 | 1.00 | 12.15 | A |
| ATOM | 1061 | CA | ALA | A | 739 | 4.918 | 5.207 | −5.831 | 1.00 | 12.85 | A |
| ATOM | 1062 | CB | ALA | A | 739 | 6.054 | 6.219 | −6.067 | 1.00 | 9.40 | A |
| ATOM | 1063 | C | ALA | A | 739 | 4.354 | 4.728 | −7.170 | 1.00 | 10.80 | A |
| ATOM | 1064 | O | ALA | A | 739 | 3.374 | 5.277 | −7.679 | 1.00 | 13.80 | A |
| ATOM | 1065 | N | ALA | A | 740 | 4.980 | 3.708 | −7.736 | 1.00 | 8.81 | A |
| ATOM | 1066 | CA | ALA | A | 740 | 4.526 | 3.169 | −9.009 | 1.00 | 11.12 | A |
| ATOM | 1067 | CB | ALA | A | 740 | 5.508 | 2.129 | −9.514 | 1.00 | 9.71 | A |
| ATOM | 1068 | C | ALA | A | 740 | 3.151 | 2.532 | −8.830 | 1.00 | 12.53 | A |
| ATOM | 1069 | O | ALA | A | 740 | 2.262 | 2.721 | −9.655 | 1.00 | 9.41 | A |
| ATOM | 1070 | N | GLY | A | 741 | 2.992 | 1.748 | −7.765 | 1.00 | 10.66 | A |
| ATOM | 1071 | CA | GLY | A | 741 | 1.700 | 1.138 | −7.516 | 1.00 | 10.59 | A |
| ATOM | 1072 | C | GLY | A | 741 | 0.642 | 2.211 | −7.310 | 1.00 | 10.74 | A |
| ATOM | 1073 | O | GLY | A | 741 | −0.460 | 2.097 | −7.838 | 1.00 | 11.88 | A |
| ATOM | 1074 | N | MET | A | 742 | 0.988 | 3.260 | −6.564 | 1.00 | 9.32 | A |
| ATOM | 1075 | CA | MET | A | 742 | 0.057 | 4.342 | −6.276 | 1.00 | 10.60 | A |
| ATOM | 1076 | CE | MET | A | 742 | 0.593 | 5.246 | −5.170 | 1.00 | 9.60 | A |
| ATOM | 1077 | CG | MET | A | 742 | 0.530 | 4.658 | −3.753 | 1.00 | 15.00 | A |
| ATOM | 1078 | SD | MET | A | 742 | −1.113 | 4.092 | −3.272 | 1.00 | 12.37 | A |
| ATOM | 1079 | CE | MET | A | 742 | −1.973 | 5.605 | −3.201 | 1.00 | 7.46 | A |
| ATOM | 1080 | C | MET | A | 742 | −0.274 | 5.184 | −7.506 | 1.00 | 11.90 | A |
| ATOM | 1081 | O | MET | A | 742 | −1.396 | 5.681 | −7.636 | 1.00 | 11.73 | A |
| ATOM | 1082 | N | LYS | A | 743 | 0.710 | 5.362 | −8.382 | 1.00 | 12.27 | A |
| ATOM | 1083 | CA | LYS | A | 743 | 0.510 | 6.128 | −9.606 | 1.00 | 14.85 | A |
| ATOM | 1084 | CB | LYS | A | 743 | 1.828 | 6.206 | −10.386 | 1.00 | 14.51 | A |
| ATOM | 1085 | CG | LYS | A | 743 | 1.892 | 7.305 | −11.431 | 1.00 | 17.54 | A |
| ATOM | 1086 | CD | LYS | A | 743 | 1.282 | 6.871 | −12.720 | 1.00 | 18.67 | A |
| ATOM | 1087 | CE | LYS | A | 743 | 1.404 | 7.987 | −13.780 | 1.00 | 21.38 | A |
| ATOM | 1088 | NZ | LYS | A | 743 | 0.863 | 7.526 | −15.088 | 1.00 | 19.61 | A |
| ATOM | 1089 | C | LYS | A | 743 | −0.554 | 5.375 | −10.406 | 1.00 | 14.04 | A |
| ATOM | 1090 | O | LYS | A | 743 | −1.503 | 5.971 | −10.918 | 1.00 | 15.53 | A |
| ATOM | 1091 | N | TYR | A | 744 | −0.401 | 4.055 | −10.474 | 1.00 | 11.88 | A |
| ATOM | 1092 | CA | TYR | A | 744 | −1.341 | 3.213 | −11.194 | 1.00 | 11.73 | A |
| ATOM | 1093 | CB | TYR | A | 744 | −0.884 | 1.747 | −11.168 | 1.00 | 10.89 | A |
| ATOM | 1094 | CG | TYR | A | 744 | −1.920 | 0.774 | −11.699 | 1.00 | 12.71 | A |
| ATOM | 1095 | CD1 | TYR | A | 744 | −2.013 | 0.478 | −13.063 | 1.00 | 11.56 | A |
| ATOM | 1096 | CE1 | TYR | A | 744 | −2.969 | −0.441 | −13.543 | 1.00 | 12.70 | A |
| ATOM | 1097 | CD2 | TYR | A | 744 | −2.807 | 0.144 | −10.832 | 1.00 | 11.18 | A |
| ATOM | 1098 | CE2 | TYR | A | 744 | −3.756 | −0.760 | −11.299 | 1.00 | 12.54 | A |
| ATOM | 1099 | CZ | TYR | A | 744 | −3.830 | −1.054 | −12.644 | 1.00 | 12.73 | A |
| ATOM | 1100 | OH | TYR | A | 744 | −4.728 | −2.010 | −13.051 | 1.00 | 13.10 | A |
| ATOM | 1101 | C | TYR | A | 744 | −2.761 | 3.333 | −10.600 | 1.00 | 13.06 | A |
| ATOM | 1102 | O | TYR | A | 744 | −3.725 | 3.511 | −11.339 | 1.00 | 12.71 | A |
| ATOM | 1103 | N | LEU | A | 745 | −2.892 | 3.239 | −9.273 | 1.00 | 11.93 | A |
| ATOM | 1104 | CA | LEU | A | 745 | −4.203 | 3.364 | −8.634 | 1.00 | 9.55 | A |
| ATOM | 1105 | CE | LEU | A | 745 | −4.098 | 3.129 | −7.117 | 1.00 | 12.28 | A |
| ATOM | 1106 | CG | LEU | A | 745 | −3.548 | 1.739 | −6.716 | 1.00 | 10.40 | A |
| ATOM | 1107 | CD1 | LEU | A | 745 | −3.313 | 1.648 | −5.206 | 1.00 | 11.47 | A |
| ATOM | 1108 | CD2 | LEU | A | 745 | −4.550 | 0.682 | −7.149 | 1.00 | 13.02 | A |
| ATOM | 1109 | C | LEU | A | 745 | −4.808 | 4.755 | −8.898 | 1.00 | 12.15 | A |
| ATOM | 1110 | O | LEU | A | 745 | −5.991 | 4.876 | −9.231 | 1.00 | 11.72 | A |
| ATOM | 1111 | N | ALA | A | 746 | −4.000 | 5.796 | −8.732 | 1.00 | 13.75 | A |
| ATOM | 1112 | CA | ALA | A | 746 | −4.447 | 7.159 | −8.972 | 1.00 | 13.62 | A |
| ATOM | 1113 | CB | ALA | A | 746 | −3.289 | 8.153 | −8.704 | 1.00 | 14.55 | A |
| ATOM | 1114 | C | ALA | A | 746 | −4.930 | 7.270 | −10.425 | 1.00 | 14.12 | A |
| ATOM | 1115 | O | ALA | A | 746 | −5.965 | 7.877 | −10.694 | 1.00 | 16.20 | A |
| ATOM | 1116 | N | ASP | A | 747 | −4.194 | 6.668 | −11.356 | 1.00 | 13.55 | A |
| ATOM | 1117 | CA | ASP | A | 747 | −4.583 | 6.692 | −12.773 | 1.00 | 14.00 | A |
| ATOM | 1118 | CB | ASP | A | 747 | −3.563 | 5.961 | −13.641 | 1.00 | 14.56 | A |
| ATOM | 1119 | CG | ASP | A | 747 | −2.358 | 6.820 | −14.001 | 1.00 | 10.40 | A |
| ATOM | 1120 | OD1 | ASP | A | 747 | −2.345 | 8.018 | −13.679 | 1.00 | 11.42 | A |
| ATOM | 1121 | OD2 | ASP | A | 747 | −1.438 | 6.263 | −14.625 | 1.00 | 13.31 | A |
| ATOM | 1122 | C | ASP | A | 747 | −5.939 | 6.023 | −12.995 | 1.00 | 17.40 | A |
| ATOM | 1123 | O | ASP | A | 747 | −6.680 | 6.391 | −13.921 | 1.00 | 13.00 | A |
| ATOM | 1124 | N | MET | A | 748 | −6.238 | 5.032 | −12.151 | 1.00 | 15.80 | A |
| ATOM | 1125 | CA | MET | A | 748 | −7.491 | 4.263 | −12.210 | 1.00 | 17.56 | A |
| ATOM | 1126 | CB | MET | A | 748 | −7.333 | 2.914 | −11.491 | 1.00 | 18.98 | A |
| ATOM | 1127 | CG | MET | A | 748 | −6.414 | 1.918 | −12.149 | 1.00 | 24.30 | A |
| ATOM | 1128 | SD | MET | A | 748 | −7.172 | 1.067 | −13.532 | 1.00 | 32.75 | A |
| ATOM | 1129 | CE | MET | A | 748 | −8.491 | 0.141 | −12.704 | 1.00 | 26.32 | A |
| ATOM | 1130 | C | MET | A | 748 | −8.593 | 5.022 | −11.493 | 1.00 | 16.57 | A |
| ATOM | 1131 | O | MET | A | 748 | −9.744 | 4.557 | −11.402 | 1.00 | 17.25 | A |
| ATOM | 1132 | N | ASN | A | 749 | −8.223 | 6.175 | −10.954 | 1.00 | 15.84 | A |
| ATOM | 1133 | CA | ASN | A | 749 | −9.148 | 7.007 | −10.198 | 1.00 | 17.28 | A |
| ATOM | 1134 | CB | ASN | A | 749 | −10.408 | 7.295 | −11.017 | 1.00 | 22.09 | A |
| ATOM | 1135 | CG | ASN | A | 749 | −11.210 | 8.431 | −10.447 | 1.00 | 25.81 | A |
| ATOM | 1136 | CD1 | ASN | A | 749 | −10.647 | 9.371 | −9.892 | 1.00 | 29.95 | A |
| ATOM | 1137 | ND2 | ASN | A | 749 | −12.528 | 8.363 | −10.580 | 1.00 | 31.13 | A |
| ATOM | 1138 | C | ASN | A | 749 | −9.528 | 6.329 | −8.875 | 1.00 | 16.80 | A |
| ATOM | 1139 | O | ASN | A | 749 | −10.660 | 6.444 | −6.388 | 1.00 | 14.69 | A |
| ATOM | 1140 | N | TYR | A | 750 | −8.579 | 5.608 | −8.293 | 1.00 | 16.08 | A |
| ATOM | 1141 | CA | TYR | A | 750 | −8.827 | 4.949 | −7.007 | 1.00 | 13.01 | A |
| ATOM | 1142 | CB | TYR | A | 750 | −8.345 | 3.498 | −7.042 | 1.00 | 13.41 | A |
| ATOM | 1143 | CG | TYR | A | 750 | −8.556 | 2.791 | −5.721 | 1.00 | 13.96 | A |
| ATOM | 1144 | CD1 | TYR | A | 750 | −9.792 | 2.249 | −5.402 | 1.00 | 11.49 | A |
| ATOM | 1145 | CE1 | TYR | A | 750 | −10.022 | 1.633 | −4.175 | 1.00 | 13.84 | A |
| ATOM | 1146 | CD2 | TYR | A | 750 | −7.530 | 2.704 | −4.774 | 1.00 | 13.22 | A |
| ATOM | 1147 | CE2 | TYR | A | 750 | −7.749 | 2.088 | −3.534 | 1.00 | 13.77 | A |
| ATOM | 1148 | CZ | TYR | A | 750 | −9.003 | 1.559 | −3.251 | 1.00 | 14.92 | A |
| ATOM | 1149 | OH | TYR | A | 750 | −9.262 | 0.963 | −2.028 | 1.00 | 15.35 | A |
| ATOM | 1150 | C | TYR | A | 750 | −8.039 | 5.699 | −5.934 | 1.00 | 13.00 | A |
| ATOM | 1151 | O | TYR | A | 750 | −6.814 | 5.785 | −6.012 | 1.00 | 13.01 | A |
| ATOM | 1152 | N | VAL | A | 751 | −8.743 | 6.256 | −4.955 | 1.00 | 14.41 | A |
| ATOM | 1153 | CA | VAL | A | 751 | −8.111 | 6.968 | −3.843 | 1.00 | 15.71 | A |
| ATOM | 1154 | CB | VAL | A | 751 | −8.968 | 8.151 | −3.365 | 1.00 | 17.55 | A |
| ATOM | 1155 | CG1 | VAL | A | 751 | −8.324 | 8.792 | −2.143 | 1.00 | 20.77 | A |
| ATOM | 1156 | CG2 | VAL | A | 751 | −9.123 | 9.181 | −4.491 | 1.00 | 18.56 | A |
| ATOM | 1157 | C | VAL | A | 751 | −8.027 | 5.946 | −2.715 | 1.00 | 16.81 | A |
| ATOM | 1158 | O | VAL | A | 751 | −9.058 | 5.449 | −2.267 | 1.00 | 15.55 | A |
| ATOM | 1159 | N | HIS | A | 752 | −6.814 | 5.643 | −2.258 | 1.00 | 14.86 | A |
| ATOM | 1160 | CA | HIS | A | 752 | −6.612 | 4.645 | −1.209 | 1.00 | 13.56 | A |
| ATOM | 1161 | CB | HIS | A | 752 | −5.123 | 4.316 | −1.085 | 1.00 | 11.05 | A |
| ATOM | 1162 | CG | HIS | A | 752 | −4.852 | 3.078 | −0.290 | 1.00 | 8.71 | A |
| ATOM | 1163 | CD2 | HIS | A | 752 | −4.529 | 1.825 | −0.681 | 1.00 | 10.79 | A |
| ATOM | 1164 | ND1 | HIS | A | 752 | −4.946 | 3.039 | 1.084 | 1.00 | 10.98 | A |
| ATOM | 1165 | CE1 | HIS | A | 752 | −4.688 | 1.614 | 1.505 | 1.00 | 11.77 | A |
| ATOM | 1166 | NE2 | HIS | A | 752 | −4.431 | 1.060 | 0.454 | 1.00 | 10.75 | A |
| ATOM | 1167 | C | HIS | A | 752 | −7.149 | 5.044 | 0.161 | 1.00 | 14.58 | A |
| ATOM | 1168 | O | HIS | A | 752 | −7.821 | 4.251 | 0.830 | 1.00 | 16.10 | A |
| ATOM | 1169 | N | ARG | A | 753 | −6.825 | 6.269 | 0.573 | 1.00 | 15.29 | A |
| ATOM | 1170 | CA | ARG | A | 753 | −7.251 | 6.827 | 1.855 | 1.00 | 17.04 | A |
| ATOM | 1171 | CB | ARG | A | 753 | −8.755 | 6.598 | 2.068 | 1.00 | 21.60 | A |
| ATOM | 1172 | CG | ARG | A | 753 | −9.654 | 7.287 | 1.056 | 1.00 | 25.77 | A |
| ATOM | 1173 | CD | ARG | A | 753 | −11.110 | 7.212 | 1.484 | 1.00 | 31.38 | A |
| ATOM | 1174 | NE | ARG | A | 753 | −11.969 | 8.087 | 0.685 | 1.00 | 33.80 | A |
| ATOM | 1175 | CZ | ARG | A | 753 | −13.158 | 8.533 | 1.082 | 1.00 | 36.41 | A |
| ATOM | 1176 | NH1 | ARG | A | 753 | −13.637 | 8.187 | 2.271 | 1.00 | 35.80 | A |
| ATOM | 1177 | NH2 | ARG | A | 753 | −13.864 | 9.336 | 0.295 | 1.00 | 37.87 | A |
| ATOM | 1178 | C | ARG | A | 753 | −6.503 | 6.326 | 3.097 | 1.00 | 16.08 | A |
| ATOM | 1179 | O | ARG | A | 753 | −6.555 | 6.972 | 4.144 | 1.00 | 15.20 | A |
| ATOM | 1180 | N | ASP | A | 754 | −5.819 | 5.189 | 3.008 | 1.00 | 14.97 | A |
| ATOM | 1181 | CA | ASP | A | 754 | −5.101 | 4.667 | 4.180 | 1.00 | 16.09 | A |
| ATOM | 1182 | CB | ASP | A | 754 | −5.941 | 3.555 | 4.826 | 1.00 | 18.24 | A |
| ATOM | 1183 | CG | ASP | A | 754 | −5.413 | 3.098 | 6.188 | 1.00 | 23.23 | A |
| ATOM | 1184 | OD1 | ASP | A | 754 | −4.927 | 3.920 | 6.990 | 1.00 | 25.55 | A |
| ATOM | 1185 | OD2 | ASP | A | 754 | −5.515 | 1.881 | 6.469 | 1.00 | 28.99 | A |
| ATOM | 1186 | C | ASP | A | 754 | −3.702 | 4.161 | 3.796 | 1.00 | 14.92 | A |
| ATOM | 1187 | O | ASP | A | 754 | −3.280 | 3.078 | 4.201 | 1.00 | 13.06 | A |
| ATOM | 1188 | N | LEU | A | 755 | −2.993 | 4.955 | 2.998 | 1.00 | 14.02 | A |
| ATOM | 1189 | CA | LEU | A | 755 | −1.652 | 4.594 | 2.572 | 1.00 | 13.51 | A |
| ATOM | 1190 | CB | LEU | A | 755 | −1.212 | 5.454 | 1.384 | 1.00 | 9.06 | A |
| ATOM | 1191 | CD | LEU | A | 755 | 0.216 | 5.254 | 0.884 | 1.00 | 10.93 | A |
| ATOM | 1192 | CD1 | LEU | A | 755 | 0.452 | 3.797 | 0.483 | 1.00 | 9.97 | A |
| ATOM | 1193 | CD2 | LEU | A | 755 | 0.458 | 6.186 | −0.312 | 1.00 | 8.65 | A |
| ATOM | 1194 | C | LEU | A | 755 | −0.705 | 4.772 | 3.758 | 1.00 | 13.01 | A |
| ATOM | 1195 | O | LEU | A | 755 | −0.596 | 5.859 | 4.346 | 1.00 | 13.78 | A |
| ATOM | 1196 | N | ALA | A | 756 | −0.043 | 3.675 | 4.110 | 1.00 | 12.68 | A |
| ATOM | 1197 | CA | ALA | A | 756 | 0.876 | 3.623 | 5.247 | 1.00 | 11.51 | A |
| ATOM | 1198 | CB | ALA | A | 756 | 0.068 | 3.568 | 6.560 | 1.00 | 9.75 | A |
| ATOM | 1199 | C | ALA | A | 756 | 1.732 | 2.361 | 5.094 | 1.00 | 10.18 | A |
| ATOM | 1200 | O | ALA | A | 756 | 1.303 | 1.398 | 4.465 | 1.00 | 7.97 | A |
| ATOM | 1201 | N | ALA | A | 757 | 2.930 | 2.346 | 5.671 | 1.00 | 7.85 | A |
| ATOM | 1202 | CA | ALA | A | 757 | 3.802 | 1.186 | 5.514 | 1.00 | 8.61 | A |
| ATOM | 1203 | CB | ALA | A | 757 | 5.153 | 1.430 | 6.239 | 1.00 | 7.83 | A |
| ATOM | 1204 | C | ALA | A | 757 | 3.148 | −0.117 | 6.016 | 1.00 | 8.24 | A |
| ATOM | 1205 | O | ALA | A | 757 | 3.423 | −1.189 | 5.490 | 1.00 | 10.34 | A |
| ATOM | 1206 | N | ARG | A | 758 | 2.279 | −0.026 | 7.016 | 1.00 | 10.60 | A |
| ATOM | 1207 | CA | ARG | A | 758 | 1.607 | −1.219 | 7.537 | 1.00 | 12.89 | A |
| ATOM | 1208 | CB | ARG | A | 758 | 0.806 | −0.895 | 8.809 | 1.00 | 14.94 | A |
| ATOM | 1209 | CG | ARG | A | 758 | −0.235 | 0.190 | 8.616 | 1.00 | 20.57 | A |
| ATOM | 1210 | CD | ARG | A | 758 | −1.226 | 0.256 | 9.775 | 1.00 | 23.51 | A |
| ATOM | 1211 | NE | ARG | A | 758 | −2.251 | 1.267 | 9.517 | 1.00 | 26.29 | A |
| ATOM | 1212 | CZ | ARG | A | 758 | −2.017 | 2.574 | 9.514 | 1.00 | 26.89 | A |
| ATOM | 1213 | NH1 | ARG | A | 758 | −0.794 | 3.029 | 9.762 | 1.00 | 28.84 | A |
| ATOM | 1214 | NH2 | ARG | A | 758 | −2.999 | 3.425 | 9.259 | 1.00 | 29.97 | A |
| ATOM | 1215 | C | ARG | A | 758 | 0.667 | −1.782 | 6.479 | 1.00 | 13.53 | A |
| ATOM | 1216 | O | ARG | A | 758 | 0.245 | −2.942 | 6.555 | 1.00 | 11.71 | A |
| ATOM | 1217 | N | ASN | A | 759 | 0.348 | −0.960 | 5.486 | 1.00 | 12.82 | A |
| ATOM | 1218 | CA | ASN | A | 759 | −0.542 | −1.401 | 4.421 | 1.00 | 14.76 | A |
| ATOM | 1219 | CB | ASN | A | 759 | −1.659 | −0.368 | 4.217 | 1.00 | 13.84 | A |
| ATOM | 1220 | CD | ASN | A | 759 | −2.575 | −0.315 | 5.409 | 1.00 | 17.87 | A |
| ATOM | 1221 | OD1 | ASN | A | 759 | −2.929 | −1.366 | 5.947 | 1.00 | 14.41 | A |
| ATOM | 1222 | ND2 | ASN | A | 759 | −2.942 | 0.888 | 5.853 | 1.00 | 16.21 | A |
| ATOM | 1223 | C | ASN | A | 759 | 0.175 | −1.727 | 3.117 | 1.00 | 14.12 | A |
| ATOM | 1224 | O | ASN | A | 759 | −0.450 | −1.885 | 2.067 | 1.00 | 16.83 | A |
| ATOM | 1225 | N | ILE | A | 760 | 1.499 | −1.823 | 3.194 | 1.00 | 12.17 | A |
| ATOM | 1226 | CA | ILE | A | 760 | 2.316 | −2.195 | 2.045 | 1.00 | 10.80 | A |
| ATOM | 1227 | CB | ILE | A | 760 | 3.503 | −1.198 | 1.811 | 1.00 | 10.66 | A |
| ATOM | 1228 | CG2 | ILE | A | 760 | 4.335 | −1.671 | 0.629 | 1.00 | 7.31 | A |
| ATOM | 1229 | CG1 | ILE | A | 760 | 2.992 | 0.233 | 1.571 | 1.00 | 9.62 | A |
| ATOM | 1230 | CD1 | ILE | A | 760 | 2.035 | 0.369 | 0.357 | 1.00 | 10.93 | A |
| ATOM | 1231 | C | ILE | A | 760 | 2.905 | −3.587 | 2.374 | 1.00 | 12.66 | A |
| ATOM | 1232 | O | ILE | A | 760 | 3.390 | −3.801 | 3.496 | 1.00 | 14.01 | A |
| ATOM | 1233 | N | LEU | A | 761 | 2.846 | −4.534 | 1.431 | 1.00 | 10.97 | A |
| ATOM | 1234 | CA | LEU | A | 761 | 3.409 | −5.874 | 1.673 | 1.00 | 13.49 | A |
| ATOM | 1235 | CB | LEU | A | 761 | 2.452 | −6.983 | 1.222 | 1.00 | 12.03 | A |
| ATOM | 1236 | CG | LEU | A | 761 | 1.135 | −7.005 | 1.998 | 1.00 | 15.46 | A |
| ATOM | 1237 | CD1 | LEU | A | 761 | 0.168 | −8.010 | 1.371 | 1.00 | 15.87 | A |
| ATOM | 1238 | CD2 | LEU | A | 761 | 1.418 | −7.372 | 3.443 | 1.00 | 20.01 | A |
| ATOM | 1239 | C | LEU | A | 761 | 4.741 | −6.004 | 0.936 | 1.00 | 12.83 | A |
| ATOM | 1240 | O | LEU | A | 761 | 4.955 | −5.356 | −0.085 | 1.00 | 11.29 | A |
| ATOM | 1241 | N | VAL | A | 762 | 5.623 | −6.853 | 1.457 | 1.00 | 11.95 | A |
| ATOM | 1242 | CA | VAL | A | 762 | 6.957 | −7.030 | 0.885 | 1.00 | 10.52 | A |
| ATOM | 1243 | CB | VAL | A | 762 | 8.017 | −6.525 | 1.894 | 1.00 | 11.59 | A |
| ATOM | 1244 | CG1 | VAL | A | 762 | 9.400 | −6.502 | 1.267 | 1.00 | 10.27 | A |
| ATOM | 1245 | CG2 | VAL | A | 762 | 7.627 | −5.142 | 2.388 | 1.00 | 11.18 | A |
| ATOM | 1246 | C | VAL | A | 762 | 7.243 | −8.488 | 0.525 | 1.00 | 11.69 | A |
| ATOM | 1247 | O | VAL | A | 762 | 7.014 | −9.390 | 1.341 | 1.00 | 11.49 | A |
| ATOM | 1248 | N | ASN | A | 763 | 7.740 | −8.734 | −0.688 | 1.00 | 10.00 | A |
| ATOM | 1249 | CA | ASN | A | 763 | 8.028 | −10.105 | −1.082 | 1.00 | 13.20 | A |
| ATOM | 1250 | CB | ASN | A | 763 | 7.586 | −10.369 | −2.543 | 1.00 | 14.49 | A |
| ATOM | 1251 | CG | ASN | A | 763 | 8.550 | −9.829 | −3.587 | 1.00 | 15.38 | A |
| ATOM | 1252 | OD1 | ASN | A | 763 | 9.605 | −9.278 | −3.274 | 1.00 | 17.52 | A |
| ATOM | 1253 | ND2 | ASN | A | 763 | 8.186 | −10.010 | −4.855 | 1.00 | 17.84 | A |
| ATOM | 1254 | C | ASN | A | 763 | 9.487 | −10.470 | −0.842 | 1.00 | 14.94 | A |
| ATOM | 1255 | O | ASN | A | 763 | 10.259 | −9.643 | −0.358 | 1.00 | 15.44 | A |
| ATOM | 1256 | N | SER | A | 764 | 9.862 | −11.711 | −1.142 | 1.00 | 17.21 | A |
| ATOM | 1257 | CA | SER | A | 764 | 11.233 | −12.153 | −0.897 | 1.00 | 19.91 | A |
| ATOM | 1258 | CB | SER | A | 764 | 11.372 | −13.649 | −1.186 | 1.00 | 20.57 | A |
| ATOM | 1259 | OG | SER | A | 764 | 11.104 | −13.926 | −2.547 | 1.00 | 24.62 | A |
| ATOM | 1260 | C | SER | A | 764 | 12.274 | −11.369 | −1.687 | 1.00 | 21.35 | A |
| ATOM | 1261 | O | SER | A | 764 | 13.443 | −11.319 | −1.305 | 1.00 | 22.51 | A |
| ATOM | 1262 | N | ASN | A | 765 | 11.857 | −10.746 | −2.780 | 1.00 | 19.26 | A |
| ATOM | 1263 | CA | ASN | A | 765 | 12.790 | −9.969 | −3.571 | 1.00 | 19.42 | A |
| ATOM | 1264 | CB | ASN | A | 765 | 12.513 | −10.164 | −5.061 | 1.00 | 21.15 | A |
| ATOM | 1265 | CG | ASN | A | 765 | 12.836 | −11.572 | −5.524 | 1.00 | 24.10 | A |
| ATOM | 1266 | OD1 | ASN | A | 765 | 13.910 | −12.096 | −5.210 | 1.00 | 27.80 | A |
| ATOM | 1267 | ND2 | ASN | A | 765 | 11.917 | −12.194 | −6.271 | 1.00 | 26.16 | A |
| ATOM | 1268 | C | ASN | A | 765 | 12.738 | −8.489 | −3.198 | 1.00 | 19.25 | A |
| ATOM | 1269 | O | ASN | A | 765 | 13.266 | −7.631 | −3.915 | 1.00 | 19.32 | A |
| ATOM | 1270 | N | LEU | A | 766 | 12.125 | −8.198 | −2.053 | 1.00 | 15.23 | A |
| ATOM | 1271 | CA | LEU | A | 766 | 12.004 | −6.830 | −1.552 | 1.00 | 15.03 | A |
| ATOM | 1272 | CB | LEU | A | 766 | 13.386 | −6.151 | −1.451 | 1.00 | 14.51 | A |
| ATOM | 1273 | CG | LEU | A | 766 | 14.497 | −6.907 | −0.699 | 1.00 | 16.00 | A |
| ATOM | 1274 | CD1 | LEU | A | 766 | 15.704 | −5.956 | −0.565 | 1.00 | 17.05 | A |
| ATOM | 1275 | CD2 | LEU | A | 766 | 14.030 | −7.346 | 0.691 | 1.00 | 17.26 | A |
| ATOM | 1276 | C | LEU | A | 766 | 11.054 | −5.951 | −2.376 | 1.00 | 13.94 | A |
| ATOM | 1277 | O | LEU | A | 766 | 11.021 | −4.735 | −2.196 | 1.00 | 13.56 | A |
| ATOM | 1278 | N | VAL | A | 767 | 10.289 | −6.562 | −3.279 | 1.00 | 12.87 | A |
| ATOM | 1279 | CA | VAL | A | 767 | 9.322 | −5.808 | −4.070 | 1.00 | 11.98 | A |
| ATOM | 1280 | CB | VAL | A | 767 | 8.714 | −6.648 | −5.202 | 1.00 | 9.61 | A |
| ATOM | 1281 | CG1 | VAL | A | 767 | 7.574 | −5.866 | −5.857 | 1.00 | 9.58 | A |
| ATOM | 1282 | CG2 | VAL | A | 767 | 9.783 | −6.976 | −6.240 | 1.00 | 9.18 | A |
| ATOM | 1283 | C | VAL | A | 767 | 8.190 | −5.409 | −3.130 | 1.00 | 13.34 | A |
| ATOM | 1284 | O | VAL | A | 767 | 7.635 | −6.265 | −2.436 | 1.00 | 12.17 | A |
| ATOM | 1285 | N | CYS | A | 768 | 7.860 | −4.118 | −3.099 | 1.00 | 12.87 | A |
| ATOM | 1286 | CA | CYS | A | 768 | 6.786 | −3.594 | −2.255 | 1.00 | 12.04 | A |
| ATOM | 1287 | CB | CYS | A | 768 | 7.213 | −2.257 | −1.649 | 1.00 | 12.99 | A |
| ATOM | 1288 | SG | CYS | A | 768 | 8.599 | −2.417 | −0.477 | 1.00 | 15.84 | A |
| ATOM | 1289 | C | CYS | A | 768 | 5.475 | −3.421 | −3.027 | 1.00 | 13.36 | A |
| ATOM | 1290 | O | CYS | A | 768 | 5.454 | −2.834 | −4.116 | 1.00 | 13.26 | A |
| ATOM | 1291 | N | LYS | A | 769 | 4.376 | −3.894 | −2.440 | 1.00 | 12.25 | A |
| ATOM | 1292 | CA | LYS | A | 769 | 3.073 | −3.814 | −3.101 | 1.00 | 12.28 | A |
| ATOM | 1293 | CB | LYS | A | 769 | 2.659 | −5.209 | −3.562 | 1.00 | 9.87 | A |
| ATOM | 1294 | CG | LYS | A | 769 | 3.672 | −5.857 | −4.462 | 1.00 | 13.00 | A |
| ATOM | 1295 | CD | LYS | A | 769 | 3.170 | −7.163 | −5.105 | 1.00 | 14.19 | A |
| ATOM | 1296 | CE | LYS | A | 769 | 4.242 | −7.757 | −6.022 | 1.00 | 16.77 | A |
| ATOM | 1297 | NZ | LYS | A | 769 | 3.710 | −8.749 | −6.980 | 1.00 | 16.50 | A |
| ATOM | 1298 | C | LYS | A | 769 | 1.978 | −3.202 | −2.245 | 1.00 | 13.01 | A |
| ATOM | 1299 | O | LYS | A | 769 | 1.844 | −3.531 | −1.063 | 1.00 | 14.29 | A |
| ATOM | 1300 | N | VAL | A | 770 | 1.184 | −2.309 | −2.835 | 1.00 | 12.10 | A |
| ATOM | 1301 | CA | VAL | A | 770 | 0.101 | −1.677 | −2.089 | 1.00 | 11.10 | A |
| ATOM | 1302 | CB | VAL | A | 770 | −0.512 | −0.499 | −2.889 | 1.00 | 10.58 | A |
| ATOM | 1303 | CG1 | VAL | A | 770 | −1.644 | 0.112 | −2.100 | 1.00 | 5.30 | A |
| ATOM | 1304 | CG2 | VAL | A | 770 | 0.570 | 0.551 | −3.186 | 1.00 | 6.99 | A |
| ATOM | 1305 | C | VAL | A | 770 | −1.000 | −2.696 | −1.796 | 1.00 | 11.89 | A |
| ATOM | 1306 | O | VAL | A | 770 | −1.353 | −3.491 | −2.662 | 1.00 | 12.00 | A |
| ATOM | 1307 | N | SER | A | 771 | −1.527 | −2.667 | −0.576 | 1.00 | 11.80 | A |
| ATOM | 1308 | CA | SER | A | 771 | −2.590 | −3.578 | −0.150 | 1.00 | 14.70 | A |
| ATOM | 1309 | CB | SER | A | 771 | −2.002 | −4.666 | 0.765 | 1.00 | 17.32 | A |
| ATOM | 1310 | OG | SER | A | 771 | −2.990 | −5.600 | 1.144 | 1.00 | 19.91 | A |
| ATOM | 1311 | C | SER | A | 771 | −3.655 | −2.795 | 0.630 | 1.00 | 14.68 | A |
| ATOM | 1312 | O | SER | A | 771 | −3.709 | −1.562 | 0.544 | 1.00 | 15.55 | A |
| ATOM | 1313 | N | ASP | A | 772 | −4.508 | −3.504 | 1.378 | 1.00 | 14.81 | A |
| ATOM | 1314 | CA | ASP | A | 772 | −5.530 | −2.865 | 2.212 | 1.00 | 16.39 | A |
| ATOM | 1315 | CB | ASP | A | 772 | −4.852 | −1.845 | 3.134 | 1.00 | 19.62 | A |
| ATOM | 1316 | CG | ASP | A | 772 | −5.816 | −1.172 | 4.110 | 1.00 | 22.71 | A |
| ATOM | 1317 | OD1 | ASP | A | 772 | −5.738 | 0.078 | 4.232 | 1.00 | 26.71 | A |
| ATOM | 1318 | 0D2 | ASP | A | 772 | −6.622 | −1.863 | 4.762 | 1.00 | 20.60 | A |
| ATOM | 1319 | C | ASP | A | 772 | −6.633 | −2.184 | 1.391 | 1.00 | 15.48 | A |
| ATOM | 1320 | O | ASP | A | 772 | −6.867 | −0.983 | 1.528 | 1.00 | 15.52 | A |
| ATOM | 1321 | N | PHE | A | 773 | −7.302 | −2.962 | 0.546 | 1.00 | 16.06 | A |
| ATOM | 1322 | CA | PHE | A | 773 | −8.376 | −2.449 | −0.293 | 1.00 | 17.22 | A |
| ATOM | 1323 | CB | PHE | A | 773 | −8.295 | −3.087 | −1.679 | 1.00 | 16.56 | A |
| ATOM | 1324 | CG | PHE | A | 773 | −7.062 | −2.696 | −2.441 | 1.00 | 14.03 | A |
| ATOM | 1325 | CD1 | PHE | A | 773 | −6.005 | −3.593 | −2.611 | 1.00 | 12.33 | A |
| ATOM | 1326 | CD2 | PHE | A | 773 | −6.927 | −1.396 | −2.927 | 1.00 | 12.51 | A |
| ATOM | 1327 | CE1 | PHE | A | 773 | −4.824 | −3.188 | −3.251 | 1.00 | 14.35 | A |
| ATOM | 1328 | CE2 | PHE | A | 773 | −5.757 | −0.989 | −3.563 | 1.00 | 13.76 | A |
| ATOM | 1329 | CZ | PHE | A | 773 | −4.706 | −1.885 | −3.723 | 1.00 | 11.63 | A |
| ATOM | 1330 | C | PHE | A | 773 | −9.748 | −2.693 | 0.325 | 1.00 | 21.10 | A |
| ATOM | 1331 | O | PHE | A | 773 | −10.023 | −3.784 | 0.824 | 1.00 | 25.25 | A |
| ATOM | 1332 | N | PRO | A | 797 | −4.563 | 6.529 | 12.016 | 1.00 | 37.73 | A |
| ATOM | 1333 | CD | PRO | A | 797 | −5.935 | 5.995 | 12.099 | 1.00 | 39.20 | A |
| ATOM | 1334 | CA | PRO | A | 797 | −4.285 | 7.010 | 10.661 | 1.00 | 36.62 | A |
| ATOM | 1335 | CB | PRO | A | 797 | −5.445 | 6.428 | 9.856 | 1.00 | 37.71 | A |
| ATOM | 1336 | CG | PRO | A | 797 | −6.572 | 6.522 | 10.818 | 1.00 | 37.28 | A |
| ATOM | 1337 | C | PRO | A | 797 | −4.206 | 8.537 | 10.558 | 1.00 | 34.49 | A |
| ATOM | 1338 | O | PRO | A | 797 | −3.764 | 9.071 | 9.543 | 1.00 | 33.47 | A |
| ATOM | 1339 | N | ILE | A | 798 | −4.629 | 9.236 | 11.609 | 1.00 | 32.85 | A |
| ATOM | 1340 | CA | ILE | A | 798 | −4.588 | 10.695 | 11.606 | 1.00 | 29.61 | A |
| ATOM | 1341 | CB | ILE | A | 798 | −4.905 | 11.269 | 12.997 | 1.00 | 30.95 | A |
| ATOM | 1342 | CG2 | ILE | A | 798 | −4.587 | 12.766 | 13.042 | 1.00 | 29.66 | A |
| ATOM | 1343 | CG1 | ILE | A | 798 | −6.373 | 11.020 | 13.327 | 1.00 | 29.77 | A |
| ATOM | 1344 | CD1 | ILE | A | 798 | −6.754 | 11.463 | 14.701 | 1.00 | 32.23 | A |
| ATOM | 1345 | C | ILE | A | 798 | −3.241 | 11.252 | 11.157 | 1.00 | 28.44 | A |
| ATOM | 1346 | O | ILE | A | 798 | −3.192 | 12.111 | 10.282 | 1.00 | 28.15 | A |
| ATOM | 1347 | N | ARG | A | 799 | −2.154 | 10.768 | 11.752 | 1.00 | 26.63 | A |
| ATOM | 1348 | CA | ARG | A | 799 | −0.818 | 11.247 | 11.403 | 1.00 | 26.62 | A |
| ATOM | 1349 | CB | ARG | A | 799 | 0.193 | 10.798 | 12.466 | 1.00 | 28.00 | A |
| ATOM | 1350 | CG | ARG | A | 799 | 0.416 | 9.294 | 12.553 | 1.00 | 28.23 | A |
| ATOM | 1351 | CD | ARG | A | 799 | 1.120 | 8.966 | 13.857 | 1.00 | 29.46 | A |
| ATOM | 1352 | NE | ARG | A | 799 | 0.298 | 9.380 | 14.993 | 1.00 | 30.72 | A |
| ATOM | 1353 | CZ | ARG | A | 799 | 0.771 | 9.878 | 16.129 | 1.00 | 30.22 | A |
| ATOM | 1354 | NH1 | ARG | A | 799 | 2.074 | 10.036 | 16.303 | 1.00 | 28.37 | A |
| ATOM | 1355 | NE2 | ARG | A | 799 | −0.066 | 10.228 | 17.093 | 1.00 | 31.49 | A |
| ATOM | 1356 | C | ARG | A | 799 | −0.350 | 10.789 | 10.018 | 1.00 | 24.48 | A |
| ATOM | 1357 | O | ARG | A | 799 | 0.781 | 11.053 | 9.622 | 1.00 | 24.93 | A |
| ATOM | 1358 | N | TRP | A | 800 | −1.226 | 10.102 | 9.292 | 1.00 | 23.10 | A |
| ATOM | 1359 | CA | TRP | A | 800 | −0.924 | 9.619 | 7.943 | 1.00 | 22.60 | A |
| ATOM | 1360 | CB | TRP | A | 800 | −1.212 | 8.118 | 7.836 | 1.00 | 21.43 | A |
| ATOM | 1361 | CG | TRP | A | 800 | −0.067 | 7.225 | 8.213 | 1.00 | 21.86 | A |
| ATOM | 1362 | CD2 | TRP | A | 800 | 0.187 | 6.640 | 9.499 | 1.00 | 24.24 | A |
| ATOM | 1363 | CE2 | TRP | A | 800 | 1.379 | 5.894 | 9.388 | 1.00 | 23.31 | A |
| ATOM | 1364 | CE3 | TRP | A | 800 | −0.477 | 6.673 | 10.734 | 1.00 | 24.72 | A |
| ATOM | 1365 | CD1 | TRP | A | 800 | 0.953 | 6.822 | 7.404 | 1.00 | 20.84 | A |
| ATOM | 1366 | NE1 | TRP | A | 800 | 1.824 | 6.023 | 8.099 | 1.00 | 21.54 | A |
| ATOM | 1367 | CZ2 | TRP | A | 800 | 1.924 | 5.186 | 10.464 | 1.00 | 25.57 | A |
| ATOM | 1368 | CZ3 | TRP | A | 800 | 0.067 | 5.966 | 11.807 | 1.00 | 26.76 | A |
| ATOM | 1369 | CH2 | TRP | A | 800 | 1.255 | 5.234 | 11.662 | 1.00 | 25.91 | A |
| ATOM | 1370 | C | TRP | A | 800 | −1.818 | 10.349 | 6.950 | 1.00 | 22.35 | A |
| ATOM | 1371 | O | TRP | A | 800 | −1.623 | 10.276 | 5.741 | 1.00 | 22.75 | A |
| ATOM | 1372 | N | THR | A | 801 | −2.805 | 11.054 | 7.480 | 1.00 | 22.40 | A |
| ATOM | 1373 | CA | THR | A | 801 | −3.781 | 11.739 | 6.653 | 1.00 | 21.32 | A |
| ATOM | 1374 | CE | THR | A | 801 | −5.177 | 11.517 | 7.242 | 1.00 | 20.93 | A |
| ATOM | 1375 | OG1 | THR | A | 801 | −5.356 | 10.114 | 7.520 | 1.00 | 19.55 | A |
| ATOM | 1376 | CG2 | THR | A | 801 | −6.253 | 11.971 | 6.264 | 1.00 | 19.42 | A |
| ATOM | 1377 | C | THR | A | 801 | −3.521 | 13.224 | 6.483 | 1.00 | 22.83 | A |
| ATOM | 1378 | O | THR | A | 801 | −3.073 | 13.901 | 7.405 | 1.00 | 24.26 | A |
| ATOM | 1379 | N | ALA | A | 802 | −3.806 | 13.719 | 5.285 | 1.00 | 23.67 | A |
| ATOM | 1380 | CA | ALA | A | 802 | −3.616 | 15.125 | 4.961 | 1.00 | 24.60 | A |
| ATOM | 1381 | CB | ALA | A | 802 | −3.827 | 15.349 | 3.472 | 1.00 | 21.86 | A |
| ATOM | 1382 | C | ALA | A | 802 | −4.567 | 16.006 | 5.755 | 1.00 | 25.32 | A |
| ATOM | 1383 | O | ALA | A | 802 | −5.664 | 15.586 | 6.121 | 1.00 | 26.16 | A |
| ATOM | 1384 | N | PRO | A | 803 | −4.155 | 17.250 | 6.032 | 1.00 | 26.55 | A |
| ATOM | 1385 | CD | PRO | A | 803 | −2.823 | 17.825 | 5.768 | 1.00 | 25.55 | A |
| ATOM | 1386 | CA | PRO | A | 803 | −4.991 | 18.183 | 6.791 | 1.00 | 27.54 | A |
| ATOM | 1387 | CE | PRO | A | 803 | −4.158 | 19.462 | 6.789 | 1.00 | 29.43 | A |
| ATOM | 1388 | CG | PRO | A | 803 | −2.743 | 18.934 | 6.788 | 1.00 | 29.14 | A |
| ATOM | 1389 | C | PRO | A | 803 | −6.384 | 18.396 | 6.188 | 1.00 | 28.40 | A |
| ATOM | 1390 | O | PRO | A | 803 | −7.387 | 18.318 | 6.897 | 1.00 | 28.43 | A |
| ATOM | 1391 | N | GLU | A | 804 | −6.445 | 18.658 | 4.884 | 1.00 | 29.91 | A |
| ATOM | 1392 | CA | GLU | A | 804 | −7.730 | 18.897 | 4.231 | 1.00 | 31.54 | A |
| ATOM | 1393 | CB | GLU | A | 804 | −7.536 | 19.320 | 2.764 | 1.00 | 29.69 | A |
| ATOM | 1394 | CG | GLU | A | 804 | −7.162 | 18.217 | 1.779 | 1.00 | 29.36 | A |
| ATOM | 1395 | CD | GLU | A | 804 | −5.671 | 17.936 | 1.723 | 1.00 | 26.95 | A |
| ATOM | 1396 | OE1 | GLU | A | 804 | −4.921 | 18.476 | 2.563 | 1.00 | 26.91 | A |
| ATOM | 1397 | OE2 | GLU | A | 804 | −5.254 | 17.164 | 0.838 | 1.00 | 27.14 | A |
| ATOM | 1398 | C | GLU | A | 804 | −8.634 | 17.671 | 4.318 | 1.00 | 32.75 | A |
| ATOM | 1399 | O | GLU | A | 804 | −9.857 | 17.793 | 4.406 | 1.00 | 32.85 | A |
| ATOM | 1400 | N | ALA | A | 805 | −8.022 | 16.492 | 4.314 | 1.00 | 34.37 | A |
| ATOM | 1401 | CA | ALA | A | 805 | −8.768 | 15.244 | 4.397 | 1.00 | 36.04 | A |
| ATOM | 1402 | CB | ALA | A | 805 | −7.859 | 14.069 | 4.054 | 1.00 | 36.25 | A |
| ATOM | 1403 | C | ALA | A | 805 | −9.364 | 15.050 | 5.787 | 1.00 | 37.08 | A |
| ATOM | 1404 | O | ALA | A | 805 | −10.422 | 14.444 | 5.942 | 1.00 | 37.44 | A |
| ATOM | 1405 | N | ILE | A | 806 | −8.687 | 15.564 | 6.802 | 1.00 | 38.80 | A |
| ATOM | 1406 | CA | ILE | A | 806 | −9.180 | 15.425 | 8.162 | 1.00 | 40.65 | A |
| ATOM | 1407 | CB | ILE | A | 806 | −8.035 | 15.591 | 9.179 | 1.00 | 40.28 | A |
| ATOM | 1408 | CG2 | ILE | A | 806 | −8.582 | 15.572 | 10.601 | 1.00 | 39.56 | A |
| ATOM | 1409 | CG1 | ILE | A | 806 | −7.019 | 14.463 | 8.988 | 1.00 | 39.83 | A |
| ATOM | 1410 | CD1 | ILE | A | 806 | −5.798 | 14.585 | 9.870 | 1.00 | 41.52 | A |
| ATOM | 1411 | C | ILE | A | 806 | −10.267 | 16.449 | 8.457 | 1.00 | 42.79 | A |
| ATOM | 1412 | O | ILE | A | 806 | −11.265 | 16.137 | 9.108 | 1.00 | 42.39 | A |
| ATOM | 1413 | N | GLN | A | 807 | −10.080 | 17.665 | 7.952 | 1.00 | 44.65 | A |
| ATOM | 1414 | CA | GLN | A | 807 | −11.032 | 18.746 | 8.183 | 1.00 | 46.54 | A |
| ATOM | 1415 | CB | GLN | A | 807 | −10.345 | 20.091 | 7.959 | 1.00 | 48.25 | A |
| ATOM | 1416 | CG | GLN | A | 807 | −11.184 | 21.287 | 8.352 | 1.00 | 51.25 | A |
| ATOM | 1417 | CD | GLN | A | 807 | −10.330 | 22.490 | 8.710 | 1.00 | 52.70 | A |
| ATOM | 1418 | OE1 | GLN | A | 807 | −9.503 | 22.940 | 7.914 | 1.00 | 52.66 | A |
| ATOM | 1419 | NE2 | GLN | A | 807 | −10.524 | 23.015 | 9.917 | 1.00 | 53.69 | A |
| ATOM | 1420 | C | GLN | A | 807 | −12.306 | 18.679 | 7.348 | 1.00 | 47.43 | A |
| ATOM | 1421 | O | GLN | A | 807 | −13.407 | 18.637 | 7.899 | 1.00 | 48.05 | A |
| ATOM | 1422 | N | TYR | A | 808 | −12.161 | 18.673 | 6.025 | 1.00 | 48.14 | A |
| ATOM | 1412 | CA | TYR | A | 808 | −13.319 | 18.630 | 5.134 | 1.00 | 47.86 | A |
| ATOM | 1424 | CB | TYR | A | 808 | −13.151 | 19.628 | 3.981 | 1.00 | 49.76 | A |
| ATOM | 1425 | CG | TYR | A | 808 | −12.690 | 21.004 | 4.407 | 1.00 | 51.92 | A |
| ATOM | 1426 | CD1 | TYR | A | 808 | −11.335 | 21.288 | 4.555 | 1.00 | 52.77 | A |
| ATOM | 1427 | CE1 | TYR | A | 808 | −10.905 | 22.540 | 4.976 | 1.00 | 54.43 | A |
| ATOM | 1428 | CD2 | TYR | A | 808 | −13.610 | 22.013 | 4.689 | 1.00 | 53.07 | A |
| ATOM | 1429 | CE2 | TYR | A | 808 | −13.191 | 23.271 | 5.113 | 1.00 | 54.30 | A |
| ATOM | 1430 | CZ | TYR | A | 808 | −11.836 | 23.527 | 5.256 | 1.00 | 54.97 | A |
| ATOM | 1431 | OH | TYR | A | 808 | −11.404 | 24.760 | 5.693 | 1.00 | 55.34 | A |
| ATOM | 1432 | C | TYR | A | 808 | −13.556 | 17.244 | 4.547 | 1.00 | 46.87 | A |
| ATOM | 1433 | O | TYR | A | 808 | −14.334 | 17.092 | 3.608 | 1.00 | 45.84 | A |
| ATOM | 1434 | N | ARG | A | 809 | −12.884 | 16.240 | 5.103 | 1.00 | 45.55 | A |
| ATOM | 1435 | CA | ARG | A | 809 | −13.004 | 14.863 | 4.632 | 1.00 | 44.26 | A |
| ATOM | 1436 | CB | ARG | A | 809 | −14.346 | 14.259 | 5.060 | 1.00 | 45.12 | A |
| ATOM | 1437 | CG | ARG | A | 809 | −14.499 | 14.086 | 6.563 | 0.00 | 45.61 | A |
| ATOM | 1438 | CD | ARG | A | 809 | −15.764 | 13.313 | 6.903 | 0.00 | 46.19 | A |
| ATOM | 1439 | NE | ARG | A | 809 | −15.851 | 13.007 | 8.328 | 0.00 | 46.63 | A |
| ATOM | 1440 | CZ | ARG | A | 809 | −16.810 | 12.268 | 8.878 | 0.00 | 46.86 | A |
| ATOM | 1441 | NH1 | ARG | A | 809 | −17.771 | 11.756 | 8.122 | 0.00 | 47.00 | A |
| ATOM | 1442 | NH2 | ARG | A | 809 | −16.806 | 12.038 | 10.184 | 0.00 | 47.00 | A |
| ATOM | 1443 | C | ARG | A | 809 | −12.849 | 14.761 | 3.115 | 1.00 | 42.45 | A |
| ATOM | 1444 | O | ARG | A | 809 | −13.488 | 13.928 | 2.469 | 1.00 | 42.37 | A |
| ATOM | 1445 | N | LYS | A | 810 | −11.992 | 15.610 | 2.557 | 1.00 | 40.09 | A |
| ATOM | 1446 | CA | LYS | A | 810 | −11.737 | 15.617 | 1.120 | 1.00 | 38.13 | A |
| ATOM | 1447 | CB | LYS | A | 810 | −11.373 | 17.030 | 0.649 | 1.00 | 39.28 | A |
| ATOM | 1448 | CG | LYS | A | 810 | −12.443 | 18.075 | 0.917 | 1.00 | 41.84 | A |
| ATOM | 1449 | CD | LYS | A | 810 | −12.071 | 19.415 | 0.293 | 1.00 | 43.21 | A |
| ATOM | 1450 | CE | LYS | A | 810 | −13.210 | 20.414 | 0.425 | 1.00 | 43.84 | A |
| ATOM | 1451 | NZ | LYS | A | 810 | −12.950 | 21.652 | −0.356 | 1.00 | 44.49 | A |
| ATOM | 1452 | C | LYS | A | 810 | −10.594 | 14.665 | 0.770 | 1.00 | 35.30 | A |
| ATOM | 1453 | O | LYS | A | 810 | −9.429 | 15.050 | 0.826 | 1.00 | 34.59 | A |
| ATOM | 1454 | N | PHE | A | 811 | −10.928 | 13.427 | 0.417 | 1.00 | 32.42 | A |
| ATOM | 1455 | CA | PHE | A | 811 | −9.912 | 12.445 | 0.056 | 1.00 | 29.29 | A |
| ATOM | 1456 | CB | PHE | A | 811 | −10.296 | 11.053 | 0.539 | 1.00 | 29.39 | A |
| ATOM | 1457 | CG | PHE | A | 811 | −10.238 | 10.888 | 2.023 | 1.00 | 29.16 | A |
| ATOM | 1458 | CD1 | PHE | A | 811 | −11.317 | 11.258 | 2.822 | 1.00 | 30.55 | A |
| ATOM | 1459 | CD2 | PHE | A | 811 | −9.110 | 10.342 | 2.624 | 1.00 | 28.31 | A |
| ATOM | 1460 | CE1 | PHE | A | 811 | −11.273 | 11.078 | 4.202 | 1.00 | 31.51 | A |
| ATOM | 1461 | CE2 | PHE | A | 811 | −9.051 | 10.157 | 3.998 | 1.00 | 29.55 | A |
| ATOM | 1462 | CZ | PHE | A | 811 | −10.135 | 10.526 | 4.792 | 1.00 | 31.35 | A |
| ATOM | 1463 | C | PHE | A | 811 | −9.705 | 12.400 | −1.444 | 1.00 | 27.52 | A |
| ATOM | 1464 | O | PHE | A | 811 | −10.646 | 12.167 | −2.198 | 1.00 | 26.39 | A |
| ATOM | 1465 | N | THR | A | 812 | −8.462 | 12.617 | −1.862 | 1.00 | 23.72 | A |
| ATOM | 1466 | CA | THR | A | 812 | −8.089 | 12.617 | −3.271 | 1.00 | 23.05 | A |
| ATOM | 1467 | CB | THR | A | 812 | −7.970 | 14.054 | −3.790 | 1.00 | 23.97 | A |
| ATOM | 1468 | OG1 | THR | A | 812 | −6.932 | 14.727 | −3.067 | 1.00 | 23.73 | A |
| ATOM | 1469 | CG2 | THR | A | 812 | −9.283 | 14.818 | −3.564 | 1.00 | 25.01 | A |
| ATOM | 1470 | C | THR | A | 812 | −6.721 | 11.964 | −3.424 | 1.00 | 21.01 | A |
| ATOM | 1471 | O | THR | A | 812 | −6.102 | 11.577 | −2.437 | 1.00 | 20.62 | A |
| ATOM | 1472 | N | SER | A | 813 | −6.237 | 11.850 | −4.655 | 1.00 | 20.38 | A |
| ATOM | 1473 | CA | SER | A | 813 | −4.918 | 11.270 | −4.859 | 1.00 | 20.11 | A |
| ATOM | 1474 | CB | SER | A | 813 | −4.616 | 11.071 | −6.350 | 1.00 | 18.76 | A |
| ATOM | 1475 | OG | SER | A | 813 | −5.445 | 10.061 | −6.898 | 1.00 | 18.07 | A |
| ATOM | 1476 | C | SER | A | 813 | −3.872 | 12.185 | −4.241 | 1.00 | 20.46 | A |
| ATOM | 1477 | O | SER | A | 813 | −2.818 | 11.719 | −3.809 | 1.00 | 19.84 | A |
| ATOM | 1478 | N | ALA | A | 814 | −4.171 | 13.482 | −4.178 | 1.00 | 19.76 | A |
| ATOM | 1479 | CA | ALA | A | 814 | −3.239 | 14.450 | −3.600 | 1.00 | 20.26 | A |
| ATOM | 1480 | CB | ALA | A | 814 | −3.710 | 15.882 | −3.875 | 1.00 | 19.22 | A |
| ATOM | 1481 | C | ALA | A | 814 | −3.182 | 14.195 | −2.110 | 1.00 | 19.65 | A |
| ATOM | 1482 | O | ALA | A | 814 | −2.178 | 14.478 | −1.446 | 1.00 | 18.61 | A |
| ATOM | 1483 | N | SER | A | 815 | −4.289 | 13.691 | −1.579 | 1.00 | 19.76 | A |
| ATOM | 1484 | CA | SER | A | 815 | −4.355 | 13.364 | −0.164 | 1.00 | 19.99 | A |
| ATOM | 1485 | CB | SER | A | 815 | −5.803 | 13.086 | 0.244 | 1.00 | 19.81 | A |
| ATOM | 1486 | OG | SER | A | 815 | −5.869 | 12.896 | 1.640 | 1.00 | 27.92 | A |
| ATOM | 1487 | C | SER | A | 815 | −3.490 | 12.114 | 0.062 | 1.00 | 18.44 | A |
| ATOM | 1488 | O | SER | A | 815 | −2.786 | 12.010 | 1.065 | 1.00 | 19.10 | A |
| ATOM | 1489 | N | ASP | A | 816 | −3.544 | 11.169 | −0.875 | 1.00 | 17.07 | A |
| ATOM | 1490 | CA | ASP | A | 816 | −2.734 | 9.947 | −0.774 | 1.00 | 15.75 | A |
| ATOM | 1491 | CB | ASP | A | 816 | −3.041 | 8.953 | −1.901 | 1.00 | 13.79 | A |
| ATOM | 1492 | CG | ASP | A | 816 | −4.343 | 8.184 | −1.691 | 1.00 | 12.11 | A |
| ATOM | 1493 | OD1 | ASP | A | 816 | −4.823 | 8.097 | −0.543 | 1.00 | 14.33 | A |
| ATOM | 1494 | OD2 | ASP | A | 816 | −4.864 | 7.646 | −2.685 | 1.00 | 13.38 | A |
| ATOM | 1495 | C | ASP | A | 816 | −1.259 | 10.307 | −0.887 | 1.00 | 16.60 | A |
| ATOM | 1496 | O | ASP | A | 816 | −0.399 | 9.571 | −0.392 | 1.00 | 14.48 | A |
| ATOM | 1497 | N | VAL | A | 817 | −0.961 | 11.419 | −1.557 | 1.00 | 13.18 | A |
| ATOM | 1498 | CA | VAL | A | 817 | 0.422 | 11.826 | −1.721 | 1.00 | 13.34 | A |
| ATOM | 1499 | CB | VAL | A | 817 | 0.566 | 12.960 | −2.788 | 1.00 | 11.51 | A |
| ATOM | 1500 | CG1 | VAL | A | 817 | 1.944 | 13.589 | −2.704 | 1.00 | 9.74 | A |
| ATOM | 1501 | CG2 | VAL | A | 817 | 0.376 | 12.363 | −4.179 | 1.00 | 11.50 | A |
| ATOM | 1502 | C | VAL | A | 817 | 1.005 | 12.286 | −0.395 | 1.00 | 12.99 | A |
| ATOM | 1503 | O | VAL | A | 817 | 2.172 | 12.046 | −0.108 | 1.00 | 13.15 | A |
| ATOM | 1504 | N | TRP | A | 818 | 0.189 | 12.938 | 0.421 | 1.00 | 14.23 | A |
| ATOM | 1505 | CA | TRP | A | 818 | 0.654 | 13.378 | 1.726 | 1.00 | 15.45 | A |
| ATOM | 1506 | CB | TRP | A | 818 | −0.447 | 14.188 | 2.410 | 1.00 | 15.49 | A |
| ATOM | 1507 | CG | TRP | A | 818 | −0.133 | 14.556 | 3.801 | 1.00 | 19.74 | A |
| ATOM | 1508 | CD2 | TRP | A | 818 | 0.180 | 15.863 | 4.295 | 1.00 | 20.03 | A |
| ATOM | 1509 | CE2 | TRP | A | 818 | 0.436 | 15.733 | 5.676 | 1.00 | 21.03 | A |
| ATOM | 1510 | CE3 | TRP | A | 818 | 0.269 | 17.131 | 3.706 | 1.00 | 21.31 | A |
| ATOM | 1511 | CD1 | TRP | A | 818 | −0.060 | 13.715 | 4.869 | 1.00 | 19.62 | A |
| ATOM | 1512 | NE1 | TRP | A | 818 | 0.283 | 14.410 | 5.998 | 1.00 | 21.47 | A |
| ATOM | 1513 | CZ2 | TRP | A | 818 | 0.774 | 16.823 | 6.481 | 1.00 | 20.05 | A |
| ATOM | 1514 | CZ3 | TRP | A | 818 | 0.606 | 18.217 | 4.506 | 1.00 | 19.00 | A |
| ATOM | 1515 | CH2 | TRP | A | 818 | 0.854 | 18.055 | 5.878 | 1.00 | 20.15 | A |
| ATOM | 1516 | C | TRP | A | 818 | 0.996 | 12.121 | 2.531 | 1.00 | 15.99 | A |
| ATOM | 1517 | O | TRP | A | 818 | 2.033 | 12.048 | 3.210 | 1.00 | 15.51 | A |
| ATOM | 1518 | N | SER | A | 819 | 0.118 | 11.130 | 2.428 | 1.00 | 14.95 | A |
| ATOM | 1519 | CA | SER | A | 819 | 0.281 | 9.855 | 3.122 | 1.00 | 15.25 | A |
| ATOM | 1520 | CB | SER | A | 819 | −0.929 | 8.950 | 2.852 | 1.00 | 14.79 | A |
| ATOM | 1521 | OG | SER | A | 819 | −2.112 | 9.536 | 3.348 | 1.00 | 16.39 | A |
| ATOM | 1522 | C | SER | A | 819 | 1.550 | 9.181 | 2.629 | 1.00 | 14.02 | A |
| ATOM | 1523 | O | SER | A | 819 | 2.325 | 8.640 | 3.414 | 1.00 | 16.48 | A |
| ATOM | 1524 | N | TYR | A | 820 | 1.765 | 9.229 | 1.320 | 1.00 | 13.71 | A |
| ATOM | 1525 | CA | TYR | A | 820 | 2.956 | 8.636 | 0.726 | 1.00 | 14.58 | A |
| ATOM | 1526 | CB | TYR | A | 820 | 2.956 | 8.842 | −0.799 | 1.00 | 13.45 | A |
| ATOM | 1527 | CG | TYR | A | 820 | 4.197 | 8.297 | −1.453 | 1.00 | 12.89 | A |
| ATOM | 1528 | CD1 | TYR | A | 820 | 4.336 | 6.931 | −1.732 | 1.00 | 15.07 | A |
| ATOM | 1529 | CE1 | TYR | A | 820 | 5.541 | 6.419 | −2.219 | 1.00 | 12.78 | A |
| ATOM | 1530 | CD2 | TYR | A | 820 | 5.285 | 9.128 | −1.690 | 1.00 | 12.38 | A |
| ATOM | 1531 | CE2 | TYR | A | 820 | 6.466 | 8.633 | −2.166 | 1.00 | 11.32 | A |
| ATOM | 1532 | CZ | TYR | A | 820 | 6.600 | 7.288 | −2.426 | 1.00 | 12.29 | A |
| ATOM | 1533 | OH | TYR | A | 820 | 7.814 | 6.840 | −2.859 | 1.00 | 10.71 | A |
| ATOM | 1534 | C | TYR | A | 820 | 4.210 | 9.280 | 1.338 | 1.00 | 13.53 | A |
| ATOM | 1535 | O | TYR | A | 820 | 5.223 | 8.616 | 1.545 | 1.00 | 13.71 | A |
| ATOM | 1536 | N | GLY | A | 821 | 4.137 | 10.574 | 1.628 | 1.00 | 14.63 | A |
| ATOM | 1537 | CA | GLY | A | 821 | 5.275 | 11.245 | 2.218 | 1.00 | 13.52 | A |
| ATOM | 1538 | C | GLY | A | 821 | 5.563 | 10.662 | 3.585 | 1.00 | 14.77 | A |
| ATOM | 1539 | O | GLY | A | 821 | 6.719 | 10.454 | 3.959 | 1.00 | 13.85 | A |
| ATOM | 1540 | N | ILE | A | 822 | 4.509 | 10.408 | 4.347 | 1.00 | 15.25 | A |
| ATOM | 1541 | CA | ILE | A | 822 | 4.690 | 9.820 | 5.666 | 1.00 | 17.12 | A |
| ATOM | 1542 | CB | ILE | A | 822 | 3.344 | 9.674 | 6.420 | 1.00 | 17.21 | A |
| ATOM | 1543 | CG2 | ILE | A | 822 | 3.569 | 9.002 | 7.768 | 1.00 | 17.52 | A |
| ATOM | 1544 | CG1 | ILE | A | 822 | 2.689 | 11.050 | 6.577 | 1.00 | 17.04 | A |
| ATOM | 1545 | OD1 | ILE | A | 822 | 3.528 | 12.069 | 7.309 | 1.00 | 15.74 | A |
| ATOM | 1546 | C | ILE | A | 822 | 5.326 | 8.443 | 5.495 | 1.00 | 15.76 | A |
| ATOM | 1547 | O | ILE | A | 822 | 6.174 | 8.059 | 6.293 | 1.00 | 17.81 | A |
| ATOM | 1548 | N | VAL | A | 823 | 4.920 | 7.710 | 4.457 | 1.00 | 13.84 | A |
| ATOM | 1549 | CA | VAL | A | 823 | 5.459 | 6.375 | 4.199 | 1.00 | 12.67 | A |
| ATOM | 1550 | CB | VAL | A | 823 | 4.738 | 5.711 | 2.989 | 1.00 | 12.77 | A |
| ATOM | 1551 | CG1 | VAL | A | 823 | 5.403 | 4.386 | 2.633 | 1.00 | 10.23 | A |
| ATOM | 1552 | CG2 | VAL | A | 823 | 3.271 | 5.494 | 3.328 | 1.00 | 13.23 | A |
| ATOM | 1553 | C | VAL | A | 823 | 6.961 | 6.454 | 3.918 | 1.00 | 14.16 | A |
| ATOM | 1554 | O | VAL | A | 823 | 7.745 | 5.594 | 4.339 | 1.00 | 12.18 | A |
| ATOM | 1555 | N | MET | A | 824 | 7.352 | 7.488 | 3.188 | 1.00 | 12.40 | A |
| ATOM | 1556 | CA | MET | A | 824 | 8.755 | 7.710 | 2.883 | 1.00 | 12.87 | A |
| ATOM | 1557 | CB | MET | A | 824 | 8.938 | 8.982 | 2.070 | 1.00 | 13.48 | A |
| ATOM | 1558 | CG | MET | A | 824 | 8.457 | 8.904 | 0.641 | 1.00 | 11.06 | A |
| ATOM | 1559 | SD | MET | A | 824 | 8.815 | 10.500 | −0.170 | 1.00 | 15.45 | A |
| ATOM | 1560 | CE | MET | A | 824 | 10.625 | 10.460 | −0.293 | 1.00 | 14.21 | A |
| ATOM | 1561 | C | MET | A | 824 | 9.506 | 7.876 | 4.181 | 1.00 | 11.22 | A |
| ATOM | 1562 | O | MET | A | 824 | 10.632 | 7.411 | 4.318 | 1.00 | 12.72 | A |
| ATOM | 1563 | N | TRP | A | 825 | 8.882 | 8.550 | 5.135 | 1.00 | 13.43 | A |
| ATOM | 1564 | CA | TRP | A | 825 | 9.532 | 8.760 | 6.416 | 1.00 | 16.39 | A |
| ATOM | 1565 | CB | TRP | A | 825 | 8.733 | 9.758 | 7.251 | 1.00 | 17.69 | A |
| ATOM | 1566 | CG | TRP | A | 825 | 9.485 | 10.300 | 8.429 | 1.00 | 21.01 | A |
| ATOM | 1567 | CD2 | TRP | A | 825 | 9.404 | 9.836 | 9.783 | 1.00 | 22.74 | A |
| ATOM | 1568 | CE2 | TRP | A | 825 | 10.267 | 10.646 | 10.556 | 1.00 | 24.73 | A |
| ATOM | 1569 | CE3 | TRP | A | 825 | 8.686 | 8.816 | 10.417 | 1.00 | 24.55 | A |
| ATOM | 1570 | CD1 | TRP | A | 825 | 10.372 | 11.338 | 8.433 | 1.00 | 22.10 | A |
| ATOM | 1571 | NE1 | TRP | A | 825 | 10.842 | 11.554 | 9.708 | 1.00 | 23.09 | A |
| ATOM | 1572 | CZ2 | TRP | A | 825 | 10.429 | 10.469 | 11.937 | 1.00 | 23.91 | A |
| ATOM | 1573 | CZ3 | TRP | A | 825 | 8.848 | 8.641 | 11.791 | 1.00 | 24.02 | A |
| ATOM | 1574 | CH2 | TRP | A | 825 | 9.713 | 9.464 | 12.532 | 1.00 | 24.58 | A |
| ATOM | 1575 | C | TRP | A | 825 | 9.651 | 7.415 | 7.143 | 1.00 | 16.44 | A |
| ATOM | 1576 | O | TRP | A | 825 | 10.718 | 7.074 | 7.648 | 1.00 | 15.71 | A |
| ATOM | 1577 | N | GLU | A | 826 | 8.564 | 6.645 | 7.185 | 1.00 | 15.79 | A |
| ATOM | 1578 | CA | GLU | A | 826 | 8.597 | 5.338 | 7.854 | 1.00 | 15.47 | A |
| ATOM | 1579 | CB | GLU | A | 826 | 7.266 | 4.609 | 7.685 | 1.00 | 15.67 | A |
| ATOM | 1580 | CG | GLU | A | 826 | 6.066 | 5.409 | 8.128 | 1.00 | 17.54 | A |
| ATOM | 1581 | CD | GLU | A | 826 | 4.797 | 4.603 | 8.027 | 1.00 | 17.43 | A |
| ATOM | 1582 | OE1 | GLU | A | 826 | 4.561 | 3.760 | 8.918 | 1.00 | 17.18 | A |
| ATOM | 1583 | OE2 | GLU | A | 826 | 4.047 | 4.793 | 7.054 | 1.00 | 19.83 | A |
| ATOM | 1584 | C | GLU | A | 826 | 9.698 | 4.459 | 7.297 | 1.00 | 15.72 | A |
| ATOM | 1585 | O | GLU | A | 826 | 10.430 | 3.809 | 8.043 | 1.00 | 14.73 | A |
| ATOM | 1586 | N | VAL | A | 827 | 9.807 | 4.448 | 5.973 | 1.00 | 14.75 | A |
| ATOM | 1587 | CA | VAL | A | 827 | 10.812 | 3.652 | 5.293 | 1.00 | 13.80 | A |
| ATOM | 1588 | CB | VAL | A | 827 | 10.605 | 3.688 | 3.776 | 1.00 | 13.84 | A |
| ATOM | 1589 | CG1 | VAL | A | 827 | 11.864 | 3.166 | 3.064 | 1.00 | 15.75 | A |
| ATOM | 1590 | CG2 | VAL | A | 827 | 9.395 | 2.845 | 3.404 | 1.00 | 12.52 | A |
| ATOM | 1591 | C | VAL | A | 827 | 12.241 | 4.107 | 5.598 | 1.00 | 15.22 | A |
| ATOM | 1592 | O | VAL | A | 827 | 13.114 | 3.282 | 5.855 | 1.00 | 15.68 | A |
| ATOM | 1593 | N | MET | A | 828 | 12.488 | 5.411 | 5.571 | 1.00 | 15.64 | A |
| ATOM | 1594 | CA | MET | A | 828 | 13.840 | 5.885 | 5.830 | 1.00 | 16.82 | A |
| ATOM | 1595 | CB | MET | A | 828 | 14.020 | 7.304 | 5.282 | 1.00 | 16.69 | A |
| ATOM | 1596 | CG | MET | A | 828 | 13.812 | 7.400 | 3.764 | 1.00 | 15.12 | A |
| ATOM | 1597 | SD | MET | A | 828 | 14.617 | 6.077 | 2.862 | 1.00 | 17.16 | A |
| ATOM | 1598 | CE | MET | A | 828 | 16.377 | 6.500 | 3.177 | 1.00 | 16.75 | A |
| ATOM | 1599 | C | MET | A | 828 | 14.182 | 5.815 | 7.319 | 1.00 | 17.74 | A |
| ATOM | 1600 | O | MET | A | 828 | 15.353 | 5.818 | 7.697 | 1.00 | 17.30 | A |
| ATOM | 1601 | N | SER | A | 829 | 13.146 | 5.731 | 8.146 | 1.00 | 16.25 | A |
| ATOM | 1602 | CA | SER | A | 829 | 13.287 | 5.639 | 9.602 | 1.00 | 19.49 | A |
| ATOM | 1603 | CS | SER | A | 829 | 12.129 | 6.360 | 10.296 | 1.00 | 19.11 | A |
| ATOM | 1604 | OG | SER | A | 829 | 12.153 | 7.747 | 10.028 | 1.00 | 26.80 | A |
| ATOM | 1605 | C | SER | A | 829 | 13.263 | 4.186 | 10.069 | 1.00 | 17.50 | A |
| ATOM | 1606 | O | SER | A | 829 | 13.340 | 3.921 | 11.264 | 1.00 | 19.93 | A |
| ATOM | 1607 | N | TYR | A | 830 | 13.157 | 3.265 | 9.118 | 1.00 | 15.75 | A |
| ATOM | 1608 | CA | TYR | A | 830 | 13.053 | 1.837 | 9.382 | 1.00 | 15.79 | A |
| ATOM | 1609 | CB | TYR | A | 830 | 14.364 | 1.250 | 9.930 | 1.00 | 15.94 | A |
| ATOM | 1610 | CG | TYR | A | 830 | 15.392 | 0.987 | 8.861 | 1.00 | 15.43 | A |
| ATOM | 1611 | CD1 | TYR | A | 830 | 16.370 | 1.925 | 8.566 | 1.00 | 19.30 | A |
| ATOM | 1612 | CE1 | TYR | A | 830 | 17.330 | 1.682 | 7.577 | 1.00 | 17.62 | A |
| ATOM | 1613 | CD2 | TYR | A | 830 | 15.387 | −0.202 | 8.141 | 1.00 | 15.96 | A |
| ATOM | 1614 | CE2 | TYR | A | 830 | 16.332 | −0.454 | 7.156 | 1.00 | 17.55 | A |
| ATOM | 1615 | CZ | TYR | A | 830 | 17.304 | 0.494 | 6.883 | 1.00 | 16.47 | A |
| ATOM | 1616 | OH | TYR | A | 830 | 18.260 | 0.225 | 5.930 | 1.00 | 15.91 | A |
| ATOM | 1617 | C | TYR | A | 830 | 11.889 | 1.459 | 10.298 | 1.00 | 16.77 | A |
| ATOM | 1618 | O | TYR | A | 830 | 12.051 | 0.715 | 11.273 | 1.00 | 14.45 | A |
| ATOM | 1619 | N | GLY | A | 831 | 10.707 | 1.979 | 9.979 | 1.00 | 14.09 | A |
| ATOM | 1620 | CA | GLY | A | 831 | 9.521 | 1.635 | 10.742 | 1.00 | 17.12 | A |
| ATOM | 1621 | C | GLY | A | 831 | 9.224 | 2.407 | 12.003 | 1.00 | 19.41 | A |
| ATOM | 1622 | O | GLY | A | 831 | 8.410 | 1.977 | 12.827 | 1.00 | 19.28 | A |
| ATOM | 1623 | N | GLU | A | 832 | 9.883 | 3.545 | 12.171 | 1.00 | 21.71 | A |
| ATOM | 1624 | CA | GLU | A | 832 | 9.633 | 4.365 | 13.340 | 1.00 | 23.50 | A |
| ATOM | 1625 | CB | GLU | A | 832 | 10.627 | 5.522 | 13.384 | 1.00 | 25.11 | A |
| ATOM | 1626 | CG | GLU | A | 832 | 10.396 | 6.521 | 14.496 | 1.00 | 26.95 | A |
| ATOM | 1627 | CD | GLU | A | 832 | 10.411 | 5.869 | 15.874 | 1.00 | 32.36 | A |
| ATOM | 1628 | OE1 | GLU | A | 832 | 9.344 | 5.380 | 16.326 | 1.00 | 31.18 | A |
| ATOM | 1629 | OE2 | GLU | A | 832 | 11.497 | 5.834 | 16.500 | 1.00 | 32.72 | A |
| ATOM | 1630 | C | GLU | A | 832 | 8.210 | 4.897 | 13.216 | 1.00 | 24.54 | A |
| ATOM | 1631 | O | GLU | A | 832 | 7.668 | 4.981 | 12.111 | 1.00 | 22.49 | A |
| ATOM | 1632 | N | ARG | A | 833 | 7.608 | 5.249 | 14.347 | 1.00 | 24.48 | A |
| ATOM | 1633 | CA | ARG | A | 833 | 6.252 | 5.780 | 14.351 | 1.00 | 26.11 | A |
| ATOM | 1634 | CB | ARG | A | 833 | 5.597 | 5.564 | 15.720 | 1.00 | 28.74 | A |
| ATOM | 1635 | CG | ARG | A | 833 | 4.146 | 6.014 | 15.794 | 1.00 | 31.71 | A |
| ATOM | 1636 | CD | ARG | A | 833 | 3.443 | 5.386 | 16.985 | 1.00 | 34.54 | A |
| ATOM | 1637 | NE | ARG | A | 833 | 2.023 | 5.716 | 17.036 | 1.00 | 36.70 | A |
| ATOM | 1638 | CZ | ARG | A | 833 | 1.538 | 6.853 | 17.521 | 1.00 | 38.93 | A |
| ATOM | 1639 | NH1 | ARG | A | 833 | 2.360 | 7.776 | 17.998 | 1.00 | 40.22 | A |
| ATOM | 1640 | NH2 | ARG | A | 833 | 0.230 | 7.063 | 17.538 | 1.00 | 39.85 | A |
| ATOM | 1641 | C | ARG | A | 833 | 6.280 | 7.266 | 14.019 | 1.00 | 26.85 | A |
| ATOM | 1642 | O | ARG | A | 833 | 7.007 | 8.038 | 14.641 | 1.00 | 28.24 | A |
| ATOM | 1643 | N | PRO | A | 834 | 5.495 | 7.683 | 13.018 | 1.00 | 25.28 | A |
| ATOM | 1644 | CD | PRO | A | 834 | 4.678 | 6.857 | 12.112 | 1.00 | 26.97 | A |
| ATOM | 1645 | CA | PRO | A | 834 | 5.450 | 9.090 | 12.624 | 1.00 | 24.70 | A |
| ATOM | 1646 | CB | PRO | A | 834 | 4.381 | 9.108 | 11.534 | 1.00 | 25.64 | A |
| ATOM | 1647 | CG | PRO | A | 834 | 4.533 | 7.759 | 10.896 | 1.00 | 24.55 | A |
| ATOM | 1648 | C | PRO | A | 834 | 5.086 | 9.984 | 13.802 | 1.00 | 25.05 | A |
| ATOM | 1649 | O | PRO | A | 834 | 4.091 | 9.742 | 14.487 | 1.00 | 23.06 | A |
| ATOM | 1650 | N | TYR | A | 835 | 5.902 | 11.013 | 14.023 | 1.00 | 26.38 | A |
| ATOM | 1651 | CA | TYR | A | 835 | 5.704 | 11.977 | 15.102 | 1.00 | 26.19 | A |
| ATOM | 1652 | CD | TYR | A | 835 | 4.303 | 12.584 | 15.002 | 1.00 | 26.82 | A |
| ATOM | 1653 | CG | TYR | A | 835 | 4.015 | 13.202 | 13.648 | 1.00 | 27.32 | A |
| ATOM | 1654 | CD1 | TYR | A | 835 | 4.405 | 14.511 | 13.358 | 1.00 | 26.52 | A |
| ATOM | 1655 | CE1 | TYR | A | 835 | 4.175 | 15.069 | 12.101 | 1.00 | 26.25 | A |
| ATOM | 1656 | CD2 | TYR | A | 835 | 3.387 | 12.464 | 12.647 | 1.00 | 25.35 | A |
| ATOM | 1657 | CE2 | TYR | A | 835 | 3.153 | 13.008 | 11.386 | 1.00 | 25.46 | A |
| ATOM | 1658 | CZ | TYR | A | 835 | 3.550 | 14.314 | 11.116 | 1.00 | 26.18 | A |
| ATOM | 1659 | OH | TYR | A | 835 | 3.325 | 14.859 | 9.867 | 1.00 | 25.65 | A |
| ATOM | 1660 | C | TYR | A | 835 | 5.933 | 11.349 | 16.477 | 1.00 | 27.36 | A |
| ATOM | 1661 | O | TYR | A | 835 | 5.554 | 11.912 | 17.505 | 1.00 | 28.19 | A |
| ATOM | 1662 | N | TRP | A | 836 | 6.554 | 10.174 | 16.475 | 1.00 | 26.91 | A |
| ATOM | 1663 | CA | TRP | A | 836 | 6.885 | 9.454 | 17.699 | 1.00 | 28.58 | A |
| ATOM | 1664 | CB | TRP | A | 836 | 8.097 | 10.114 | 18.356 | 1.00 | 27.99 | A |
| ATOM | 1665 | CG | TRP | A | 836 | 9.233 | 10.282 | 17.406 | 1.00 | 29.96 | A |
| ATOM | 1666 | CD2 | TRP | A | 836 | 9.533 | 11.449 | 16.631 | 1.00 | 29.72 | A |
| ATOM | 1667 | CE2 | TRP | A | 836 | 10.666 | 11.149 | 15.847 | 1.00 | 30.62 | A |
| ATOM | 1668 | CE3 | TRP | A | 836 | 8.953 | 12.722 | 16.525 | 1.00 | 31.28 | A |
| ATOM | 1669 | CD1 | TRP | A | 836 | 10.169 | 9.346 | 17.069 | 1.00 | 29.72 | A |
| ATOM | 1670 | NE1 | TRP | A | 836 | 11.034 | 9.859 | 16.133 | 1.00 | 29.17 | A |
| ATOM | 1671 | CZ2 | TRP | A | 836 | 11.235 | 12.076 | 14.965 | 1.00 | 30.51 | A |
| ATOM | 1672 | CZ3 | TRP | A | 836 | 9.520 | 13.646 | 15.648 | 1.00 | 30.96 | A |
| ATOM | 1673 | CH2 | TRP | A | 836 | 10.649 | 13.315 | 14.882 | 1.00 | 31.70 | A |
| ATOM | 1674 | C | TRP | A | 836 | 5.727 | 9.370 | 18.688 | 1.00 | 29.62 | A |
| ATOM | 1675 | O | TRP | A | 836 | 4.627 | 8.945 | 18.328 | 1.00 | 29.84 | A |
| ATOM | 1676 | N | ASP | A | 837 | 5.977 | 9.789 | 19.927 | 1.00 | 31.12 | A |
| ATOM | 1677 | CA | ASP | A | 837 | 4.972 | 9.743 | 20.992 | 1.00 | 34.13 | A |
| ATOM | 1678 | CB | ASP | A | 837 | 5.659 | 9.720 | 22.365 | 1.00 | 34.74 | A |
| ATOM | 1679 | CG | ASP | A | 837 | 6.593 | 8.536 | 22.533 | 0.00 | 35.23 | A |
| ATOM | 1680 | OD1 | ASP | A | 837 | 6.132 | 7.384 | 22.389 | 0.00 | 35.57 | A |
| ATOM | 1681 | OD2 | ASP | A | 837 | 7.790 | 8.758 | 22.811 | 0.00 | 35.57 | A |
| ATOM | 1682 | C | ASP | A | 837 | 3.954 | 10.879 | 20.969 | 1.00 | 35.08 | A |
| ATOM | 1683 | O | ASP | A | 837 | 3.129 | 10.998 | 21.875 | 1.00 | 35.37 | A |
| ATOM | 1684 | N | MET | A | 838 | 4.009 | 11.722 | 19.946 | 1.00 | 36.64 | A |
| ATOM | 1685 | CA | MET | A | 838 | 3.056 | 12.821 | 19.845 | 1.00 | 37.58 | A |
| ATOM | 1686 | CB | MET | A | 838 | 3.315 | 13.648 | 18.594 | 1.00 | 37.74 | A |
| ATOM | 1687 | CG | MET | A | 838 | 4.207 | 14.831 | 18.800 | 1.00 | 37.49 | A |
| ATOM | 1688 | SD | MET | A | 838 | 4.120 | 15.864 | 17.349 | 1.00 | 40.87 | A |
| ATOM | 1689 | CE | MET | A | 838 | 5.651 | 15.422 | 16.541 | 1.00 | 37.80 | A |
| ATOM | 1690 | C | MET | A | 838 | 1.638 | 12.295 | 19.771 | 1.00 | 38.85 | A |
| ATOM | 1691 | O | MET | A | 838 | 1.392 | 11.235 | 19.196 | 1.00 | 38.49 | A |
| ATOM | 1692 | N | THR | A | 839 | 0.708 | 13.052 | 20.345 | 1.00 | 40.77 | A |
| ATOM | 1693 | CA | THR | A | 839 | −0.707 | 12.691 | 20.332 | 1.00 | 42.26 | A |
| ATOM | 1694 | CB | THR | A | 839 | −1.470 | 13.372 | 21.493 | 1.00 | 42.77 | A |
| ATOM | 1695 | OG1 | THR | A | 839 | −1.599 | 14.773 | 21.224 | 0.00 | 42.95 | A |
| ATOM | 1696 | CG2 | THR | A | 839 | −0.719 | 13.188 | 22.806 | 0.00 | 42.95 | A |
| ATOM | 1697 | C | THR | A | 839 | −1.289 | 13.184 | 19.007 | 1.00 | 42.46 | A |
| ATOM | 1698 | O | THR | A | 839 | −0.688 | 14.030 | 18.348 | 1.00 | 42.49 | A |
| ATOM | 1699 | N | ASN | A | 840 | −2.450 | 12.660 | 18.616 | 1.00 | 43.26 | A |
| ATOM | 1700 | CA | ASN | A | 840 | −3.078 | 13.075 | 17.363 | 1.00 | 44.27 | A |
| ATOM | 1701 | CB | ASN | A | 840 | −4.419 | 12.360 | 17.154 | 1.00 | 43.58 | A |
| ATOM | 1702 | CG | ASN | A | 840 | −4.256 | 10.885 | 16.855 | 1.00 | 43.42 | A |
| ATOM | 1703 | OD1 | ASN | A | 840 | −3.230 | 10.460 | 16.328 | 1.00 | 42.91 | A |
| ATOM | 1704 | ND2 | ASN | A | 840 | −5.278 | 10.097 | 17.172 | 1.00 | 44.00 | A |
| ATOM | 1705 | C | ASN | A | 840 | −3.299 | 14.585 | 17.327 | 1.00 | 44.84 | A |
| ATOM | 1706 | O | ASN | A | 840 | −3.017 | 15.236 | 16.321 | 1.00 | 45.54 | A |
| ATOM | 1707 | N | GLN | A | 841 | −3.798 | 15.141 | 18.427 | 1.00 | 45.95 | A |
| ATOM | 1708 | CA | GLN | A | 841 | −4.046 | 16.573 | 18.497 | 1.00 | 46.24 | A |
| ATOM | 1709 | CB | GLN | A | 841 | −4.718 | 16.947 | 19.818 | 1.00 | 47.54 | A |
| ATOM | 1710 | CG | GLN | A | 841 | −5.283 | 18.356 | 19.818 | 1.00 | 48.27 | A |
| ATOM | 1711 | CD | GLN | A | 841 | −6.207 | 18.594 | 18.638 | 1.00 | 49.43 | A |
| ATOM | 1712 | OE1 | GLN | A | 841 | −7.199 | 17.884 | 18.460 | 1.00 | 49.85 | A |
| ATOM | 1713 | NE2 | GLN | A | 841 | −5.882 | 19.591 | 17.820 | 1.00 | 49.98 | A |
| ATOM | 1714 | C | GLN | A | 841 | −2.744 | 17.347 | 18.350 | 1.00 | 46.35 | A |
| ATOM | 1715 | O | GLN | A | 841 | −2.702 | 18.373 | 17.673 | 1.00 | 46.78 | A |
| ATOM | 1716 | N | ASP | A | 842 | −1.682 | 16.861 | 18.984 | 1.00 | 45.35 | A |
| ATOM | 1717 | CA | ASP | A | 842 | −0.396 | 17.531 | 18.878 | 1.00 | 45.11 | A |
| ATOM | 1718 | CB | ASP | A | 842 | 0.651 | 16.836 | 19.745 | 1.00 | 47.46 | A |
| ATOM | 1719 | CG | ASP | A | 842 | 0.324 | 16.913 | 21.224 | 1.00 | 49.71 | A |
| ATOM | 1720 | OD1 | ASP | A | 842 | −0.182 | 17.971 | 21.667 | 1.00 | 50.47 | A |
| ATOM | 1721 | OD2 | ASP | A | 842 | 0.583 | 15.923 | 21.942 | 1.00 | 50.96 | A |
| ATOM | 1722 | C | ASP | A | 842 | 0.059 | 17.535 | 17.424 | 1.00 | 43.89 | A |
| ATOM | 1723 | O | ASP | A | 842 | 0.540 | 18.549 | 16.923 | 1.00 | 43.19 | A |
| ATOM | 1724 | N | VAL | A | 843 | −0.099 | 16.397 | 16.752 | 1.00 | 42.58 | A |
| ATOM | 1725 | CA | VAL | A | 843 | 0.285 | 16.281 | 15.351 | 1.00 | 41.17 | A |
| ATOM | 1726 | CB | VAL | A | 843 | −0.012 | 14.869 | 14.795 | 1.00 | 41.26 | A |
| ATOM | 1727 | CG1 | VAL | A | 843 | 0.278 | 14.828 | 13.308 | 1.00 | 39.84 | A |
| ATOM | 1728 | CG2 | VAL | A | 843 | 0.831 | 13.829 | 15.530 | 1.00 | 41.01 | A |
| ATOM | 1729 | C | VAL | A | 843 | −0.481 | 17.301 | 14.518 | 1.00 | 40.78 | A |
| ATOM | 1730 | O | VAL | A | 843 | 0.103 | 18.029 | 13.714 | 1.00 | 38.98 | A |
| ATOM | 1731 | N | ILE | A | 844 | −1.795 | 17.340 | 14.717 | 1.00 | 40.52 | A |
| ATOM | 1732 | CA | ILE | A | 844 | −2.657 | 18.269 | 14.003 | 1.00 | 41.21 | A |
| ATOM | 1733 | CB | ILE | A | 844 | −4.123 | 18.129 | 14.470 | 1.00 | 40.73 | A |
| ATOM | 1734 | CG2 | ILE | A | 844 | −4.995 | 19.190 | 13.800 | 1.00 | 40.70 | A |
| ATOM | 1735 | CG1 | ILE | A | 844 | −4.625 | 16.716 | 14.160 | 1.00 | 40.24 | A |
| ATOM | 1736 | CD1 | ILE | A | 844 | −6.004 | 16.413 | 14.694 | 1.00 | 39.42 | A |
| ATOM | 1737 | C | ILE | A | 844 | −2.196 | 19.706 | 14.237 | 1.00 | 42.20 | A |
| ATOM | 1738 | O | ILE | A | 844 | −2.035 | 20.478 | 13.289 | 1.00 | 42.32 | A |
| ATOM | 1739 | N | ASN | A | 845 | −1.988 | 20.059 | 15.502 | 1.00 | 42.47 | A |
| ATOM | 1740 | CA | ASN | A | 845 | −1.550 | 21.405 | 15.845 | 1.00 | 43.99 | A |
| ATOM | 1741 | CB | ASN | A | 845 | −1.525 | 21.606 | 17.361 | 1.00 | 45.43 | A |
| ATOM | 1742 | CG | ASN | A | 845 | −2.885 | 21.417 | 17.998 | 1.00 | 46.85 | A |
| ATOM | 1743 | OD1 | ASN | A | 845 | −3.903 | 21.851 | 17.458 | 1.00 | 47.77 | A |
| ATOM | 1744 | ND2 | ASN | A | 845 | −2.908 | 20.779 | 19.163 | 1.00 | 48.25 | A |
| ATOM | 1745 | C | ASN | A | 845 | −0.162 | 21.664 | 15.286 | 1.00 | 43.59 | A |
| ATOM | 1746 | O | ASN | A | 845 | 0.123 | 22.750 | 14.784 | 1.00 | 43.60 | A |
| ATOM | 1747 | N | ALA | A | 846 | 0.702 | 20.660 | 15.376 | 1.00 | 42.78 | A |
| ATOM | 1748 | CA | ALA | A | 846 | 2.057 | 20.793 | 14.870 | 1.00 | 42.00 | A |
| ATOM | 1749 | CB | ALA | A | 846 | 2.829 | 19.497 | 15.092 | 1.00 | 41.98 | A |
| ATOM | 1750 | C | ALA | A | 846 | 2.021 | 21.142 | 13.386 | 1.00 | 41.78 | A |
| ATOM | 1751 | O | ALA | A | 846 | 2.662 | 22.100 | 12.954 | 1.00 | 41.69 | A |
| ATOM | 1752 | N | ILE | A | 847 | 1.269 | 20.364 | 12.609 | 1.00 | 41.74 | A |
| ATOM | 1753 | CA | ILE | A | 847 | 1.155 | 20.600 | 11.173 | 1.00 | 41.99 | A |
| ATOM | 1754 | CB | ILE | A | 847 | 0.250 | 19.540 | 10.492 | 1.00 | 41.47 | A |
| ATOM | 1755 | CG2 | ILE | A | 847 | 0.083 | 19.863 | 9.013 | 1.00 | 41.65 | A |
| ATOM | 1756 | CG1 | ILE | A | 847 | 0.869 | 18.148 | 10.641 | 1.00 | 41.58 | A |
| ATOM | 1757 | CD1 | ILE | A | 847 | 2.243 | 18.014 | 10.006 | 1.00 | 41.53 | A |
| ATOM | 1758 | C | ILE | A | 847 | 0.584 | 21.990 | 10.906 | 1.00 | 42.01 | A |
| ATOM | 1759 | O | ILE | A | 847 | 1.032 | 22.685 | 9.996 | 1.00 | 41.61 | A |
| ATOM | 1760 | N | GLU | A | 848 | −0.402 | 22.388 | 11.704 | 1.00 | 42.81 | A |
| ATOM | 1761 | CA | GLU | A | 848 | −1.022 | 23.700 | 11.563 | 1.00 | 43.79 | A |
| ATOM | 1762 | CB | GLU | A | 848 | −2.158 | 23.866 | 12.576 | 1.00 | 43.97 | A |
| ATOM | 1763 | CG | GLU | A | 848 | −3.380 | 23.013 | 12.281 | 0.00 | 44.75 | A |
| ATOM | 1764 | CD | GLU | A | 848 | −4.518 | 23.276 | 13.247 | 0.00 | 45.09 | A |
| ATOM | 1765 | OE1 | GLU | A | 848 | −4.985 | 24.432 | 13.317 | 0.00 | 45.34 | A |
| ATOM | 1766 | OE2 | GLU | A | 848 | −4.947 | 22.326 | 13.935 | 0.00 | 45.34 | A |
| ATOM | 1767 | C | GLU | A | 848 | 0.011 | 24.806 | 11.760 | 1.00 | 44.25 | A |
| ATOM | 1768 | O | GLU | A | 848 | −0.033 | 25.830 | 11.082 | 1.00 | 44.97 | A |
| ATOM | 1769 | N | GLN | A | 849 | 0.939 | 24.588 | 12.689 | 1.00 | 43.53 | A |
| ATOM | 1770 | CA | GLN | A | 849 | 1.997 | 25.552 | 12.974 | 1.00 | 43.30 | A |
| ATOM | 1771 | CE | GLN | A | 849 | 2.554 | 25.328 | 14.380 | 1.00 | 43.26 | A |
| ATOM | 1772 | CG | GLN | A | 849 | 1.549 | 25.528 | 15.492 | 0.00 | 44.28 | A |
| ATOM | 1773 | CD | GLN | A | 849 | 1.042 | 26.951 | 15.557 | 0.00 | 44.64 | A |
| ATOM | 1774 | OE1 | GLN | A | 849 | 1.816 | 27.889 | 15.745 | 0.00 | 44.94 | A |
| ATOM | 1775 | NE2 | GLN | A | 849 | −0.264 | 27.121 | 15.399 | 0.00 | 44.94 | A |
| ATOM | 1776 | C | GLN | A | 849 | 3.126 | 25.402 | 11.959 | 1.00 | 43.40 | A |
| ATOM | 1777 | O | GLN | A | 849 | 4.252 | 25.842 | 12.199 | 1.00 | 43.15 | A |
| ATOM | 1778 | N | ASP | A | 850 | 2.814 | 24.770 | 10.830 | 1.00 | 43.13 | A |
| ATOM | 1779 | CA | ASP | A | 850 | 3.780 | 24.545 | 9.760 | 1.00 | 42.79 | A |
| ATOM | 1780 | CE | ASP | A | 850 | 4.287 | 25.888 | 9.222 | 1.00 | 43.87 | A |
| ATOM | 1781 | CG | ASP | A | 850 | 3.260 | 26.586 | 8.350 | 1.00 | 45.00 | A |
| ATOM | 1782 | OD1 | ASP | A | 850 | 2.980 | 26.080 | 7.245 | 1.00 | 47.53 | A |
| ATOM | 1783 | OD2 | ASP | A | 850 | 2.724 | 27.634 | 8.767 | 1.00 | 45.98 | A |
| ATOM | 1784 | C | ASP | A | 850 | 4.957 | 23.652 | 10.162 | 1.00 | 41.93 | A |
| ATOM | 1785 | O | ASP | A | 850 | 6.064 | 23.796 | 9.651 | 1.00 | 42.38 | A |
| ATOM | 1786 | N | TYR | A | 851 | 4.709 | 22.718 | 11.073 | 1.00 | 40.80 | A |
| ATOM | 1787 | CA | TYR | A | 851 | 5.752 | 21.795 | 11.512 | 1.00 | 39.73 | A |
| ATOM | 1788 | CE | TYR | A | 851 | 5.481 | 21.311 | 12.930 | 1.00 | 39.91 | A |
| ATOM | 1789 | CG | TYR | A | 851 | 6.348 | 20.139 | 13.323 | 1.00 | 39.84 | A |
| ATOM | 1790 | CD1 | TYR | A | 851 | 7.674 | 20.327 | 13.710 | 1.00 | 40.12 | A |
| ATOM | 1791 | CE1 | TYR | A | 851 | 8.478 | 19.249 | 14.062 | 1.00 | 39.79 | A |
| ATOM | 1792 | CD2 | TYR | A | 851 | 5.847 | 18.837 | 13.294 | 1.00 | 40.19 | A |
| ATOM | 1793 | CE2 | TYR | A | 851 | 6.643 | 17.751 | 13.642 | 1.00 | 39.38 | A |
| ATOM | 1794 | CZ | TYR | A | 851 | 7.955 | 17.964 | 14.027 | 1.00 | 39.62 | A |
| ATOM | 1795 | OH | TYR | A | 851 | 8.742 | 16.899 | 14.388 | 1.00 | 36.75 | A |
| ATOM | 1796 | C | TYR | A | 851 | 5.811 | 20.577 | 10.592 | 1.00 | 37.96 | A |
| ATOM | 1797 | O | TYR | A | 851 | 4.778 | 20.053 | 10.185 | 1.00 | 39.15 | A |
| ATOM | 1798 | N | ARG | A | 852 | 7.018 | 20.127 | 10.273 | 1.00 | 35.12 | A |
| ATOM | 1799 | CA | ARG | A | 852 | 7.185 | 18.961 | 9.419 | 1.00 | 33.58 | A |
| ATOM | 1800 | CE | ARG | A | 852 | 7.568 | 19.394 | 8.003 | 1.00 | 32.00 | A |
| ATOM | 1801 | CG | ARG | A | 852 | 6.478 | 20.173 | 7.287 | 1.00 | 31.55 | A |
| ATOM | 1802 | CD | ARG | A | 852 | 5.271 | 19.293 | 7.006 | 1.00 | 29.23 | A |
| ATOM | 1803 | NE | ARC | A | 852 | 4.254 | 19.976 | 6.206 | 1.00 | 30.58 | A |
| ATOM | 1804 | CZ | ARG | A | 852 | 3.358 | 20.835 | 6.688 | 1.00 | 28.76 | A |
| ATOM | 1805 | NE1 | ARG | A | 852 | 3.336 | 21.132 | 7.980 | 1.00 | 27.10 | A |
| ATOM | 1806 | NH2 | ARC | A | 852 | 2.476 | 21.393 | 5.876 | 1.00 | 28.74 | A |
| ATOM | 1807 | C | ARG | A | 852 | 8.253 | 18.033 | 9.992 | 1.00 | 32.81 | A |
| ATOM | 1808 | O | ARG | A | 852 | 9.260 | 18.490 | 10.524 | 1.00 | 33.32 | A |
| ATOM | 1809 | N | LEU | A | 853 | 8.025 | 16.729 | 9.881 | 1.00 | 31.16 | A |
| ATOM | 1810 | CA | LEU | A | 853 | 8.963 | 15.741 | 10.392 | 1.00 | 29.31 | A |
| ATOM | 1811 | CB | LEU | A | 853 | 8.493 | 14.330 | 10.040 | 1.00 | 28.71 | A |
| ATOM | 1812 | CG | LEU | A | 853 | 7.220 | 13.837 | 10.730 | 1.00 | 31.23 | A |
| ATOM | 1813 | CD1 | LEU | A | 853 | 6.775 | 12.514 | 10.090 | 1.00 | 29.70 | A |
| ATOM | 1814 | CD2 | LEU | A | 853 | 7.472 | 13.662 | 12.227 | 1.00 | 31.09 | A |
| ATOM | 1815 | C | LEU | A | 853 | 10.360 | 15.959 | 9.832 | 1.00 | 29.46 | A |
| ATOM | 1816 | O | LEU | A | 853 | 10.534 | 16.191 | 8.633 | 1.00 | 27.80 | A |
| ATOM | 1817 | N | PRO | A | 854 | 11.379 | 15.878 | 10.700 | 1.00 | 29.14 | A |
| ATOM | 1818 | CD | PRO | A | 854 | 11.277 | 15.586 | 12.144 | 1.00 | 29.31 | A |
| ATOM | 1819 | CA | PRO | A | 854 | 12.775 | 16.063 | 10.302 | 1.00 | 29.67 | A |
| ATOM | 1820 | CB | PRO | A | 854 | 13.487 | 16.207 | 11.641 | 1.00 | 29.68 | A |
| ATOM | 1821 | CG | PRO | A | 854 | 12.715 | 15.259 | 12.514 | 1.00 | 29.75 | A |
| ATOM | 1822 | C | PRO | A | 854 | 13.278 | 14.861 | 9.511 | 1.00 | 29.68 | A |
| ATOM | 1823 | O | PRO | A | 854 | 12.659 | 13.803 | 9.520 | 1.00 | 29.00 | A |
| ATOM | 1824 | N | PRO | A | 855 | 14.415 | 15.008 | 8.820 | 1.00 | 30.63 | A |
| ATOM | 1825 | CD | PRO | A | 855 | 15.242 | 16.220 | 8.668 | 1.00 | 30.11 | A |
| ATOM | 1826 | CA | PRO | A | 855 | 14.965 | 13.897 | 8.040 | 1.00 | 30.85 | A |
| ATOM | 1827 | CB | PRO | A | 855 | 16.089 | 14.561 | 7.246 | 1.00 | 31.70 | A |
| ATOM | 1828 | CG | PRO | A | 855 | 16.548 | 15.653 | 8.163 | 1.00 | 29.94 | A |
| ATOM | 1829 | C | PRO | A | 855 | 15.479 | 12.757 | 8.919 | 1.00 | 32.90 | A |
| ATOM | 1830 | O | PRO | A | 855 | 16.201 | 12.990 | 9.886 | 1.00 | 33.00 | A |
| ATOM | 1831 | N | PRO | A | 856 | 15.092 | 11.508 | 8.602 | 1.00 | 33.31 | A |
| ATOM | 1832 | CD | PRO | A | 856 | 14.065 | 11.113 | 7.620 | 1.00 | 32.70 | A |
| ATOM | 1833 | CA | PRO | A | 856 | 15.542 | 10.348 | 9.380 | 1.00 | 34.07 | A |
| ATOM | 1834 | CM | PRO | A | 856 | 14.894 | 9.172 | 8.649 | 1.00 | 33.02 | A |
| ATOM | 1835 | CG | PRO | A | 856 | 13.617 | 9.767 | 8.158 | 1.00 | 33.24 | A |
| ATOM | 1836 | C | PRO | A | 856 | 17.073 | 10.258 | 9.374 | 1.00 | 34.59 | A |
| ATOM | 1837 | O | PRO | A | 856 | 17.732 | 10.793 | 8.484 | 1.00 | 34.67 | A |
| ATOM | 1838 | N | MET | A | 857 | 17.633 | 9.571 | 10.363 | 1.00 | 35.13 | A |
| ATOM | 1839 | CA | MET | A | 857 | 19.081 | 9.429 | 10.467 | 1.00 | 34.34 | A |
| ATOM | 1840 | CM | MET | A | 857 | 19.432 | 8.523 | 11.650 | 1.00 | 34.60 | A |
| ATOM | 1841 | CG | MET | A | 857 | 20.891 | 8.620 | 12.117 | 1.00 | 36.07 | A |
| ATOM | 1842 | SD | MET | A | 857 | 21.222 | 7.511 | 13.500 | 0.00 | 36.17 | A |
| ATOM | 1843 | CE | MET | A | 857 | 20.786 | 8.554 | 14.888 | 0.00 | 36.58 | A |
| ATOM | 1844 | C | MET | A | 857 | 19.673 | 8.865 | 9.178 | 1.00 | 33.94 | A |
| ATOM | 1845 | O | MET | A | 857 | 19.195 | 7.858 | 8.643 | 1.00 | 32.79 | A |
| ATOM | 1846 | N | ASP | A | 858 | 20.713 | 9.533 | 8.685 | 1.00 | 32.95 | A |
| ATOM | 1847 | CA | ASP | A | 858 | 21.398 | 9.120 | 7.463 | 1.00 | 32.12 | A |
| ATOM | 1848 | CB | ASP | A | 858 | 21.957 | 7.697 | 7.631 | 1.00 | 33.12 | A |
| ATOM | 1849 | CG | ASP | A | 858 | 22.948 | 7.591 | 8.778 | 0.00 | 33.41 | A |
| ATOM | 1850 | OD1 | ASP | A | 858 | 23.981 | 8.292 | 8.740 | 0.00 | 33.74 | A |
| ATOM | 1851 | OD2 | ASP | A | 858 | 22.694 | 6.809 | 9.719 | 0.00 | 33.74 | A |
| ATOM | 1852 | C | ASP | A | 858 | 20.500 | 9.181 | 6.227 | 1.00 | 30.87 | A |
| ATOM | 1853 | O | ASP | A | 858 | 20.787 | 8.546 | 5.212 | 1.00 | 31.21 | A |
| ATOM | 1854 | N | CYS | A | 859 | 19.420 | 9.951 | 6.299 | 1.00 | 29.14 | A |
| ATOM | 1855 | CA | CYS | A | 859 | 18.514 | 10.056 | 5.158 | 1.00 | 28.23 | A |
| ATOM | 1856 | CM | CYS | A | 859 | 17.091 | 10.370 | 5.620 | 1.00 | 27.03 | A |
| ATOM | 1857 | SG | CYS | A | 859 | 15.950 | 10.675 | 4.219 | 1.00 | 25.20 | A |
| ATOM | 1858 | C | CYS | A | 859 | 18.951 | 11.128 | 4.172 | 1.00 | 26.49 | A |
| ATOM | 1859 | O | CYS | A | 859 | 19.063 | 12.297 | 4.535 | 1.00 | 27.20 | A |
| ATOM | 1860 | N | PRO | A | 860 | 19.187 | 10.747 | 2.905 | 1.00 | 25.74 | A |
| ATOM | 1861 | CD | PRO | A | 860 | 19.019 | 9.402 | 2.329 | 1.00 | 25.44 | A |
| ATOM | 1862 | CA | PRO | A | 860 | 19.610 | 11.703 | 1.877 | 1.00 | 25.49 | A |
| ATOM | 1863 | CB | PRO | A | 860 | 19.450 | 10.911 | 0.584 | 1.00 | 26.82 | A |
| ATOM | 1864 | CG | PRO | A | 860 | 19.754 | 9.518 | 1.013 | 1.00 | 25.74 | A |
| ATOM | 1865 | C | PRO | A | 860 | 18.750 | 12.965 | 1.875 | 1.00 | 25.80 | A |
| ATOM | 1866 | O | PRO | A | 860 | 17.528 | 12.887 | 2.037 | 1.00 | 23.12 | A |
| ATOM | 1867 | N | SER | A | 861 | 19.383 | 14.123 | 1.685 | 1.00 | 24.04 | A |
| ATOM | 1868 | CA | SER | A | 861 | 18.651 | 15.390 | 1.661 | 1.00 | 24.93 | A |
| ATOM | 1869 | CB | SER | A | 861 | 19.597 | 16.579 | 1.437 | 1.00 | 24.17 | A |
| ATOM | 1870 | OG | SER | A | 861 | 20.464 | 16.754 | 2.533 | 1.00 | 28.72 | A |
| ATOM | 1871 | C | SER | A | 861 | 17.596 | 15.405 | 0.558 | 1.00 | 22.46 | A |
| ATOM | 1872 | O | SER | A | 861 | 16.491 | 15.887 | 0.765 | 1.00 | 23.97 | A |
| ATOM | 1873 | N | ALA | A | 862 | 17.937 | 14.886 | −0.612 | 1.00 | 22.99 | A |
| ATOM | 1874 | CA | ALA | A | 862 | 16.990 | 14.879 | −1.715 | 1.00 | 23.51 | A |
| ATOM | 1875 | CB | ALA | A | 862 | 17.626 | 14.254 | −2.962 | 1.00 | 25.30 | A |
| ATOM | 1876 | C | ALA | A | 862 | 15.720 | 14.121 | −1.327 | 1.00 | 23.45 | A |
| ATOM | 1877 | O | ALA | A | 862 | 14.619 | 14.549 | −1.667 | 1.00 | 22.65 | A |
| ATOM | 1878 | N | LEU | A | 863 | 15.870 | 13.004 | −0.615 | 1.00 | 21.36 | A |
| ATOM | 1879 | CA | LEU | A | 863 | 14.703 | 12.232 | −0.186 | 1.00 | 20.64 | A |
| ATOM | 1880 | CB | LEU | A | 863 | 15.110 | 10.907 | 0.470 | 1.00 | 17.21 | A |
| ATOM | 1881 | CG | LEU | A | 863 | 15.473 | 9.807 | −0.522 | 1.00 | 17.62 | A |
| ATOM | 1882 | CD1 | LEU | A | 863 | 15.995 | 8.573 | 0.220 | 1.00 | 19.11 | A |
| ATOM | 1883 | CD2 | LEU | A | 863 | 14.236 | 9.465 | −1.353 | 1.00 | 16.32 | A |
| ATOM | 1884 | C | LEU | A | 863 | 13.821 | 13.010 | 0.774 | 1.00 | 19.07 | A |
| ATOM | 1885 | O | LEU | A | 863 | 12.595 | 13.005 | 0.641 | 1.00 | 20.62 | A |
| ATOM | 1886 | N | HIS | A | 864 | 14.434 | 13.683 | 1.741 | 1.00 | 19.85 | A |
| ATOM | 1887 | CA | HIS | A | 864 | 13.643 | 14.448 | 2.686 | 1.00 | 18.66 | A |
| ATOM | 1888 | CB | HIS | A | 864 | 14.496 | 14.954 | 3.847 | 1.00 | 19.81 | A |
| ATOM | 1889 | CG | HIS | A | 864 | 13.700 | 15.626 | 4.922 | 1.00 | 19.98 | A |
| ATOM | 1890 | CD2 | HIS | A | 864 | 12.826 | 15.127 | 5.828 | 1.00 | 20.76 | A |
| ATOM | 1891 | ND1 | HIS | A | 864 | 13.735 | 16.988 | 5.133 | 1.00 | 21.33 | A |
| ATOM | 1892 | CE1 | HIS | A | 864 | 12.919 | 17.299 | 6.125 | 1.00 | 22.47 | A |
| ATOM | 1893 | NE2 | HIS | A | 864 | 12.355 | 16.187 | 6.564 | 1.00 | 22.76 | A |
| ATOM | 1894 | C | HIS | A | 864 | 12.968 | 15.619 | 1.983 | 1.00 | 19.62 | A |
| ATOM | 1895 | O | HIS | A | 864 | 11.897 | 16.052 | 2.366 | 1.00 | 19.61 | A |
| ATOM | 1896 | N | GLN | A | 865 | 13.641 | 16.143 | 0.952 | 1.00 | 19.53 | A |
| ATOM | 1897 | CA | GLN | A | 865 | 13.039 | 17.259 | 0.238 | 1.00 | 19.79 | A |
| ATOM | 1898 | CB | GLN | A | 865 | 13.977 | 17.808 | −0.837 | 1.00 | 19.80 | A |
| ATOM | 1899 | CG | GLN | A | 865 | 13.379 | 18.989 | −1.567 | 1.00 | 21.23 | A |
| ATOM | 1900 | CD | GLN | A | 865 | 13.013 | 20.114 | −0.618 | 1.00 | 22.98 | A |
| ATOM | 1901 | OE1 | GLN | A | 865 | 13.868 | 20.627 | 0.105 | 1.00 | 26.38 | A |
| ATOM | 1902 | NE2 | GLN | A | 865 | 11.739 | 20.504 | −0.613 | 1.00 | 21.44 | A |
| ATOM | 1903 | C | GLN | A | 865 | 11.766 | 16.750 | −0.416 | 1.00 | 19.00 | A |
| ATOM | 1904 | O | GLN | A | 865 | 10.735 | 17.419 | −0.396 | 1.00 | 19.67 | A |
| ATOM | 1905 | N | LEU | A | 866 | 11.841 | 15.559 | −0.997 | 1.00 | 19.26 | A |
| ATOM | 1906 | CA | LEU | A | 866 | 10.663 | 14.988 | −1.632 | 1.00 | 18.45 | A |
| ATOM | 1907 | CB | LEU | A | 866 | 11.014 | 13.649 | −2.283 | 1.00 | 18.07 | A |
| ATOM | 1908 | CG | LEU | A | 866 | 9.887 | 12.930 | −3.021 | 1.00 | 19.78 | A |
| ATOM | 1909 | CD1 | LEU | A | 866 | 9.168 | 13.903 | −3.967 | 1.00 | 18.91 | A |
| ATOM | 1910 | CD2 | LEU | A | 866 | 10.467 | 11.754 | −3.775 | 1.00 | 20.97 | A |
| ATOM | 1911 | C | LEU | A | 866 | 9.546 | 14.824 | −0.589 | 1.00 | 18.47 | A |
| ATOM | 1912 | O | LEU | A | 866 | 8.367 | 15.026 | −0.890 | 1.00 | 17.51 | A |
| ATOM | 1913 | N | MET | A | 867 | 9.921 | 14.476 | 0.641 | 1.00 | 18.09 | A |
| ATOM | 1914 | CA | MET | A | 867 | 8.946 | 14.318 | 1.711 | 1.00 | 18.01 | A |
| ATOM | 1915 | CB | MET | A | 867 | 9.632 | 13.865 | 3.005 | 1.00 | 19.66 | A |
| ATOM | 1916 | CG | MET | A | 867 | 10.077 | 12.419 | 2.991 | 1.00 | 20.12 | A |
| ATOM | 1917 | SD | MET | A | 867 | 10.968 | 11.976 | 4.495 | 1.00 | 22.40 | A |
| ATOM | 1918 | CE | MET | A | 867 | 12.235 | 10.954 | 3.814 | 1.00 | 22.54 | A |
| ATOM | 1919 | C | MET | A | 867 | 8.228 | 15.640 | 1.965 | 1.00 | 18.98 | A |
| ATOM | 1920 | O | MET | A | 867 | 6.996 | 15.686 | 2.058 | 1.00 | 17.83 | A |
| ATOM | 1921 | N | LEU | A | 868 | 9.007 | 16.712 | 2.089 | 1.00 | 18.92 | A |
| ATOM | 1922 | CA | LEU | A | 868 | 8.447 | 18.042 | 2.326 | 1.00 | 20.03 | A |
| ATOM | 1923 | CB | LEU | A | 868 | 9.566 | 19.083 | 2.455 | 1.00 | 21.62 | A |
| ATOM | 1924 | CG | LEU | A | 868 | 10.538 | 18.913 | 3.628 | 1.00 | 22.08 | A |
| ATOM | 1925 | CD1 | LEU | A | 868 | 11.607 | 20.012 | 3.578 | 1.00 | 21.68 | A |
| ATOM | 1926 | CD2 | LEU | A | 868 | 9.772 | 18.989 | 4.942 | 1.00 | 18.97 | A |
| ATOM | 1927 | C | LEU | A | 868 | 7.506 | 18.444 | 1.191 | 1.00 | 19.33 | A |
| ATOM | 1928 | O | LEU | A | 868 | 6.501 | 19.111 | 1.420 | 1.00 | 20.63 | A |
| ATOM | 1929 | N | ASP | A | 869 | 7.836 | 18.037 | −0.031 | 1.00 | 17.79 | A |
| ATOM | 1930 | CA | ASP | A | 869 | 7.001 | 18.360 | −1.183 | 1.00 | 18.56 | A |
| ATOM | 1931 | CB | ASP | A | 869 | 7.707 | 17.971 | −2.484 | 1.00 | 17.65 | A |
| ATOM | 1932 | CG | ASP | A | 869 | 8.988 | 18.762 | −2.703 | 1.00 | 18.80 | A |
| ATOM | 1933 | OD1 | ASP | A | 869 | 9.175 | 19.790 | −2.021 | 1.00 | 16.81 | A |
| ATOM | 1934 | OD2 | ASP | A | 869 | 9.799 | 18.364 | −3.557 | 1.00 | 18.79 | A |
| ATOM | 1935 | C | ASP | A | 869 | 5.664 | 17.651 | −1.072 | 1.00 | 20.40 | A |
| ATOM | 1936 | O | ASP | A | 869 | 4.631 | 18.197 | −1.460 | 1.00 | 21.71 | A |
| ATOM | 1937 | N | CYS | A | 870 | 5.682 | 16.439 | −0.524 | 1.00 | 18.78 | A |
| ATOM | 1938 | CA | CYS | A | 870 | 4.456 | 15.670 | −0.354 | 1.00 | 19.36 | A |
| ATOM | 1939 | CB | CYS | A | 870 | 4.769 | 14.201 | −0.039 | 1.00 | 15.21 | A |
| ATOM | 1940 | SO | CYS | A | 870 | 5.477 | 13.278 | −1.422 | 1.00 | 17.93 | A |
| ATOM | 1941 | C | CYS | A | 870 | 3.616 | 16.256 | 0.769 | 1.00 | 16.15 | A |
| ATOM | 1942 | O | CYS | A | 870 | 2.390 | 16.090 | 0.785 | 1.00 | 20.18 | A |
| ATOM | 1943 | N | TRP | A | 871 | 4.259 | 16.956 | 1.702 | 1.00 | 19.04 | A |
| ATOM | 1944 | CA | TRP | A | 871 | 3.529 | 17.534 | 2.833 | 1.00 | 20.40 | A |
| ATOM | 1945 | CB | TRP | A | 871 | 4.306 | 17.335 | 4.151 | 1.00 | 20.01 | A |
| ATOM | 1946 | CG | TRP | A | 871 | 4.663 | 15.890 | 4.480 | 1.00 | 21.12 | A |
| ATOM | 1947 | CD2 | TRP | A | 871 | 5.874 | 15.424 | 5.095 | 1.00 | 19.64 | A |
| ATOM | 1948 | CE2 | TRP | A | 871 | 5.778 | 14.014 | 5.204 | 1.00 | 18.64 | A |
| ATOM | 1949 | CE3 | TRP | A | 871 | 7.029 | 16.060 | 5.563 | 1.00 | 18.54 | A |
| ATOM | 1950 | CD1 | TRP | A | 871 | 3.897 | 14.770 | 4.254 | 1.00 | 21.26 | A |
| ATOM | 1951 | NE1 | TRP | A | 871 | 4.567 | 13.642 | 4.685 | 1.00 | 19.40 | A |
| ATOM | 1952 | CZ2 | TRP | A | 871 | 6.797 | 13.232 | 5.762 | 1.00 | 18.78 | A |
| ATOM | 1953 | CZ3 | TRP | A | 871 | 8.045 | 15.282 | 6.120 | 1.00 | 19.69 | A |
| ATOM | 1954 | CH2 | TRP | A | 871 | 7.920 | 13.878 | 6.214 | 1.00 | 19.97 | A |
| ATOM | 1955 | C | TRP | A | 871 | 3.168 | 19.012 | 2.669 | 1.00 | 20.88 | A |
| ATOM | 1956 | O | TRP | A | 871 | 2.999 | 19.736 | 3.654 | 1.00 | 21.06 | A |
| ATOM | 1957 | N | GLN | A | 872 | 3.055 | 19.459 | 1.424 | 1.00 | 22.74 | A |
| ATOM | 1958 | CA | GLN | A | 872 | 2.680 | 20.846 | 1.157 | 1.00 | 25.25 | A |
| ATOM | 1959 | CB | GLN | A | 872 | 2.768 | 21.158 | −0.337 | 1.00 | 23.93 | A |
| ATOM | 1960 | CG | GLN | A | 872 | 4.174 | 21.383 | −0.816 | 1.00 | 29.27 | A |
| ATOM | 1961 | CD | GLN | A | 872 | 4.857 | 22.495 | −0.047 | 1.00 | 32.45 | A |
| ATOM | 1962 | OE1 | GLN | A | 872 | 4.377 | 23.628 | −0.015 | 1.00 | 34.77 | A |
| ATOM | 1963 | NE2 | GLN | A | 872 | 5.979 | 22.178 | 0.579 | 1.00 | 35.29 | A |
| ATOM | 1964 | C | GLN | A | 872 | 1.260 | 21.073 | 1.639 | 1.00 | 25.69 | A |
| ATOM | 1965 | O | GLN | A | 872 | 0.351 | 20.310 | 1.301 | 1.00 | 26.55 | A |
| ATOM | 1966 | N | LYS | A | 873 | 1.081 | 22.112 | 2.445 | 1.00 | 26.47 | A |
| ATOM | 1967 | CA | LYS | A | 873 | 0.224 | 22.463 | 2.990 | 1.00 | 27.95 | A |
| ATOM | 1968 | CB | LYS | A | 873 | 0.139 | 23.849 | 3.639 | 1.00 | 28.61 | A |
| ATOM | 1969 | CG | LYS | A | 873 | 1.390 | 24.304 | 4.360 | 1.00 | 29.84 | A |
| ATOM | 1970 | CD | LYS | A | 873 | 1.188 | 25.685 | 4.968 | 0.00 | 30.40 | A |
| ATOM | 1971 | CE | LYS | A | 873 | 2.412 | 26.130 | 5.752 | 0.00 | 30.90 | A |
| ATOM | 1972 | NZ | LYS | A | 873 | 2.223 | 27.477 | 6.360 | 0.00 | 31.23 | A |
| ATOM | 1973 | C | LYS | A | 873 | 1.281 | 22.446 | 1.886 | 1.00 | 29.41 | A |
| ATOM | 1974 | O | LYS | A | 873 | 2.320 | 21.800 | 2.022 | 1.00 | 28.03 | A |
| ATOM | 1975 | N | ASP | A | 874 | 1.008 | 23.156 | 0.793 | 1.00 | 30.47 | A |
| ATOM | 1976 | CA | ASP | A | 874 | 1.930 | 23.211 | −0.340 | 1.00 | 32.13 | A |
| ATOM | 1977 | CB | ASP | A | 874 | 1.585 | 24.399 | −1.247 | 1.00 | 34.07 | A |
| ATOM | 1978 | CD | ASP | A | 874 | 2.640 | 24.656 | −2.304 | 1.00 | 36.22 | A |
| ATOM | 1979 | OD1 | ASP | A | 874 | 3.160 | 23.678 | −2.887 | 1.00 | 36.33 | A |
| ATOM | 1980 | OD2 | ASP | A | 874 | 2.944 | 25.844 | −2.560 | 1.00 | 38.96 | A |
| ATOM | 1981 | C | ASP | A | 874 | 1.806 | 21.919 | −1.146 | 1.00 | 30.91 | A |
| ATOM | 1982 | O | ASP | A | 874 | 0.758 | 21.653 | −1.727 | 1.00 | 30.73 | A |
| ATOM | 1983 | N | ARG | A | 875 | 2.876 | 21.134 | −1.207 | 1.00 | 30.76 | A |
| ATOM | 1984 | CA | ARG | A | 875 | 2.837 | 19.870 | −1.939 | 1.00 | 30.91 | A |
| ATOM | 1985 | CB | ARG | A | 875 | 4.177 | 19.138 | −1.832 | 1.00 | 32.82 | A |
| ATOM | 1986 | CG | ARG | A | 875 | 5.316 | 19.755 | −2.635 | 1.00 | 35.08 | A |
| ATOM | 1987 | CD | ARG | A | 875 | 6.413 | 18.722 | −2.844 | 1.00 | 40.15 | A |
| ATOM | 1988 | NE | ARG | A | 875 | 7.555 | 19.234 | −3.603 | 1.00 | 43.73 | A |
| ATOM | 1989 | CZ | ARG | A | 875 | 8.448 | 20.094 | −3.125 | 1.00 | 45.20 | A |
| ATOM | 1990 | NH1 | ARG | A | 875 | 8.334 | 20.544 | −1.883 | 1.00 | 45.87 | A |
| ATOM | 1991 | NH2 | ARG | A | 875 | 9.457 | 20.502 | −3.885 | 1.00 | 47.30 | A |
| ATOM | 1992 | C | ARG | A | 875 | 2.477 | 20.028 | −3.412 | 1.00 | 28.33 | A |
| ATOM | 1993 | O | ARG | A | 875 | 1.965 | 19.100 | −4.033 | 1.00 | 26.80 | A |
| ATOM | 1994 | N | ASN | A | 876 | 2.756 | 21.199 | −3.972 | 1.00 | 27.63 | A |
| ATOM | 1995 | CA | ASN | A | 876 | 2.463 | 21.457 | −5.375 | 1.00 | 27.11 | A |
| ATOM | 1996 | CB | ASN | A | 876 | 3.223 | 22.703 | −5.845 | 1.00 | 29.67 | A |
| ATOM | 1997 | CG | ASN | A | 876 | 4.663 | 22.396 | −6.237 | 1.00 | 32.23 | A |
| ATOM | 1998 | OD1 | ASN | A | 876 | 5.558 | 23.221 | −6.060 | 1.00 | 36.30 | A |
| ATOM | 1999 | ND2 | ASP | A | 876 | 4.887 | 21.212 | −6.786 | 1.00 | 33.53 | A |
| ATOM | 2000 | C | ASN | A | 876 | 0.972 | 21.621 | −5.632 | 1.00 | 25.93 | A |
| ATOM | 2001 | O | ASN | A | 876 | 0.500 | 21.396 | −6.746 | 1.00 | 26.98 | A |
| ATOM | 2002 | N | HIS | A | 877 | 0.235 | 21.996 | −4.592 | 1.00 | 24.62 | A |
| ATOM | 2003 | CA | HIS | A | 877 | 1.206 | 22.203 | −4.690 | 1.00 | 23.95 | A |
| ATOM | 2004 | CB | HIS | A | 877 | 1.666 | 23.185 | −3.605 | 1.00 | 25.91 | A |
| ATOM | 2005 | CG | HIS | A | 877 | 1.103 | 24.566 | −3.758 | 1.00 | 28.62 | A |
| ATOM | 2006 | CD2 | HIS | A | 877 | 0.390 | 25.134 | −4.760 | 1.00 | 29.02 | A |
| ATOM | 2007 | ND1 | HIS | A | 877 | 1.268 | 25.548 | −2.803 | 1.00 | 30.92 | A |
| ATOM | 2008 | CE1 | HIS | A | 877 | 0.682 | 26.659 | −3.212 | 1.00 | 32.07 | A |
| ATOM | 2009 | NE2 | HIS | A | 877 | 0.142 | 26.434 | −4.396 | 1.00 | 30.42 | A |
| ATOM | 2010 | C | HIS | A | 877 | 1.987 | 20.893 | −4.555 | 1.00 | 24.02 | A |
| ATOM | 2011 | O | HIS | A | 877 | 3.169 | 20.821 | −4.919 | 1.00 | 21.47 | A |
| ATOM | 2012 | N | ARG | A | 878 | 1.342 | 19.860 | −4.017 | 1.00 | 22.45 | A |
| ATOM | 2013 | CA | ARG | A | 878 | 2.022 | 18.574 | −3.858 | 1.00 | 20.77 | A |
| ATOM | 2014 | CE | ARG | A | 878 | 1.196 | 17.618 | −2.995 | 1.00 | 18.58 | A |
| ATOM | 2015 | CG | ARG | A | 878 | 0.878 | 18.140 | −1.609 | 1.00 | 17.53 | A |
| ATOM | 2016 | CD | AEG | A | 878 | 0.163 | 17.267 | −0.922 | 1.00 | 17.61 | A |
| ATOM | 2017 | NE | ARG | A | 878 | 0.678 | 17.923 | 0.273 | 1.00 | 15.99 | A |
| ATOM | 2018 | CZ | ARG | A | 878 | 1.898 | 17.746 | 0.763 | 1.00 | 17.79 | A |
| ATOM | 2019 | NH1 | ARG | A | 878 | 2.743 | 16.913 | 0.165 | 1.00 | 18.10 | A |
| ATOM | 2020 | NH2 | ARG | A | 878 | 2.282 | 18.440 | 1.830 | 1.00 | 20.88 | A |
| ATOM | 2021 | C | ARG | A | 878 | 2.264 | 17.922 | −5.208 | 1.00 | 20.33 | A |
| ATOM | 2022 | O | ARG | A | 878 | 1.473 | 18.058 | −6.137 | 1.00 | 21.44 | A |
| ATOM | 2023 | N | PRO | A | 879 | 3.377 | 17.200 | −5.334 | 1.00 | 19.46 | A |
| ATOM | 2024 | CD | PRO | A | 879 | 4.395 | 16.893 | −4.307 | 1.00 | 20.47 | A |
| ATOM | 2025 | CA | PRO | A | 879 | 3.687 | 16.530 | −6.592 | 1.00 | 18.32 | A |
| ATOM | 2026 | CB | PRO | A | 879 | 5.116 | 16.050 | −6.374 | 1.00 | 19.77 | A |
| ATOM | 2027 | CG | PRO | A | 879 | 5.106 | 15.696 | −4.907 | 1.00 | 18.07 | A |
| ATOM | 2028 | C | PRO | A | 879 | 2.727 | 15.362 | −6.801 | 1.00 | 18.71 | A |
| ATOM | 2029 | O | PRO | A | 879 | 2.135 | 14.846 | −5.849 | 1.00 | 17.95 | A |
| ATOM | 2030 | N | LYS | A | 880 | 2.569 | 14.957 | −8.051 | 1.00 | 18.17 | A |
| ATOM | 2031 | CA | LYS | A | 880 | 1.705 | 13.835 | −8.373 | 1.00 | 19.50 | A |
| ATOM | 2032 | CB | LYS | A | 880 | 1.118 | 14.000 | −9.775 | 1.00 | 20.53 | A |
| ATOM | 2033 | CG | LYS | A | 880 | 0.082 | 15.125 | −9.888 | 1.00 | 23.92 | A |
| ATOM | 2034 | CD | LYS | A | 880 | 0.316 | 15.362 | −11.334 | 1.00 | 26.32 | A |
| ATOM | 2035 | CB | LYS | A | 880 | 1.444 | 16.384 | −11.434 | 1.00 | 29.77 | A |
| ATOM | 2036 | NZ | LYS | A | 880 | 1.732 | 16.761 | −12.845 | 1.00 | 33.61 | A |
| ATOM | 2037 | C | LYS | A | 880 | 2.545 | 12.572 | −8.315 | 1.00 | 18.71 | A |
| ATOM | 2038 | O | LYS | A | 880 | 3.775 | 12.637 | −8.369 | 1.00 | 15.94 | A |
| ATOM | 2039 | N | PHE | A | 881 | 1.887 | 11.421 | −8.207 | 1.00 | 19.36 | A |
| ATOM | 2040 | CA | PHE | A | 881 | 2.625 | 10.174 | −8.148 | 1.00 | 18.63 | A |
| ATOM | 2041 | CB | PHE | A | 881 | 1.679 | 8.980 | −7.961 | 1.00 | 18.14 | A |
| ATOM | 2042 | CG | PHE | A | 881 | 1.154 | 8.856 | −6.561 | 1.00 | 15.85 | A |
| ATOM | 2043 | CD1 | PHE | A | 681 | 0.182 | 9.091 | −6.281 | 1.00 | 15.19 | A |
| ATOM | 2044 | CD2 | PHE | A | 881 | 2.017 | 8.573 | −5.503 | 1.00 | 14.91 | A |
| ATOM | 2045 | CE1 | PHE | A | 881 | 0.661 | 9.055 | −4.974 | 1.00 | 14.06 | A |
| ATOM | 2046 | CE2 | PHE | A | 881 | 1.545 | 8.536 | −4.186 | 1.00 | 14.78 | A |
| ATOM | 2047 | CZ | PHE | A | 881 | 0.201 | 8.780 | −3.923 | 1.00 | 16.56 | A |
| ATOM | 2048 | C | PHE | A | 881 | 3.500 | 9.975 | −9.365 | 1.00 | 18.17 | A |
| ATOM | 2049 | O | PHE | A | 881 | 4.570 | 9.381 | −9.266 | 1.00 | 17.98 | A |
| ATOM | 2050 | N | GLY | A | 882 | 3.065 | 10.476 | −10.517 | 1.00 | 17.95 | A |
| ATOM | 2051 | CA | GLY | A | 882 | 3.879 | 10.329 | −11.710 | 1.00 | 17.50 | A |
| ATOM | 2052 | C | GLY | A | 882 | 5.173 | 11.112 | −11.559 | 1.00 | 17.01 | A |
| ATOM | 2053 | O | GLY | A | 882 | 6.256 | 10.660 | −11.953 | 1.00 | 15.84 | A |
| ATOM | 2054 | N | GLN | A | 883 | 5.066 | 12.295 | −10.974 | 1.00 | 16.68 | A |
| ATOM | 2055 | CA | GLN | A | 883 | 6.240 | 13.129 | −10.776 | 1.00 | 18.91 | A |
| ATOM | 2056 | CE | GLN | A | 883 | 5.813 | 14.551 | −10.391 | 1.00 | 19.76 | A |
| ATOM | 2057 | CG | GLN | A | 883 | 4.850 | 15.188 | −11.387 | 1.00 | 25.47 | A |
| ATOM | 2058 | CD | GLN | A | 883 | 4.415 | 16.571 | −10.949 | 1.00 | 26.44 | A |
| ATOM | 2059 | OE1 | GLN | A | 883 | 3.799 | 16.739 | −9.896 | 1.00 | 24.34 | A |
| ATOM | 2060 | NE2 | GLN | A | 883 | 4.748 | 17.577 | −11.756 | 1.00 | 28.51 | A |
| ATOM | 2061 | C | GLN | A | 883 | 7.116 | 12.519 | −9.677 | 1.00 | 17.08 | A |
| ATOM | 2062 | O | GLN | A | 883 | 8.336 | 12.625 | −9.719 | 1.00 | 16.89 | A |
| ATOM | 2063 | N | ILE | A | 884 | 6.484 | 11.872 | −8.700 | 1.00 | 16.63 | A |
| ATOM | 2064 | CA | ILE | A | 884 | 7.221 | 11.240 | −7.612 | 1.00 | 15.31 | A |
| ATOM | 2065 | CB | ILE | A | 884 | 6.267 | 10.698 | −6.516 | 1.00 | 16.28 | A |
| ATOM | 2066 | CG2 | ILE | A | 884 | 7.001 | 9.708 | −5.594 | 1.00 | 15.49 | A |
| ATOM | 2067 | CG1 | ILE | A | 884 | 5.700 | 11.869 | −5.714 | 1.00 | 15.76 | A |
| ATOM | 2068 | CD1 | ILE | A | 884 | 4.588 | 11.471 | −4.743 | 1.00 | 19.15 | A |
| ATOM | 2069 | C | ILE | A | 884 | 8.058 | 10.102 | −8.166 | 1.00 | 14.47 | A |
| ATOM | 2070 | O | ILE | A | 884 | 9.229 | 9.988 | −7.839 | 1.00 | 14.80 | A |
| ATOM | 2071 | N | VAL | A | 885 | 7.466 | 9.275 | −9.024 | 1.00 | 13.59 | A |
| ATOM | 2072 | CA | VAL | A | 885 | 8.223 | 8.177 | −9.601 | 1.00 | 16.17 | A |
| ATOM | 2073 | CB | VAL | A | 885 | 7.352 | 7.284 | −10.502 | 1.00 | 16.96 | A |
| ATOM | 2074 | CG1 | VAL | A | 885 | 8.216 | 6.248 | −11.178 | 1.00 | 15.86 | A |
| ATOM | 2075 | CG2 | VAL | A | 885 | 6.252 | 6.588 | −9.676 | 1.00 | 16.66 | A |
| ATOM | 2076 | C | VAL | A | 885 | 9.414 | 8.683 | −10.416 | 1.00 | 19.15 | A |
| ATOM | 2077 | O | VAL | A | 885 | 10.479 | 8.062 | −10.417 | 1.00 | 16.03 | A |
| ATOM | 2078 | N | ASN | A | 886 | 9.241 | 9.808 | −11.110 | 1.00 | 19.66 | A |
| ATOM | 2079 | CA | ASN | A | 886 | 10.320 | 10.343 | −11.936 | 1.00 | 21.37 | A |
| ATOM | 2080 | CB | ASN | A | 886 | 9.819 | 11.509 | −12.793 | 1.00 | 23.10 | A |
| ATOM | 2081 | CD | ASN | A | 886 | 8.698 | 11.102 | −13.734 | 1.00 | 26.10 | A |
| ATOM | 2082 | OD1 | ASN | A | 886 | 8.773 | 10.056 | −14.398 | 1.00 | 26.24 | A |
| ATOM | 2083 | ND2 | ASN | A | 886 | 7.654 | 11.926 | −13.804 | 1.00 | 27.46 | A |
| ATOM | 2084 | C | ASN | A | 886 | 11.500 | 10.807 | −11.097 | 1.00 | 20.89 | A |
| ATOM | 2085 | O | ASN | A | 886 | 12.655 | 10.562 | −11.446 | 1.00 | 21.26 | A |
| ATOM | 2086 | N | THR | A | 887 | 11.189 | 11.479 | −9.995 | 1.00 | 19.17 | A |
| ATOM | 2087 | CA | THR | A | 887 | 12.180 | 11.991 | −9.069 | 1.00 | 20.28 | A |
| ATOM | 2088 | CE | THR | A | 887 | 11.500 | 12.789 | −7.939 | 1.00 | 22.23 | A |
| ATOM | 2089 | OG1 | THR | A | 887 | 10.751 | 13.872 | −8.504 | 1.00 | 24.34 | A |
| ATOM | 2090 | CG2 | THR | A | 887 | 12.525 | 13.327 | −6.968 | 1.00 | 22.10 | A |
| ATOM | 2091 | C | THR | A | 887 | 12.961 | 10.838 | −8.450 | 1.00 | 20.51 | A |
| ATOM | 2092 | O | THR | A | 887 | 14.182 | 10.906 | −8.339 | 1.00 | 20.72 | A |
| ATOM | 2093 | N | LEU | A | 888 | 12.259 | 9.778 | −8.055 | 1.00 | 17.54 | A |
| ATOM | 2094 | CA | LEU | A | 888 | 12.926 | 8.619 | −7.458 | 1.00 | 16.74 | A |
| ATOM | 2095 | CB | LEU | A | 888 | 11.906 | 7.629 | −6.882 | 1.00 | 13.99 | A |
| ATOM | 2096 | CG | LEU | A | 888 | 11.102 | 8.102 | −5.660 | 1.00 | 16.09 | A |
| ATOM | 2097 | CD1 | LEU | A | 888 | 9.998 | 7.118 | −5.344 | 1.00 | 12.23 | A |
| ATOM | 2098 | CD2 | LEU | A | 888 | 12.037 | 8.236 | −4.444 | 1.00 | 15.84 | A |
| ATOM | 2099 | C | LEU | A | 888 | 13.791 | 7.939 | −8.507 | 1.00 | 17.83 | A |
| ATOM | 2100 | O | LEU | A | 888 | 14.914 | 7.509 | −8.215 | 1.00 | 16.99 | A |
| ATOM | 2101 | N | ASP | A | 889 | 13.278 | 7.846 | −9.731 | 1.00 | 18.45 | A |
| ATOM | 2102 | CA | ASP | A | 889 | 14.047 | 7.231 | −10.809 | 1.00 | 19.88 | A |
| ATOM | 2103 | CB | ASP | A | 889 | 13.216 | 7.089 | −12.092 | 1.00 | 20.92 | A |
| ATOM | 2104 | CG | ASP | A | 889 | 12.212 | 5.954 | −12.031 | 1.00 | 24.40 | A |
| ATOM | 2105 | OD1 | ASP | A | 889 | 12.496 | 4.901 | −11.417 | 1.00 | 23.95 | A |
| ATOM | 2106 | OD2 | ASP | A | 889 | 11.127 | 6.106 | −12.620 | 1.00 | 26.43 | A |
| ATOM | 2107 | C | ASP | A | 889 | 15.303 | 8.053 | −11.128 | 1.00 | 20.04 | A |
| ATOM | 2108 | O | ASP | A | 889 | 16.341 | 7.491 | −11.459 | 1.00 | 20.12 | A |
| ATOM | 2109 | N | LYS | A | 890 | 15.221 | 9.377 | −11.035 | 1.00 | 20.98 | A |
| ATOM | 2110 | CA | LYS | A | 890 | 16.398 | 10.193 | −11.320 | 1.00 | 24.03 | A |
| ATOM | 2111 | CB | LYS | A | 890 | 16.042 | 11.679 | −11.353 | 1.00 | 23.49 | A |
| ATOM | 2112 | CD | LYS | A | 890 | 15.102 | 12.062 | −12.480 | 0.00 | 24.55 | A |
| ATOM | 2113 | CD | LYS | A | 890 | 14.792 | 13.544 | −12.453 | 0.00 | 25.01 | A |
| ATOM | 2114 | CE | LYS | A | 890 | 13.901 | 13.935 | −13.615 | 0.00 | 25.39 | A |
| ATOM | 2115 | NZ | LYS | A | 890 | 13.587 | 15.388 | −13.598 | 0.00 | 25.67 | A |
| ATOM | 2116 | C | LYS | A | 890 | 17.455 | 9.935 | −10.255 | 1.00 | 23.54 | A |
| ATOM | 2117 | O | LYS | A | 890 | 18.644 | 9.911 | −10.555 | 1.00 | 23.88 | A |
| ATOM | 2118 | N | MET | A | 891 | 17.014 | 9.744 | −9.012 | 1.00 | 23.82 | A |
| ATOM | 2119 | CA | MET | A | 891 | 17.939 | 9.457 | −7.913 | 1.00 | 22.98 | A |
| ATOM | 2120 | CB | MET | A | 891 | 17.192 | 9.425 | −6.573 | 1.00 | 22.51 | A |
| ATOM | 2121 | CD | MET | A | 891 | 16.487 | 10.746 | −6.206 | 1.00 | 19.86 | A |
| ATOM | 2122 | SD | MET | A | 891 | 15.428 | 10.557 | −4.742 | 1.00 | 22.11 | A |
| ATOM | 2123 | CE | MET | A | 891 | 15.002 | 12.246 | −4.390 | 1.00 | 18.78 | A |
| ATOM | 2124 | C | MET | A | 891 | 18.639 | 8.120 | −8.163 | 1.00 | 23.81 | A |
| ATOM | 2125 | O | MET | A | 891 | 19.853 | 8.009 | −7.996 | 1.00 | 23.38 | A |
| ATOM | 2126 | N | ILE | A | 892 | 17.874 | 7.113 | −8.581 | 1.00 | 22.60 | A |
| ATOM | 2127 | CA | ILE | A | 892 | 18.451 | 5.806 | −8.853 | 1.00 | 23.72 | A |
| ATOM | 2128 | CS | ILE | A | 892 | 17.362 | 4.765 | −9.203 | 1.00 | 22.69 | A |
| ATOM | 2129 | CG2 | ILE | A | 892 | 18.001 | 3.453 | −9.665 | 1.00 | 21.79 | A |
| ATOM | 2130 | CG1 | ILE | A | 892 | 16.501 | 4.487 | −7.969 | 1.00 | 21.70 | A |
| ATOM | 2131 | CD1 | ILE | A | 892 | 15.347 | 3.569 | −8.236 | 1.00 | 21.58 | A |
| ATOM | 2132 | C | ILE | A | 892 | 19.454 | 5.892 | −10.004 | 1.00 | 25.56 | A |
| ATOM | 2133 | O | ILE | A | 892 | 20.508 | 5.264 | −9.964 | 1.00 | 24.56 | A |
| ATOM | 2134 | N | ARG | A | 893 | 19.126 | 6.672 | −11.029 | 1.00 | 27.68 | A |
| ATOM | 2135 | CA | ARG | A | 893 | 20.006 | 6.826 | −12.183 | 1.00 | 28.93 | A |
| ATOM | 2136 | CS | ARG | A | 893 | 19.246 | 7.486 | −13.338 | 1.00 | 29.77 | A |
| ATOM | 2137 | CG | ARG | A | 893 | 18.033 | 6.702 | −13.801 | 0.00 | 30.52 | A |
| ATOM | 2138 | CD | ARG | A | 893 | 17.167 | 7.518 | −14.745 | 0.00 | 31.20 | A |
| ATOM | 2139 | NE | ARG | A | 893 | 16.022 | 6.747 | −15.220 | 0.00 | 31.76 | A |
| ATOM | 2140 | CZ | ARG | A | 893 | 15.065 | 7.231 | −16.006 | 0.00 | 32.04 | A |
| ATOM | 2141 | NH1 | ARG | A | 893 | 15.109 | 8.492 | −16.413 | 0.00 | 32.22 | A |
| ATOM | 2142 | NH2 | ARG | A | 893 | 14.065 | 6.449 | −16.388 | 0.00 | 32.22 | A |
| ATOM | 2143 | C | ARG | A | 893 | 21.237 | 7.659 | −11.828 | 1.00 | 28.29 | A |
| ATOM | 2144 | O | ARG | A | 893 | 22.304 | 7.472 | −12.410 | 1.00 | 30.73 | A |
| ATOM | 2145 | N | ASN | A | 894 | 21.090 | 8.574 | −10.873 | 1.00 | 27.77 | A |
| ATOM | 2146 | CA | ASN | A | 894 | 22.206 | 9.421 | −10.454 | 1.00 | 28.66 | A |
| ATOM | 2147 | CS | ASN | A | 894 | 21.928 | 10.883 | −10.791 | 1.00 | 32.18 | A |
| ATOM | 2148 | CG | ASN | A | 894 | 21.446 | 11.064 | −12.204 | 1.00 | 35.93 | A |
| ATOM | 2149 | OD2 | ASN | A | 894 | 20.309 | 10.708 | −12.536 | 1.00 | 38.24 | A |
| ATOM | 2150 | ND2 | ASN | A | 894 | 22.305 | 11.613 | −13.056 | 1.00 | 37.07 | A |
| ATOM | 2151 | C | ASN | A | 894 | 22.444 | 9.295 | −8.960 | 1.00 | 27.18 | A |
| ATOM | 2152 | O | ASN | A | 894 | 22.256 | 10.257 | −8.205 | 1.00 | 24.38 | A |
| ATOM | 2153 | N | PRO | A | 895 | 22.878 | 8.102 | −8.517 | 1.00 | 27.24 | A |
| ATOM | 2154 | CD | PRO | A | 895 | 23.287 | 6.996 | −9.402 | 1.00 | 27.14 | A |
| ATOM | 2155 | CA | PRO | A | 895 | 23.162 | 7.774 | −7.116 | 1.00 | 27.82 | A |
| ATOM | 2156 | CS | PRO | A | 895 | 23.990 | 6.496 | −7.222 | 1.00 | 27.28 | A |
| ATOM | 2157 | CG | PRO | A | 895 | 23.441 | 5.847 | −8.435 | 1.00 | 29.74 | A |
| ATOM | 2158 | C | PRO | A | 895 | 23.888 | 8.859 | −6.324 | 1.00 | 27.80 | A |
| ATOM | 2159 | O | PRO | A | 895 | 23.705 | 8.974 | −5.112 | 1.00 | 26.04 | A |
| ATOM | 2160 | N | ASN | A | 896 | 24.718 | 9.655 | −6.988 | 1.00 | 29.72 | A |
| ATOM | 2161 | CA | ASN | A | 896 | 25.426 | 10.694 | −6.257 | 1.00 | 31.84 | A |
| ATOM | 2162 | CB | ASN | A | 896 | 26.449 | 11.392 | −7.152 | 1.00 | 35.12 | A |
| ATOM | 2163 | CG | ASN | A | 896 | 27.619 | 10.495 | −7.491 | 1.00 | 37.45 | A |
| ATOM | 2164 | OD1 | ASN | A | 896 | 28.189 | 9.842 | −6.610 | 1.00 | 37.32 | A |
| ATOM | 2165 | ND2 | ASN | A | 896 | 27.988 | 10.455 | −8.769 | 1.00 | 38.75 | A |
| ATOM | 2166 | C | ASN | A | 896 | 24.471 | 11.715 | −5.652 | 1.00 | 31.58 | A |
| ATOM | 2167 | O | ASN | A | 896 | 24.789 | 12.337 | −4.642 | 1.00 | 32.05 | A |
| ATOM | 2168 | N | SER | A | 897 | 23.295 | 11.876 | −6.250 | 1.00 | 31.96 | A |
| ATOM | 2169 | CA | SER | A | 897 | 22.322 | 12.828 | −5.724 | 1.00 | 31.92 | A |
| ATOM | 2170 | CS | SER | A | 897 | 21.100 | 12.903 | −6.639 | 1.00 | 32.67 | A |
| ATOM | 2171 | OG | SER | A | 897 | 20.427 | 11.657 | −6.699 | 1.00 | 32.92 | A |
| ATOM | 2172 | C | SER | A | 897 | 21.872 | 12.442 | −4.320 | 1.00 | 32.25 | A |
| ATOM | 2173 | O | SER | A | 897 | 21.288 | 13.253 | −3.601 | 1.00 | 32.16 | A |
| ATOM | 2174 | N | LEU | A | 898 | 22.150 | 11.200 | −3.939 | 1.00 | 32.39 | A |
| ATOM | 2175 | CA | LEU | A | 898 | 21.767 | 10.684 | −2.629 | 1.00 | 33.14 | A |
| ATOM | 2176 | CS | LEU | A | 898 | 21.338 | 9.223 | −2.755 | 1.00 | 31.76 | A |
| ATOM | 2177 | CG | LEU | A | 898 | 20.167 | 8.938 | −3.702 | 1.00 | 30.43 | A |
| ATOM | 2178 | CD1 | LEU | A | 898 | 20.050 | 7.442 | −3.949 | 1.00 | 28.81 | A |
| ATOM | 2179 | CD2 | LEU | A | 898 | 18.886 | 9.489 | −3.101 | 1.00 | 29.32 | A |
| ATOM | 2180 | C | LEU | A | 898 | 22.915 | 10.791 | −1.633 | 1.00 | 35.96 | A |
| ATOM | 2181 | O | LEU | A | 898 | 22.780 | 10.410 | −0.466 | 1.00 | 35.50 | A |
| ATOM | 2182 | N | LYS | A | 899 | 24.049 | 11.304 | −2.100 | 1.00 | 37.16 | A |
| ATOM | 2183 | CA | LYS | A | 899 | 25.218 | 11.453 | −1.245 | 1.00 | 39.32 | A |
| ATOM | 2184 | CB | LYS | A | 899 | 26.458 | 11.783 | −2.083 | 1.00 | 39.91 | A |
| ATOM | 2185 | CG | LYS | A | 899 | 26.875 | 10.675 | −3.035 | 1.00 | 40.95 | A |
| ATOM | 2186 | CD | LYS | A | 899 | 27.176 | 9.392 | −2.279 | 1.00 | 42.25 | A |
| ATOM | 2187 | CE | LYS | A | 899 | 27.521 | 8.254 | −3.230 | 1.00 | 43.26 | A |
| ATOM | 2188 | NZ | LYS | A | 899 | 27.655 | 6.962 | −2.498 | 1.00 | 43.52 | A |
| ATOM | 2189 | C | LYS | A | 899 | 24.996 | 12.540 | −0.204 | 1.00 | 39.53 | A |
| ATOM | 2190 | O | LYS | A | 899 | 25.502 | 12.451 | 0.913 | 1.00 | 39.99 | A |
| ATOM | 2191 | N | ALA | A | 900 | 24.238 | 13.566 | −0.574 | 1.00 | 40.31 | A |
| ATOM | 2192 | CA | ALA | A | 900 | 23.952 | 14.672 | 0.332 | 1.00 | 41.45 | A |
| ATOM | 2193 | CB | ALA | A | 900 | 23.297 | 15.816 | −0.436 | 1.00 | 42.71 | A |
| ATOM | 2194 | C | ALA | A | 900 | 23.047 | 14.225 | 1.477 | 1.00 | 42.06 | A |
| ATOM | 2195 | O | ALA | A | 900 | 23.462 | 14.222 | 2.638 | 1.00 | 43.54 | A |
| ATOM | 2196 | O | HOH | A | 1 | 14.457 | −2.301 | −3.629 | 1.00 | 11.91 | A |
| ATOM | 2197 | O | HOH | A | 2 | −4.397 | 11.098 | 3.237 | 1.00 | 16.01 | A |
| ATOM | 2198 | O | HOH | A | 3 | −4.918 | 7.667 | −5.486 | 1.00 | 16.02 | A |
| ATOM | 2199 | O | HOH | A | 4 | −18.623 | −6.192 | −4.002 | 1.00 | 15.98 | A |
| ATOM | 2200 | O | HOH | A | 5 | −1.021 | 11.333 | −8.745 | 1.00 | 17.20 | A |
| ATOM | 2201 | O | HOH | A | 6 | 2.429 | 1.976 | 9.154 | 1.00 | 11.10 | A |
| ATOM | 2202 | O | HOH | A | 7 | 9.183 | −2.818 | −7.644 | 1.00 | 12.10 | A |
| ATOM | 2203 | O | HOH | A | 8 | −2.277 | −6.184 | −9.377 | 1.00 | 16.99 | A |
| ATOM | 2204 | O | HOH | A | 9 | −3.892 | 7.522 | 2.129 | 1.00 | 11.77 | A |
| ATOM | 2205 | O | HOH | A | 10 | −24.765 | −1.312 | −3.431 | 1.00 | 11.31 | A |
| ATOM | 2206 | O | HOH | A | 11 | −18.960 | −7.652 | −1.790 | 1.00 | 14.87 | A |
| ATOM | 2207 | O | HOH | A | 12 | 1.251 | −7.943 | −7.973 | 1.00 | 15.91 | A |
| ATOM | 2208 | O | HOH | A | 13 | −5.135 | 9.220 | 4.569 | 1.00 | 25.85 | A |
| ATOM | 2209 | O | HOH | A | 14 | 0.868 | 13.830 | 8.869 | 1.00 | 19.70 | A |
| ATOM | 2210 | O | HOH | A | 15 | −4.469 | −6.600 | −8.030 | 1.00 | 17.02 | A |
| ATOM | 2211 | O | HOH | A | 16 | −23.949 | 2.424 | −2.051 | 1.00 | 16.20 | A |
| ATOM | 2212 | O | HOH | A | 17 | 16.570 | −0.194 | −6.147 | 1.00 | 20.96 | A |
| ATOM | 2213 | O | HOH | A | 18 | 0.212 | 10.493 | −11.249 | 1.00 | 32.03 | A |
| ATOM | 2214 | O | HOH | A | 19 | 18.352 | −5.806 | −3.576 | 1.00 | 21.26 | A |
| ATOM | 2215 | O | HOH | A | 20 | −1.189 | 18.011 | −6.297 | 1.00 | 21.54 | A |
| ATOM | 2216 | O | HOH | A | 21 | −2.174 | −6.079 | 3.946 | 1.00 | 21.58 | A |
| ATOM | 2217 | O | HOH | A | 22 | 2.660 | 2.991 | −12.285 | 1.00 | 10.78 | A |
| ATOM | 2218 | O | HOH | A | 23 | 6.194 | 0.599 | 12.618 | 1.00 | 26.49 | A |
| ATOM | 2219 | O | HOH | A | 24 | 6.270 | 2.873 | 10.787 | 1.00 | 20.59 | A |
| ATOM | 2220 | O | HOH | A | 25 | −30.350 | −2.848 | −5.086 | 1.00 | 21.38 | A |
| ATOM | 2221 | O | HOH | A | 26 | −5.973 | 10.069 | 1.056 | 1.00 | 21.49 | A |
| ATOM | 2222 | O | HOH | A | 27 | 7.351 | −13.578 | −1.294 | 1.00 | 24.35 | A |
| ATOM | 2223 | O | HOH | A | 28 | −16.655 | −18.205 | 6.142 | 1.00 | 32.77 | A |
| ATOM | 2224 | O | HOH | A | 29 | −23.065 | −6.820 | 10.522 | 1.00 | 24.13 | A |
| ATOM | 2225 | O | HOH | A | 30 | 14.170 | 15.934 | −4.146 | 1.00 | 22.96 | A |
| ATOM | 2226 | O | HOH | A | 31 | −5.570 | −1.784 | −15.428 | 1.00 | 26.29 | A |
| ATOM | 2227 | O | HOH | A | 32 | −12.382 | −1.657 | −13.385 | 1.00 | 22.13 | A |
| ATOM | 2228 | O | HOH | A | 33 | 11.773 | −2.825 | −3.978 | 1.00 | 17.40 | A |
| ATOM | 2229 | O | HOH | A | 34 | 24.033 | 2.469 | 1.013 | 1.00 | 28.57 | A |
| ATOM | 2230 | O | HOH | A | 35 | 5.376 | 16.007 | 8.519 | 1.00 | 24.65 | A |
| ATOM | 2231 | O | HOH | A | 36 | −9.608 | −13.304 | −9.571 | 1.00 | 27.67 | A |
| ATOM | 2232 | O | HOH | A | 37 | −5.225 | −8.904 | −9.144 | 1.00 | 18.25 | A |
| ATOM | 2233 | O | HOH | A | 38 | 11.257 | −0.407 | −7.511 | 1.00 | 29.30 | A |
| ATOM | 2234 | O | HOH | A | 39 | 0.499 | −15.307 | 6.874 | 1.00 | 26.00 | A |
| ATOM | 2235 | O | HOH | A | 40 | −11.598 | −8.243 | 3.065 | 1.00 | 18.53 | A |
| ATOM | 2236 | O | HOH | A | 41 | 2.939 | 23.776 | 3.343 | 1.00 | 30.46 | A |
| ATOM | 2237 | O | HOH | A | 42 | −10.147 | −9.966 | 7.681 | 1.00 | 26.73 | A |
| ATOM | 2238 | O | HOH | A | 43 | 9.258 | −2.252 | −4.749 | 1.00 | 18.60 | A |
| ATOM | 2239 | O | HOH | A | 44 | −7.990 | 1.495 | 0.072 | 1.00 | 32.79 | A |
| ATOM | 2240 | O | HOH | A | 45 | −21.791 | −2.653 | 3.395 | 1.00 | 30.17 | A |
| ATOM | 2241 | O | HOH | A | 46 | −12.258 | 8.515 | −1.865 | 1.00 | 32.59 | A |
| ATOM | 2242 | O | HOH | A | 47 | 17.934 | −29.751 | 44.878 | 1.00 | 37.91 | A |
| ATOM | 2243 | O | HOH | A | 48 | −5.849 | 14.783 | −6.753 | 1.00 | 25.08 | A |
| ATOM | 2244 | O | HOH | A | 49 | −0.415 | −15.909 | −10.041 | 1.00 | 30.96 | A |
| ATOM | 2245 | O | HOH | A | 50 | 2.276 | −3.698 | 10.859 | 1.00 | 31.49 | A |
| ATOM | 2246 | O | HOH | A | 51 | 13.707 | −1.745 | 14.947 | 1.00 | 26.81 | A |
| ATOM | 2247 | O | HOH | A | 52 | −11.484 | 5.994 | −4.734 | 1.00 | 28.94 | A |
| ATOM | 2248 | O | HOH | A | 53 | 15.597 | 8.025 | 12.778 | 1.00 | 25.87 | A |
| ATOM | 2249 | O | HOH | A | 54 | −12.859 | −0.373 | −16.318 | 1.00 | 38.15 | A |
| ATOM | 2250 | O | HOH | A | 55 | −21.136 | −9.246 | −1.882 | 1.00 | 23.94 | A |
| ATOM | 2251 | O | HOH | A | 56 | 10.996 | 16.960 | 15.947 | 1.00 | 36.67 | A |
| ATOM | 2252 | O | HOH | A | 57 | −6.591 | 9.869 | −9.281 | 1.00 | 26.49 | A |
| ATOM | 2253 | O | HOH | A | 58 | 13.911 | −11.174 | 2.291 | 1.00 | 30.38 | A |
| ATOM | 2254 | O | HOH | A | 59 | −9.562 | −10.942 | −10.916 | 1.00 | 27.88 | A |
| ATOM | 2255 | O | HOH | A | 60 | 4.745 | −19.626 | −5.080 | 1.00 | 29.55 | A |
| ATOM | 2256 | O | HOH | A | 61 | −1.717 | −8.359 | −10.786 | 1.00 | 24.62 | A |
| ATOM | 2257 | O | HOH | A | 62 | −10.559 | −3.268 | −13.949 | 1.00 | 24.21 | A |
| ATOM | 2258 | O | HOH | A | 63 | −0.660 | 15.194 | −5.517 | 1.00 | 22.53 | A |
| ATOM | 2259 | O | HOH | A | 64 | 9.037 | −0.135 | −9.783 | 1.00 | 36.19 | A |
| ATOM | 2260 | O | HOH | A | 65 | −23.460 | −16.827 | 8.631 | 1.00 | 29.91 | A |
| ATOM | 2261 | O | HOH | A | 66 | −24.192 | −1.276 | 5.295 | 1.00 | 27.96 | A |
| ATOM | 2262 | O | HOH | A | 67 | −16.353 | 4.624 | −11.962 | 1.00 | 32.05 | A |
| ATOM | 2263 | O | HOH | A | 68 | −17.396 | 3.801 | −9.450 | 1.00 | 28.58 | A |
| ATOM | 2264 | O | HOH | A | 69 | 10.752 | −11.093 | 7.051 | 1.00 | 37.06 | A |
| ATOM | 2265 | O | HOH | A | 70 | 1.620 | −15.965 | −4.603 | 1.00 | 24.63 | A |
| ATOM | 2266 | O | HOH | A | 71 | −8.238 | 12.427 | −6.673 | 1.00 | 33.82 | A |
| ATOM | 2267 | O | HOH | A | 72 | −16.577 | −14.805 | −9.290 | 1.00 | 29.62 | A |
| ATOM | 2268 | O | HOH | A | 73 | 9.083 | −13.189 | −4.221 | 1.00 | 35.45 | A |
| ATOM | 2269 | O | HOH | A | 74 | 10.287 | 7.919 | −14.356 | 1.00 | 33.56 | A |
| ATOM | 2270 | O | HOH | A | 75 | −20.538 | −21.060 | 2.242 | 1.00 | 45.67 | A |
| ATOM | 2271 | O | HOH | A | 76 | −1.640 | 14.388 | 9.613 | 1.00 | 30.93 | A |
| ATOM | 2272 | O | HOH | A | 77 | 40.049 | −7.010 | 13.782 | 1.00 | 30.14 | A |
| ATOM | 2273 | O | HOH | A | 78 | 18.418 | 12.809 | −8.404 | 1.00 | 37.11 | A |
| ATOM | 2274 | O | HOH | A | 79 | −25.942 | −2.805 | −10.608 | 1.00 | 34.88 | A |
| ATOM | 2275 | 0 | HOH | A | 80 | 16.293 | −4.489 | 17.257 | 1.00 | 25.34 | A |
| ATOM | 2276 | O | HOH | A | 81 | −16.209 | −8.340 | 11.406 | 1.00 | 40.96 | A |
| ATOM | 2277 | O | HOH | A | 82 | 11.439 | 19.084 | 12.127 | 1.00 | 33.B2 | A |
| ATOM | 2278 | O | HOH | A | 83 | 20.159 | 2.221 | 5.570 | 1.00 | 28.54 | A |
| ATOM | 2279 | O | HOH | A | 84 | −13.713 | 5.569 | −10.421 | 1.00 | 29.53 | A |
| ATOM | 2280 | O | HOH | A | 85 | −7.262 | 16.174 | −0.684 | 1.00 | 29.54 | A |
| ATOM | 2281 | O | HOH | A | 86 | 9.742 | −10.617 | −7.415 | 1.00 | 25.62 | A |
| ATOM | 2282 | O | HOH | A | 87 | −20.632 | 0.152 | 7.971 | 1.00 | 39.94 | A |
| ATOM | 2283 | O | HOH | A | 88 | 1.339 | 18.421 | −9.397 | 1.00 | 43.65 | A |
| ATOM | 2284 | 0 | HOH | A | 89 | −4.943 | 13.752 | 20.778 | 1.00 | 37.74 | A |
| ATOM | 2285 | O | HOH | A | 90 | −3.157 | 10.534 | 20.505 | 1.00 | 40.33 | A |
| ATOM | 2286 | O | HOH | A | 91 | 20.471 | 14.004 | −1.018 | 1.00 | 22.59 | A |
| ATOM | 2287 | O | HOH | A | 92 | −3.126 | −6.909 | 11.806 | 1.00 | 31.06 | A |
| ATOM | 2288 | O | HOH | A | 93 | −14.587 | −14.560 | 24.267 | 1.00 | 48.31 | A |
| ATOM | 2289 | O | HOH | A | 94 | 4.029 | −14.349 | 32.347 | 1.00 | 56.74 | A |
| ATOM | 2290 | O | HOH | A | 95 | 7.949 | −15.761 | −2.076 | 1.00 | 40.76 | A |
| ATOM | 2291 | O | HOH | A | 96 | −2.357 | 13.362 | −6.866 | 1.00 | 25.37 | A |
| ATOM | 2292 | O | HOH | A | 97 | 0.273 | 12.223 | −13.491 | 1.00 | 30.12 | A |
| ATOM | 2293 | O | HOH | A | 98 | 23.889 | −3.917 | 11.357 | 1.00 | 25.67 | A |
| ATOM | 2294 | 0 | HOH | A | 99 | −4.748 | −9.020 | −11.939 | 1.00 | 46.38 | A |
| ATOM | 2295 | O | HOH | A | 100 | −1.430 | −10.359 | −13.316 | 1.00 | 31.41 | A |
| ATOM | 2296 | O | HOH | A | 101 | 10.739 | −23.422 | −9.199 | 1.00 | 39.50 | A |
| ATOM | 2297 | O | HOH | A | 102 | −3.937 | 14.980 | −8.334 | 1.00 | 24.93 | A |
| ATOM | 2298 | O | HOH | A | 103 | −7.054 | −10.787 | −10.296 | 1.00 | 33.68 | A |
| ATOM | 2299 | O | HOH | A | 104 | 13.492 | 0.660 | 13.579 | 1.00 | 31.04 | A |
| ATOM | 2300 | O | HOH | A | 105 | −6.920 | −14.447 | −11.281 | 1.00 | 45.39 | A |
| ATOM | 2301 | O | HOH | A | 106 | 13.348 | 22.708 | 2.254 | 1.00 | 36.30 | A |
| ATOM | 2302 | O | HOH | A | 107 | 5.408 | −11.711 | −5.405 | 1.00 | 31.10 | A |
| ATOM | 2303 | O | HOH | A | 108 | 18.256 | −2.341 | 15.534 | 1.00 | 28.95 | A |
| ATOM | 2304 | O | HOH | A | 109 | −8.503 | 0.787 | 3.249 | 1.00 | 41.16 | A |
| ATOM | 2305 | O | HOH | A | 110 | 14.317 | 3.040 | −11.205 | 1.00 | 42.00 | A |
| ATOM | 2306 | O | HOH | A | 111 | 11.881 | 17.271 | −4.308 | 1.00 | 37.83 | A |
| ATOM | 2307 | O | HOH | A | 112 | 19.020 | 15.966 | 5.172 | 1.00 | 45.54 | A |
| ATOM | 2308 | O | HOH | A | 113 | 0.998 | −12.806 | −8.453 | 1.00 | 43.81 | A |
| ATOM | 2309 | O | HOH | A | 114 | 13.315 | −10.545 | 8.002 | 1.00 | 33.76 | A |
| ATOM | 2310 | O | HOH | A | 115 | −10.798 | 3.629 | 0.360 | 1.00 | 29.35 | A |
| ATOM | 2311 | O | HOH | A | 116 | 26.244 | 9.778 | 1.025 | 1.00 | 38.48 | A |
| ATOM | 2312 | O | HOH | A | 117 | −18.933 | −5.540 | 7.951 | 1.00 | 21.69 | A |
| ATOM | 2313 | O | HOH | A | 118 | −2.346 | 9.089 | 14.123 | 1.00 | 28.55 | A |
| ATOM | 2314 | O | HOH | A | 119 | 12.331 | −3.683 | −6.285 | 1.00 | 28.50 | A |
| ATOM | 2315 | O | HOH | A | 120 | 17.652 | 7.204 | 6.269 | 1.00 | 33.34 | A |
| ATOM | 2316 | O | HOH | A | 121 | −20.972 | −5.394 | 10.153 | 1.00 | 29.22 | A |
| ATOM | 2317 | O | HOH | A | 122 | 1.126 | −29.138 | 3.663 | 1.00 | 54.55 | A |
| ATOM | 2318 | O | HOH | A | 123 | 19.859 | 5.269 | 4.518 | 1.00 | 48.81 | A |
| ATOM | 2319 | O | HOH | A | 124 | 16.235 | −4.760 | 13.356 | 1.00 | 24.74 | A |
| ATOM | 2320 | O | HOH | A | 125 | −1.781 | 27.895 | −1.429 | 1.00 | 49.94 | A |
| ATOM | 2321 | O | HOH | A | 126 | 0.978 | 3.103 | −14.170 | 1.00 | 33.01 | A |
| ATOM | 2322 | O | HOH | A | 127 | 23.932 | 8.429 | −15.415 | 1.00 | 43.20 | A |
| ATOM | 2323 | O | HOH | A | 128 | −15.975 | −7.260 | 14.283 | 1.00 | 35.46 | A |
| ATOM | 2324 | O | HOH | A | 129 | −5.663 | −12.681 | 1.284 | 1.00 | 25.44 | A |
| ATOM | 2325 | O | HOH | A | 130 | −9.888 | −12.927 | 7.395 | 1.00 | 35.96 | A |
| ATOM | 2326 | O | HOH | A | 131 | 25.472 | 3.261 | −1.345 | 1.00 | 31.42 | A |
| ATOM | 2327 | O | HOH | A | 132 | 0.225 | −4.710 | 9.057 | 1.00 | 40.52 | A |
| ATOM | 2328 | O | HOH | A | 133 | 1.153 | −8.592 | −10.392 | 1.00 | 41.49 | A |
| ATOM | 2329 | O | HOH | A | 134 | 0.318 | −2.638 | −15.320 | 1.00 | 36.09 | A |
| ATOM | 2330 | O | HOH | A | 135 | −6.483 | 17.186 | −5.700 | 1.00 | 31.77 | A |
| ATOM | 2331 | O | HOH | A | 136 | −11.747 | 5.541 | −2.116 | 1.00 | 37.63 | A |
| ATOM | 2332 | O | HOH | A | 137 | 3.033 | 5.741 | −16.256 | 1.00 | 60.91 | A |
| ATOM | 2333 | O | HOH | A | 138 | 15.857 | −9.756 | 3.393 | 1.00 | 42.40 | A |
| ATOM | 2334 | O | HOH | A | 139 | 1.315 | 24.812 | 0.302 | 1.00 | 37.92 | A |
| ATOM | 2335 | O | HOH | A | 140 | 18.429 | −8.563 | 6.665 | 1.00 | 40.06 | A |
| ATOM | 2336 | O | HOH | A | 141 | 8.361 | 17.182 | −5.869 | 1.00 | 35.77 | A |
| ATOM | 2337 | O | HOH | A | 142 | −3.830 | −1.160 | 8.545 | 1.00 | 30.75 | A |
| ATOM | 2338 | O | HOH | A | 143 | −11.080 | −0.626 | −21.190 | 1.00 | 42.20 | A |
| ATOM | 2339 | O | HOH | A | 144 | −25.620 | −5.418 | −10.124 | 1.00 | 24.22 | A |
| ATOM | 2340 | N9 | ANE | A | 400 | −0.759 | −10.830 | 5.271 | 1.00 | 36.71 | A |
| ATOM | 2341 | C8 | ANE | A | 400 | −1.712 | −10.252 | 4.469 | 1.00 | 37.40 | A |
| ATOM | 2342 | N7 | ANE | A | 400 | −1.529 | −10.440 | 3.198 | 1.00 | 37.33 | A |
| ATOM | 2343 | Cs | ANE | A | 400 | −0.400 | −11.181 | 3.134 | 1.00 | 37.65 | A |
| ATOM | 2344 | C6 | ANE | A | 400 | 0.318 | −11.704 | 2.059 | 1.00 | 38.63 | A |
| ATOM | 2345 | N6 | ANE | A | 400 | −0.086 | −11.507 | 0.797 | 1.00 | 39.54 | A |
| ATOM | 2346 | M1 | ANE | A | 400 | 1.443 | −12.423 | 2.375 | 1.00 | 37.38 | A |
| ATOM | 2347 | C2 | ANE | A | 400 | 1.792 | −12.580 | 3.654 | 1.00 | 38.46 | A |
| ATOM | 2348 | N3 | ANE | A | 400 | 1.207 | −12.139 | 4.749 | 1.00 | 39.20 | A |
| ATOM | 2349 | C4 | ANE | A | 400 | 0.099 | −11.435 | 4.400 | 1.00 | 37.67 | A |
| END | | | | | | | | | | | |

### LIST OF REFERENCES

Abrahams, J. P. and Leslie, A. G. W. (1996). Methods used in the structure determination of bovine mitochondrial F1 ATPase. Acta Cryst. D52, 30-42.

Andersson L. C. (1998) c-kit gain-of-function mutations and human tumors. Japn. J. of Cancer Res., 89:1.

Adams, R. H., Wilkinson, G. A., Weiss, C., Diella, F., Gale, N. W., Deutsch, U., Risau, W., and Klein, R (1999) Roles of ephrinB ligands and EphB receptors in cardiovascular development: demarcation of arterial/venous domains, vascular morphogenesis, and sprouting angiogenesis. Genes Dev. 13, 295-306.

Barker, S. C., Kassel D. B., Weigl, D., Huang, X., Luther, M. A., and Knight, W. B. (1995). Characterization of pp60c-src tyrosine kinase activities using a continuous assay: autoactivation of the enzyme is an intermolecular autophosphorylation process. Biochem. 34, 14843-14851.

Baxter, R. M., Secrist, J. P., Vaillancourt, R. R., and Kazlauskas, A. (1998). Full activation of the platelet-derived growth factor β-receptor kinase involves multiple events. J. Biol. Chem. 273, 17050-17055.

Binns, K. L., Taylor, P. P., Sicheri, F., Pawson, T., and Holland, S. J. (2000). Phosphorylation of tyrosine residues in the kinase domain and juxtamembrane region regulates the biological and catalytic activities of Eph receptors. Mol. Cell. Biol. 20, 4791-4805.

Brown, A., Yates, P. A., Burrola, P., Ortuno, D., Vaidya, A., Jessell, T. M., Pfaff, S. L., O'Leary, D. D., and Lemke, G. (2000). Topographic mapping from the retina to the midbrain is controlled by relative but not absolute levels of EphA receptor signaling. Cell 102, 77-88.

Bruckner, K., Pasquale, E. B., and Klein, R (1997). Tyrosine phosphorylation of transmembrane ligands for Eph receptors. Science 275, 1640-1643.

Brunger, A. T., Adams, P. D., Clore, G. M., Gros, P., Grosse-Kuntsleve, R. W., Jiang, J. -S., Kuszewski, J., Nilges, M., Pannu, N. S., Read, R. J., and et al. (1998). Crystallography and NMR system: a new software suite for macromolecular structure determination. Acta Crystallogr. D54, 905-921.

Cann, A. D., Bishop, S. M., Ablooglu, A. J., and Kohanski, R. A. (2000). Partial activation of the insulin receptor kinase domain by juxtamembrane autophosphorylation. Biochem. 37, 11289-11300.

Carson, M. (1991). Ribbons 2.0. J. Appl. Crystallgr. 24, 958-961.

Collaborative Computational Project, Number 4. (1994). The CCP4 suite: programs for protein crystallography. Acta Crystallogr. D50, 760-763.

Chin-Sang, I. D., George, S. E., Ding, M., Moseley, S. L., Lynch, A. S., and Chisholm, A. D. (1999). The ephrin VAB-2/EFN-1 functions in neuronal signaling to regulate epidermal morphogenesis in C. elegans. Cell 99, 781-790.

Choi, S. and Park, S. (1999). Phosphorylation at Tyr-838 in the kinase domain of EphA8 modulates Fyn binding to the Tyr-615 site by enhancing tyrosine kinase activity. Oncogene 18, 5413-5422.

Connor, R. J. and Pasquale, E. B. (1995). Genomic organization and alternatively processed forms of Cek5, a receptor protein-tyrosine kinase of the Eph family. Oncogene 11, 2429-2438.

Dodelet, V. C., Pazzagli, C., Zisch, A. H., Hauser, C. A., and Pasquale, E. B. (1999). A novel signaling intermediate, SHEP1, directly couples Eph receptors to R-Ras and Rap1A. J. Biol. Chem. 274, 31941-31946.

Drescher, U., Kremoser, C., Handwerker, C. J. L., Noda, M., and Bonhoeffer, F. (1995). In vitro guidance of retinal ganglion cell exons by RAGS, a 25 kDa tectal protein related to ligands for Eph receptor tyrosine kinases. Cell 82, 359-370.

Ellis, C., Kasmi F., Ganju, P., Walls E., and Panayotou (1996). A juxtamembrane autophosphorylation site in the Eph family receptor tyrosine kinase, Sek, mediates high affinity interaction with p59fyn. Oncogene 12, 1727-1736.

Eph Nomenclature Committee (1997). Unified nomenclature for Eph family receptors and their ligands, the ephrins. Cell 90, 403-404.

Gale, N. W., Holland, S. J., Valenzuela, D. M., Flenniken, A., Pan, L., Ryan, T. E., Henkemeyer, M., Strebhardt, K., Hirai, H., Wilkinson, D. G., Pawson, T., Davis, S., and Yancopoulos, G. D. (1996). Eph receptors and ligands comprise two major specificity subclasses and are reciprocally compartmentalized during embryogenesis. Neuron 17, 9-19.

George, S. E., Simokat K., Hardin, J., and Chisholm, A. D. (1998). The VAB-1 Eph receptor tyrosine kinase functions in neural and epithelial morphogenesis in C. elegans. Cell 92, 633-643.

Gerety, S. S., Wang, H. U., Chen, Z. F., and Anderson, D. J. (1999). Symmetrical mutant phenotypes of the receptor EphB4 and its specific transmembrane ligand ephrin-B2 in cardiovascular development. Mol. Cell 4, 403-414.

Hanks, S. K., Quinn, A. M., and Hunter, T. (1988). The protein kinase family: Conserved features and deduced phylogeny of the catalytic domains. Science 241, 42-52.

Hayakawa F., Towatari M., Kiyoi H., Tanimoto M., Kitamura T., Saito H. and Naoe T. (2000) Tandem-duplicated Flt3 constitutively activates STAT5 and MAP kinase and introduces autonomous cell growth in IL-3 dependent cell lines. Oncogene, 19:624-631.

Heldin, C. H. (1995). Dimerization of cell surface receptors in signal transduction. Cell 80, 213-223.

Henkemeyer, M., Marengere, L. E. M., McGlade, J., Olivier, J. P., Conlon, R. A., Holmyard, D. P., Letwin, K, and Pawson, T. (1994). Immunolocalization of the Nuk receptor tyrosine kinase suggests roles in segmental patterning of the brain and axonogenesis. Oncogene 9, 1001-1014.

Himanen, J. P., Henkemeyer, M., and Nikolov, D. B. (1998). Crystal structure of the ligand-binding domain of the receptor tyrosine kinase EphB2. Nature 396, 486-491.

Hirota, S., Isozaki, K., Moriyama, Y., Hashimoto, K., Nishida, T., Ishiguro, S., Kawano, K., Hanada, M., Kurata, A., Takeda, M., Tunio, G. M., Matsuzawa, Y., Kanakura, Y., Shinomura, Y., and Kitamura, Y. (1998). Gain-of-function mutations of c-kit in human gastrointestinal stromal tumors. Science 279, 577-580.

Hock, B., Bohme, B., Karn, T., Yamamoto, T., Kaibuchi, K., Holtrich, U., Holland, S., Pawson, T., Rubsamen-Waigmann, H., and Strebhardt, K. (1998). PDZ-domain-mediated interaction of the Eph-related receptor tyrosine kinase EphB3 and the ras-binding protein AF6 depends of the kinase activity of the receptor. Proc. Natl. Acad. Sci. USA 95, 9779-9784.

Holder, N. and Klein, R (1999). Eph receptors and ephrins: effectors of morphogenesis. Development 126, 2033-2044.

Holland, S. J., Gale, N. W., Gish, G. D., Roth, R. A., Songyang, Z., Cantley, L. C., Henkemeyer, M., Yancopoulos, G. D., and Pawson, T. (1997). Juxtamembrane tyrosine residues couple the Eph family receptor Eph B2/Nuk to specific SH2 domain proteins in neuronal cells. EMBO J. 16, 3877-3888.

Holland, S. J., Gale, N. W., Mbamalu, G., Yancopoulos, G. D., Henkemeyer, M., and Pawson, T. (1996). Bi-directional signalling through the Eph family receptor Nuk and its transmembrane ligands. Nature 383, 722-725.

Holland, S. J., Peles, E., Pawson, T., and Schlessinger, J. (1998). Cell-contact-dependent signalling in axon growth and guidance: Eph receptor tyrosine kinases and receptor protein tyrosine phosphatase □. Curr. Opin. Neurobiol. 8, 117-127.

Hubbard, S. R., Wei L., Ellis L., and Hendrickson, W. A. (1994). Crystal structure of the tyrosine kinase domain of the human insulin receptor. Nature 372, 746-754.

Hubbard, S. R. (1997). Crystal structure of the activated insulin receptor tyrosine kinase in complex with peptide substrate and ATP analog. EMBO J. 16, 5572-5581.

Hubbard, S. R. and Till, J. H. (2000). Protein tyrosine kinase structure and function. Annu. Rev. Biochem. 69, 373-398.

Huse, M., Chen, Y. G., Massague, J., and Kuriyan, J. (1999). Crystal structure of the cytoplasmic domain of the type I TGF β receptor in complex with FKBP12. Cell 96, 425-436.

Irusta, P. M. and DiMaio, D. (1998). A single amino acid substitution in a WW-like domain of diverse members of the PDGF receptor subfamily of tyrosine kinases causes constitutive receptor activation. EMBO J. 17, 6912-6923.

Johnson, L. N., Noble, M. E., and Owen, D. J. (1996). Active and inactive protein kinases: structural basis for regulation. Cell 85, 149-158.

Jones, T. A., Zou, J. Y., Cowan, S. W., and Kjeldgaard, M. (1991). Improved methods for binding protein models in electron density maps and the localization of errors in these models. Acta Cryst. A47, 110-119.

Kalo, M. S. and Pasquale, E. B. (1999). Multiple in vivo tyrosine phosphorylation sites in EphB receptors. Biochem. 38, 14396-14408.

Kitayama, H., Kanakura, Y., Furitsu, T., Tsujimura, T., Oritani, K., Ikeda, H., Sugahara, H., Mitsui, H., Kanayama, Y., Kitamura, Y., and Matsuzawa, Y. (1995). Constitutively activating mutations of c-kit receptor tyrosine kinase confer factor-independent growth and tumorigenicity of factor-dependent hematopoietic cell lines. Blood 85, 790-798.

Knighton, D. R., Zheng, J., Ten Eyck L. F., Ashford, V. A., Xuong, N. -H., Taylor, S. S., and Sowadski, J. M. (1991). Crystal structure of the catalytic subunit of cyclic adenosine monophosphate-dependent protein kinase. Science 253, 407-414.

Krull, C. E., Lansford, R., Gale, N. W., Collazo A., Marcelle, C., Yancopoulos, G. D., Fraser, S. E., and Bronner-Fraser, M. (1997). Interactions of Eph-related receptors and ligands confer rostrocaudal pattern to trunk neural crest migration. Curr. Biol. 7, 571-580.

Kuriyan, J. and Cowburn, D. (1997). Modular peptide recognition domains in eukaryotic signaling. Annu. Rev. Biophys. Biomol. Struct. 26, 259-288.

La Fortelle, E. de. and Bricogne, G. (1997). Maximum-likelihood heavy-atom parameter in the MIR and MAD methods. Methods Enzymol. 276, 472-494.

Labrador, J. P., Brambilla, R., and Klein, R. (1997). The N-terminal globular domain of Eph receptors is sufficient for ligand binding and receptor signaling. EMBO J. 16, 3889-3897.

Laskowski, R. A., MacArthur, M. W., Moss, D. S., and Thornton, J. M. (1993). PROCHECK: a program to check the stereochemical quality of protein structures. J. Appl. Crystallogr. 26, 283-291.

Mellitzer, G., Xu, Q., and Wilkinson, D. G. (1999). Eph receptors and ephrins restrict cell intermingling and communication. Nature 400, 77-81.

Miller, R., Gallo, S. M., Khalak, H. G., and Weeks, C. M. (1994). SnB: crystal structure determination via Shake-and-Bake. J. Appl. Crystl. 27, 613-621.

Mohammadi, M., Schlessinger, J., and Hubbard, S. R. (1996). Structure of the FGF receptor tyrosine kinase domain reveals a novel autoinhibitory mechanism. Cell 86, 577-587.

Myles, G. M., Brandt, C. S., Carlberg, K., and Rohrschneider, L. R. (1994). Tyrosine 569 in the c-Fms juxtamembrane domain is essential for kinase activity and macrophage colony-stimulating factor-dependent internalization. Mol. Cell Biol. 14, 4843-4854.

Nakahara M., Isozaki K., Hirota S., Miyagawa J., Hase-Sawada N., Taniguchi M., Nishida T., Kanayama S., Kitamura Y., Shinomura Y., and Matsuzawa Y. (1998) A novel gain-of-function mutation of c-kit in gastrointestinal stromal tumors. Gastroenterology, 115:1090-1095.

Nakao M., Yokota S., Iwai T., Kaneko H., Horiike S., Kashima K., Sonoda Y., Fujimoto T., and Misaw S. (1996) Internal tandem duplication of the flt3 gene found in acute myeloid leukemia. Leukemia 10: 1911-1918.

Nakamoto, M., Cheng, H. J., Friedman, G. C., McLaughlin, T., Hansen, M. J., Yoon, C. H., O'Leary, D. D., and Flanagan, J. G. (1996). Topographically specific effects of ELF-1 on retinal axon guidance in vitro and retinal axon mapping in vivo. Cell 86, 755-766.

Navaza, J. (1994). Automated Package for Molecular Replacement. Acta Cryst. A50, 157-183.

Nicholls, A., Sharp, K. A., and Honig, B. (1991). Protein folding and association: insights from the interfacial and thermodynamic properties of hydrocarbons. Proteins Struct. Funct. Genet. 11, 281-296.

Otwinowski, Z. and Minor, W. (1997). Processing of X-ray diffraction data collected in oscillation mode. Methods Enzymol. 276, 307-326.

Pasquale, E. B. (1991). Identification of chicken embryo kinase 5, a developmentally regulated receptor-type tyrosine kinase of the Eph family. Cell Regul 2, 523-534.

Pawson, T. and Scott, J. D. (1997). Signaling through scaffold, anchoring, and adaptor proteins. Science 278, 2075-2080.

Remy, I., Wilson, I. A., and Michnick, S. W. (1999). Erythropoietin receptor activation by a ligand-induced conformation change. Science 283, 990-993.

Schindler, T., Bornmann, W., Pellicena, P., Miller, W. T., Clarkson, B., and Kuriyan, J. (2000). Structural mechanism for STI-571 inhibition of abelson tyrosine kinase. Science 289, 1938-1942.

Schlessinger, J. (2000). Cell signaling by receptor tyrosine kinases. Cell 103, 211-225.

Shewchuk, L. M., Hassell, A. M., Ellis, B., Holmes, W. D., Davis, R., Horne E. L., Kadwell, S. H., McKee, D. D., and Moore, J. T. (2000). Structure of the Tie2 RTK domain. Self-inhibition by the nucleotide binding loop, activation loop, and C-terminal tail. Structure Fold Des 8, 1105-1113.

Sicheri, F., Moarefi, I., and Kuriyan, J. (1997). Crystal structure of the Src family tyrosine kinase Hck. Nature 385, 602-609.

Stapleton, D., Balan, I., Pawson, T., and Sicheri, F. (1999). The crystal structure of an Eph receptor SAM domain reveals a mechanism for modular dimerization. Nature Struct Biol. 6, 44-49.

Thanos, C. D., Goodwill, K. E., and Bowie, J. U. (1999). Oligomeric structure of the human EphB2 receptor SAM domain. Science 283, 833-836.

Torres, R., Firestein, B. L., Dong, H., Staudinger, J., Olson, E. N., Huganir, R. L., Bredt, D. S., Gale, N. W., and Yancopoulos, G. D. (1998). PDZ proteins bind, cluster, and synaptically colocalize with Eph receptors and their ephrin ligands. Neuron 21, 1453-1463.

Tsujimura, T., Morimoto, M., Hashinoto, K., Moriyama, Y., Kitayama, H., Matsuzawa, Y., Kitamura, Y., and Kanakura, Y. (1996). Constitutive activation of c-kit in FMA3 murine mastocytoma cells caused be deletion of seven amino acids at the juxtamembrane domain. Blood 87, 273-283.

van der Geer, P. and Hunter, T. (1994). Receptor protein-tyosine kinases and their signal transduction pathways. Ann. Rev. Cell Biol. 10, 251-337.

Wang, H. U. and Anderson, D. J. (1997). Eph family transmembrane ligands can mediate repulsive guidance of trunk neural crest migration and motor axon outgrowth. Neuron 18, 383-396.

Wang, H. U., Chen, Z. F., and Anderson, D. J. (1999b). Molecular distinction and angiogenic interaction between embryonic arteries and veins revealed by ephrin-B2 and its receptor Eph-B4. Cell 93, 741-753.

Wang, X., Roy, P. J., Holland, S., Zhang, L. W., Culotti J. G., and Pawson, T. (1999a). Multiple ephrins control cell organization in C. elegans through kinase-dependent and kinase-independent functions of the VAB-1 Eph receptor. Molecular Cell 4, 903-913.

Weinmaster, G., Zoller, M. J., Smith, M., Hinze, E., and Pawson, T. (1984). Mutagenesis of fujinami sarcoma virus: evidence that tyrosine phosphorylation of p130^{pag-fps} modulates its biological activity. Cell 37, 559-568.

Xu, Q., Mellitzer, G., Robinson, V., and Wilkinson, D. G. (1999). In vivo cell sorting in complementary segmental domains mediated by Eph receptors and ephrins. Nature 399, 267-271.

Xu, W., Harrison, S. C., and Eck, M. J. (1997). Three-dimensional structure of the tyrosine kinase c-Src. Nature 385, 595-602.

Yokota S., Kiyoi H., Nakao M., Iwai T., Misawa S., Okuda T., Sonoda Y., Abe T., Kabsima K, Matsu Y., Naoe T. (1997) Internal tandem duplication of the FLT3 gene is preferentially seen in acute myeloid leukemia and myelodysplastic syndrome among various hematological malignancies. A study on a large series of patients and cell lines. Leukemia, 11:1605-1609.

Zisch, A. H., Kalo, M. S., Chong, L. D., and Pasquale E. B. (1998). Complex formation between EphB2 and Src requires phosphorylation of tyrosine 611 in the EphB2 juxtamembrane region. Oncogene 16, 2657-2670.

Zisch, A. H., Pazzagli, C., Freeman, A. L., Schneller, M., Hadman, M., Smith, J. W., Ruoslahti, E., and Pasquale, E. B. (2000). Replacing two conserved tyrosines of the EphB2 receptors with glutamic acid prevents binding of SH2 domains without abrogating kinase activity and biological responses. Oncogene 19, 177-187.

1. An isolated binding pocket of a receptor tyrosine kinase (RTK) that regulates the kinase domain of the receptor tyrosine kinase.

2. An isolated binding pocket as claimed in claim 1 wherein the RTK is an Eph receptor, preferably an EphB2 receptor.

3. Molecules or molecular complexes that comprise all or parts of either one or more of a binding pocket as claimed in claim 1 or 2, or a homolog of the binding pocket that has similar structure and shape.

4. A crystal comprising a binding pocket of an RTK that regulates the kinase domain of the RTK.

5. A crystal as claimed in claim 4 wherein the binding pocket is in an autoinhibited state.

6. A crystal comprising a juxtamembrane region and/or kinase domain of an RTK, or part thereof.

7. A crystal formed by a juxtamembrane region and a kinase domain of an RTK in an autoinhibited state.

8. A crystal comprising a binding pocket of an RTK that regulates the kinase domain of the RTK, in association with a ligand.

9. A crystal comprising a binding pocket of an RTK as claimed in claim 1 or 2 complexed or associated with a ligand.

10. A crystal as claimed in claim 9 wherein the ligand is a nucleotide or analogue thereof, a substrate or analogue thereof, a cofactor, and/or heavy metal atom.

11. A crystal as claimed in claim 9 wherein the ligand is a modulator of the activity of an RTK.

12. A crystal as claimed in any of the preceding claims wherein the shape and structure of the binding pocket is defined by one or more atomic interactions or enzyme atomic contacts in Table 2.

13. A crystal comprising a binding pocket of an Eph receptor.

14. A crystal comprising a binding pocket of an Eph receptor and a nucleotide or analogue thereof, from which it is possible to derive structural data for the nucleotide.

15. A crystal according to any preceding claim wherein the Eph receptor is derivable from a human cell.

16. A crystal according to any preceding claim, wherein the an Eph receptor is EphB2.

17. A crystal according to any preceding claim wherein the crystal comprises a binding pocket of an Eph receptor having a mutation in the part of the enzyme which is involved in phosphorylation.

18. A crystal according to any preceding claim wherein the crystal comprises a binding pocket of an Eph receptor having a mutation in one or more tyrosine residues.

19. A crystal according to any preceding claim wherein the binding pocket is in association with a cofactor.

20. A crystal according to any preceding claim having the structural coordinates shown in Table 3.

21. A model of a binding pocket of an RTK made using a crystal according to any preceding claim.

22. A model of: (a) a binding pocket of an RTK that is involved in maintaining an autoinhibited state or active state of an RTK or regulates the kinase domain of an RTK; and (b) a modification of the model of (a).

23. A model of a binding pocket of the present invention that substantially represents the structural coordinates specified in Table 3

24. A computer-readable medium having stored thereon a crystal or model according to any of the preceding claims.

25. A method of determining the secondary and/or tertiary structures of a polypeptide comprising the step of using a crystal or model according to any of the preceding claims.

26. A method of screening for a ligand capable of associating with a binding pocket and/or inhibiting or enhancing the atomic contacts of interactions in a binding pocket, comprising the use of a crystal or model according to any of the preceding claims.

27. A ligand identified by a method according to claim 26.

28. A ligand identified by a method according to claim 26 that is a modulator capable of modulating the activity of the RTK.

29. A method of identifying a modulator of an RTK comprising determining if a test agent inhibits or potentiates an autoinhibited state or active state of a kinase domain of the RTK.

30. A method as claimed in claim 29 comprising one or more of the following additional steps:
(a) testing whether the modulator is a modulator of the activity of a RTK, preferably testing the activity of the modulator in cellular assays and animal model assays;
(b) modifying the modulator;
(c) optionally rerunning steps (a) or (b); and
(d) preparing a pharmaceutical composition comprising the modulator.

31. A method of conducting a drug discovery business comprising:
(a) providing one or more systems employing the atomic interactions, atomic contacts, or structural coordinates of a binding pocket of an RTK, for identifying agents by their ability to inhibit or potentiate the atomic interactions or atomic contacts of a binding pocket; and
(b) conducting therapeutic profiling of agents identified in step (a), or further analogs thereof, for efficacy and toxicity in animals; and
(d) formulating a pharmaceutical preparation including one or more agents identified in step (b) as having an acceptable therapeutic profile.

32. A method of conducting a drug discovery business comprising
(a) providing one or more systems for identifying agents by their ability to inhibit or potentiate an autoinhibited state or active state of a kinase domain of an RTK; and
(b) conducting therapeutic profiling of agents identified in step (a), or further analogs thereof, for efficacy and toxicity in animals; and
(c) formulating a pharmaceutical preparation including one or more agents identified in step (b). as having an acceptable therapeutic profile.

33. A method of conducting a target discovery business comprising:
(a) providing one or more systems employing the atomic interactions, atomic contacts, or structural coordinates of a binding pocket of an RTK, for identifying agents by their ability to inhibit or potentiate the atomic interactions or atomic contacts, or providing one or more systems for identifying agents by their ability to inhibit or potentiate an autoinhibited state or active state of a kinase domain of an RTK;
(b) optionally conducting therapeutic profiling of agents identified in step (a) for efficacy and toxicity in animals; and
(c) licensing, to a third party, the rights for further drug development and/or sales for agents identified in step (a), or analogs thereof.

34. A method for regulating the kinase domain of an RTK by changing a binding domain or pocket of a RTK that regulates the kinase domain, from an autoinhibited state to an active state or from an active state to an autoinhibited state

35. A method for inhibiting kinase activity of an RTK comprising maintaining the RTK or a binding pocket thereof involved in regulating the kinase domain in an autoinhibited state, or potentiating an autoinhibited state for the RTK or binding pocket thereof involved in regulating the kinase domain.

36. Use of a modulator according to any preceding claim in the manufacture of a medicament to treat and/or prevent a disease in a mammalian patient.

37. A pharmaceutical composition comprising a ligand or modulator according to any preceding claim, and optionally a pharmaceutically acceptable carrier, diluent, excipient or adjuvant or any combination thereof.

38. A method of treating and/or preventing a disease comprising administering a ligand, modulator, or pharmaceutical composition according to any preceding claim to a mammalian patient.

39. A method of treating or preventing a condition or disease associated with an RTK in a cellular organism, comprising:
(a) administering a pharmaceutical composition as claimed in claim 38; and
(b) activating or inhibiting the RTK to treat or prevent the disease.

40. A method for treating or preventing a condition or disease involving increased RTK activity comprising maintaining the RTK, or a binding pocket thereof involved in regulating the kinase domain of the RTK, in an autoinhibited state

41. A crystal comprising an RTK binding pocket, substantially as described herein and with reference to the accompanying figures.
